(19) United States
(12) Patent Application Publication
(10) Pub. No.: US 2026/0354975 A1
(43) Pub. Date: Nov. 12, 2026

(54) GUIDANCE OF A TISSUE-ADJUSTMENT COIL

(71) Applicant: Edwards Lifesciences Innovation (Israel) Ltd., Caesarea (IL)

(72) Inventors: Bezalel Haberman-Browns, Pardes Hanna (IL); Noam Nir, Pardes-Hanna (IL); Ido Avrahamov, Tel-Aviv (IL); Haim Brauon, Beit Dagan (IL); Eyal German, Tel Aviv (IL); Yaron Herman, Givat Ada (IL); Oren Yehuda, Pardes Hana (IL); Tal Sheps, Givat Shmuel (IL); Aviv Galon, Tel Aviv (IL); Natan Anatoly Bukhdruker, Tirat Carmel (IL); Ofry Efraim Yossef, Tel-Aviv (IL); Ido Halabi, Givatayim (IL); Eran Hoffer, Yahud (IL); Mor Ovadia Popper, Shoham (IL); Gal Aviram, Kiryat Tiv'on (IL); Bhumica A. Amin, Modesto, CA (US)

(21) Appl. No.: 19/292,698

(22) Filed: Aug. 6, 2025

Related U.S. Application Data
(63) Continuation of application No. PCT/IB2024/051018, filed on Feb. 5, 2024.
(60) Provisional application No. 63/483,513, filed on Feb. 6, 2023.
(60) Provisional application No. 63/452,680, filed on Mar. 16, 2023.
(60) Provisional application No. 63/613,561, filed on Dec. 21, 2023.

(51) Int. Cl. A61F 2/24; A61B 17/04

(52) U.S. Cl. CPC A61F 2/2466; A61B 17/0469; A61B 17/0482; A61B 2017/0498; A61F 2230/0091

(57) ABSTRACT

A system usable with tissue of a heart comprises a flexible helical member that defines a plurality of turns. The system also comprises a frame that is intracardially expandable into an expanded state, and a guide rail, intracardially transitionable into a guide arrangement such that the guide rail extends: (i) distally through an interior of the frame, exiting the frame at an exit site, and (ii) from the exit site, latitudinally around an exterior of the frame. A driver is configured to advance the helical member over and along the guide rail while the guide rail is in the guide arrangement. Other implementations are also described.

### CROSS-REFERENCES TO RELATED APPLICATIONS

The present application is a Continuation of International Patent Application PCT/IB2024/051018 to Avrahamov et al., filed Feb. 5, 2024 and titled “Guidance of a tissue-adjustment coil,” which published as WO 2024/165964, and which claims priority to:
Provisional U.S. Patent Application 63/483,513 to Haberman-Browns et al., filed Feb. 6, 2023;
Provisional U.S. Patent Application 63/452,680 to Haberman-Browns et al., filed Mar. 16, 2023; and
Provisional U.S. Patent Application 63/613,561 to Haberman-Browns et al., filed Dec. 21, 2023.

Each of the above references is incorporated herein by reference.

### BACKGROUND

Hearts or portions thereof may grow enlarged under certain conditions. Dilation of an annulus of a heart valve may occur due to various heart conditions, such as an enlarged heart chamber or a leaking heart valve. A heart remodeling or annuloplasty procedure may be necessary to reshape, reinforce or tighten the heart and/or annulus. Annuloplasty may be performed by implanting an annuloplasty implant to re-shape and/or re-size the annulus, for example, to reduce the size of the annulus.

### SUMMARY

This summary is meant to provide some examples and is not intended to be limiting of the scope of the disclosure in any way. For example, any feature included in an example of this summary is not required by the claims, unless the claims explicitly recite the features. Also, the features described can be combined in a variety of ways. Various features and steps as described elsewhere in this disclosure can be included in the examples summarized here.

In some implementations, methods, systems, apparatuses, devices, assemblies, etc. for implanting an implant along tissue, e.g., along an annulus of a heart, etc. and/or for guiding the implantation using various guide assemblies, as will be described hereinbelow.

In some implementations, the implant is adapted to adjust (e.g., reduce) a dimension (e.g., a circumference) of a tissue (e.g., of an annulus). For example, the implant can be an annuloplasty implant, configured to reduce regurgitation of an atrioventricular valve of the heart.

In some implementations, the implant can comprise a helical member (e.g., a coil), such as an anchor having a helical shape, that is adapted to be anchored along tissue of a valve annulus, and to be subsequently axially contracted (e.g., compressed), in order to contract (e.g., compress) the tissue. For example, this contraction can be used to circumferentially reduce the size of a valve annulus.

In some implementations, the helical member can be adapted to be anchored into the tissue via rotation (e.g., screwed into the tissue). In some implementations, a screw axis of the helical member can be disposed substantially parallel with the surface of the tissue, such that following implantation of the helical member, each turn of the helix can be disposed partly within the tissue and partly outside of the tissue.

In some implementations, a guide assembly is used that includes a guide rail that is adapted to position and guide the implantation of the helical member along the tissue, e.g., by assuming, at least in part, a shape that the helical member will assume upon its implantation.

In some implementations, the guide rail can extend along the tissue such that it provides a track along which the helical member progresses. In some implementations, once the guide assembly is in place, the helical member is advanced along the guide rail (e.g., becoming progressively threaded onto the guide rail) such that the guide rail directs the implantation of the implant along the tissue.

In some implementations, the guide rail can arc around at least part of an annulus of a valve of the heart, and the helical member can thereby be anchored in an arc around at least part of the annulus.

In some implementations, the guide assembly can also include a guide frame that is adapted to position the guide rail along the annulus. While expanded and at the native valve, the guide frame can press against tissue of the annulus.

In some implementations, the valve can continue to function at least in part even when the guide frame is expanded and/or pressing against tissue, e.g., because the guide frame is open and allows blood flow therethrough, and/or because the leaflets of the valve remain partially functional.

In some implementations, in order for the guide frame to facilitate positioning of the guide rail along the annulus, the guide assembly can comprise multiple fasteners, distributed along a part of the guide frame (e.g., collectively describing an arc around the midsection of the guide frame).

In some implementations, each of these fasteners forms a respective loop, looped around a piece (e.g., a strut) of the guide frame and the guide rail.

In some implementations, the guide rail can extend through the loops (e.g., by being threaded through the loops), such the guide rail extends circumferentially around at least part of the guide frame (e.g., in an arc). Thus, when the guide frame is expanded and placed in the native valve (with the guide rail positioned along the midsection of the guide frame), this arrangement positions the guide rail along the tissue of the annulus (e.g., in contact with an atrial surface of the annulus).

In some implementations, the fasteners (e.g., the loops) are formed from one or more longitudinal members (e.g., a thread, suture, ribbon, rope, wire, cable, or string), e.g., each longitudinal member defining a respective loop.

In some implementations, the longitudinal members extend, from an extracorporeal proximal portion of a delivery assembly of the system, through the guide assembly, to form the loops at the guide frame, e.g., at an outer surface thereof. From the interior of the guide frame, the longitudinal members can extend out of the guide frame (e.g., between struts thereof), looping around the guide rail to define the loops, and back into the interior of the guide frame.

In some implementations, the longitudinal members can then return through the guide assembly and back out of the subject (e.g., a living subject or simulation).

In some implementations, the release of the loops from the implant (e.g., once the implant is fully implanted along the tissue) is achieved simply by releasing one end of each longitudinal member and pulling the other end of the longitudinal member (e.g., from outside of the subject) until the longitudinal member unloops from the guide rail.

In some implementations, the guide assembly comprises a plurality of spacers that maintain spacing between the guide rail and the guide frame, even while the loops pull the guide rail toward the guide frame. In some implementations, this spacing may advantageously facilitate anchoring of the helical member by allowing a sharpened tip of the helical member to pass between the guide rail and the guide frame as the helical member is rotated. For example, in some implementations, this spacing may reduce a likelihood of the helical member catching or threading onto the guide frame, and/or fastening the guide frame to the tissue.

In some implementations, the spacers are wires that extend longitudinally along an outer surface of at least the midsection of the guide frame (e.g., perpendicular to the midsection). In some implementations, each individual wire is looped to form two spacers, e.g., on opposite sides of the guide frame. For example, each individual wire can loop around a downstream end of the guide frame.

In some implementations, the guide rail includes a plurality of imaging markers (e.g., fluoroscopic markers and/or echogenic markers), spaced along the guide rail at predetermined intervals.

In some implementations, the imaging markers can be used to visualize the procedure and/or to verify particular steps in the procedure, e.g., before proceeding to a subsequent step. For example, the imaging markers can be used to verify the position of the guide rail around the guide frame (e.g., around the midsection thereof), e.g., to verify that the guide rail is positioned in a manner that facilitates placement of the guide rail along (e.g., parallel with) the annulus.

In some implementations, the positioning of the guide rail is guided by the detection of electrophysiological signals produced by the heart and/or bioimpedance of the tissues. In some such implementations, the guide rail includes a plurality of electrodes spaced along the guide rail. For example, in some implementations, the electrodes can be used to verify the position of the guide rail around the guide frame (e.g., around the midsection thereof), e.g., to verify that the guide rail is positioned in a manner that facilitates placement of the guide rail along (e.g., parallel with) the annulus.

In some implementations, a data-processing system can be adapted to associate various electrical signals detected by the electrode(s) with corresponding locations of the electrode(s) within the heart (e.g., different tissues of the heart, or different locations along an atrioventricular axis of the heart) so that it can be determined, prior to the implantation of the helical member, that the guide rail is satisfactorily positioned at the heart (e.g., along the annulus, such as in alignment with an atrial surface of the annulus), prior to driving the helical member through tissue of the annulus.

In some implementations, the imaging markers can be electrically conductive, such that the imaging markers serve, in addition to facilitating the visualization of the procedure, as the electrodes described herein.

In some implementations, the imaging markers and/or the electrodes can provide the user with information regarding the desired size of the implant, prior to delivery of the implant to the heart. For example, once the guide rail is positioned within the heart, the desired length of the helical member can be determined based on the length of the guide rail that is positioned along the annulus. In some implementations, the extent of this length can be obtained by determining the proportion of the guide rail that is positioned along the annulus, e.g., by visualizing the imaging markers spaced along the guide rail, and/or detecting electrical signals via the electrodes that are spaced along the guide rail.

In some implementations, during delivery (e.g., transluminal delivery) of the guide assembly to the heart, the guide assembly is constrained in a delivery state, e.g., within a sheath. In the delivery state, the guide frame is compressed radially inwards (e.g., in a substantially narrow and/or elongate form).

In some implementations, in the delivery state, the guide rail is disposed alongside (e.g., substantially parallel with) the compressed guide frame. In some implementations, in the delivery state, the guide rail curves (e.g., helically) at least partway around the compressed guide frame.

In some implementations, once the guide assembly is positioned within the heart (e.g., once deployed out of the sheath), the guide frame can be expanded radially within the heart.

In some implementations, the fasteners (e.g., the loops) can then be tightened in a manner that pulls the guide rail toward alignment along the midsection, e.g., such that the guide rail extends circumferentially around at least part of the midsection. As noted hereinabove, for implementations in which the guide assembly includes the spacers, the spacers maintain spacing between the guide rail and the guide frame, e.g., becoming sandwiched between the guide rail and the guide frame upon tightening of the loops.

In some implementations, the tightening of the fasteners is achieved by, for each fastener (e.g., for each loop) sequentially, pulling one or both ends of the longitudinal member of that loop from outside the subject (e.g., living subject or simulation).

In some implementations, prior to the guide assembly reaching its deployed state, different points along the guide rail can be disposed at different distances from the midsection. Thus, the loops can have different exposed lengths (i.e., the length exposed outside of the guide frame) to each other, e.g., in order to accommodate the resulting different distances between the guide rail and the exit sites of the loops from the guide frame. Thus, during tightening of the loops, the longitudinal member of each loop can be pulled by a different amount in order to take up the different exposed lengths so as to draw the guide rail into alignment with the midsection.

In some implementations, the adjustment of the guide rail can be guided by the electrodes that are spaced therealong. For example, during tightening of the loops, electrical signals detected by the electrodes can guide the amount that the longitudinal member of each loop is pulled by, so as to facilitate the drawing of the guide rail into alignment with the midsection, e.g., by determining an orientation of the guide rail along the atrioventricular axis.

Alternative and/or additional techniques for guiding a helical member along tissue of the annulus using a guide assembly are also described. In some implementations, the guide assembly does not include a guide frame for the positioning of a guide rail of a guide assembly.

In some implementations, a guide rail (e.g., similar to that described hereinabove) is positioned around the annulus using any of the techniques described hereinbelow.

In some implementations in which the guide frame is used, the guide rail can be positioned around the annulus (e.g., along the entirety of the path along which the helical member is to be anchored), prior to advancing the helical member along the guide rail.

In some implementations in which the guide assembly does not comprise a guide frame, the guide rail can be advanced along the annulus incrementally with the anchoring of the helical member.

In some implementations, for each such increment, a leading (e.g., distal) segment of the guide rail can be advanced beyond the helical member and placed along a part of the annulus, and the helical member is subsequently advanced helically over the leading segment such that it becomes anchored along that part of the annulus. This can be repeated iteratively until the helical member is anchored along the desired stretch of annulus. Such incremental advancement of the guide rail may advantageously provide enhanced control and/or positioning of the guide rail and/or the helical member, e.g., in place of the guide frame described hereinabove.

In some implementations in which the guide rail is incrementally advanced along the annulus along with the anchoring of the helical member, the leading segment of the guide rail that is extended beyond the helical member can be positioned (e.g., into a desired alignment with respect to the annulus) prior to the screwing of the helical member. Several techniques for such positioning are disclosed.

In some implementations, an electromagnet is used to guide the leading segment of the guide rail against the tissue, e.g., using magnetic repulsion and/or attraction between the electromagnet and the leading segment of the guide rail. In some implementations, the electromagnet can be moved incrementally along with the guide rail, e.g., as the helical member is advanced.

In some implementations, an example of positioning the leading segment of the guide rail includes adjusting a curvature of the leading segment by heating the leading segment. In some implementations, this can be provided by one or more heating elements positioned along the leading segment of the guide rail.

In some implementations, the guide rail (e.g., the leading segment thereof) can include or be made from a shape-memory alloy such as nitinol, with a transition temperature set to above body temperature, such that it curves or straightens responsively to heating.

In some implementations, positioning the leading segment of the guide rail along the annulus includes a guide rail having an inner tube and an outer tube having a different at-rest curvature to the inner tube (e.g., the inner tube at rest being straighter than the outer tube at rest). In some implementations, axially sliding the inner tube through the outer tube alters the curvature of the leading segment of the guide rail, e.g., by increasing and decreasing the influence of the inner and outer tubes on each other.

In some implementations, the positioning of components of the guide assembly (e.g., the guide rail thereof) prior to anchoring of the helical member along the annulus can be guided by detection of electrophysiological signals produced by the heart and/or bioimpedance of the tissues. Similar techniques can be used to guide and/or monitor anchoring of the helical member itself. For example, electrodes on the guide assembly (e.g., on the guide rail) and/or on the helical member can be used for such detection. Such electrodes can be discrete components, but for some implementations components of the implant can themselves serve as electrodes (e.g., due to being inherently electrically conductive).

In some implementations, electrodes on the guide assembly (e.g., on the guide rail) and/or on the helical member can be used for such detection. Such electrodes can be discrete components, but in some implementations components of the implant can themselves serve as electrodes (e.g., due to being inherently electrically conductive).

In some implementations, before implanting of the implant in the heart, a controller (e.g., a surgeon, an interventionalist, and/or a data-processing system) can receive information regarding the desired implantation site of the implant within the heart. In some implementations, it may be important for the helical member to be anchored precisely, according to the surgical plan, in order for the implant to serve the desired purpose.

In some implementations, in which the implant is an annuloplasty implant, for implanting around an annulus of a valve of the heart, it may be desirable to anchor the helical member into the annulus, e.g., to drive the helical member through an atrial surface of the annulus. In some implementations it may thus be undesirable to anchor the helical member to other tissues, such as into the wall of the atrium upstream of the valve or the wall of the ventricle downstream of the valve.

In some implementations, prior to and/or during driving of the helical member into the tissue, the helical member (e.g., a distal tip thereof) can be used as an electrode via which a data-processing system, electrically connected to the helical member, can acquire electrical signals produced by the heart. In some implementations, based on this information, the data-processing system can provide an indication of the location of the helical member within the heart, which the operator (e.g., physician) can use to facilitate optimal anchoring and/or positioning of the helical member.

In some implementations, the data-processing system can be adapted to associate various electrical signals with corresponding locations of the electrode within the heart (e.g., different tissues of the heart, or different locations along an atrioventricular axis of the heart).

In some implementations, a signal acquired from a helical member placed against tissue of the annulus is different from a signal acquired from the same helical member placed against tissue of the atrium or placed against tissue of the ventricle.

In some implementations, a continuous signal is provided during the anchoring of the helical member along the tissue, such that the data-processing system is able to detect changes in the signal as indicating that the helical member has been anchored sufficiently deep into the tissue at each turn, and/or whether the path of the helical member has deviated from its intended path along the annulus. For example, a sudden change in the signal and/or pattern of the signal may indicate a deviation of the helical member into tissue of the atrium or leaflets of the valve.

In some implementations, the data-processing system is electronically coupled to the helical member via a driver, used to drive the helical member into the tissue. For example, a wire with a connector (e.g., a crocodile clip) at its end, can extend from the data-processing system, and can be mechanically and electrically connected to (e.g., clipped onto) a part of an electrically conductive shaft of the driver that is disposed outside of the subject (e.g., living subject or simulation).

In some implementations, the data-processing system also receives an electrical signal (e.g., a second electrical signal) from an additional component of the implantation system (e.g., from the guide assembly of the implant, such as from the guide rail and/or an additional component of the implant itself), e.g., in addition to the electrical signal from the helical member (which can be referred to as the first electrical signal).

In some implementations, this additional component can be considered to be a second electrode, with the helical member being referred to as a first electrode. From the first and second signals, the data-processing system can be configured to derive a refined signal, which is improved (e.g., to have a better signal-to-noise ratio) compared to the first signal alone.

Additionally and/or alternatively to using the helical member as an electrode, in some implementations, in order to implant the helical member along the annulus as desired, it may be important to determine, prior to the implantation, that components of the guide assembly are satisfactorily positioned at the heart, so that the helical member will become correctly positioned within the heart upon implantation.

In some implementations, in which the implant is an annuloplasty implant, for implanting around an annulus of a valve of the heart, it may be desirable to position the guide rail of the guide assembly along the annulus (e.g., in alignment with an atrial surface of the annulus), prior to driving the helical member through tissue of the annulus.

In some implementations, the guide rail can include an electrode via which a data-processing system, electrically connected to the guide rail, can acquire electrical signals produced by the heart. In some implementations, based on this information, the data-processing system can provide an indication of the location of the guide rail within the heart, which the operator (e.g., physician) can use to facilitate optimal positioning of the guide rail.

Techniques for positioning an implant, such as a replacement heart valve, at an annulus of the heart, guided by electrodes disposed on the implant, are also disclosed. Similarly to the technique described hereinabove, in which a plurality of electrodes are positioned around a guide rail such that the positioning of the guide rail along the annulus can be guided by the electrodes, an implant or replacement heart valve can include a plurality of electrodes that are distributed along a circumference (e.g., an outer circumference) of the implant or replacement valve such that the electrodes can be used to verify tissue-contact with the annulus.

In some implementations, before and/or during implantation of the implant or replacement valve at the heart, a controller (e.g., a surgeon, an interventionalist, and/or a data-processing system) can receive information regarding the desired implantation site of the implant/valve within the heart. It may be important for the implant or replacement valve to be secured precisely, according to the surgical plan, in order for the implant to serve the desired purpose. For example, it may be desirable to secure the implant or replacement valve at a particular height within the native valve.

In some implementations, a data-processing system can be adapted to associate various electrical signals with corresponding locations of the electrode within the heart (e.g., different tissues of the heart, or different locations along an atrioventricular axis of the heart), such that the operator can verify that the implant or replacement valve is satisfactorily positioned, e.g., prior to anchoring the implant or replacement valve to the annulus.

In accordance with some implementations, a system and/or an apparatus (which can be used with a heart, e.g., of a living subject or of a simulation) includes an implant, and/or a delivery assembly. The delivery assembly can include a guide assembly, a distal part of which can be transluminally advanceable to the heart while in a delivery state.

In some implementations, the guide assembly can include a guide frame and/or a guide rail.

In some implementations, the guide frame can be intracardially expandable toward an expanded state.

In some implementations, the delivery assembly can include multiple fasteners that are intracardially tightenable, from a proximal extracorporeal portion of the delivery assembly, in a manner that draws the guide rail into a guide arrangement around at least part of the guide frame.

In some implementations, the delivery assembly can include a driver, configured to advance the implant along the guide rail in the guide arrangement.

In some implementations, each of the fasteners is a suture.

In some implementations, the guide rail defines a plurality of imaging markers.

In some implementations, the delivery assembly is configured to facilitate the guide assembly withdrawing the guide rail and the guide frame from the heart while the implant remains in the heart.

In some implementations, in the delivery state, the guide rail is disposed alongside the guide frame.

In some implementations, the guide rail is a hypotube.

In some implementations, the helical member has an axial length of 5-12 cm.

In some implementations, the guide assembly is configured to expand the guide frame prior to the guide rail becoming drawn into the guide arrangement.

In some implementations, the guide assembly is configured to expand the guide frame subsequently to the guide rail becoming drawn into the guide arrangement.

In some implementations, the guide frame is self-expanding.

In some implementations, the implant is sterile.

In some implementations, at least the distal part of the guide assembly is sterile.

In some implementations, the driver is sterile.

In some implementations, the multiple fasteners are tightenable independently of a state of expansion of the guide frame.

In some implementations, the guide rail includes a series of electrodes, spaced along the guide rail, and electrically connected to an extracorporeal portion of the delivery assembly.

In some implementations, the electrodes are radiopaque.

In some implementations, each of the electrodes is a ring electrode that is positioned around the guide rail.

In some implementations, the system further includes a data-processing system, electrically connectable to the one or more electrodes by being connected to a terminal at the extracorporeal portion of the delivery assembly, and configured: (i) to receive an electrical signal from the one or more electrodes, and/or (ii) to, responsively to the electrical signal, provide an output indicative of a position of the guide rail within the heart.

In some implementations: (i) the position includes a proximity of the guide rail to a tissue surface, and/or (ii) the data-processing system is configured to, responsively to the electrical signal, provide the output indicative of the proximity of the guide rail to the tissue surface.

In some implementations: (i) the position includes contact of the guide rail with a tissue surface, and/or (ii) the data-processing system is configured to, responsively to the electrical signal, provide the output indicative of the contact of the guide rail to the tissue surface.

In some implementations: (i) the position is a position along an atrioventricular axis of the heart, and/or (ii) the data-processing system is configured to, responsively to the electrical signal, provide the output indicative of the position of the guide rail along the atrioventricular axis.

In some implementations: (i) the output is indicative of a tissue-type with which the guide rail is in contact, and/or (ii) the data-processing system is configured to, responsively to the electrical signal, provide the output indicative of the tissue-type.

In some implementations: (i) the electrical signal is an ECG signal, and/or (ii) the data-processing system is configured to receive the ECG signal.

In some implementations: (i) the electrical signal is an exogenous signal, and/or (ii) the data-processing system is configured to receive the exogenous signal.

In some implementations: (i) based on the exogenous signal, the data-processing system is configured to determine a bioimpedance of the tissue, and/or (ii) the data-processing system is configured to provide the output responsively to the bioimpedance.

In some implementations, the data-processing system is configured to drive the exogenous signal between at least two of the electrodes of the series.

In some implementations, the guide assembly includes multiple spacers, arranged around the guide frame, and configured to maintain a spacing between the guide rail and the guide frame.

In some implementations: (i) in the guide arrangement, the guide rail lies around a midsection of the guide frame, the midsection disposed longitudinally between an upstream section of the frame and a downstream section of the frame, and/or (ii) the multiple spacers are collectively defined by an elongate member that extends in a serpentine manner around the midsection of the guide frame.

In some implementations: (i) in the guide arrangement, the guide rail lies around a midsection of the guide frame, the midsection disposed longitudinally between an upstream section of the frame and a downstream section of the frame, and/or (ii) each spacer extends longitudinally alongside part of the upstream section, the entire midsection, and part of the downstream section.

In some implementations, each of the spacers has: (i) an upstream terminus that is attached to the guide frame at the upstream section, and/or (ii) a downstream terminus that is disposed at the downstream section.

In some implementations, each of the spacers is in the form of a ribbon.

In some implementations, the downstream terminus is attached to the guide frame at the downstream section.

In some implementations, the downstream terminus is unattached to the guide frame at the downstream section.

In some implementations: (i) in the guide arrangement, the guide rail lies around a midsection of the guide frame, the midsection disposed axially between an upstream section of the frame and a downstream section of the frame, and/or (ii) the spacers collectively form a shield around the midsection, the shield covering the midsection.

In some implementations, the spacers are actuatable to expand the guide frame.

In some implementations, each of the spacers is woven along the guide frame.

In some implementations, the spacers are actuatable to compress the guide frame.

In some implementations, each of the spacers is a wire, extending longitudinally alongside the guide frame.

In some implementations, the guide assembly includes multiple wires, extending distally alongside the guide frame, around a distal end of the guide frame, and returning proximally alongside the guide frame, such that each of the multiple wires defines a pair of the spacers on opposing sides of the guide frame.

In some implementations, each of the multiple fasteners defines a loop around the guide rail.

In some implementations, each of the loops is looped around a respective strut of the guide frame.

In some implementations, each of the multiple fasteners is configured to release the guide rail by being unlooped from around the guide rail.

In some implementations, each fastener of the multiple fasteners is defined by a longitudinal member that extends, from the extracorporeal portion to the distal part, where the longitudinal member forms the loop.

In some implementations, the guide assembly is positionable in a position within the heart such that: (i) the guide rail is disposed along an exterior of the guide frame, at an upstream section of the guide frame, (ii) one or more fasteners of the multiple fasteners exits the guide frame at a downstream section of the guide frame, and extends along the exterior of the guide frame to the guide rail, and/or (iii) the one or more fasteners are intracardially tightenable in a manner that draws the guide rail into the guide arrangement by the one or more fasteners pulling the guide rail toward the downstream section.

In some implementations, in the position of the guide assembly within the heart: (a) the upstream section and the guide rail are upstream of the tissue, and/or (b) the downstream section is downstream of the tissue. In some implementations, while the guide assembly remains in the position, the fasteners are intracardially tightenable in a manner that draws the guide rail into the guide arrangement by the fasteners pulling the guide rail against an upstream surface of the tissue.

In some implementations, in the delivery state, each of the multiple fasteners has an exposed length by which the fastener extends out of the guide frame to the guide rail, the multiple fasteners having different exposed lengths to each other.

In some implementations, the multiple fasteners are arranged in a series around the guide frame. In some implementations, in the delivery state, along the series, each successive fastener has a greater exposed length than the preceding fastener.

In some implementations, the tissue is tissue of an annulus of a valve of the heart, and the guide assembly is configured to position the guide rail, in the guide arrangement, against the annulus.

In some implementations, the guide assembly is configured to position the guide rail against the annulus subsequently to the guide rail being positioned in the guide arrangement.

In some implementations, the guide assembly is configured to position the guide frame to traverse the valve with an upstream section of the guide frame upstream of the valve and a downstream section of the guide frame downstream of the valve.

In some implementations: (a) each fastener of the multiple fasteners extends out of the guide frame at the downstream section of the guide frame, (b) the guide assembly is positionable in the heart such that the guide rail is upstream of the annulus, and/or (c) the fasteners are intracardially tightenable in a manner that draws the guide rail against the annulus by the fasteners pulling the guide rail against an upstream surface of the annulus.

In some implementations, the guide assembly is configured to expand the guide frame while the guide frame remains traversing the valve.

In some implementations, the guide assembly is configured to expand the guide frame prior to positioning the guide frame to traverse the valve.

In some implementations, the guide assembly is configured to apply an expanding force to guide frame in order to intracardially expand the guide frame toward the expanded state.

In some implementations, the guide assembly includes a mechanical actuator that is actuatable to apply the expanding force.

In some implementations, the guide assembly includes a balloon that is inflatable to apply the expanding force.

In some implementations, the implant includes a flexible helical member that defines a plurality of turns.

In some implementations, the helical member has a constant pitch.

In some implementations, the helical member has a sharpened distal tip.

In some implementations, the implant has a head at a proximal end of the helical member. In some implementations, the driver is configured to engage the implant by engaging the head. In some implementations, the helical member has a thickness that is greater toward the proximal end than toward the distal tip.

In some implementations, the thickness of the helical member is tapered to become progressively greater from the distal tip toward the proximal end.

In some implementations, the helical member has a stiffness that is greater toward the proximal end than toward the distal tip.

In some implementations, the driver is configured to anchor the implant along the tissue, over and along the guide rail, by rotating the helical member in a first direction such that the distal tip penetrates the tissue. In some implementations, the helical member is deliverable towards the tissue over and along the guide rail while rotating the helical member in a second direction, the second direction being opposite to the first direction.

In some implementations, the guide assembly is configured to position, along a surface of the tissue, the guide rail in the guide arrangement.

In some implementations, the driver is configured to, while the guide rail in the guide arrangement is positioned along the surface of the tissue, anchor the implant along the tissue by screwing the helical member along the guide rail and the tissue such that part of each turn of the helical member becomes embedded within the tissue, and another part of each turn lies above a surface of the tissue.

In some implementations, the guide rail defines a groove, and the driver is configured to anchor the implant along the tissue by screwing the helical member over and along the guide rail while the helical member is threadedly engaged with, and recessed within, the groove.

In some implementations, the extracorporeal portion of the delivery assembly is electrically connected to the helical member and is adapted to apply electrical energy to the helical member. In some implementations, the helical member is configured to contract responsively to the application of electrical energy, in a manner that draws the turns of the helical member closer to each other.

In some implementations, the extracorporeal portion includes a power source, configured to provide the electrical energy.

In some implementations, the extracorporeal portion: (i) includes a terminal, configured to be electrically and mechanically connected to a power source, and/or (ii) is configured to derive the electrical energy from the power source.

In some implementations, the driver is electrically conductive and electrically connects the extracorporeal portion to the helical member.

In some implementations, the helical member is sufficiently flexible to follow a curvature of the guide rail in the guide arrangement.

In some implementations, the guide rail is configured to limit a depth of penetration of the helical member into the tissue.

In some implementations, the implant further includes a tensile member.

In some implementations, in the guide arrangement, the tensile member extends through the guide rail.

In some implementations, the multiple turns circumscribe a central channel of the helical member, and the delivery assembly is configured to, while the helical member remains anchored along the tissue, retract the guide rail out of the helical member, leaving a tensile member extended through the central channel, the tensile member configured to, upon being tensioned, axially contract the helical member.

In some implementations, the system further includes a stopper coupled to a distal end of the tensile member, such that tension applied to the tensile member longitudinally contracts the helical member by the stopper inhibiting sliding of the tensile member through the central channel.

In some implementations, the system further includes a tensioning tool that is configured to contract the tissue along which the helical member is anchored by axially contracting the helical member by applying tension to the tensile member.

In some implementations, the system further includes a stopper, wherein the tensioning tool is configured to fix the tension in the tensile member by locking the stopper to the tensile member.

In some implementations, the driver is configured to advance the implant along the guide rail by sliding the helical member over and along the guide rail.

In some implementations, the multiple turns circumscribe a central channel, and the guide rail extends through the central channel.

In some implementations, the central channel has a diameter, and the guide rail has a thickness that is at least 50 percent of the diameter of the central channel.

In some implementations, the driver is configured to advance the implant along the guide rail while screwing the helical member into and along the tissue.

In some implementations, the driver is engaged with a proximal end of the helical member and is configured to screw the implant into the tissue by applying torque to the proximal end of the implant.

In some implementations, the implant includes a head coupled to a proximal end of the helical member, the driver being reversibly engaged with the head.

In some implementations, the guide frame has a longitudinal axis, and is intracardially expandable toward the expanded state by being expanded radially away from the longitudinal axis. In some implementations, in the guide arrangement, the guide rail extends latitudinally around at least part of the guide frame.

In some implementations, in the delivery state, the guide rail is disposed parallel with the longitudinal axis.

In some implementations, the system further includes a sheath, the distal part of the guide assembly being transluminally advanceable to the heart while in the delivery state within the sheath.

In some implementations, in the guide arrangement, the guide rail extends, from the sheath, through an interior of the guide frame, and out of the guide frame at an exit site to lie around an exterior of the guide frame.

In some implementations, an invaginating part of the guide frame is configured to invaginate to form an invagination upon the guide frame being intracardially expanded toward its expanded state.

In some implementations, the guide assembly includes a control shaft that is coupled to the guide frame at the invaginating part of the guide frame.

In some implementations, in the delivery state, the guide frame is constrained within the sheath.

In some implementations, the guide frame is configured to automatically self-expand within the heart upon becoming deployed out of the sheath.

In some implementations, the guide frame comprises (e.g., is formed from) a braided filament.

In some implementations, the filament is a wire.

In some implementations, each of the fasteners is defined by a longitudinal member that extends, from the extracorporeal portion to the distal part, where the fastener is engaged with the guide rail.

In some implementations, the guide frame defines an interior and has an exterior. In some implementations, at the distal part, each of the longitudinal members extends from the interior, through the guide frame to the exterior, where the fastener is engaged with the guide rail.

In some implementations, the guide frame has a longitudinal axis, and is intracardially expandable toward the expanded state by being expanded radially away from the longitudinal axis.

In some implementations, at the distal part, each of the longitudinal members extends from the interior, laterally through the guide frame to the exterior, where the fastener is engaged with the guide rail.

In some implementations, in the guide arrangement, the guide rail lies around a midsection of the guide frame, the midsection disposed axially between an upstream section of the frame and a downstream section of the frame. In some implementations, the guide frame is a braided structure defined by a plurality of struts.

In some implementations, at the midsection, each strut is twisted together with an adjacent strut to form a twisted-strut-pair, each twisted-strut-pair defining an eyelet therethrough. In some implementations, each longitudinal member extends, from the interior, through a respective one of the eyelets, to the exterior, where the fastener is engaged with the guide rail.

In some implementations, each twisted-strut-pair is parallel with the longitudinal axis.

In some implementations, the guide assembly includes multiple rods, each of the multiple rods being tubular.

In some implementations, at the distal part, each of the longitudinal members extends out of a respective rod, through the guide frame to the exterior, where the fastener is engaged with the guide rail.

In some implementations, each of the rods has a distal opening that is disposed at an inner surface of the guide frame.

In some implementations, the multiple rods are flexible.

In some implementations, the multiple rods are longitudinally incompressible.

In some implementations, the multiple rods extend distally within the interior.

In some implementations, the guide assembly is configured to position the guide rail, in the guide arrangement, along a surface of the tissue. In some implementations, the driver is configured to anchor the implant along the tissue by advancing the implant helically along the guide rail in the guide arrangement.

In some implementations, the driver has: (i) at a distal end of the driver, a drivehead that is reversibly engaged with the implant, (ii) a driveshaft, the driver being configured to advance the implant helically along the guide rail by torque being applied to the driveshaft, and/or (iii) a neck that connects the driveshaft to the drivehead in a manner that transfers torque from the driveshaft to the drivehead.

In some implementations, the driveshaft is 50-150 cm long.

In some implementations, the neck is 0.5-5 cm long.

In some implementations, both the driveshaft and the neck are formed from a unitary tube that has a first cut pattern along the neck, and a second cut pattern cut along the driveshaft, the second cut pattern being different from the first cut pattern.

In some implementations, the unitary tube further forms the drivehead.

In some implementations, the first cut pattern segments the neck into discrete vertebrae, and the neck is bendable via movement of the vertebrae with respect to each other.

In some implementations, the vertebrae are articulatably coupled to each other via the first cut pattern.

In some implementations, the second cut pattern includes multiple transverse slits along the driveshaft, and the driveshaft is bendable via deformation of the tube and the slits.

In some implementations, the first cut pattern includes multiple tortuous cuts distributed along the neck, each of the tortuous cuts completely circumscribing the tube.

In some implementations, the second cut pattern includes multiple slits distributed along the driveshaft, each of the slits incompletely circumscribing the tube.

In some implementations, the delivery assembly further includes a fixation wire that is connected to a connector of the guide rail in a manner that fastens the connector to a connection location on the guide frame.

In some implementations, the connector is an eyelet defined by a distal end portion of the guide rail, and the fixation wire fastens the connector to the connection location on the guide frame by extending out of the guide frame and looping through the eyelet.

In some implementations, the fixation wire is intracardially withdrawable from the connector of the guide rail to decouple the guide rail from the guide frame.

In some implementations, the connector is disposed at a distal end portion of the guide rail.

In some implementations, each of the fasteners is defined by a longitudinal member that extends, from the extracorporeal portion to the distal part of the guide assembly, where the fastener loops around the guide rail. In some implementations, the fastening, by the fixation wire, of the connector to the connection location inhibits the guide rail from sliding out from the fasteners.

In some implementations, in the guide arrangement, the guide rail lies around a midsection of the guide frame, the midsection disposed axially between an upstream section of the frame and a downstream section of the guide frame. In some implementations, in the delivery state, the connection location is disposed at the downstream section. In some implementations, the delivery assembly is adapted to transition the guide rail towards the guide arrangement by moving the distal end portion towards the midsection.

In some implementations, the multiple fasteners are arranged in a series around the guide frame. In some implementations, the delivery assembly is adapted to move the distal end portion towards the midsection by tightening a distalmost fastener of the series.

In some implementations, the fixation wire is intracardially loosenable to facilitate the movement of the distal end portion away from the connection location and toward the midsection.

In some implementations, in the delivery state, the guide rail extends distally through an interior of the guide frame, and out of the guide frame at an exit site to lie along an exterior of the guide frame.

In some implementations, the guide frame has a longitudinal axis, and is intracardially expandable toward the expanded state by being expanded radially away from the longitudinal axis. In some implementations, in the guide arrangement, the guide rail extends distally through the interior of the guide frame, and out of the guide frame at the exit site to curve around the exterior of the guide frame and the longitudinal axis.

In some implementations, in the guide arrangement, the guide rail extends distally through an interior of the guide frame, and out of the guide frame at an exit site to lie around an exterior of the guide frame.

In some implementations, the delivery assembly further includes a tube, the guide rail extending through the tube. In some implementations, in the guide arrangement: (a) the tube extends distally through the interior of the guide frame, and out of the guide frame at the exit site, (b) within the tube, the guide rail extends distally through the interior of the guide frame and out of the guide frame at the exit site, and (c) at the exterior of the guide frame, the guide rail exits the tube to lie, exposed from the tube, around the exterior of the guide frame.

In some implementations, the tube has a distal section that exits the guide frame at the exit site, and at least the distal section of the tube is a flexible sleeve.

In some implementations, the delivery assembly further includes a tube, the guide rail extending through the tube. In some implementations, in the guide arrangement, the guide rail extends distally through and out of the tube to lie, exposed from the tube, around an exterior of the guide frame.

In some implementations, at least a distal section of the tube is a flexible sleeve.

In some implementations, the sleeve includes a polymer.

In some implementations, the sleeve is a fabric sleeve.

In some implementations, the guide assembly is configured to position the guide rail, in the guide arrangement, along a surface of the tissue. In some implementations, the implant includes a suture. In some implementations, the driver is configured to stitch the suture along the tissue by advancing the suture helically along the guide rail in the guide arrangement.

In some implementations, the implant further includes a tensile member. In some implementations, the driver is configured to stitch the suture along the tissue such that the suture defines a helix (e.g., a series of turns) along the tissue, with the tensile member extending along an interior of the helix. In some implementations, the implant is configured such that tensioning of the tensile member adjusts a dimension of the tissue by the tensile member pulling on the suture.

In some implementations, the driver is configured to stitch the suture such that the helix is disposed in a curved path along the tissue, the curved path having a radius of curvature. In some implementations, the implant is configured such that tensioning of the tensile member adjusts the dimension of the tissue by reducing the radius of curvature of the curved path.

In some implementations, the driver is configured to stitch the suture such that the helix is disposed in a curved path along the tissue, the curved path having a length. In some implementations, the implant is configured such that tensioning of the tensile member adjusts the dimension of the tissue by reducing the length of the curved path.

In some implementations, the tensile member is disposed within a lumen of the guide rail, and the delivery assembly is configured to retract the guide rail proximally out from the helix, leaving the tensile member exposed within the helix.

In some implementations, the driver includes a helical member, and is adapted to stitch the suture along the tissue by driving the helical member helically along the guide rail in the guide arrangement, such that the helical member becomes temporarily stitched along the tissue. In some implementations, the delivery assembly is configured to leave the suture stitched along the tissue with the tensile member extending along the interior of the helix by: (a) unstitching the helical member from the tissue by retracting the helical member helically, and/or (b) retracting the guide rail linearly.

In some implementations, the driver includes a helical member that is adapted to stitch the suture along the tissue by driving the helical member helically along the guide rail in the guide arrangement.

In some implementations, the suture is attached to an exterior of the helical member.

In some implementations, the suture is detachable from the helical member once the suture is stitched along the tissue.

In some implementations, the suture is attached to a distal end portion of the helical member, and the driver is adapted to stitch the suture along the tissue by withdrawing the helical member from the tissue such that the suture is drawn into and along the tissue by the withdrawal of the helical member.

In some implementations, the driver is adapted to stitch the suture into the tissue alongside the helical member.

In some implementations, the helical member defines multiple turns, and the driver is configured to advance the implant along the tissue by screwing the helical member and the suture into the tissue such that part of each turn of the helical member becomes embedded within the tissue, and another part of each turn lies above the surface of the tissue.

In some implementations, the helical member is a hollow helical needle defining a channel therethrough, and the driver is configured to stitch the suture along the tissue by screwing the helical member along the tissue while the suture is disposed within the channel.

In some implementations, the driver is configured to be unscrewed from the tissue, leaving the suture stitched along the tissue.

In some implementations, in the guide arrangement, the guide rail lies around a midsection of the guide frame, the midsection disposed axially between an upstream section of the frame and a downstream section of the frame. In some implementations, in the expanded state of the guide frame, the upstream section is wider than the downstream section.

In some implementations, the guide assembly further includes a control shaft that extends from the extracorporeal portion and is coupled to the upstream section of the guide frame. In some implementations, the guide assembly includes multiple actuator wires, operably coupled to the extracorporeal portion.

In some implementations, each of the actuator wires extends, from the control shaft, distally through an interior of the upstream section of the guide frame, and weavingly along the downstream section of the guide frame, such that tensioning the actuator wires by operation of the extracorporeal portion radially expands the guide frame.

In some implementations, the upstream section is wider than the midsection.

In some implementations, in the expanded state, the guide frame is mushroom-shaped.

In some implementations, in the expanded state, the upstream section protrudes radially outwards over the midsection.

In some implementations, the driver is configured to implant the implant along the tissue, over and along the guide rail. In some implementations, the guide frame is positionable within the heart, in the expanded state, such that the upstream section protrudes radially outwards over an upstream surface of the tissue.

In some implementations, the guide assembly is configured to move the guide frame, in the expanded state, in a downstream direction until the upstream section protrudes radially outwards over the upstream surface of the tissue.

In some implementations, the guide assembly is configured to move the guide frame, in the expanded state, in an upstream direction such that the upstream section squeezes past the tissue to protrude radially outwards over the upstream surface of the tissue.

In some implementations, the guide frame defines an upstream section and a downstream section, and a concave waist disposed axially between the upstream section and the downstream section. In some implementations, in the expanded state of the guide frame, the waist has a smaller circumference than both the upstream section and the downstream section.

In some implementations, the guide assembly further includes a control shaft that extends from the extracorporeal portion and is coupled to the upstream section of the guide frame.

In some implementations, the guide assembly includes multiple actuator wires, operably coupled to the extracorporeal portion, and extending via the control shaft to the guide frame. In some implementations, each of the actuator wires extends, from the control shaft, distally through an interior of the upstream section of the guide frame, and weavingly along the downstream section of the guide frame, such that tensioning the actuator wires by operation of the extracorporeal portion radially expands the guide frame.

In some implementations, in the guide arrangement, the guide rail lies around the waist of the guide frame.

In some implementations, the guide assembly is adapted to position the guide frame at the tissue such that the tissue becomes sandwiched between the upstream section and the downstream section.

In some implementations, the guide assembly is adapted to position the guide frame at the tissue such that the tissue becomes gripped between the upstream section and the downstream section.

In some implementations, a distal end of the guide frame is invaginated to form an invagination.

In some implementations, the invagination is secured by a crimp.

In some implementations, the guide frame defines a plurality of struts, and the struts are gathered together at a distal end of the guide frame to form an atraumatic distal end of the guide frame.

In some implementations, the distal end is coated with a coating.

In some implementations, the distal end is secured by a crimp.

In some implementations, the struts are invaginated to form the atraumatic distal end.

In some implementations, the guide assembly includes a control shaft, a distal end of the control shaft being attached to the guide frame in a manner that facilitates transluminal control of the guide frame via the control shaft.

In some implementations, the control shaft includes a tube. In some implementations, at a distal region of the control shaft, the control shaft defines a flex zone in which cuts in the tube confer flexibility to the tube. In some implementations, the control shaft further includes a strip that has greater tensile strength than the flex zone. In some implementations, a first end of the strip is attached to the tube distally from the flex zone, and a second end of the strip is attached to the tube proximally from the flex zone, such that the strip lies slack alongside the flex zone.

In some implementations, each of the first end of the strip and the second end of the strip are attached to the tube by welding.

In some implementations: (i) the strip is a first strip, (ii) the control shaft further includes a second strip having greater tensile strength than the flex zone, (iii) a first end of the second strip is attached to the tube distally from the flex zone, opposite the first end of the first strip, and a second end of the second strip is attached to the tube proximally from the flex zone, opposite the second end of first strip, such that the strip lies slack alongside the flex zone, on an opposing side of the flex zone to the first strip.

In some implementations, the tube is a hypotube.

In some implementations, the delivery assembly is configured to facilitate withdrawal of the guide frame via pulling of the control shaft in a manner that tensions the strip.

In some implementations: (i) the control shaft includes a tube, (ii) at a distal region of the control shaft, the control shaft defines a flex zone in which a cut pattern in the tube confers flexibility to the tube, the cut pattern including multiple slits distributed along the flex zone, each of the slits incompletely circumscribing the tube such that an uncut axial strip remains along the flex zone.

In some implementations: (i) the strip is a first strip, (ii) the cut pattern defines a second uncut axial strip along the flex zone, the second strip being disposed on an opposing side of the flex zone to the first strip.

In some implementations, the tube is a hypotube.

In some implementations, the guide assembly includes multiple actuator wires, operably coupled to the extracorporeal portion, woven longitudinally along at least part of the guide frame, and attached to a downstream part of the guide frame, such that tensioning the actuator wires from the extracorporeal portion radially expands the guide frame.

In some implementations, in the guide arrangement, the guide rail lies around a midsection of the guide frame, the midsection disposed longitudinally between an upstream section of the frame and a downstream section of the frame. In some implementations, the guide assembly further includes a control shaft that extends from the extracorporeal portion and is coupled to the upstream section of the guide frame.

In some implementations, each of the actuator wires extends, from the control shaft, distally through an interior of the upstream section of the guide frame, and weavingly along the downstream section of the guide frame.

In some implementations, in the expanded state of the guide frame, at least part of the upstream section is wider than the downstream section.

In some implementations, in the expanded state of the guide frame, at least part of the upstream section is wider than the midsection.

In some implementations, the guide assembly further includes a control shaft that extends from the extracorporeal portion and is coupled to the upstream section of the guide frame. In some implementations, the guide assembly is configured such that the guide frame is pivotable with respect to the control shaft via differential tensioning of the actuator wires.

In some implementations, the extracorporeal portion includes at least one controller to which the actuator wires are operatively coupled. In some implementations, the extracorporeal portion is configured to differentially actuate the actuator wires via actuation of the at least one controller.

In some implementations, the guide frame is configured to radially expand responsively to balanced tension in the actuator wires. In some implementations, the extracorporeal portion is configured to apply the balanced tension to the actuator wires.

In some implementations, the at least one controller is configured with: (i) a first actuation mode that applies the balanced tension to the actuator wires, and/or (ii) a second actuation mode that applies the differential tension to the actuator wires.

In some implementations, in the guide arrangement, the guide rail lies around a midsection of the guide frame, the midsection disposed axially between an upstream section of the frame and a downstream section of the frame. In some implementations, the guide assembly includes a shield that is disposed around the midsection such that, in the guide arrangement, the shield is disposed between the guide rail and the guide frame.

In some implementations, the shield includes an elastic material.

In some implementations, the shield is defined by a fabric.

In some implementations, the shield is defined by a film.

In some implementations, the shield is defined by a net.

In some implementations, the shield has a hypotube-type structure.

In some implementations, the shield is a ribbon that, in the delivery state of the guide assembly, is wrapped around the guide frame, and expanding the guide frame towards the expanded state causes the ribbon to slide over itself in a manner that reduces the wrapping around the guide frame.

In some implementations, the shield is defined by multiple ribbons distributed circumferentially around the midsection.

In some implementations, in the guide arrangement, each ribbon contacts its neighboring ribbons, such that the ribbons collectively cover the midsection.

In some implementations, in the delivery state, the ribbons are imbricated around the midsection. In some implementations, the ribbons are configured to facilitate expansion of the guide frame towards the expanded state by the ribbons sliding over each other while collectively covering the midsection.

In some implementations, in the expanded state, the ribbons remain imbricated around the midsection.

In some implementations, in the expanded state, the ribbons are arranged edge-to-edge around the midsection.

In some implementations, each ribbon is polymeric.

In some implementations, each ribbon is metallic.

In some implementations, in the guide arrangement, the guide rail lies around a midsection of the guide frame, the midsection disposed axially between an upstream section of the frame and a downstream section of the frame. In some implementations, the guide frame is a braided structure defined by a plurality of struts. In some implementations, at the midsection, each strut is twisted together with an adjacent strut to form a twisted-strut-pair.

In some implementations, each twisted-strut-pair is covered with a covering.

In some implementations, the guide frame defines a longitudinal axis between the upstream section and the downstream section, and each twisted-strut-pair is substantially parallel to the longitudinal axis.

In some implementations, the guide rail defines an external thread. In some implementations, the driver is configured to advance the implant along the guide rail by advancing the implant threadedly along the external thread.

In some implementations, the external thread defines a groove. In some implementations, the implant includes a helical member defining a plurality of turns. In some implementations, the driver is configured to screw the helical member helically along the thread while the helical member is recessed within the groove.

In some implementations, the guide rail defines a central guide-rail axis, and has a tissue-facing surface that, along the guide rail, is disposed closer than the external thread to the central guide-rail axis. In some implementations, in the guide arrangement, the external thread faces medially toward the guide frame, and the tissue-facing surface faces radially away from the guide frame.

In some implementations, the tissue-facing surface is unthreaded, and runs parallel with the external thread.

In some implementations, the tissue-facing surface is substantially flat.

In some implementations, the tissue-facing surface is concave.

In some implementations, the guide assembly includes a rider that is slidably mounted to the guide rail such that, as the driver advances the implant along the guide rail, a leading end of the implant pushes the rider along the guide rail, while the rider shields the guide frame from the leading end of the implant.

In some implementations, the implant includes a helical member defining a sharpened tip at the leading end. In some implementations, the rider defines a lobe that, as the implant pushes the rider along the guide rail, the lobe remains disposed between the leading end and the guide frame, thereby shielding the guide frame from the sharpened tip.

In some implementations, the lobe is rotationally locked with respect to the guide rail.

In some implementations, the lobe is rotationally locked with respect to the guide rail via keying between the rider and the guide rail.

In accordance with some implementations, a method (which can be used with a heart, e.g., of a living subject or of a simulation) includes transluminally advancing a guide frame to the heart while the guide frame is secured to a guide rail via multiple fasteners that extend out of the guide frame to the guide rail. In some implementations, the method includes expanding the guide frame within the heart.

In some implementations, the guide rail can be drawn into a guide arrangement around at least part of the guide frame by tightening at least one of the multiple fasteners.

In some implementations, while the guide rail remains in the guide arrangement, an implant can be positioned along a tissue of the heart, guided by the guide rail.

In some implementations, the method further includes sterilizing the implant.

In some implementations, the method further includes sterilizing the guide frame.

In some implementations, the method further includes sterilizing the guide rail.

In some implementations, drawing the guide rail into the guide arrangement includes drawing the guide rail into the guide arrangement by tightening: by a first amount, a first of the multiple fasteners, and/or by a second amount, a second of the multiple fasteners, the second amount being greater than the first amount.

In some implementations, the method further includes, subsequently to positioning the implant along the tissue, withdrawing the guide rail and the guide frame from the heart while the implant remains disposed along the tissue.

In some implementations, advancing the guide frame to the heart includes advancing the guide frame to the heart while the guide rail is disposed alongside the guide frame.

In some implementations, transluminally advancing the guide frame to the heart includes transluminally advancing the guide frame to the heart while the guide frame is constrained in a delivery state within a sheath.

In some implementations, the guide rail defines multiple imaging markers, and drawing the guide rail into the guide arrangement includes drawing the guide rail into the guide arrangement guided by at least one image that includes the imaging markers.

In some implementations, the method further includes, prior to positioning the implant along the tissue, determining a desired size of the implant guided by the at least one image that includes the imaging markers.

In some implementations, the method further includes, prior to positioning the implant along the tissue, selecting the implant from a selection of implants, responsively to the at least one image that includes the imaging markers.

In some implementations, the method further includes, prior to positioning the implant along the tissue, adjusting a size of the implant responsively to the at least one image that includes the imaging markers.

In some implementations, the method further includes determining a position of the guide rail within the heart responsively to an electrical signal detected via the guide rail.

In some implementations, the method further includes, prior to positioning the implant along the tissue, selecting the implant from a selection of implants, responsively to the electrical signal.

In some implementations, the method further includes, prior to positioning the implant along the tissue, adjusting a size of the implant responsively to the electrical signal.

In some implementations, the guide rail defines multiple imaging markers. In some implementations, the electrical signal is detected via the imaging markers serving as electrodes. In some implementations, drawing the guide rail into the guide arrangement includes drawing the guide rail into the guide arrangement guided by at least one image that includes the imaging markers.

In some implementations, the electrical signal is an endogenous electrical signal, and determining the position of the guide rail responsively to the electrical signal includes determining the position of the guide rail responsively to the endogenous electrical signal.

In some implementations, the endogenous electrical signal is an ECG signal, and determining the position of the guide rail responsively to the endogenous electrical signal includes determining the position of the guide rail responsively to the ECG signal.

In some implementations, the electrical signal is an exogenous electrical signal, and determining the position of the guide rail responsively to the electrical signal includes determining the position of the guide rail responsively to the exogenous electrical signal.

In some implementations, the method further includes applying the exogenous electrical signal to the subject (e.g., living subject or simulation).

In some implementations, determining the position of the guide rail responsively to the exogenous electrical signal includes determining the position of the guide rail responsively to sensing a bioimpedance of the tissue.

In some implementations, the method further includes, subsequently to drawing the guide rail into the guide arrangement around the part of the guide frame and prior to positioning the implant along the tissue, responsively to the electrical signal, positioning the guide rail within the heart.

In some implementations, positioning the guide rail within the heart includes determining a position of the guide rail along an atrioventricular axis of the heart.

In some implementations, determining the position of the guide rail within the heart includes verifying contact between at least a portion of the guide rail and the tissue.

In some implementations, the guide rail has a series of electrodes spaced along the guide rail, the electrical signal is one of multiple electrical signals received via the series of electrodes. In some implementations, positioning the guide rail within the heart includes positioning the guide rail within the heart responsively to the multiple electrical signals.

In some implementations, positioning the implant along the tissue includes positioning the implant along a stretch of the tissue.

In some implementations, adjusting the position of the guide rail includes adjusting the position of the guide rail such that (i) electrical signals received from a first portion of the guide rail that is to be positioned along the stretch indicate a presence of tissue contact, and (ii) electrical signals received from a second portion of the guide rail that is to be positioned away from the tissue indicate an absence of tissue contact.

In some implementations, transluminally advancing the guide frame to the heart includes transluminally advancing the guide frame to the heart while the guide frame is constrained in a delivery state within a sheath.

In some implementations, expanding the guide frame within the heart includes deploying the guide frame out of the sheath such that the guide frame automatically self-expands within the heart.

In some implementations, the guide frame has a longitudinal axis, and expanding the guide frame includes expanding the guide frame radially away from the longitudinal axis.

In some implementations, in the guide arrangement, the guide rail extends latitudinally around at least part of the guide frame.

In some implementations, advancing the guide frame to the heart includes advancing the guide frame to the heart while the guide rail is disposed parallel with the longitudinal axis.

In some implementations, expanding the guide frame within the heart includes applying an expanding force to the guide frame within the heart.

In some implementations, applying the expanding force includes actuating a mechanical actuator within the heart.

In some implementations, applying the expanding force includes inflating a balloon within the heart.

In some implementations, positioning the implant along the tissue includes screwing the implant into and along the tissue in a manner in which the implant becomes progressively threaded around the guide rail.

In some implementations, the implant further includes a helical member defining multiple turns, and the method further includes applying energy to the helical member while the helical member remains screwed into and along the tissue such that, responsively to the application of the energy, the helical member contracts in a manner that draws the turns of the helical member toward to each other.

In some implementations, applying the energy to the helical member includes applying electrical energy to the helical member from an extracorporeal power source that is electrically connected to the helical member.

In some implementations, screwing the helical member into the tissue includes screwing the helical member into the tissue by applying torque to a proximal end of the helical member.

In some implementations, the helical member defines a sharpened distal tip. In some implementations, the helical member has a thickness that is greater toward the proximal end than toward the distal tip. In some implementations, applying torque to the proximal end of the helical member includes applying torque to the proximal end toward which the thickness of the helical member is greater.

In some implementations, the thickness of the helical member is tapered to become progressively greater from the distal tip toward the proximal end, and screwing the helical member into the tissue includes screwing the tapered helical member into the tissue.

In some implementations, the helical member defines a sharpened distal tip. In some implementations, the helical member has a stiffness that is greater toward the proximal end than toward the distal tip. In some implementations, applying torque to the proximal end of the helical member includes applying torque to the proximal end toward which the stiffness of the helical member is greater.

In some implementations, applying torque to the proximal end of the helical member includes applying torque to the proximal end of the helical member in a first direction such that a distal tip of the helical member penetrates the tissue. In some implementations, the method further includes, prior to screwing the helical member along the tissue, delivering the helical member towards the tissue over and along the guide rail while rotating the helical member in a second direction, the second direction being opposite to the first direction.

In some implementations, screwing the helical member into the tissue includes screwing the helical member into the tissue such that a screw axis of the helical member is disposed along a surface of the tissue.

In some implementations, the method further includes, subsequently to positioning the implant along the tissue, retracting the guide rail from out of the implant, leaving the implant implanted in the heart.

In some implementations, retracting the guide rail from out of the implant includes sliding the guide rail proximally through the implant.

In some implementations, the implant includes a helical member defining multiple turns that circumscribe a central channel. In some implementations, screwing the implant into and along the tissue includes screwing the helical member into the tissue, such that part of each turn of the helical member becomes embedded within the tissue, and another part of each turn lies above a surface of the tissue.

In some implementations, the helical member defines a sharpened distal tip and screwing the helical member into the tissue includes repeatedly driving the sharpened distal tip into and out of the tissue.

In some implementations, the implant further includes a tensile member, and the method further includes, subsequently to implanting the helical member along the tissue, axially contracting the helical member by tensioning the tensile member.

In some implementations, the helical member includes a head, and screwing the helical member into the tissue includes screwing the helical member into the tissue using a driver that is engaged with the head.

In some implementations, the tissue is tissue of an annulus of a valve of the heart, and/or positioning the implant along the tissue, guided by the guide rail includes advancing the implant along the tissue of the annulus, guided by the guide rail.

In some implementations, positioning the implant along the tissue includes screwing the implant into and along an atrial surface of the tissue of the annulus.

In some implementations, expanding the guide frame includes expanding the guide frame while the guide frame traverses the valve with an upstream section of the guide frame upstream of the valve and a downstream section of the guide frame downstream of the valve.

In some implementations, the guide frame has a midsection between the upstream section and the downstream section. In some implementations, expanding the guide frame includes expanding the guide frame such that the midsection presses radially against the valve.

In some implementations, the heart has an atrium upstream of the valve, and a ventricle downstream of the valve. In some implementations, drawing the guide rail into the guide arrangement includes drawing the guide rail into the guide arrangement while an upstream section of the guide frame is disposed within the atrium.

In some implementations, drawing the guide rail into the guide arrangement includes drawing the guide rail into the guide arrangement while a downstream section of the guide frame is disposed in the ventricle.

In some implementations, each fastener of the multiple fasteners extends out of the guide frame at the downstream section of the guide frame.

In some implementations, drawing the guide rail into the guide arrangement by tightening the at least one fastener includes drawing the guide rail into the guide arrangement while the guide rail is within the atrium, such that the fasteners pull the guide rail against an upstream surface of the annulus.

In some implementations, drawing the guide rail into the guide arrangement includes drawing the guide rail into the guide arrangement while a downstream section of the guide frame is disposed in the atrium.

In some implementations, the method further includes, subsequently to drawing the guide rail into the guide arrangement, advancing the guide frame ventricularly through the valve until the guide rail abuts the annulus.

In some implementations, expanding the guide frame within the heart includes expanding the guide frame within the atrium, and the method further includes, prior to drawing the guide frame into the guide arrangement, advancing a downstream section of the guide frame into the ventricle.

In some implementations, the method further includes, subsequently to positioning the implant along the annulus, contracting the annulus by contracting the implant.

In some implementations, contracting the implant includes applying tension to a tensile member extending through a central channel of the implant.

In some implementations, applying tension to the tensile member includes pulling the tensile member using a tensioning tool.

In some implementations, the method further includes locking the tension in the tensile member by locking a stopper to the tensile member.

In some implementations, the method further includes trimming excess tensile member that is proximal to the stopper.

In some implementations, drawing the guide rail into the guide arrangement includes drawing the guide rail into the guide arrangement while the tensile member is extended through a lumen of the guide rail.

In some implementations, advancing the implant along the tissue includes advancing the implant over and along the guide rail such that the guide rail progressively becomes disposed within the central channel of the implant.

In some implementations, the method further includes, subsequently to advancing the implant along the tissue, and prior to contracting the implant, withdrawing the guide rail out of the central channel, leaving the tensile member extending through the central channel.

In some implementations, positioning the implant along the tissue includes advancing the implant along the guide rail.

In some implementations, the implant includes a helical member defining a plurality of turns, and advancing the implant along the guide rail includes advancing the implant along the guide rail by screwing the helical member into the tissue, such that part of each turn of the helical member becomes embedded within the tissue, and another part of each turn lies above a surface of the tissue.

In some implementations, each fastener of the multiple fasteners defines a loop around the guide rail. In some implementations, tightening the multiple fasteners includes, for each fastener of the multiple fasteners, tightening the respective loop of the fastener.

In some implementations, drawing the guide rail into the guide arrangement includes drawing the guide rail into the guide arrangement while: (i) the guide rail is disposed along an exterior of the guide frame, at an upstream section of the guide frame, and/or (ii) each fastener of the multiple fasteners exits the guide frame at a downstream section of the guide frame, and extends along the exterior of the guide frame to the guide rail, such that tightening the at least one fastener pulls the guide rail toward the downstream section.

In some implementations, each fastener of the multiple fasteners is defined by a longitudinal member that extends, from outside of the subject (e.g., living subject or simulation) to the heart, where the longitudinal member forms the loop. In some implementations, for each fastener of the multiple fasteners, tightening the respective loop of the fastener includes pulling the longitudinal member from outside of the subject (e.g., living subject or simulation).

In some implementations, advancing the guide frame includes advancing the guide frame while each of the multiple fasteners has a respective exposed length by which the fastener extends out of the guide frame to the guide rail, the multiple fasteners having different exposed lengths to each other.

In some implementations, the multiple fasteners are arranged in a series around the guide frame. In some implementations, advancing the guide frame includes advancing the guide frame while, along the series, each successive fastener has a greater exposed length than the preceding fastener.

In some implementations, drawing the guide rail into the guide arrangement includes, for each fastener of the multiple fasteners, pulling the longitudinal member from outside the subject until the loop of the fastener draws the guide rail against the guide frame.

In some implementations, the method further includes, subsequently to positioning the implant along the tissue, for each of the fasteners, opening the loop.

In some implementations, opening the loop includes unlooping the loop from around the guide rail.

In some implementations, opening the loop includes unlooping the loop from around the implant.

In some implementations, drawing the guide rail into the guide arrangement includes drawing the guide rail into the guide arrangement such that multiple spacers, disposed alongside the guide frame, become sandwiched between the guide rail and the guide frame.

In some implementations, each of the spacers is defined by a wire, and drawing the guide rail into the guide arrangement includes drawing the guide rail into the guide arrangement such that the wire becomes sandwiched between the guide rail and the guide frame.

In some implementations, the method further includes, while the guide rail remains in the guide arrangement, retracting the spacers from between the guide rail and the guide frame.

In some implementations, expanding the guide frame includes expanding the guide frame by actuating the spacers.

In some implementations, the method further includes, subsequently to positioning the implant along the tissue, compressing the guide frame by actuating the spacers.

In some implementations, positioning the implant along the tissue includes advancing the implant helically along the guide rail in the guide arrangement such that the implant becomes implanted along a surface of the tissue.

In some implementations, advancing the implant helically along the guide rail includes advancing the implant helically along the guide rail by applying torque to the implant using a driver, a drivehead of the driver being reversibly engaged with the implant, the driver having: (a) a driveshaft, and/or (b) a neck that connects the driveshaft to the drivehead.

In some implementations, both the driveshaft and the neck are formed from a unitary tube having a first cut pattern along the neck, and a second cut pattern cut along the driveshaft, the second cut pattern being different from the first cut pattern, and transluminally advancing the implant to the tissue while the implant is reversibly engaged with the driver includes transluminally advancing the implant to the tissue along a tortuous path while the implant is reversibly engaged with the driver such that the neck bends responsively to the tortuous path, facilitated by the first cut pattern.

In some implementations, the unitary tube further forms the drivehead, and applying torque to the unitary tube includes applying torque to the driveshaft of the unitary tube while the drivehead of the unitary tube is engaged with the implant.

In some implementations, the first cut pattern segments the neck into discrete vertebrae, and transluminally advancing the implant to the tissue while the implant is reversibly engaged with the driver such that the neck bends responsively to the tortuous path includes transluminally advancing the implant to the tissue while the implant is reversibly engaged with the driver such that vertebrae move with respect to each other, responsively to the tortuous path.

In some implementations, transluminally advancing the guide frame to the heart includes transluminally advancing the guide frame to the heart while a fixation wire that is connected to a connector of the guide rail fastens the connector to a connection location on the guide frame.

In some implementations, the connector is an eyelet defined by a distal end portion of the guide rail.

In some implementations, the fixation wire fastens the connector to the connection location on the guide frame by extending out of the guide frame and looping through the eyelet.

In some implementations, the method further includes, prior to drawing the guide rail into the guide arrangement, intracardially loosening the fixation wire to facilitate movement of the guide rail away from the connection location and into the guide arrangement.

In some implementations, the method further includes intracardially withdrawing the fixation wire from the connector of the guide rail to decouple the guide rail from the guide frame.

In some implementations, the connector is disposed at a distal end portion of the guide rail.

In some implementations, in the guide arrangement, the guide rail lies around a midsection of the guide frame, the midsection disposed axially between an upstream section of the frame and a downstream section of the frame.

In some implementations, during advancement of the guide frame to the heart, the connection location is disposed at the downstream section.

In some implementations, drawing the guide rail into the guide arrangement includes moving the distal end portion towards the midsection.

In some implementations, each of the fasteners is defined by a longitudinal member that extends, from an extracorporeal end of the longitudinal member to the guide frame, where the fastener loops around the guide rail.

In some implementations, the fastening, by the fixation wire, of the connector to the connection location inhibits the guide rail from sliding out from the fasteners.

In some implementations, transluminally advancing the guide frame to the heart includes transluminally advancing the guide frame to the heart while the guide rail is inhibited, by the fastening, from sliding out from the fasteners.

In some implementations, the multiple fasteners are arranged in a series around the guide frame.

In some implementations, moving the distal end portion towards the midsection includes tightening a distalmost fastener of the series.

In some implementations, the method further includes intracardially loosening the fixation wire to facilitate the movement of the distal end portion away from the connection location and toward the midsection.

In some implementations, transluminally advancing the guide frame to the heart includes transluminally advancing the guide frame to the heart while the guide rail extends distally through an interior of the guide frame, and out of the guide frame at an exit site to lie along an exterior of the guide frame.

In some implementations: (i) the guide frame has a longitudinal axis, and/or (ii) expanding the guide frame includes expanding the guide frame radially away from the longitudinal axis.

In some implementations, positioning the implant along the tissue includes positioning the implant along the tissue while the guide rail extends distally through an interior of the guide frame, and laterally out of the guide frame at an exit site to lie along an exterior of the guide frame.

In some implementations, positioning the implant along the tissue includes advancing the implant over and along the guide rail such that the implant passes through the interior of the guide frame, out of the guide frame at the exit site, and along the exterior of the guide frame and the tissue.

In some implementations, drawing the guide rail into the guide arrangement includes drawing the guide rail into the guide arrangement such that: (a) within the interior of the guide frame, the guide rail is ensheathed within a tube that extends to the exterior of the guide frame, and/or (b) at the exterior of the guide frame, the guide rail is exposed from the tube.

In some implementations, advancing the implant over and along the guide rail includes advancing the implant over and along the guide rail such that: (a) within the interior of the guide frame, the implant advances over and along the guide rail within the tube, and/or (b) at the exterior of the guide frame, the implant exits the tube to advance over and along the guide rail, exposed from the tube.

In some implementations, the implant includes a suture.

In some implementations, positioning the implant along the tissue includes stitching the suture along the tissue by advancing the suture helically along the guide rail in the guide arrangement.

In some implementations, the implant further includes a tensile member.

In some implementations, stitching the suture along the tissue includes stitching the suture along the tissue and the tensile member such that the suture defines a helix along the tissue, with the tensile member extending along an interior of the helix.

In some implementations, the method further includes tensioning the tensile member to adjust a dimension of the tissue by the tensile member pulling on the suture.

In some implementations, stitching the suture along the tissue includes stitching the suture along the tissue such that the helix is disposed in a curved path along the tissue, the curved path having a radius of curvature.

In some implementations, tensioning the tensile member to adjust the dimension of the tissue includes tensioning the tensile member to adjust the dimension of the tissue by reducing the radius of curvature of the curved path.

In some implementations, stitching the suture along the tissue includes stitching the suture along the tissue such that the helix is disposed in a curved path along the tissue, the curved path having a length.

In some implementations, tensioning the tensile member to adjust the dimension of the tissue includes tensioning the tensile member to adjust the dimension of the tissue by reducing the length of the curved path.

In some implementations, stitching the suture along the tissue includes stitching the suture along the tissue while the tensile member is disposed within a lumen of the guide rail, and, the method further includes retracting the guide rail proximally out from the helix, leaving the tensile member exposed within the helix.

In some implementations, stitching the suture along the tissue includes stitching the suture along the tissue using a helical member to stitch the suture helically along the guide rail in the guide arrangement, such that the helical member becomes temporarily stitched along the tissue.

In some implementations, the method further includes unstitching the helical member from the tissue by retracting the helical member helically, leaving the suture stitched along the tissue with the tensile member extending along the interior of the helix.

In some implementations, stitching the suture along the tissue includes stitching the suture along the tissue using a helical member to stitch the suture helically along the guide rail in the guide arrangement, such that the helical member becomes temporarily stitched along the tissue.

In some implementations, the method further includes unstitching the helical member from the tissue by retracting the helical member helically, leaving the suture stitched along the tissue.

In some implementations, stitching the suture along the tissue includes stitching the suture along the tissue while the suture is attached to an exterior of the helical member.

In some implementations, the method further includes detaching the suture from the helical member once the suture is stitched along the tissue.

In some implementations, the suture is attached to a distal end portion of the helical member, and stitching the suture along the tissue includes stitching the suture along the tissue by withdrawing the helical member from the tissue such that the suture is drawn into and along the tissue by the withdrawal of the helical member.

In some implementations, stitching the suture along the tissue includes using the helical member to stitch the suture along the tissue while the suture is alongside the helical member.

In some implementations, the helical member defines multiple turns, and stitching the suture along the tissue includes stitching the suture along the tissue by screwing the helical member and the suture into the tissue such that part of each turn of the helical member becomes embedded within the tissue, and another part of each turn lies above a surface of the tissue.

In some implementations, the helical member is a hollow helical needle defining a channel therethrough, and stitching the suture along the tissue includes screwing the helical member along the tissue while the suture is disposed within the channel.

In some implementations, in the guide arrangement, the guide rail lies around a midsection of the guide frame, the midsection disposed axially between an upstream section of the frame and a downstream section of the frame.

In some implementations, expanding the guide frame within the heart includes expanding the guide frame within the heart such that the upstream section is wider than the downstream section.

In some implementations, expanding the guide frame within the heart includes expanding the guide frame within the heart such that the upstream section is wider than the midsection.

In some implementations, expanding the guide frame within the heart includes expanding the guide frame within the heart such that the upstream section protrudes radially outwards over the midsection.

In some implementations, positioning the implant along the tissue includes positioning the implant along the tissue while the upstream section protrudes radially outwards over an upstream surface of the tissue.

In some implementations, the method further includes, subsequently to expanding the guide frame, moving the guide frame in a downstream direction until the upstream section protrudes radially outwards over the upstream surface of the tissue.

In some implementations, the method further includes, subsequently to expanding the guide frame, moving the guide frame in an upstream direction such that the upstream section squeezes past the tissue to protrude radially outwards over the upstream surface of the tissue.

In some implementations, the guide frame defines an upstream section and a downstream section, and a concave waist disposed axially between the upstream section and the downstream section.

In some implementations, expanding the guide frame includes expanding the guide frame by expanding the upstream section and the downstream section to a greater extent than the waist, such that the waist has a smaller circumference than both the upstream section and the downstream section.

In some implementations, positioning the implant along the tissue includes positioning the implant along the tissue while the guide rail lies around the waist of the guide frame.

In some implementations, expanding the guide frame within the heart includes expanding the guide frame at the tissue such that the tissue becomes gripped between the upstream section and the downstream section.

In some implementations, the method further includes, subsequently to expanding the guide frame within the heart, positioning the guide frame within the heart such that the tissue becomes sandwiched between the upstream section and the downstream section.

In some implementations, expanding the guide frame within the heart includes expanding the guide frame within the heart such that an invaginating part of the guide frame invaginates to form an invagination.

In some implementations, a distal part of a control shaft is coupled to the guide frame at the invaginating part of the guide frame, and transluminally advancing the guide frame to the heart includes transluminally advancing the guide frame to the heart while the guide frame is coupled to the distal part of the control shaft.

In some implementations, expanding the guide frame within the heart includes expanding the guide frame by extracorporeally tensioning multiple actuator wires that are woven longitudinally along at least part of the guide frame.

In some implementations, in the guide arrangement, the guide rail lies around a midsection of the guide frame, the midsection disposed axially between an upstream section of the frame and a downstream section of the frame. In some implementations, transluminally advancing the guide frame to the heart includes transluminally advancing the guide frame to the heart using a control shaft that is coupled to the upstream section of the guide frame. In some implementations, each of the actuator wires extends, distally through an interior of the upstream section of the guide frame, and weavingly along the downstream section of the guide frame.

In some implementations, expanding the guide frame within the heart includes expanding the downstream section of the guide frame by tensioning the actuator wires.

In some implementations, expanding the guide frame within the heart includes expanding the guide frame such that at least part of the upstream section is wider than the downstream section.

In some implementations, expanding the guide frame within the heart includes expanding the guide frame such that at least part of the upstream section is wider than the midsection.

In some implementations, transluminally advancing the guide frame to the heart includes transluminally advancing the guide frame to the heart using a control shaft that is coupled to a proximal part of the guide frame. In some implementations, the method further includes pivoting the guide frame with respect to the control shaft by differentially tensioning the actuator wires.

In some implementations, expanding the guide frame within the heart includes expanding the guide frame by applying balanced tension to the actuator wires.

In some implementations, in the guide arrangement, the guide rail lies around a midsection of the guide frame, the midsection disposed axially between an upstream section of the frame and a downstream section of the frame. In some implementations, expanding the guide frame within the heart includes expanding the guide frame such that a shield that is disposed around the midsection expands along with the guide frame.

In some implementations, expanding the guide frame such that the shield expands along with the guide frame includes expanding the guide frame such that the shield elastically expands along with the guide frame.

In some implementations, the shield is a ribbon. In some implementations, transluminally advancing the guide frame to the heart includes transluminally advancing the guide frame to the heart while the ribbon is wrapped around the guide frame.

In some implementations, expanding the guide frame includes expanding the guide frame such that the ribbon slides over itself in a manner that reduces the wrapping around the guide frame.

In some implementations, the shield is defined by multiple ribbons distributed circumferentially around the midsection. In some implementations, transluminally advancing the guide frame to the heart includes transluminally advancing the guide frame to the heart while the ribbons are imbricated around the midsection.

In some implementations, expanding the guide frame within the heart includes expanding the guide frame within the heart such that the ribbons slide over each other while collectively covering the midsection.

In some implementations, the guide rail defines an external thread. In some implementations, positioning the implant along the tissue, guided by the guide rail includes advancing the implant threadedly along the external thread.

In some implementations, the external thread defines a groove. In some implementations, the implant includes a helical member defining a plurality of turns. In some implementations, advancing the implant threadedly along the external thread includes advancing the implant threadedly along the external thread while the helical member is recessed within the groove.

In some implementations, positioning the implant along the tissue, guided by the guide rail includes advancing the implant along the guide rail while a leading end of the implant pushes a rider that is slidably mounted to the guide rail along the guide rail, while the rider shields the guide frame from the leading end of the implant.

In some implementations, the implant includes a helical member defining a sharpened tip at the leading end. In some implementations, advancing the implant along the guide rail includes advancing the implant along the guide rail while a lobe of the rider remains disposed between the leading end and the guide frame, thereby shielding the guide frame from the sharpened tip.

In accordance with some implementations, a system and/or an apparatus (which can be used with a heart, e.g., of a living subject or of a simulation) includes an implant and/or a delivery assembly. In some implementations, the delivery assembly can include a guide assembly and/or a driver.

In some implementations, the guide assembly includes a guide rail that has a leading segment, the implant being mounted on the guide rail.

In some implementations, the driver can be engaged with or configured to engage with the implant.

In some implementations, the delivery assembly can be configured to iteratively secure the implant along the tissue by iteratively (i) advancing the leading segment distally out of a distal end of the implant into a position along a stretch of the tissue, and (ii) securing the leading segment to the stretch of the tissue by the driver screwing the implant into the tissue along the stretch.

In some implementations, the implant is sterile. In some implementations, the guide assembly is sterile. In some implementations, the guide rail is sterile. In some implementations, the driver is sterile.

In some implementations, the leading segment includes one or more imaging markers to facilitate determination of a position of the leading segment within the heart.

In some implementations, the system further includes a catheter within which the delivery assembly and the implant are transluminally advanceable to the heart.

In some implementations, the driver is configured to screw the implant into the tissue along the stretch by screwing the implant over and along the leading segment while the leading segment remains in the position along the stretch.

In some implementations, the guide assembly is configured to slide the guide rail proximally out of the implant.

In some implementations, the delivery assembly is configured to manipulate the leading segment into an alignment with respect to the stretch of the tissue.

In some implementations, the leading segment includes a magnetic material. In some implementations, the system further includes an electromagnet, advanceable to the heart, and configured to manipulate the leading segment into the alignment.

In some implementations, the system includes an electromagnet tool that includes the electromagnet, and that is configured to energize the electromagnet in a manner that manipulates the leading segment into the alignment by magnetically attracting the leading segment toward the electromagnet.

In some implementations, the system includes an electromagnet tool that includes the electromagnet, and that is configured to energize the electromagnet in a manner that manipulates the leading segment into the alignment by magnetically repelling the leading segment away from the electromagnet.

In some implementations, the system includes an electromagnet tool that includes the electromagnet, and that is configured to advance the electromagnet into an atrium of the heart, and to manipulate the leading segment from the atrium.

In some implementations, the system includes an electromagnet tool that includes the electromagnet, and that is configured to advance the electromagnet into a ventricle of the heart, and to manipulate the leading segment from the ventricle.

In some implementations, the system includes an electromagnet tool that includes the electromagnet, and that is configured to advance the electromagnet into a coronary blood vessel of the heart, and to manipulate the leading segment from the coronary blood vessel.

In some implementations, the leading segment includes a shape-memory alloy, a curvature of the leading segment being adjustable by heating the leading segment.

In some implementations, the guide rail includes multiple heating elements distributed along the leading segment, and drivable to electrically heat the leading segment.

In some implementations, each of the heating elements is drivable independently of the other heating elements so as to adjust the curvature of only a corresponding part of the leading segment.

In some implementations, the leading segment includes: an outer tube and/or an inner shaft.

In some implementations, the inner shaft is disposed inside the outer tube. In some implementations, the inner shaft is axially slidable with respect to the outer tube.

In some implementations, the inner shaft has a different at-rest curvature to the outer tube.

In some implementations, the inner shaft has a greater at-rest curvature than the outer tube. In some implementations, the outer tube has a greater at-rest curvature than the inner shaft.

In some implementations, the leading segment includes one or more electrodes electrically connected to an extracorporeal portion of the delivery assembly.

In some implementations, the system further includes a data-processing system, electrically connectable to the one or more electrodes by being connected to a terminal at the extracorporeal portion of the delivery assembly.

In some implementations, the data-processing system is configured: to receive an electrical signal from the one or more electrodes, and/or to, responsively to the electrical signal, provide an output indicative of a position of the leading segment within the heart.

In some implementations, the position includes a proximity of the leading segment to a tissue surface. In some implementations, the data-processing system is configured to, responsively to the electrical signal, provide the output indicative of the proximity of the leading segment to the tissue surface.

In some implementations, the position includes contact of the leading segment to a tissue surface. In some implementations, the data-processing system is configured to, responsively to the electrical signal, provide the output indicative of the contact of the leading segment to the tissue surface.

In some implementations, the position is a position along an atrioventricular axis of the heart. In some implementations, the data-processing system is configured to, responsively to the electrical signal, provide the output indicative of the position along the atrioventricular axis.

In some implementations, the output is indicative of a tissue-type with which the leading segment is in contact. In some implementations, the data-processing system is configured to, responsively to the electrical signal, provide the output indicative of the tissue-type.

In some implementations, the electrical signal is an ECG signal. In some implementations, the data-processing system is configured to receive the ECG signal.

In some implementations, the electrical signal is an exogenous signal. In some implementations, the data-processing system is configured to receive the exogenous signal.

In some implementations, based on the exogenous signal, the data-processing system is configured to determine a bioimpedance. In some implementations, the data-processing system is configured to provide the output responsively to the bioimpedance.

In some implementations, the one or more electrodes are multiple electrodes. In some implementations, the data-processing system is configured to drive the exogenous signal between at least two of the electrodes.

In some implementations, the implant includes one or more electrodes electrically connected to an extracorporeal portion of the delivery assembly.

In some implementations, the one or more electrodes are electrically connected to an extracorporeal portion of the driver.

In some implementations, the system further includes a data-processing system, electrically connectable to the one or more electrodes by being connected to a terminal at the extracorporeal portion of the delivery assembly.

In some implementations, the data-processing system is configured to receive an electrical signal from the one or more electrodes.

In some implementations, the data-processing system is configured to, responsively to the electrical signal, provide an output indicative of a position of the implant within the heart.

In some implementations, the position includes a proximity of the implant to a tissue surface. In some implementations, the data-processing system is configured to, responsively to the electrical signal, provide the output indicative of the proximity of the implant to the tissue surface.

In some implementations, the position includes an orientation of the implant with respect to the tissue. In some implementations, the data-processing system is configured to, responsively to the electrical signal, provide the output indicative of the orientation of the implant with respect to the tissue.

In some implementations, the position includes a depth of the implant within the tissue. In some implementations, the data-processing system is configured to, responsively to the electrical signal, provide the output indicative of the depth of the implant within the tissue.

In some implementations, the position is a position along an atrioventricular axis of the heart. In some implementations, the data-processing system is configured to, responsively to the electrical signal, provide the output indicative of the position along the atrioventricular axis.

In some implementations, the output is indicative of a tissue-type with which the implant is in contact. In some implementations, the data-processing system is configured to, responsively to the electrical signal, provide the output indicative of the tissue-type.

In some implementations, the electrical signal is an ECG signal. In some implementations, the data-processing system is configured to receive the ECG signal.

In some implementations, the electrical signal is an exogenous signal. In some implementations, the data-processing system is configured to receive the exogenous signal.

In some implementations, based on the exogenous signal, the data-processing system is configured to determine a bioimpedance. In some implementations, the data-processing system is configured to provide the output responsively to the bioimpedance.

In some implementations, the one or more electrodes are multiple electrodes. In some implementations, the data-processing system is configured to drive the exogenous signal between at least two of the electrodes.

In some implementations, the implant includes a helical member defining a plurality of turns and circumscribing a central channel.

In some implementations, the implant includes a head that is coupled to the helical member, the driver being engaged with the head of the implant and configured to screw the implant into the tissue by applying torque to the head.

In some implementations, the helical member defines a sharpened tip.

In some implementations, the implant further includes a tensile member disposed within the central channel and is configured to axially contract the helical member upon tensioning of the tensile member.

In some implementations, the tensile member extends along a lumen defined by the guide rail.

In accordance with some implementations, a method for implanting an implant along a tissue (which can be tissue of a heart, e.g., of a living subject or of a simulation) includes positioning a leading segment of a guide rail along a first stretch of the tissue, and/or securing the leading segment to the first stretch by screwing the implant into the tissue along the first stretch.

In some implementations, the method further includes subsequently advancing the leading segment of the guide rail distally out of a distal end of the implant and along a second stretch of the tissue.

In some implementations, the method further includes securing the leading segment to the second stretch by screwing the implant into the tissue along the second stretch.

In some implementations, the method further includes sterilizing the implant.

In some implementations, the method further includes sterilizing the guide rail.

In some implementations, for each of the first stretch and the second stretch, screwing the implant into the tissue along the stretch includes screwing the implant over and along the leading segment while the leading segment is disposed along the stretch.

In some implementations, screwing the implant into the tissue along the first stretch and the second stretch includes screwing the implant into the tissue in a manner in which the implant becomes progressively threaded around the guide rail.

In some implementations, the method further includes, subsequently to screwing the implant into the tissue along the second stretch, retracting the guide rail from out of the implant by sliding the guide rail proximally through the implant, such that the implant remains implanted in the heart.

In some implementations, the implant includes a helical member defining a plurality of turns and circumscribing a central channel.

In some implementations, screwing the implant into the tissue along the first stretch and the second stretch includes screwing the helical member into the tissue, such that: (i) part of each turn of the helical member becomes embedded within the tissue, and another part of each turn lies above a surface of the tissue, and/or (ii) the leading segment becomes disposed within the central channel.

In some implementations, the helical member defines a sharpened distal tip and screwing the helical member into the tissue includes screwing the helical member into the tissue facilitated by the sharpened distal tip.

In some implementations, the implant further includes a tensile member that is disposed within the central channel, and the method further includes, subsequently to screwing the implant into the tissue along the second stretch, tensioning the tensile member to reduce a circumference of the tissue.

In some implementations, the implant includes a head that is reversibly engageable by a driver, and screwing the helical member into the tissue includes screwing the helical member into the tissue, while the driver is engaged with the head, using the driver.

In some implementations, the method further includes transluminally advancing the guide rail to the heart while housed in a catheter.

In some implementations, positioning the leading segment along the first stretch includes positioning the leading segment along the first stretch by exposing the leading segment out of the catheter.

In some implementations, the method further includes, for each of the first stretch and the second stretch, while the leading segment remains positioned along the stretch, and prior to securing the leading segment to the stretch, determining a position of the leading segment within the heart.

In some implementations, determining a position of the leading segment within the heart includes determining the position of the leading segment within the heart by imaging the leading segment within the heart.

In some implementations, imaging the leading segment within the heart includes imaging the leading segment within the heart using fluoroscopy.

In some implementations, determining a position of the leading segment within the heart includes determining the position of the leading segment within the heart by sensing an electrical signal using the leading segment.

In some implementations, the electrical signal is an endogenous electrical signal, and sensing the electrical signal includes sensing the endogenous electrical signal.

In some implementations, the endogenous electrical signal is an ECG signal, and sensing the endogenous electrical signal includes sensing the ECG signal.

In some implementations, the electrical signal is an exogenous electrical signal, and sensing the electrical signal includes sensing the exogenous electrical signal.

In some implementations, the method further includes applying the exogenous electrical signal to the subject (e.g., living subject or simulation).

In some implementations, sensing the exogenous electrical signal includes sensing bioimpedance.

In some implementations, the method further includes, subsequently to positioning the leading segment along the first stretch and prior to screwing the implant into the tissue along the first stretch: (i) receiving electrophysiological signals produced by the heart that are indicative of a position of the leading segment within the heart, and/or (ii) responsively to the received signals, determining the position of the leading segment within the heart.

In some implementations, determining the position of the leading segment within the heart includes determining the position of the leading segment along an atrioventricular axis of the heart.

In some implementations, responsively to the received signals, determining the position of the leading segment within the heart includes responsively to the received signals, determining whether there is contact between the leading segment and the tissue.

In some implementations, the leading segment defines an electrode, and receiving electrophysiological signals produced by the heart includes receiving electrophysiological signals produced by the heart via the electrode.

In some implementations, the electrode is a ring electrode that is positioned around the leading segment, and receiving electrophysiological signals produced by the heart includes receiving electrophysiological signals produced by the heart via the ring electrode.

In some implementations, the method further includes, subsequently to positioning the leading segment along the first stretch and prior to screwing the implant into the tissue along the first stretch: (i) receiving electrophysiological signals produced by the heart that are indicative of the position of the implant within the heart, and/or (ii) responsively to the received signals, determining a position of the implant within the heart.

In some implementations, determining the position of the implant within the heart includes determining the position of the implant along an atrioventricular axis of the heart.

In some implementations, the implant includes a helical member defining a plurality of turns and circumscribing a central channel. In some implementations, screwing the implant into the tissue along the first stretch and the second stretch includes screwing the helical member into the tissue, such that part of each turn of the helical member becomes embedded within the tissue, and another part of each turn lies above a surface of the tissue.

In some implementations, receiving electrophysiological signals produced by the heart includes receiving electrophysiological signals produced by the heart via an electrode that is mounted on a turn of the plurality of turns of the helical member.

In some implementations, the method includes, responsively to the received signals, determining the position of the implant within the heart includes responsively to the received signals, determining whether the electrode is disposed within the tissue.

In some implementations, the implant defines an electrically conductive portion, and receiving electrophysiological signals produced by the heart includes receiving electrophysiological signals produced by the heart via the electrically conductive portion.

In some implementations, positioning the leading segment along the first stretch includes manipulating the leading segment into a desired alignment with respect to the first stretch.

In some implementations, the leading segment includes a magnetic material, and manipulating the leading segment includes manipulating the leading segment using an electromagnet.

In some implementations, manipulating the leading segment using the electromagnet includes magnetically repelling the leading segment from the electromagnet.

In some implementations, manipulating the leading segment using the electromagnet includes magnetically attracting the leading segment toward the electromagnet.

In some implementations, the electromagnet is a first electromagnet of a series of electromagnets, and manipulating the leading segment includes manipulating the leading segment using the series of electromagnets disposed at various positions within the heart.

In some implementations, the method further includes positioning the electromagnet in an atrium of the heart, and manipulating the leading segment using the electromagnet includes manipulating the leading segment using the electromagnet while the electromagnet is disposed in the atrium.

In some implementations, the method further includes positioning the electromagnet in a ventricle of the heart, and manipulating the leading segment using the electromagnet includes manipulating the leading segment using the electromagnet while the electromagnet is disposed in the ventricle.

In some implementations, the method further includes positioning the electromagnet in a coronary blood vessel of the heart adjacent the first stretch, and manipulating the leading segment using the electromagnet includes manipulating the leading segment using the electromagnet while the electromagnet is disposed in the coronary blood vessel.

In some implementations, the method further includes advancing the electromagnet along the coronary blood vessel such that the electromagnet becomes adjacent the second stretch, and positioning the leading segment along the second stretch manipulating the leading segment into a desired alignment with respect to the second stretch using the electromagnet.

In some implementations, manipulating the leading segment into a desired alignment with respect to the tissue includes adjusting a curvature of the leading segment by electrically heating the leading segment.

In some implementations, the guide rail includes multiple heating elements distributed along the leading segment, and electrically heating the leading segment includes electrically heating the leading segment by driving one or more of the multiple heating elements.

In some implementations, adjusting the curvature of the leading segment includes adjusting the curvature of only a part of the leading segment by heating only the part of the leading segment by driving only a subset of the multiple heating elements.

In some implementations, the leading segment includes an outer tube and/or an inner shaft. In some implementations, the inner shaft is disposed inside the outer tube.

In some implementations, the inner shaft has a different at-rest curvature to the outer tube.

In some implementations, the method includes manipulating the leading segment into the desired alignment with respect to the tissue includes adjusting a curvature of the leading segment by axially sliding the inner shaft with respect to the outer tube.

In some implementations, the outer tube has a greater at-rest curvature than the inner shaft, and adjusting the curvature of the leading segment includes reducing a curvature of the leading segment by axially sliding the inner shaft distally with respect to the outer tube.

In some implementations, the inner shaft has a greater at-rest curvature than the outer tube, and adjusting the curvature of the leading segment includes increasing a curvature of the leading segment by axially sliding the inner shaft distally with respect to the outer tube.

In accordance with some implementations, a method (which can be used with a heart, e.g., of a living subject or of a simulation) includes transluminally advancing a guide frame to the heart (e.g., simulated heart) while the guide frame is secured to a guide rail via multiple fasteners that extend out of the guide frame to the guide rail. In some implementations, the guide frame can be expanded within the heart.

In some implementations, the guide rail can be drawn into a guide arrangement around at least part of the guide frame by tightening at least one of the multiple fasteners.

In some implementations, while the guide rail remains in the guide arrangement, an implant can be positioned along a tissue (e.g., simulated tissue) of the heart (e.g., simulated heart), guided by the guide rail.

In accordance with some implementations, a method for implanting an implant along a tissue (e.g., along simulated tissue) of a heart (e.g., of a living subject or a simulated heart of a simulation) includes positioning a leading segment of a guide rail along a first stretch of the tissue (e.g., living tissue, simulated tissue, etc.), and/or securing the leading segment to the first stretch by screwing the implant into the tissue along the first stretch.

In some implementations, the method can further include subsequently advancing the leading segment of the guide rail distally out of a distal end of the implant and along a second stretch of the tissue (e.g., living tissue, simulated tissue, etc.).

In some implementations, the method can further include securing the leading segment to the second stretch by screwing the implant into the tissue along the second stretch.

In accordance with some implementations, a system and/or an apparatus (which can be used with a heart, e.g., of a living subject or of a simulation) includes a guide assembly, is transluminally advanceable to the heart, and includes a guide frame, deployable at a site within the heart.

In some implementations, the system can further include one or more fasteners, that are secured to the guide frame.

In some implementations, the system can further include a guide rail, threaded through the fasteners and positionable around the guide frame such that the guide rail lies along the guide frame in a guide arrangement, the guide rail having a series of electrodes arranged along the guide rail.

In some implementations, the system can further include a data-processing system, electrically connected to each electrode of the series, and configured: (i) to receive an electrical signal from each electrode of the series, and/or (ii) to, responsively to the electrical signal, provide an output indicative of a position of the guide rail within the heart.

In some implementations, the electrodes are radiopaque.

In some implementations, each electrode of the series is electrically connected to an extracorporeal portion of the guide assembly. In some implementations, the extracorporeal portion includes an electrical terminal. In some implementations, the data-processing system is electrically connected to the electrodes by being electrically connected to the terminal.

In some implementations, each of the electrodes of the series is a ring electrode that is positioned around the guide rail.

In some implementations, the position includes a proximity of the guide rail to a tissue surface. In some implementations, the data-processing system is configured to, responsively to the electrical signal, provide the output indicative of the proximity of the guide rail to the tissue surface.

In some implementations, the position includes contact of the guide rail with a tissue surface. In some implementations, the data-processing system is configured to, responsively to the electrical signal, provide the output indicative of the contact of the guide rail to the tissue surface.

In some implementations, the position includes verification of contact of the guide rail with tissue of an annulus of the heart. In some implementations, the data-processing system is configured to, responsively to the electrical signal, provide the output indicative of verification of contact of the guide rail with tissue of the annulus of the heart.

In some implementations, the position is a position along an atrioventricular axis of the heart. In some implementations, the data-processing system is configured to, responsively to the electrical signal, provide the output indicative of the position of the guide rail along the atrioventricular axis.

In some implementations, the output is indicative of a tissue-type with which the guide rail is in contact. In some implementations, the data-processing system is configured to, responsively to the electrical signal, provide the output indicative of the tissue-type.

In some implementations, the electrical signal is an ECG signal. In some implementations, the data-processing system is configured to receive the ECG signal.

In some implementations, the guide frame is intracardially expandable toward an expanded state in a manner which draws the guide rail into the guide arrangement along the guide frame.

In some implementations, the electrical signal is an exogenous signal. In some implementations, the data-processing system is configured to receive the exogenous signal.

In some implementations, based on the exogenous signal, the data-processing system is configured to determine a bioimpedance of the tissue. In some implementations, the data-processing system is configured to provide the output responsively to the bioimpedance.

In some implementations, the data-processing system is configured to drive the exogenous signal between at least two of the electrodes of the series.

In some implementations, the fasteners are arranged in a series around the guide frame. In some implementations, the guide assembly has a delivery state in which: (i) the guide assembly is transluminally advanceable to the heart, and/or (ii) along the series, each successive fastener has a greater exposed length than the preceding fastener.

In some implementations, the fasteners are intracardially tightenable, responsively to the output, from a proximal extracorporeal portion of the guide assembly, in a manner that draws the guide rail into the guide arrangement along the guide frame.

In some implementations, the system further includes an implant, and a driver, configured to advance the implant along the guide rail while the guide rail is in the guide arrangement.

In some implementations, the implant is a helical member.

In accordance with some implementations, a method (which can be used with a heart, e.g., of a living subject or of a simulation) includes transluminally advancing a guide frame and a guide rail to the heart.

In some implementations, the method can further include expanding the guide frame within the heart.

In some implementations, the method can further include determining a position of the guide rail within the heart responsively to an electrical signal detected via the guide rail.

In some implementations, the method can further include, responsively to the determining, drawing the guide rail into a guide arrangement around at least part of the guide frame.

In some implementations, the method further includes, while the guide rail remains in the guide arrangement, positioning an implant along a tissue of the heart, guided by the guide rail.

In some implementations, the method further includes, prior to positioning the implant along the tissue, adjusting a size of the implant responsively to the electrical signal.

In some implementations, transluminally advancing the guide frame and the guide rail to the heart includes transluminally advancing the guide frame and the guide rail to the heart while the guide frame is secured to the guide rail via multiple fasteners, each fastener of the multiple fasteners extending out of the guide frame and securing the guide rail within a loop of the fastener.

In some implementations, drawing the guide rail into the guide arrangement includes, for each fastener of the multiple fasteners, responsively to the determining, pulling the fastener from outside the subject (e.g., living subject or simulation) until the loop of the fastener draws the guide rail against the guide frame.

In some implementations, the guide rail has a series of electrodes spaced therealong. In some implementations, the electrical signal is a first electrical signal of multiple electrical signals, each of the multiple electrical signals being detected by a corresponding electrode of the series. In some implementations, determining the position of the guide rail includes determining the position of the guide rail responsively to the multiple electrical signals.

In some implementations, the first electrical signal is detected by a first electrode of the series, and drawing the guide rail into the guide arrangement includes drawing the guide rail into the guide arrangement by pulling: (i) by a first amount, a first of the multiple fasteners, responsively to the first electrical signal, and/or (ii) by a second amount, a second of the multiple fasteners, responsively to a second electrical signal detected by a second electrode of the series.

In some implementations, pulling the first of the multiple fasteners includes pulling the first of the multiple fasteners until the first electrical signal indicates tissue contact between the first electrode and the tissue. In some implementations, pulling the second of the multiple fasteners includes pulling the second of the multiple fasteners until the second electrical signal indicates tissue contact between the second electrode and the tissue.

In some implementations, drawing the guide rail into the guide arrangement includes determining that (i) electrical signals received from a first portion of the guide rail indicate a presence of tissue contact, and (ii) electrical signals received from a second portion of the guide rail indicate an absence of tissue contact.

In some implementations, in the guide arrangement of the guide rail, the first portion is distal to the second portion.

In some implementations, in the guide arrangement of the guide rail, the second portion is disposed, within the heart, upstream of the first portion.

In accordance with some implementations, a method usable with a heart (e.g., living or simulated) includes transluminally advancing a replacement heart valve to the heart, the replacement heart valve having a series of electrodes disposed along an outer circumference thereof.

In some implementations, the method can further include expanding the replacement heart valve within the heart.

In some implementations, the method can further include receiving an output indicative of an electrical signal detected via the series of electrodes.

In some implementations, the method can further include positioning the replacement heart valve within the heart, responsively to the indication.

In accordance with some implementations, a system and/or an apparatus (which can be used with a heart, e.g., of a living subject or of a simulation) includes a delivery assembly, and a replacement heart valve, having a series of electrodes spaced along an outer circumference thereof, and transluminally advanceable to the heart via the delivery assembly.

In some implementations, the system can further include a data-processing system, electrically connected to each electrode of the series via the delivery assembly, and configured: (i) to receive an electrical signal from the series of electrodes, and/or (ii) responsively to the electrical signal, to provide an output indicative of a position of the replacement heart valve within the heart.

In some implementations, the electrical signal is a first electrical signal of multiple electrical signals, each of the multiple electrical signals being detected by a corresponding electrode of the series, and the data-processing system is configured to provide the output responsively to the multiple electrical signals.

In some implementations, the position includes a proximity of the outer circumference to a tissue surface. In some implementations, the data-processing system is configured to, responsively to the electrical signal, provide the output indicative of the proximity of the outer circumference to the tissue surface.

In some implementations, the position includes contact of the outer circumference with a tissue surface. In some implementations, the data-processing system is configured to, responsively to the electrical signal, provide the output indicative of the contact of the outer circumference to the tissue surface.

In some implementations, the position includes verification of contact of the outer circumference with tissue of an annulus of the heart. In some implementations, the data-processing system is configured to, responsively to the electrical signal, provide the output indicative of verification of contact of the outer circumference with tissue of the annulus of the heart.

In some implementations, the position is indicative of a height of the outer circumference within a valve of the heart. In some implementations, the data-processing system is configured to, responsively to the electrical signal, provide the output indicative of a height of the outer circumference within a valve of the heart.

In some implementations, the output is indicative of a tissue-type with which the outer circumference is in contact. In some implementations, the data-processing system is configured to, responsively to the electrical signal, provide the output indicative of the tissue-type.

In some implementations, the electrical signal is an ECG signal. In some implementations, the data-processing system is configured to receive the ECG signal.

In some implementations, the electrical signal is an exogenous signal. In some implementations, the data-processing system is configured to receive the exogenous signal.

In some implementations, based on the exogenous signal, the data-processing system is configured to determine a bioimpedance of the tissue. In some implementations, the data-processing system is configured to provide the output responsively to the bioimpedance.

In some implementations, the data-processing system is configured to drive the exogenous signal between at least two of the electrodes of the series.

In some implementations, the replacement heart valve has a frame that defines a plurality of struts.

In some implementations, the frame defines a waist that is adapted to become disposed circumferentially within at an annulus of the heart. In some implementations, the outer circumference is an outer circumference of the waist, the series of electrodes being spaced therealong.

In accordance with some implementations, a system (which can be used with a tissue, e.g., of a living subject or of a simulation), the system including a tissue anchor, and/or a driver.

In some implementations, the tissue anchor includes a helical tissue-engaging element and an anchor head.

In some implementations, the driver is configured to transluminally screw the tissue-engaging element into the tissue by applying torque to the anchor head, the driver formed from a unitary tube that defines: (a) at a distal end of the driver, a drivehead configured to reversibly engage the anchor head, (b) a driveshaft, configured to receive the torque from a proximal end of the driver, the driveshaft defining a first pattern of cuts that includes multiple transverse slits along the driveshaft, the driveshaft being bendable via deformation of the tube and the slits, and/or (c) a neck that connects the driveshaft to the drivehead in a manner that transfers the torque from the driveshaft to the drivehead, the neck: (i) defining a second pattern of cuts that segments the neck into discrete vertebrae, and/or (ii) being bendable via movement of the vertebrae with respect to each other.

In accordance with some implementations, a system usable with tissue of a heart, the system including an implant, and/or a delivery assembly.

In some implementations, the delivery assembly includes a guide assembly and/or a driver.

In some implementations, the guide assembly includes, at a distal part of the guide assembly, a guide frame and a guide rail, the guide assembly: (i) having a delivery state in which the distal part is transluminally advanceable to the heart, and/or (ii) being intracardially transitionable into a guide state in which the guide rail is in a guide arrangement in which the guide rail extends: (a) through an interior of the guide frame, exiting the guide frame at an exit site, and/or (b) from the exit site, around an exterior of the guide frame.

In some implementations, the driver is configured to advance the implant along the guide rail in the guide arrangement.

In some implementations, the delivery assembly is configured to facilitate the guide assembly withdrawing the guide rail and the guide frame from the heart while the implant remains in the heart.

In some implementations, the guide assembly includes multiple fasteners that are intracardially tightenable, from a proximal extracorporeal portion of the delivery assembly, in a manner that draws the guide rail into the guide arrangement around at least part of the exterior of the guide frame.

In some implementations, the tissue is tissue of an annulus of a valve of the heart.

In some implementations, the driver is configured to advance the implant along the guide rail in the guide arrangement while the guide rail is disposed along the annulus.

In some implementations, the driver is configured to advance the implant along the guide rail by screwing the implant into and along an atrial surface of the tissue of the annulus.

In some implementations, the system includes a flexible helical member that defines a plurality of turns.

In some implementations, the guide assembly is configured to position, along a surface of the tissue, the guide rail in the guide arrangement.

In some implementations, the driver is configured to, while the guide rail in the guide arrangement is positioned along the surface of the tissue, anchor the implant along the tissue by screwing the helical member along the guide rail and the tissue such that part of each turn of the helical member becomes embedded within the tissue, and another part of each turn lies above a surface of the tissue.

In some implementations, the implant includes the helical member.

In some implementations, the implant includes a suture.

In some implementations, the driver is configured to, while the guide rail in the guide arrangement is positioned along the surface of the tissue, stitch the suture along the tissue by screwing the helical member along the guide rail and the tissue, such that the suture defines multiple turns.

In some implementations, the implant includes a tensile member that extends through each of the turns of the suture. In some implementations, the system further includes a tensioning tool that is adapted to adjust a dimension of the tissue by tensioning the tensile member such that the tensile member pulls on each of the turns of the suture.

In some implementations, the guide frame has a longitudinal axis, and is intracardially expandable toward an expanded state by being expanded radially away from the longitudinal axis.

In some implementations, in the guide arrangement, the guide rail extends distally through the interior of the guide frame, and out of the guide frame at the exit site to curve around the exterior of the guide frame and the longitudinal axis.

In some implementations, the delivery assembly further includes a tube, the guide rail extending through the tube.

In some implementations, in the guide arrangement: (a) the tube extends distally through the interior of the guide frame, and out of the guide frame at the exit site, (b) within the tube, the guide rail extends distally through the interior of the guide frame and out of the guide frame at the exit site, and/or (c) at the exterior of the guide frame, the guide rail exits the tube to lie, exposed from the tube, around the exterior of the guide frame.

In some implementations, the tube has a distal section that exits the guide frame at the exit site, and at least the distal section of the tube is a flexible sleeve.

In some implementations, the guide assembly includes a control shaft, a distal end of the control shaft being attached to the guide frame in a manner that facilitates transluminal control of the guide frame via the control shaft.

In some implementations, the guide rail extends distally from the control shaft, into the interior of the guide frame.

In some implementations, the guide rail extends, from an extracorporeal portion thereof, distally through the control shaft, to the interior of the guide frame.

In some implementations, the implant is transluminally advanceable towards the tissue: (a) over and along the guide rail, distally through the control shaft, and/or (b) out of the control shaft into the interior of the guide frame over and along the guide rail, exiting the guide frame at the exit site, and along the guide rail to the tissue.

In accordance with some implementations, an apparatus (which can be used with a tissue, e.g., of a living subject or of a simulation), the apparatus including an implant and/or a driver.

In some implementations, the implant includes a helical member that defines: (i) a sharpened distal tip, and/or (ii) a proximal end, the helical member having a thickness that is greater toward the proximal end than toward the distal tip.

In some implementations, the driver is configured to screw the implant along the tissue, distal tip first, by applying torque to the proximal end of the helical member such that part of each turn of the helical member becomes embedded within the tissue, and another part of each turn lies above a surface of the tissue.

In accordance with some implementations, a method (which can be used with a tissue, e.g., of a living subject or of a simulation) including transluminally positioning a guide rail along a surface of the tissue.

In some implementations, the method further includes using a flexible helical member, stitching a suture along the tissue by advancing the helical member helically along the guide rail, such that the suture defines a series of turns along the tissue, with part of each turn embedded within the tissue, and another part of each turn above the surface of the tissue.

In some implementations, the method further includes subsequently, adjusting a dimension of the tissue using a tensile member that is disposed along the surface of the tissue and that extends through each of the turns of the suture, by tensioning the tensile member such that the tensile member pulls on each of the turns of the suture.

In some implementations, stitching the suture along the tissue includes stitching the suture along the tissue while the tensile member is disposed along a lumen of the guide rail, and the method further includes retracting the guide rail proximally out from the series of turns, leaving the tensile member exposed within the series of turns.

In some implementations, stitching the suture along the tissue includes using the helical member to stitch the suture along the tissue while the suture is alongside the helical member.

In some implementations, the helical member is a hollow helical needle defining a channel therethrough, and stitching the suture along the tissue includes screwing the helical member along the tissue while the suture is disposed within the channel.

In some implementations, adjusting the dimension of the tissue using the tensile member includes tensioning the tensile member such that the tensile member reshapes each turn of the series of turns.

In some implementations, tensioning the tensile member such that the tensile member reshapes each turn of the series of turns includes tensioning the tensile member such that each turn of the series of turns transitions from a rounder shape to a more oval shape.

In some implementations, the tissue is tissue of an annulus of a valve of a heart, the annulus circumscribing an orifice of the valve.

In some implementations, stitching the suture along the tissue includes stitching the suture into and along tissue of the annulus, along the guide rail.

In some implementations, adjusting the dimension of the tissue using the tensile member includes tensioning the tensile member to reduce a dimension of the annulus.

In some implementations, adjusting the dimension of the tissue using the tensile member includes tensioning the tensile member such that the tensile member becomes suspended over the valve orifice.

In some implementations, stitching the suture along the tissue includes stitching the suture along the tissue while the suture is attached to an exterior of the helical member.

In some implementations, the method further includes detaching the suture from the helical member once the suture is stitched along the tissue.

In some implementations, the suture is attached to a distal end portion of the helical member, and stitching the suture along the tissue includes stitching the suture along the tissue by withdrawing the helical member from the tissue such that the suture is drawn into and along the tissue by the withdrawal of the helical member.

In some implementations, stitching the suture along the tissue includes stitching the suture along the tissue such that the helical member becomes temporarily stitched along the tissue.

In some implementations, the method further includes unstitching the helical member from the tissue by retracting the helical member helically, leaving the suture stitched along the tissue with the tensile member extending along an interior of the series of turns.

In some implementations, stitching the suture along the tissue and the tensile member includes stitching the suture along the tissue and the tensile member such that the tensile member extends along an interior of the series of turns.

In some implementations, stitching the suture along the tissue includes stitching the suture along the tissue such that the suture defines a helix that is disposed in a curved path along the tissue, the curved path having a radius of curvature.

In some implementations, adjusting the dimension of the tissue using the tensile member includes tensioning the tensile member to adjust the dimension of the tissue by reducing the radius of curvature of the curved path.

In some implementations, stitching the suture along the tissue includes stitching the suture along the tissue such that the suture defines a helix that is disposed in a curved path along the tissue, the curved path having a length.

In some implementations, adjusting the dimension of the tissue using the tensile member includes tensioning the tensile member to adjust the dimension of the tissue by reducing the length of the curved path.

In accordance with some implementations, a system (which can be used with a real or simulated tissue of a heart, e.g., of a living subject or of a simulation), the system including a suture, a tensile member; and/or a delivery assembly.

In some implementations, the delivery assembly includes a guide assembly, including a guide rail, the guide assembly configured to transluminally advance the guide rail to the heart, and to position the guide rail into a guide arrangement along a surface of the tissue.

In some implementations, the delivery assembly includes a flexible helical member.

In some implementations, the delivery assembly includes a driver configured to, while the guide rail is in the guide arrangement, stitch the suture along the tissue by advancing the flexible helical member helically along the guide rail, such that the suture defines a series of turns along the tissue, with part of each turn embedded within the tissue, another part of each turn above the surface of the tissue, and the tensile member disposed along the surface of the tissue, extending through the series of turns.

In some implementations, the tissue is tissue of an annulus of a valve of the heart, the annulus circumscribing an orifice of the valve.

In some implementations, the driver is configured to stitch the suture into and along tissue of the annulus, circumferentially around the valve orifice.

In some implementations, the driver is configured to transluminally advance the helical member towards the heart over and along the guide rail.

In some implementations, the guide assembly further includes a guide frame, intracardially expandable toward an expanded state.

In some implementations, the guide assembly is configured to position the guide rail into the guide arrangement circumferentially around at least part of the guide frame.

In some implementations, the guide assembly further includes multiple fasteners that are intracardially tightenable, from a proximal extracorporeal portion of the delivery assembly, in a manner that draws the guide rail into the guide arrangement around at least part of the guide frame.

In some implementations, the guide frame has a longitudinal axis, and is intracardially expandable toward an expanded state by being expanded radially away from the longitudinal axis.

In some implementations, in the guide arrangement, the guide rail extends distally through an interior of the guide frame, and out of the guide frame at an exit site to curve around an exterior of the guide frame and the longitudinal axis.

In some implementations, the system further includes a tensioning tool. In some implementations, the tensioning tool is configured to adjust a dimension of the tissue by tensioning the tensile member such that the tensile member pulls on each of the turns of the suture.

In some implementations, the driver is configured to stitch the suture such that the suture becomes disposed in a helix that is disposed in a curved path along the tissue, the curved path having a radius of curvature.

In some implementations, the tensioning tool is configured to adjust a dimension of the tissue by tensioning the tensile member such that the radius of curvature of the curved path is reduced.

In some implementations, the driver is configured to stitch the suture such that the suture becomes disposed in a helix that is disposed in a curved path along the tissue, the curved path having a length.

In some implementations, the tensioning tool is configured to adjust a dimension of the tissue by tensioning the tensile member such that the length of the curved path is reduced.

In some implementations, the tensile member is disposed along a lumen of the guide rail, and the delivery assembly is configured to retract the guide rail proximally out from the series of turns, leaving the tensile member exposed along the series of turns.

In some implementations, the driver is configured to stitch the suture along the tissue such that the helical member becomes temporarily stitched along the tissue. In some implementations, the delivery assembly is configured to leave the suture stitched along the tissue with the tensile member extending along the series of turns by: (i) unstitching the helical member from the tissue by retracting the helical member helically, and/or (ii) retracting the guide rail linearly.

In some implementations, the driver is configured to stitch the suture along the tissue while the suture is attached to the helical member.

In some implementations, the suture is detachable from the helical member once the suture is stitched along the tissue.

In some implementations, the suture is attached to a distal end portion of the helical member, and the driver is adapted to stitch the suture along the tissue by withdrawing the driver from the tissue such that the suture is drawn into and along the tissue by the withdrawal of the driver.

In some implementations, the driver is adapted to stitch the suture into the tissue alongside the helical member.

In some implementations, the helical member is a hollow helical needle defining a channel therethrough, and the driver is configured to stitch the suture along the tissue by screwing the helical member along the tissue while the suture is disposed within the channel.

In some implementations, the helical member is configured to be unscrewed from the tissue, leaving the suture stitched along the tissue.

In accordance with some implementations, a system (which can be used with tissue of a heart, e.g., of a living subject or of a simulation) includes: (i) a suture; (ii) a tensile member; and/or (iii) a delivery assembly.

In some implementations, the delivery assembly includes a guide assembly, including a guide rail, the guide assembly configured to transluminally advance the guide rail to the heart, and to position the guide rail into a guide arrangement along a surface of the tissue.

In some implementations, the delivery assembly includes a flexible helical member.

In some implementations, the delivery assembly includes a driver, coupled to the flexible helical member, wherein the delivery assembly is configured to (i) arrange the suture in a series of turns stitched along the tissue by, while the guide rail is in the guide arrangement, the driver advancing the flexible helical member helically along the guide rail, and (ii) dispose the tensile member along the surface of the tissue, extending through the series of turns.

In accordance with some implementations, a system (which can be used with tissue of a heart, e.g., of a living subject or of a simulation), the system including an implant, and/or a delivery assembly.

In some implementations, the implant includes a suture.

In some implementations, the delivery assembly includes a guide assembly, having a distal part that is transluminally advanceable to the heart while in a delivery state, the guide assembly including: (i) a guide frame, intracardially expandable toward an expanded state, and/or (ii) a guide rail, the guide assembly configured to position the guide rail into a guide arrangement circumferentially around at least part of the guide frame.

In some implementations, the delivery assembly includes a flexible helical member defining multiple turns, configured to stitch the suture along the tissue by advancing the suture helically over and along the guide rail, while the guide rail is in the guide arrangement, such that the suture defines a series of turns along the tissue, with part of each turn of the suture embedded within the tissue, and another part of each turn of the suture lying above a surface of the tissue.

In some implementations, the implant further includes a tensile member. In some implementations, the flexible helical member is configured to stitch the suture along the tissue such that the suture defines a series of turns along the tissue, with the tensile member extending along an interior of the series of turns. In some implementations, the implant is configured such that tensioning of the tensile member adjusts a dimension of the tissue by the tensile member pulling on the suture.

In some implementations, the flexible helical member is configured to stitch the suture along the tissue by advancing the suture helically over and along the guide rail while the guide rail is in the guide arrangement and the tensile member is disposed along a lumen of the guide rail.

In some implementations, the guide assembly includes multiple fasteners that are intracardially tightenable via an extracorporeal portion of the delivery assembly, in a manner that draws the guide rail into the guide arrangement around at least part of the guide frame.

In accordance with some implementations, a system, including a tube, a first strip, and/or a second strip.

In some implementations, the tube is transluminally advanceable to a body orifice (e.g., a real or simulated body orifice), the tube defining a flex zone in which cuts in the tube confer flexibility to the tube.

In some implementations, the first strip that has greater tensile strength than the flex zone, a first end of the first strip being attached to the tube distally from the flex zone, and a second end of the first strip being attached to the tube proximally from the flex zone, such that, in a relaxed state of the tube, the first strip lies slack alongside the flex zone.

In some implementations, the second strip that has greater tensile strength than the flex zone, a first end of the second strip being attached to the tube distally from the flex zone, and a second end of the second strip being attached to the tube proximally from the flex zone, such that, in a relaxed state of the tube, the second strip lies slack alongside the flex zone.

In some implementations, each of the first end of the first strip and the second end of the first strip are attached to the tube by welding.

In some implementations, the tube is a hypotube.

In some implementations, pulling the tube proximally tensions each of the first strip and the second strip, such that each strip presses against the flex zone.

In accordance with some implementations, a method including manufacturing a guide frame usable and/or for use with a real or simulated cardiovascular system by obtaining a frame defined by a braid arrangement that can comprise and/or is formed from multiple wires braided together, the frame having a proximal part and a distal part, wherein each of the wires has a first end at the proximal part of the frame and helically extends distally along the frame.

In some implementations, the method further includes, at the distal part of the frame, invaginating the braid arrangement such that a second end of each of the wires becomes disposed in an interior of the frame and the distal part of the frame becomes atraumatically contoured.

In some implementations, the method further includes binding the second ends of the wires together in the interior of the frame.

In some implementations, the method further includes, subsequently to manufacturing the guide frame, coupling a guide rail along an exterior of the guide frame using multiple fasteners that extend out of the guide frame to the guide rail.

In accordance with some implementations, a method including manufacturing a guide frame usable and/or for use with a real or simulated cardiovascular system by obtaining a frame defined by a braid arrangement, the frame having a proximal part and a distal part.

In some implementations, the method further includes, at the proximal part of the frame, invaginating the braid arrangement to form an invagination such that the proximal part of the frame becomes contoured and disposed within an interior of the frame.

In some implementations, the method further includes heat setting the proximal part to set the invagination such that expanding the guide frame via a flexible control shaft having a distal end that is attached to the invagination disposes the control shaft within the invagination.

In some implementations, the method further includes, subsequently to manufacturing the guide frame, coupling a guide rail along an exterior of the guide frame using multiple fasteners that extend out of the guide frame to the guide rail.

In accordance with some implementations, a system and/or an apparatus (which can be used in a real or simulated cardiovascular system of a subject), the apparatus including a guide frame and/or a flexible control shaft.

In some implementations, the guide frame has an invaginating part, and being expandable into an expanded state in which the invaginating part forms an invagination.

In some implementations, the flexible control shaft has a distal end that is coupled to the guide frame at the invaginating part, and configured to: (i) transluminally advance the frame through the cardiovascular system, and/or (ii) expand the frame into its expanded state within the cardiovascular system such that, in the expanded state, the distal end of the control shaft is disposed within the invagination.

In accordance with some implementations, an apparatus usable and/or for use in a real or simulated cardiovascular system of a subject, the apparatus including: a frame and/or a control shaft.

In some implementations, the frame is defined by a braid arrangement that can comprise and/or is formed from multiple wires braided together, the frame having a proximal part and a distal part, wherein: (i) each of the wires has a first end at the proximal part of the frame and helically extends distally along the frame, (ii) at the distal part of the frame, the braid arrangement is invaginated such that a second end of each of the wires is disposed in an interior of the frame and the distal part of the frame is atraumatically contoured, and/or (iii) the second ends of the wires are bound together in the interior of the frame.

In some implementations, the flexible control shaft is coupled to the proximal part of the frame and configured to: (a) transluminally advance the frame distally through the cardiovascular system, and/or (b) expand the frame within the cardiovascular system.

In accordance with some implementations, a system and/or an apparatus (which can be used with tissue of a heart, e.g., of a living subject or of a simulation) includes a helical member defining multiple turns, and/or a driver, configured to screw the helical member along the tissue.

In some implementations, the driver is configured to screw the helical member along the tissue such that part of each turn of the helical member becomes embedded within the tissue, and another part of each turn lies above a surface of the tissue.

In some implementations, the apparatus further includes an extracorporeal portion, electrically connected to the helical member via the driver, and adapted to alter the tissue by applying electrical energy to the tissue via the helical member while the helical member remains screwed along the tissue, the driver being configured to unscrew the helical member from the tissue, leaving the tissue altered.

In some implementations, the helical member includes nitinol.

In some implementations, the tissue is tissue of an annulus of a valve of the heart. In some implementations, the driver is configured to screw the helical member along the annulus.

In some implementations, the helical member is heat set to contract to a predetermined shape upon application of the electrical energy to the helical member.

In some implementations, the helical member is constructed from a shape-memory material.

In some implementations, the extracorporeal portion includes a power source, configured to provide the electrical energy.

In some implementations, the extracorporeal portion: (i) includes a terminal, configured to be electrically and mechanically connected to a power source, and/or (ii) is configured to derive the electrical energy from the power source.

In some implementations, the apparatus further includes a guide assembly including a guide rail, and the driver is configured to screw the helical member along the tissue by advancing the helical member helically over and along the guide rail while the guide rail is disposed along the tissue.

In some implementations, the guide assembly further includes a guide frame, and multiple fasteners that are intracardially tightenable via the extracorporeal portion in a manner that draws the guide rail along the tissue, around at least part of the guide frame.

In accordance with some implementations, a system and/or an apparatus (which can be used with tissue of a heart, e.g., of a living subject or of a simulation) includes a helical member defining multiple turns, and/or a guide assembly.

In some implementations, the guide assembly has a distal part that is transluminally advanceable to the heart while in a delivery state. In some implementations, the guide assembly includes (i) a guide frame, intracardially expandable toward an expanded state, and/or (ii) a guide rail, intracardially arrangeable into a guide arrangement around at least part of the guide frame, the guide assembly being positionable within the heart such that the guide rail, in the guide arrangement, is disposed along the tissue.

In some implementations, the apparatus further comprises a driver, configured to screw the helical member along the guide rail and the tissue while the guide rail is in the guide arrangement.

In some implementations, the apparatus further comprises an extracorporeal portion, electrically connected to the helical member via the driver, and adapted to alter the tissue by applying electrical energy to the helical member while the helical member remains screwed along the tissue.

In some implementations, the helical member includes nitinol.

In some implementations, the helical member is heat set to contract to a predetermined shape upon application of the electrical energy to the helical member.

In some implementations, the extracorporeal portion is adapted to alter the tissue by applying the electrical energy to the tissue via the helical member.

In some implementations, the helical member is constructed from a shape-memory material.

In some implementations, the extracorporeal portion includes a power source, configured to provide the electrical energy.

In some implementations, the extracorporeal portion: (i) includes a terminal, configured to be electrically and mechanically connected to a power source, and/or (ii) is configured to derive the electrical energy from the power source.

In some implementations, the apparatus includes an implant including the helical member. In some implementations, the implant further includes a lock, lockable to the implant to maintain the tissue altered.

In some implementations, the helical member is configured to contract toward a contracted state responsively to the electrical energy. In some implementations, (i) the implant further includes a tensile member, (ii) the driver is configured to screw the helical member along the tissue such that the tensile member extends along an interior of the series of turns, and/or (iii) the implant is configured such that, while the helical member remains contracted, the tensile member is tensionable to a tensioned state in a manner that maintains the helical member in the contracted state.

In some implementations, the lock is lockable to the tensile member to maintain the tensile member in the tensioned state.

In some implementations, the helical member is configured to contract toward a contracted state responsively to the electrical energy. In some implementations, the lock is lockable to the helical member, to maintain the helical member in the contracted state.

In accordance with some implementations, a method usable with a heart (e.g., living or simulated) includes screwing a flexible helical member along the tissue, such that the helical member defines a series of turns along the tissue, with part of each turn embedded within the tissue, and another part of each turn above a surface of the tissue.

In some implementations, the method further includes subsequently altering the tissue by applying electrical energy to the helical member while the helical member remains screwed along the tissue.

In some implementations, the method further includes subsequently unscrewing the helical member from the tissue, leaving the tissue altered.

In some implementations, applying electrical energy to the helical member includes applying electrical energy to the helical member from an extracorporeal power source that is electrically connected to the helical member.

In some implementations, altering the tissue by applying the electrical energy includes irreversibly altering the tissue by applying the electrical energy.

In accordance with some implementations, a method usable with a heart (e.g., living or simulated) includes transluminally advancing a guide frame to the heart while the guide frame is secured to a guide rail.

In some implementations, the method further includes expanding the guide frame within the heart.

In some implementations, the method further includes drawing the guide rail into a guide arrangement around at least a part of the guide frame.

In some implementations, the method further includes, while the guide rail remains in the guide arrangement, screwing a flexible helical member along the tissue, such that the helical member defines a series of turns along the tissue, with part of each turn embedded within the tissue, and another part of each turn above a surface of the tissue.

In some implementations, the method further includes subsequently altering the tissue by applying electrical energy to the helical member while the helical member remains screwed along the tissue.

In some implementations, altering the tissue by applying the electrical energy includes applying the electrical energy such that the helical member contracts toward a contracted state in which the turns of the helical member are closer to each other, thereby altering the tissue. In some implementations, the method further includes, while the electrical energy is applied to the helical member, locking the helical member in the contracted state, such that the tissue remains altered.

In some implementations, locking the helical member in the contracted state includes: (i) tensioning a tensile member that is disposed along the surface of the tissue and that extends through each of the turns of the helical member, and/or (ii) locking the tension in the tensile member by applying a lock to the tensile member, to maintain the helical member in the contracted state.

In some implementations, locking the helical member in the contracted state includes mechanically locking the helical member in the contracted state.

In accordance with some implementations, a system and/or an apparatus (which can be used with a cardiovascular system, e.g., of a living subject or of a simulation) includes an extracorporeal handle, a frame having a proximal part and a distal part, a control shaft that extends from the extracorporeal handle, and is coupled to the proximal part of the frame, and/or a plurality of actuator wires.

In some implementations, the frame is transluminally advanceable into the cardiovascular system. In some implementations, the plurality of actuator wires: (i) extend from the control shaft, (ii) weave distally along at least part of the frame, (iii) are attached to a distal part of the frame, and/or (iv) are actuatable from the handle to: (a) radially expand the frame towards an expanded state, and/or (b) pivot the frame with respect to the control shaft independently of the expansion of the frame.

In some implementations, the frame is a guide frame. In some implementations, the system further includes a guide rail, drawable into a guide arrangement along at least part of the guide frame.

In some implementations, in the guide arrangement, the guide rail lies around a midsection of the guide frame, the midsection disposed longitudinally between an upstream section of the frame and a downstream section of the frame.

In some implementations, each of the actuator wires extends, from the control shaft, distally through an interior of the upstream section of the guide frame, and weavingly along the downstream section of the guide frame.

In some implementations, in the expanded state of the guide frame, at least part of the upstream section is wider than the downstream section.

In some implementations, in the expanded state of the guide frame, at least part of the upstream section is wider than the midsection.

In some implementations, the frame is pivotable with respect to the control shaft via differential tensioning of the actuator wires.

In some implementations, the extracorporeal handle: (i) includes at least one controller to which the actuator wires are operatively coupled, and/or (ii) is configured to differentially tension the actuator wires via actuation of the at least one controller.

In some implementations, the frame is configured to radially expand responsively to balanced tension in the actuator wires. In some implementations, the extracorporeal handle is configured to apply the balanced tension to the actuator wires.

In some implementations, the at least one controller is configured with: (i) a first actuation mode that applies the balanced tension to the actuator wires, and/or (ii) a second actuation mode that applies the differential tension to the actuator wires.

In accordance with some implementations, a method usable with a cardiovascular system (e.g., living or simulated) includes using a handle that is attached to a frame via a control shaft, transluminally advancing the frame to the cardiovascular system, while a plurality of actuator wires extend, from the control shaft to a distal part of the frame where each actuator wire is attached.

In some implementations, the method further includes, from the handle, actuating the plurality of actuator wires to: (i) radially expand the frame within the cardiovascular system, and/or (ii) pivot the frame with respect to the control shaft, independently of the expansion of the frame.

In some implementations, actuating the plurality of actuator wires to pivot the frame with respect to the control shaft includes actuating the plurality of actuator wires via differential tensioning of the actuator wires to pivot the frame with respect to the control shaft.

In some implementations, actuating the plurality of actuator wires to radially expand the frame includes actuating the plurality of actuator wires via application of balanced tension to the actuator wires to radially expand the frame.

In some implementations, actuating the plurality of actuator wires to radially expand the frame includes actuating the plurality of actuator wires to radially expand the frame prior to actuating the actuator wires to pivot the frame.

In some implementations, actuating the plurality of actuator wires to radially expand the frame includes actuating the plurality of actuator wires to radially expand the frame subsequently to actuating the actuator wires to pivot the frame.

In some implementations, the frame is a guide frame that is a component of a guide assembly. In some implementations, the guide assembly further includes a guide rail, drawable into a guide arrangement along at least part of the guide frame.

In some implementations, in the guide arrangement, the guide rail lies around a midsection of the guide frame, the midsection disposed longitudinally between an upstream section of the frame and a downstream section of the frame.

In some implementations, each of the actuator wires extends, from the control shaft, distally through an interior of the upstream section of the guide frame, and weavingly along the downstream section of the guide frame. In some implementations, actuating the actuator wires includes actuating the actuator wires that weave along the downstream section of the guide frame.

In some implementations, expanding the guide frame includes expanding the guide frame such that at least part of the upstream section is wider than the downstream section.

In some implementations, expanding the guide frame includes expanding the guide frame such that at least part of the upstream section is wider than the midsection.

In accordance with some implementations, a system and/or an apparatus (which can be used with tissue of a heart, e.g., of a living subject or of a simulation) includes an implant, and/or a delivery assembly. In some implementations, delivery assembly includes a driver, configured to advance the implant along the tissue. In some implementations, the delivery assembly includes a guide assembly, having a distal part that is transluminally advanceable to the heart while in a delivery state.

In some implementations, the guide assembly includes: (i) a guide frame, intracardially expandable toward an expanded state, (ii) a guide rail, intracardially arrangeable into a guide arrangement around at least part of the guide frame, the driver being adapted to advance the implant along the tissue by advancing the implant along the guide rail in the guide arrangement, and/or (iii) a rider, slidably mounted on the guide rail such that, as the driver advances the implant along the tissue, a leading end of the implant pushes the rider along the guide rail, while the rider shields the guide frame from the leading end of the implant.

In some implementations, the implant includes a helical member defining a sharpened tip at the leading end. In some implementations, the rider defines a lobe that, as the implant pushes the rider along the guide rail, the lobe remains disposed between the leading end and the guide frame, thereby shielding the guide frame from the sharpened tip.

In some implementations, the lobe is rotationally locked with respect to the guide rail.

In some implementations, the lobe is rotationally locked with respect to the guide rail via keying between the rider and the guide rail.

In accordance with some implementations, a system and/or an apparatus (which can be used with tissue of a heart, e.g., of a living subject or of a simulation) includes a helical member defining a plurality of turns, and/or a delivery assembly.

In some implementations, the delivery assembly includes a guide assembly, having a distal part that is transluminally advanceable to the heart while in a delivery state, the guide assembly including: (a) a guide frame, intracardially expandable toward an expanded state, (b) a guide rail, intracardially arrangeable into a guide arrangement around at least part of a midsection of the guide frame, and/or (c) an expandable shield that is disposed around at least the part of the midsection such that: (I) expanding the guide frame towards the expanded state expands the midsection and the shield, and/or (II) in the guide arrangement, the shield is disposed between the guide rail and the guide frame at the midsection.

In some implementations, the delivery assembly includes a driver, configured to helically advance the helical member along the guide rail in the guide arrangement.

In some implementations, in the expanded state of the guide frame, the guide frame is at least twice as large as it is in the delivery state of the guide assembly. In some implementations, in the expanded state of the guide frame, the shield covers at least a majority of a circumference of the guide frame.

In some implementations, the shield includes an elastic material.

In some implementations, the shield is defined by a fabric.

In some implementations, the shield is defined by a film.

In some implementations, the shield is defined by a net.

In some implementations, the shield has a hypotube-type structure.

In some implementations, the shield is constructed from an array of interconnected struts and tessellated cells.

In some implementations, the shield is a ribbon that, in the delivery state of the guide assembly, is wrapped around the guide frame, and expanding the guide frame towards the expanded state causes the ribbon to slide over itself in a manner that reduces the wrapping around the guide frame.

In some implementations, the shield is defined by multiple ribbons distributed circumferentially around the midsection.

In some implementations, in the guide arrangement, each ribbon contacts its neighboring ribbons, such that the ribbons collectively cover the midsection.

In some implementations, each ribbon is polymeric.

In some implementations, each ribbon is metallic.

In some implementations, in the delivery state, the ribbons are imbricated around the midsection. In some implementations, expanding the guide frame towards the expanded state expands the shield by the ribbons sliding over each other while collectively covering the midsection.

In some implementations, in the expanded state, the ribbons remain imbricated around the midsection.

In some implementations, in the expanded state, the ribbons are arranged edge-to-edge around the midsection.

In accordance with some implementations, a method usable with a heart (e.g., living or simulated) includes transluminally advancing a guide frame to the heart while: (i) the guide frame is secured to a guide rail via multiple fasteners that extend out of the guide frame to the guide rail, and/or (ii) an expandable shield is disposed around a midsection of the guide frame.

In some implementations, the method further includes expanding the guide frame within the heart such that the shield expands therealong.

In some implementations, the method further includes drawing the guide rail into a guide arrangement around at least a part of the midsection by tightening at least one of the multiple fasteners such that the shield becomes disposed between the guide rail and the guide frame.

In accordance with some implementations, a system for use with tissue of a heart, the system including a helical member defining a plurality of turns, and/or a delivery assembly.

In some implementations, the delivery assembly includes a guide assembly, having a distal part that is transluminally advanceable to the heart while in a delivery state, the guide assembly including: (i) a guide frame, intracardially expandable toward an expanded state, and/or (ii) a guide rail, intracardially arrangeable into a guide arrangement around at least part of the guide frame, and defining an external thread.

In some implementations, the delivery assembly further includes a driver, configured to helically advance the helical member threadedly along the external thread while the guide rail is in the guide arrangement.

In some implementations, the external thread defines a groove. In some implementations, the driver is configured to screw the helical member helically along the thread while the helical member is recessed within the groove.

In some implementations, the guide rail defines a central guide-rail axis, and has a tissue-facing surface that, along the guide rail, is disposed closer than the external thread to the central guide-rail axis. In some implementations, in the guide arrangement, the external thread faces medially toward the guide frame, and the tissue-facing surface faces radially away from the guide frame.

In some implementations, the tissue-facing surface is unthreaded, and runs parallel with the external thread.

In some implementations, the tissue-facing surface is substantially flat.

In some implementations, the tissue-facing surface is concave.

In accordance with some implementations, a method for use with a tissue of a heart, the method including transluminally advancing a guide frame to the heart while the guide frame is secured to a guide rail that defines an external thread. In some implementations, the method further includes positioning the guide rail into a guide arrangement around at least part of the guide frame, such that at least part of the guide rail becomes positioned along the tissue.

In some implementations, the method further includes while the guide rail is in the guide arrangement, intracardially screwing a flexible helical member into the tissue by advancing the helical member threadedly along the external thread, such that the helical member defines a series of turns along the tissue, with part of each turn embedded within the tissue, and another part of each turn above a surface of the tissue.

In some implementations, the external thread defines a groove, and/or advancing the helical member threadedly along the external thread includes advancing the helical member threadedly along the external thread while the helical member is recessed within the groove.

In some implementations, the guide rail defines a central guide-rail axis, and has a tissue-facing surface that, along the guide rail, is disposed closer than the external thread to the central guide-rail axis.

In some implementations, positioning the guide rail into the guide arrangement around at least part of the guide frame includes positioning the guide rail into the guide arrangement around at least part of the guide frame such that the external thread faces medially toward the guide frame, and the tissue-facing surface faces radially away from the guide frame.

In some implementations, positioning the guide rail into the guide arrangement around at least part of the guide frame includes positioning the guide rail into the guide arrangement around at least part of the guide frame such that a crest of the external thread contacts the guide frame.

In some implementations, advancing the helical member threadedly along the external thread includes iteratively rotating the helical member threadedly such that, during each rotation of the helical member, a distal tip of the helical member: (i) leaves the groove toward the tissue as the distal tip reaches the tissue-facing surface, (ii) penetrates the tissue at the tissue-facing surface, (iii) exits the tissue toward the external thread, and/or (iv) re-enters the groove as the distal tip arrives returns to the external thread.

Any of the above method(s) and any methods of using the systems, assemblies, apparatuses, devices, etc. herein can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can optionally comprise computerized and/or physical representations.

Any of the above systems, assemblies, devices, apparatuses, components, etc. can be sterilized (e.g., with heat, radiation, ethylene oxide, hydrogen peroxide, etc.) to ensure they are safe for use with patients, and the methods herein can comprise (or additional methods comprise or consist of) sterilization of one or more systems, devices, apparatuses, components, etc. herein (e.g., with heat, radiation, ethylene oxide, hydrogen peroxide, etc.).

The present invention will be more fully understood from the following detailed description of implementations thereof, taken together with the drawings, in which:

### BRIEF DESCRIPTION OF THE DRAWINGS

FIGS. 1 and 2A-I are schematic illustrations of a system and use thereof, in accordance with some implementations;

FIGS. 3A-E are schematic illustrations that show, in further detail, some optional components and/or features of a guide assembly, in accordance with some implementations;

FIGS. 4A-B are schematic illustrations of a technique of positioning a guide rail of a guide assembly against an annulus of a valve, in accordance with some implementations;

FIG. 5 illustrates a helical member that has a variable thickness, in accordance with some implementations;

FIG. 6A-C are schematic illustrations of a guide assembly, in accordance with some implementations;

FIG. 7 shows a helical member is advanced through a flexible sleeve, in accordance with some implementations;

FIGS. 8A-B illustrate a method of delivering a helical member towards the annulus, in accordance with some implementations;

FIG. 9 is a schematic illustration of a driver, in accordance with some implementations;

FIGS. 10A-B, and 11 illustrate a distal portion of a shaft having reinforcing strips, in accordance with some implementations;

FIGS. 12A-B, and 13-14 illustrate various guide frames, in accordance with some implementations;

FIGS. 15A-C illustrate an implementation in which multiple actuator wires are used to both expand a guide frame, and to reorient the guide frame, in accordance with some implementations;

FIG. 16 illustrates an implementation in which multiple actuator wires are used that are woven along a downstream section of the guide frame, in accordance with some implementations;

FIGS. 17, 18A-B, and 19A-B illustrate various systems and methods for spacing a guide rail from a guide frame, in accordance with some implementations;

FIGS. 20-21, 22A-B, and 23 illustrate various implementations in which a guide assembly comprises a shield that is disposed around a midsection of the guide frame, in accordance with some implementations;

FIGS. 24-26 illustrates various features for reducing a likelihood of a helical member catching onto the guide frame, in accordance with some implementations;

FIGS. 27-28 illustrate various guide frames, in accordance with some implementations;

FIGS. 29A-C, 30A-B, 31, and 32A-B illustrate systems and methods of using a flexible helical member to stitch a suture of an implant along a tissue, in accordance with some implementations;

FIGS. 33A-E, 34A-B, 35A-B, 36A-B, 37, and 38 are schematic illustrations of systems and techniques for positioning a guide rail along a tissue, and for guiding a helical member along the tissue using the guide rail, in accordance with some implementations;

FIG. 39 is a schematic illustration of a guide assembly that comprises a guide rail having one or more electrodes thereon, in accordance with some implementations;

FIG. 40 is a schematic illustration showing a replacement heart valve having a plurality of electrodes disposed thereon, adapted to guide the positioning of the valve within the heart, in accordance with some implementations; and

FIGS. 41A-C and 42A-C illustrate systems and methods for using electrical energy to contract tissue of an annulus, using a helical member that is screwed along the tissue, in accordance with some implementations.

### DETAILED DESCRIPTION

The described systems, apparatuses, devices, methods, etc. should not be construed as limiting in any way. Instead, the present disclosure is directed toward all novel and nonobvious features and aspects of the various disclosed implementations and applications, alone and in various combinations and sub-combinations with one another. The disclosed systems, apparatuses, devices, methods, etc. are not limited to any specific aspect, feature, or combination thereof, nor do the disclosed systems, apparatuses, devices, methods, etc. require that any one or more specific advantages be present or problems be solved.

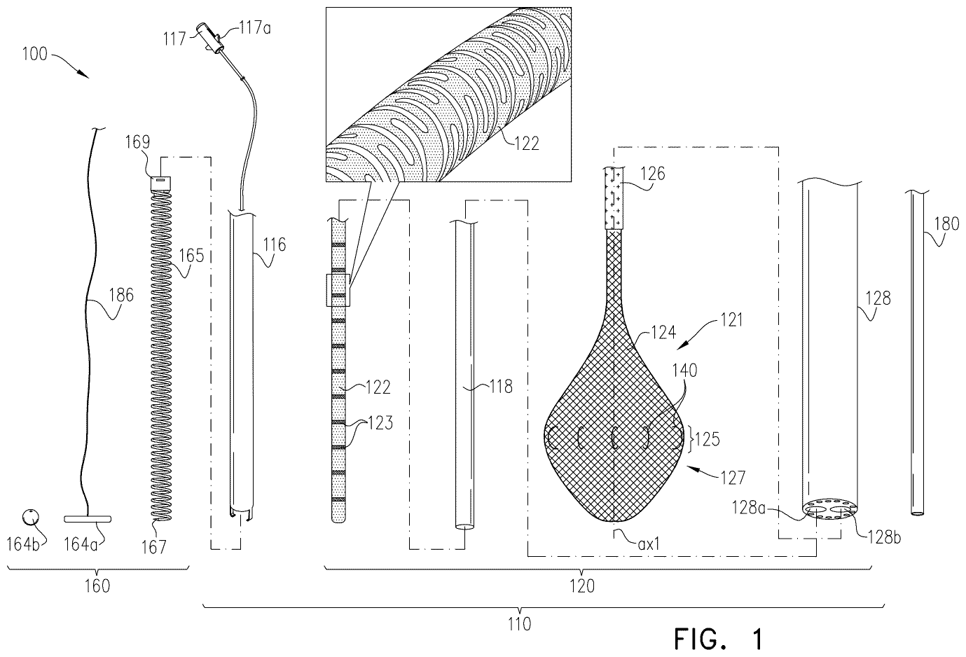

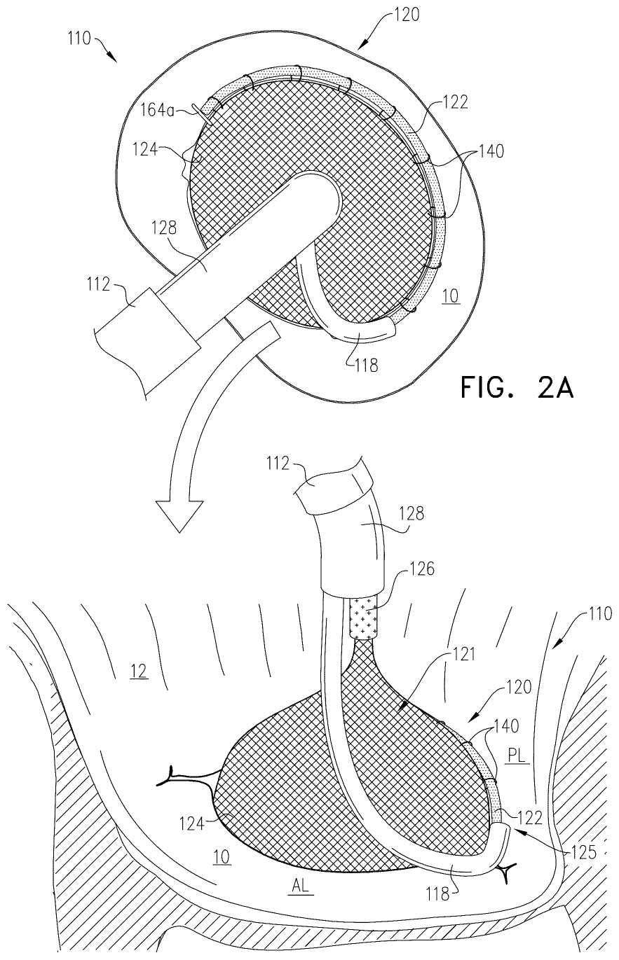

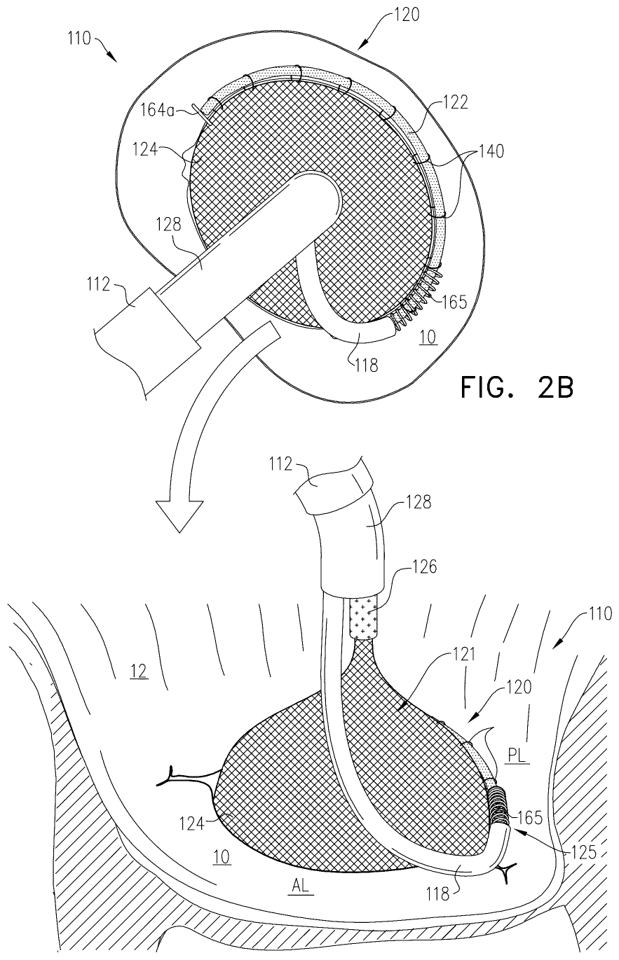

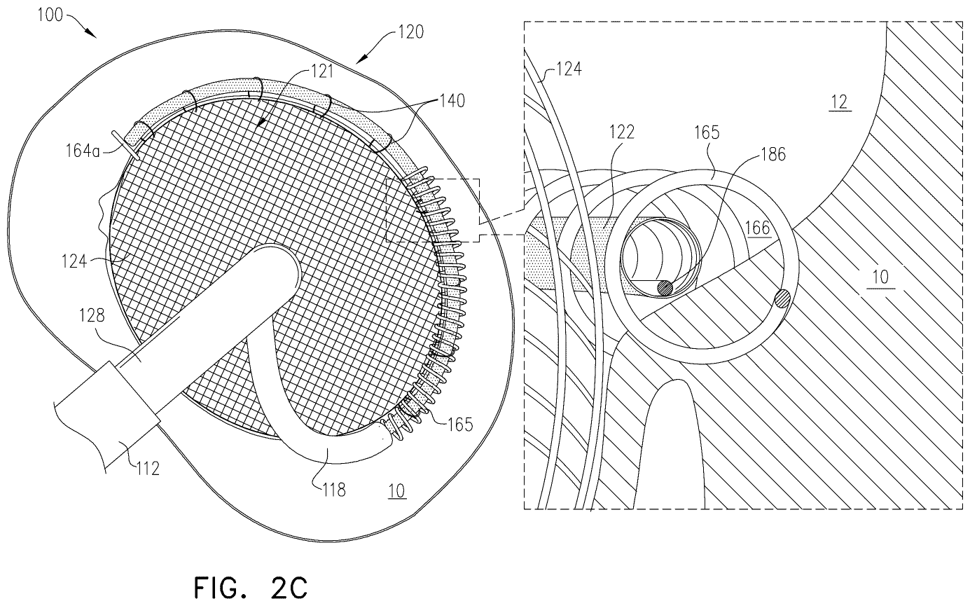

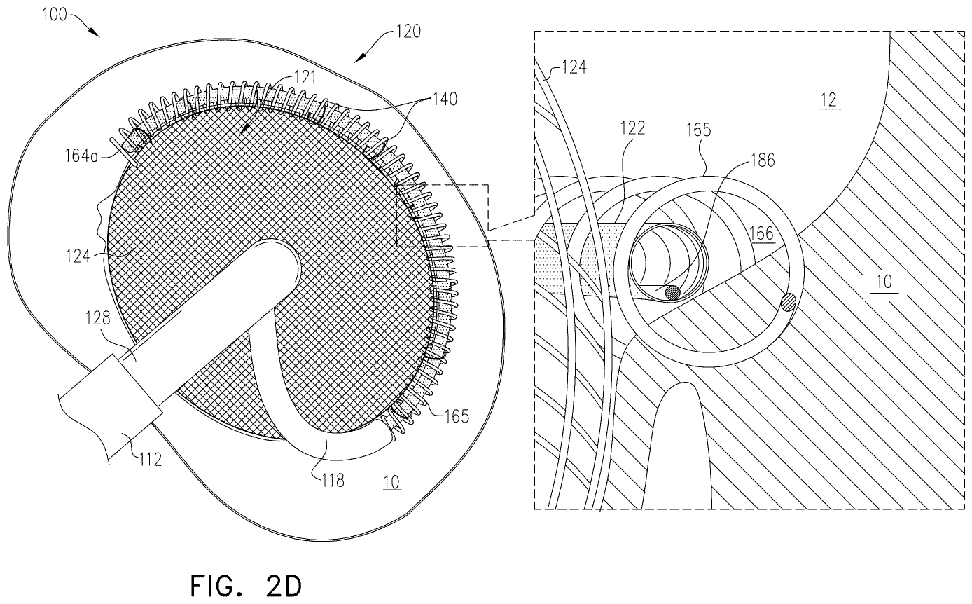

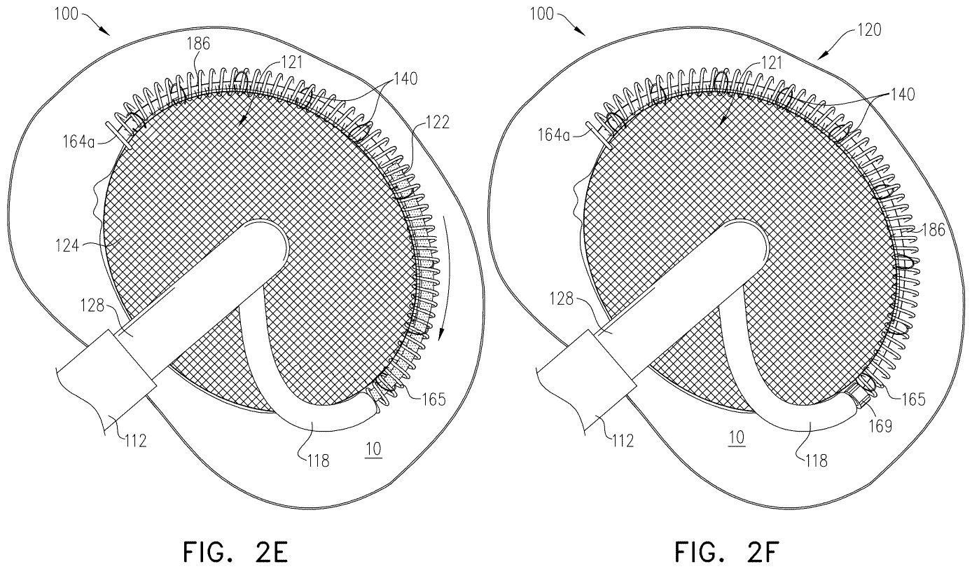

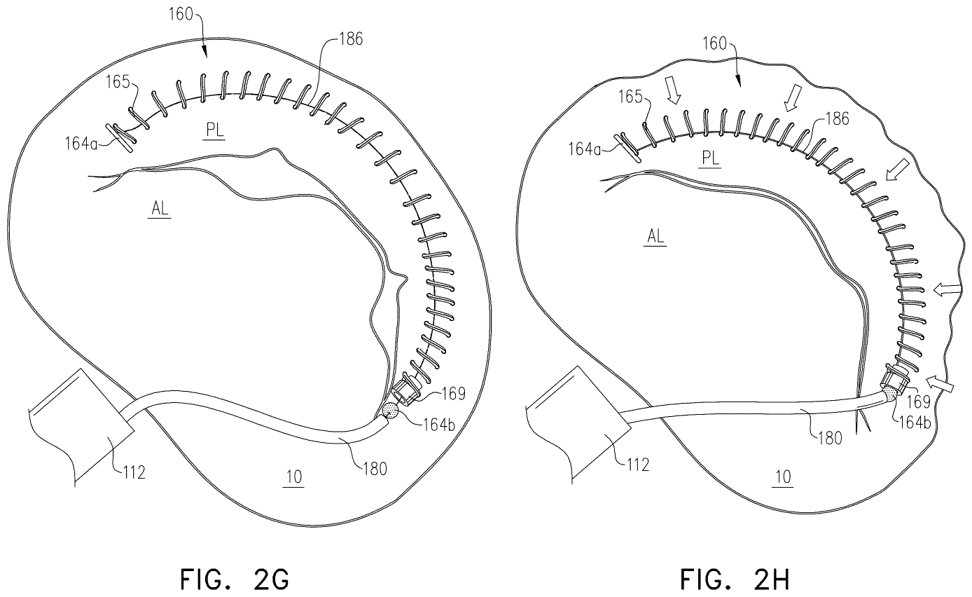

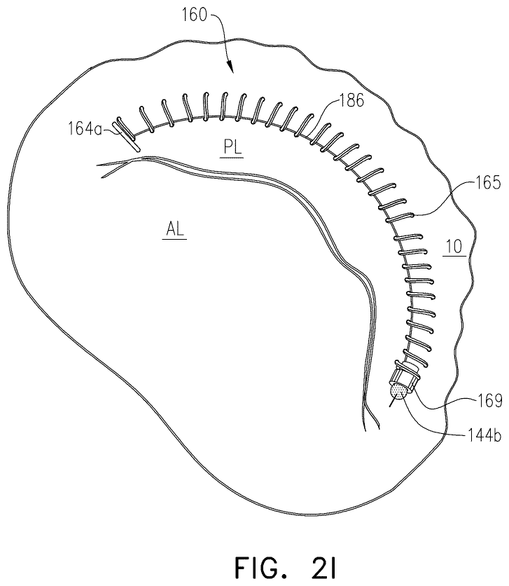

Reference is now made to FIGS. 1 and 2A-I, which are schematic illustrations of a system 100 and use thereof, in accordance with some implementations. System 100 comprises an implant 160, and a delivery assembly 110 that comprises a guide assembly 120 adapted to guide the implantation of the implant. System 100 can be used at an atrioventricular valve of a heart (e.g., of a subject, such as a living subject or a simulation, etc.), such as a mitral valve or a tricuspid valve.

In some implementations, implant 160 is adapted to reduce a dimension (e.g., a circumference) of a tissue (e.g., of an annulus 10 of a valve) of the heart. For example, implant 160 can be an annuloplasty implant, configured to reduce regurgitation of an atrioventricular valve of the heart.

FIG. 1 shows components of system 100 disassembled. FIGS. 2A-I show at least some steps in the use of system 1 to treat a valve of a heart. Although the valve shown is a mitral valve, the system and techniques can be used similarly at another valve of the heart, such as the tricuspid valve.

Implant 160 can comprise a helical member 165 (e.g., a coil), such as a helical tissue anchor, which defines a helix that extends around and along, and thereby defines, a central channel 166. Helical member 165 is adapted to be anchored along a tissue (e.g., a valve annulus 10), and to be subsequently axially contracted (e.g., compressed), in order to contract (e.g., compress) the tissue. For example, this contraction can be used to draw the tissue radially inwards to circumferentially reduce the size of a valve annulus.

Helical member 165 is adapted to be anchored into the tissue via rotation (e.g., screwed into the tissue). A screw axis of helical member 165 can be disposed substantially parallel with the surface of the tissue, e.g., within the tissue (e.g., the helical member is deeper in the tissue), outside of the tissue (e.g., the helical member is shallower in the tissue), or coincident with the surface of the tissue. Irrespective of the depth within the tissue, following implantation each turn of the helix can be disposed partly within the tissue and partly outside of the tissue.

In some implementations, helical member 165 has a sharpened tip 167, adapted to facilitate the advancement of the helical member through the tissue. Helical member 165 can be sufficiently flexible to be transluminally advanced to the heart and to curve around a valve annulus 10. However, the helical member can also be sufficiently rigid that it can be screwed into the tissue, such as by applying torque to a proximal end of the helical member. For example, helical member 165 can generally exhibit deflectional flexibility (e.g., its central longitudinal axis can be easily deflected), but may generally exhibit torsional stiffness (e.g., the helix may resist being untwisted upon being screwed into the tissue).

In some implementations, helical member 165 is configured to have a constant pitch along its length, and/or is configured such that the pitch remains generally constant during anchoring to the tissue. Nonetheless, in some implementations, and as described hereinbelow, helical member 165 can be axially contracted (e.g., reducing its pitch) subsequent to its anchoring to the tissue, in order to contract the tissue.

In some implementations, guide assembly 120 comprises a guide rail 122 that is adapted to position and guide the implantation of implant 160 (e.g., helical member 165 thereof) along annulus 10, e.g., by assuming, at least in part, a shape that implant 160 will assume upon its implantation. For example, guide rail 122 can extend along the tissue such that it provides a track along which the implant progresses.

In some implementations, once guide assembly 120 is in place, helical member 165 is advanced out of a tube 118 of delivery assembly 110 and along guide rail 122 (e.g., becoming progressively threaded onto the guide rail) such that the guide rail directs the implantation of the implant along the tissue. Guide rail 122 can thereby at least partly define the shape that implant 160 will assume upon implantation.

As shown, guide rail 122 can arc around at least part of annulus 10, and helical member 165 can thereby be anchored in an arc around at least part of the annulus.

In some implementations, guide rail 122 has a thickness that limits the depth to which helical member 165 can penetrate into the tissue, e.g., by the guide rail abutting the surface of the tissue. FIGS. 2B-D schematically illustrate an example in which the implant becomes partially embedded within the tissue during implantation, such that part of each turn of the helical member becomes submerged in the tissue, and part of each turn remains above the tissue. For example, guide rail 184 can have a thickness that is at least 25 percent (e.g., at least 40 percent, e.g., at least 50 percent, e.g., at least 70 percent) of the diameter of central channel 166 of helical member 165 (e.g., the internal diameter of the helical member).

In order to facilitate penetration of helical member 165 into the tissue, and/or withdrawal of guide rail 122 from the helical member, the thickness of the guide rail can be configured to be no more than 95 percent (e.g., no more than 90 percent, e.g., no more than 80 percent, such as no more than 70 percent) of the diameter of central channel 166.

In some implementations, guide rail 122 is resistant to medial compression, thereby retaining its thickness during implantation. In some implementations, guide rail 122 is a hypotube having sufficient flexibility (e.g., by defining slits therealong) to arc around annulus 10.

At a distal part of guide assembly 120, the guide assembly can also comprise a guide frame 124 that is adapted to facilitate positioning of guide rail 122 along annulus 10. The distal part of guide assembly 120 is transluminally advanceable to the heart of the subject, and guide frame 124 can be expandable within the heart.

In some implementations, guide frame 124 can be coupled to a control shaft 126 (e.g., to a distal end of the control shaft) that is configured to facilitate positioning and/or expansion of the guide frame.

In some implementations, during implantation of implant 160, guide frame 124 can be disposed at a distal end of a catheter 128, e.g., with control shaft 126 extending through a lumen of the catheter. In some implementations, guide frame 124 and/or control shaft 126 are positioned in this manner also during advancement to the heart.

In some implementations, guide assembly 120 does not include control shaft 126, e.g., guide frame 124 can be coupled (e.g., fixedly attached to) catheter 128.

In some implementations, guide assembly 120 can be advanced transluminally (e.g., transfemorally), via a sheath 112 of delivery assembly 110, while guide frame 124 is in a contracted state, and, once deployed out of a distal end of the sheath, guide frame 124 is expanded within the heart.

In some implementations, this expansion can be achieved merely by unconstraining the guide frame (e.g., by the guide frame being self-expanding), or by expanding the guide frame by applying an expanding force to the guide frame, such as with a mechanical actuator or a balloon.

In some implementations, guide frame 124 can be resilient (e.g., can be elastically expanded), such as being inherently biased to expand or inherently biased to contract, or can be merely flexible (e.g., can be plastically expanded).

In some implementations, guide frame 124 comprises (e.g., is formed from) a braided filament (e.g., wire). In some implementations, guide frame 124 is formed by cutting a stock material (e.g., is cut from a tube).

In some implementations, guide frame 124 can be formed from a metal (e.g., nitinol, stainless steel, and/or cobalt chrome). In some implementations, guide frame 124 can be formed from a polymer.

In some implementations, while expanded and at the native valve, guide frame 124 pushes the leaflets of the valve away from each other, and may result in the guide frame pressing against the tissue of the annulus. Nonetheless, the valve may continue to function at least in part, e.g., because guide frame 124 is open and allows blood flow therethrough, and/or because leaflets AL and PL remain partially functional (e.g., downstream of the guide frame), providing a net one-way flow of blood through the valve that may be sufficient for the duration of the procedure.

In some implementations, guide assembly 120 comprises one or more valve members, such as prosthetic leaflets, inside guide frame 124, to provide temporary valve functionality during the procedure.

In some implementations, guide frame 124 can have an upstream section 121 that is adapted to be positioned in an atrium 12 upstream of the valve and a downstream section 127 that is adapted to be positioned in a ventricle 14 downstream of the valve. Guide frame 124 can be shaped to facilitate placement of a midsection 125 of the guide frame, between the upstream section and the downstream section, at an annulus 10 of an atrioventricular valve between the atrium and the ventricle e.g., at/against an upstream surface of the annulus. For example, and as shown, while guide frame 124 is in its expanded state, downstream section 127 can taper away from midsection 125, such that the downstream section can be advanced, from the atrium, in a downstream direction through the atrioventricular valve until the midsection comes to rest against the upstream surface of the annulus of the valve. In some implementations, midsection 125 can lie generally orthogonal to a central longitudinal axis ax1 of guide frame 124 that extends from the upstream section to the downstream section.

In some implementations, frame 124 can define a concave waist at midsection 125.

In some implementations, in order for guide frame 124 to facilitate positioning of guide rail 122 along the annulus (e.g., to guide helical member 165 therealong), guide assembly 120 can comprise multiple fasteners, distributed along a part of the circumference of the midsection of guide frame 124 (e.g., collectively describing an arc around the circumference). In the example shown, each of these fasteners is in the form of (or comprises) a respective loop 140.

In some implementations, each loop 140 is looped around a piece (e.g., a strut) of the guide frame and the guide rail. In some implementations, guide rail 122 extends through loops 140 (e.g., by being threaded through the loops), such the guide rail extends circumferentially around at least part of the guide frame (e.g., in an arc). Thus, when guide frame 124 is expanded and placed in the native valve (with guide rail 122 positioned along midsection 125), this arrangement positions the guide rail along the tissue of the annulus (e.g., in contact with an atrial surface of the annulus).

In some implementations, guide rail 122 can extend along the tissue (e.g., of annulus 10) in a manner that complements (e.g., generally matches) the shape of the tissue. In some implementations, this can be facilitated by guide frame 124 being sufficiently compliant that its expanded shape is influenced by the existing shape of the tissue.

In some implementations, implant 160 can comprise a tensile member 186 (e.g., an elongate contraction member, such as a wire, cable, suture, or ribbon) that, following implantation, extends through central channel 166 of helical member 165.

In some implementations, during delivery, tensile member 186 can be disposed through guide rail 122 (such as through a lumen defined by the guide rail, as shown), or alongside the guide rail.

In some implementations, after the guide rail and the guide frame are withdrawn, tensile member 186 remains behind as a component of implant 160. In some implementations, the tensile member can be introduced after helical member 165 has been delivered and/or anchored. In some implementations, no distinct tensile member is used, e.g., helical member 165 itself adjusts the tissue.

FIGS. 2A-I represent a series of steps that can be performed by the operator, to circumferentially reduce the size of an annulus 10 of a heart valve, in accordance with some implementations. Although FIGS. 2A-I show a sequence of at least some steps in a procedure, and can, in fact, be performed in the order shown, these figures are also intended to illustrate the capability of system 100, independently of any particular sequence of steps.

FIG. 2A shows guide assembly 120 deployed within the heart, such that guide rail 122 extends along the tissue of annulus 10 (e.g., in contact with an atrial surface of the annulus). As shown, loops 140 can hold guide rail 122 along an outer surface (e.g., a circumference) of guide frame 124, in order to position the guide rail along the annulus.

Helical member 165 is then screwed into the tissue such that it becomes anchored along the tissue (FIG. 2B-D), guided by guide rail 122.

In some implementations, the guide rail extends, from around guide frame 124, into tube 118, and through the tube proximally away from the heart (e.g., to outside of the subject). In some implementations, and as shown, tube 118 extends along guide frame 124 such that guide rail 122 exits the tube at a gentle slope (e.g., at an angle between 0 and 70 degrees) with respect to the annulus, in order to facilitate screwing of helical member 145 into the tissue of the annulus. In some implementations, tube 118 extends distally out of sheath 112, e.g., as shown. In some implementations, guide assembly 120 does not comprise tube 118, e.g., guide rail 122 simply exits a distal opening of catheter 128 and extends around guide frame 124.

In some implementations, catheter 128 defines a lumen 128a via which tube 118 and/or guide rail 122 extends. Catheter 128 can define another lumen 128b via which control shaft 126 extends. Lumen 128a can run alongside control shaft 126 and/or alongside lumen 128b.

In some implementations, a driver 116 of delivery assembly 110 can be reversibly engaged with a proximal part of helical member 165 (e.g., to a head 169 thereof) in order to screw the helical member into the tissue by rotation of the helical member.

In some implementations, driver 116 extends, from the heart where it is engaged with head 169, through delivery assembly 110, to a proximal end of the driver where it is coupled to an anchor-handle 117 that comprises an anchor-release mechanism 117a.

In some implementations, anchor-handle 117 can be used to anchor the helical member to the tissue e.g., by rotating the anchor-handle to screw the helical member along the tissue.

In some implementations, the operator can release helical member 165 from driver 116 by operating anchor-release mechanism 117a, such that the driver can be withdrawn from the heart, leaving the helical member implanted along the tissue.

In some implementations, and as described hereinabove, once helical member 165 is anchored, part of each turn of the helical member is embedded in the tissue of annulus 10, and another part of each turn is disposed outside of the tissue (e.g., in atrium 12).

In some implementations in which guide assembly 120 includes tube 118, driver 116 can be advanced through the tube as it drives helical member 165 out of the tube and into the tissue.

In some implementations, subsequently to anchoring helical member 165 along the tissue (FIG. 2D) and/or subsequently to disengaging driver 116 from anchor head 169, at least part of guide assembly 120 is withdrawn from the heart, e.g., via sheath 112. For example, and as shown in FIG. 2E-F, guide rail 122 can be withdrawn from inside of helical member 165 (e.g., from central channel 166 of the helical member), leaving tensile member 186 extending through the central channel of the helical member and/or extending through loops 140. FIG. 2E shows guide rail 122 partially withdrawn, and FIG. 2F shows the guide rail fully withdrawn.

In some implementations, the withdrawal of guide rail 122 from helical member 165 can be achieved by pulling (e.g., from outside of the subject) such that the guide member slides out of central channel 166.

In some implementations, prior to contracting the tissue using tensile member 186, guide frame 124 is also withdrawn. To achieve this, loops 140 can be opened (e.g., as described in more detail hereinbelow) in order to unloop the loops such that they cease to couple tensile member 186 and/or helical member 165 to guide frame 124.

In some implementations, loops 140 are loosened (or even fully opened) prior to withdrawing guide rail 122.

FIG. 2G shows guide frame 124 having been withdrawn (e.g., subsequently to withdrawing guide rail 122), leaving tensile member 186 extending, from the distal end of anchored helical member 165, proximally through central channel 166 of the helical member and into tube 118.

In some implementations, and as shown in FIG. 2H, implant 160 is then axially contracted by applying tension to tensile member 186 (e.g., by pulling the tensile member proximally from outside of the subject). In some implementations, at least in part due to a first stopper 164a that is fixed to a distal end of guide rail 122, thereby preventing the distal end of the guide rail from sliding proximally through helical member 165, tensioning of tensile member 186 results in longitudinal contraction of the helical member.

In some implementations, first stopper 164a is a toggle “T-shaped” stopper. A tensioning tool 180 of delivery assembly 110 can be advanced (e.g., through sheath 112) and used to facilitate application of the tension (FIG. 2H), e.g., by applying a reference force during tensioning of the tensile member.

In some implementations, tensioning tool 180 can lock the tension in tensile member 186, such as by locking a second stopper 164b (e.g., a lock) onto the tensile member (e.g., such that it abuts anchor head 169).

In some implementations, second stopper 164b can be a crimp, which tensioning tool 180 crimps to tensile member 186. In some implementations, second stopper 164b (and/or tensioning tool 180) can be any of those described in the following publications, which are incorporated herein by reference:
US 2019/0274674 to Sutherland et al.
US 2020/0015971 to Brauon et al.
US 2021/0145584 to Kasher et al.

In some implementations, excess tensile member 186 can then be trimmed, e.g., by tensioning tool 180 or a dedicated cutting tool cutting the tensile member just proximally from second stopper 164b. At this point, tensioning tool 180 (and optionally the entire remainder of delivery assembly 110) can be removed from the subject (FIG. 2I).

Reference is additionally made to FIGS. 3A-E, which are schematic illustrations that show, in further detail, some optional components and/or features of guide assembly 120, in accordance with some implementations.

In some implementations, the fasteners (e.g., loops 140) are formed from one or more longitudinal members (e.g., a thread, suture, ribbon, rope, wire, cable, or string), e.g., each longitudinal member defining a respective loop.

In some implementations, the longitudinal members extend, from an extracorporeal proximal portion of delivery assembly 110 and through guide assembly 120, to form loops 140 at guide frame 124, e.g., at an outer surface thereof. The longitudinal members can extend through catheter 128 and/or control shaft 126 to guide frame 124, e.g., to an interior thereof.

In some implementations, from the interior of guide frame 124, the longitudinal members can extend out of the guide frame (e.g., between struts thereof), looping around guide rail 122 to define loops 140, and back into the interior of the guide frame. In some implementations, the longitudinal members can then return through the guide assembly and back out of the subject.

In some implementations, the release of loops 140 from implant 160 (described with reference to FIGS. 2F-G) is achieved simply by releasing one end of each longitudinal member and pulling the other end of the longitudinal member (e.g., from outside of the subject) until the longitudinal member unloops from guide rail 122.

In some implementations, guide assembly 120 can comprise a plurality of tubular rods 148 via which the longitudinal members extend, e.g., one rod per longitudinal member. Rods 148 can extend through catheter 128 and/or control shaft 126 to guide frame 124, e.g., to an interior thereof.

In some implementations, at least in some states of guide assembly 120 (e.g., during anchoring of helical member 165), a distal opening of each rod is disposed at (e.g., faces) the inner surface of guide frame 124, e.g., against a strut thereof.

In some implementations, as in the example shown, rods 148 extend out of control shaft 126 into the interior of guide frame 124, and to the inner surface of the guide frame.

In some implementations, tension on the longitudinal members holds guide rail 122 against guide frame 124, e.g., by sandwiching the guide frame between the guide rail and rods 148.

In some implementations, rods 148 are substantially longitudinally incompressible, and thereby provide a reference force that cooperates with the tension on the longitudinal members. Despite this, rods 148 can be flexible (e.g., laterally), e.g., sufficiently flexible to allow expansion and contraction of guide frame 124.

In some implementations, guide assembly 120 comprises a plurality of spacers 130 that maintain spacing (e.g. radial spacing) between guide rail 122 and guide frame 124, even while loops 140 pull the guide rail toward the guide frame. This spacing may advantageously facilitate anchoring of helical member 165 by allowing sharpened tip 167 to pass between the guide rail and the guide frame as the helical member is rotated. For example, this spacing may reduce a likelihood of helical member 165 catching or threading onto guide frame 124, and/or fastening the guide frame to the tissue.

In some implementations, spacers 130 are spacer wires that extend longitudinally along an outer surface of at least midsection 125 of the guide frame (e.g., perpendicular to the midsection). In some implementations, each individual wire is looped to form two spacers 130, e.g., on opposite sides of guide frame 124. For example, and as shown, each individual wire can loop around a downstream section 127 of guide frame 124.

In some implementations, at upstream section 121 and/or downstream section 127, the wires that define spacers 130 are disposed in the interior of guide frame 124. In some implementations, each spacer 130 weaves in and out of guide frame 124, with the spacer disposed on the outside of the guide frame at midsection 125. Such an arrangement may advantageously provide additional mechanical support to the guide frame, and/or maintain each spacer in its designated circumferential position around the guide frame.

In some implementations, one or more of spacers 130 serves as a mechanical actuator for expanding (and optionally compressing) guide frame 124 once guide assembly 120 is positioned within the heart. For example, spacers 130 can be pulled in a manner that axially compresses the guide frame by pulling the distal end of the guide frame proximally toward the proximal end of the guide frame, the guide frame radially expanding in response to this axial compression. Alternatively or additionally, spacers 130 can be pushed in a manner that causes the spacers to bow radially outward, pulling the guide frame radially outward.

For clarity and simplicity, spacers 130 have been omitted from FIGS. 1-2I.

In some implementations, rods 148 function also as spacers (e.g., spacers 130 are not used), by the rods protruding slightly through guide frame 124 and guide rail 122, thereby holding the guide rail away from the guide frame.

In some implementations, guide rail 122 includes a plurality of imaging markers 123 (e.g., fluoroscopic markers and/or echogenic markers), spaced along the guide rail at predetermined intervals. Imaging markers 123 are shown in FIG. 1, but for clarity and simplicity are omitted from the other figures. In some implementations, imaging markers 123 can be used to visualize the procedure and/or to verify particular steps in the procedure, e.g., before proceeding to a subsequent step. For example, imaging markers 123 can be used to verify the position of guide rail 122 around guide frame 124 (e.g., around midsection 125 thereof), e.g., positioned in a manner that facilitates placement of the guide rail along (e.g., parallel with) the annulus.

FIGS. 3A-D show an example technique for delivering and deploying guide assembly 120 within a heart, prior to implanting implant 160 (e.g., prior to the steps shown in FIGS. 2A-I), in accordance with some implementations. FIG. 3E shows implant 160 being implanted around the guide assembly once it has been fully deployed within the heart.

In some implementations, during delivery (e.g., transluminal delivery) of guide assembly 120 to the heart, the guide assembly is constrained in a delivery state, e.g., within sheath 112. In the delivery state, guide frame 124 is compressed radially inwards (e.g., in a substantially narrow and/or elongate form).

In some implementations, in the delivery state, guide rail 122 is disposed alongside (e.g., substantially parallel with) the compressed guide frame 124. In some implementations, in the delivery state, guide rail 122 curves (e.g., helically) at least partway around the compressed guide frame.

Irrespective of the position of guide rail 122 with respect to compressed guide frame 124 in the delivery state, loops 140 may already extend out of guide frame 124 and loop around guide rail 122. In the delivery state, the exit sites 142 at which loops 140 extend out of the guide frame may be disposed along midsection 125, e.g., extending away from the exit sites to loop around the guide rail.

In accordance with some implementations, FIG. 3A represents a state of guide assembly 120 immediately upon deployment out of sheath 112, e.g., with guide frame 124 having expanded slightly. Thus, in some implementations, FIG. 3A substantially illustrates a delivery state of guide assembly 120 in which guide rail 122 curves (e.g., helically) at least partway around the compressed guide frame.

In accordance with some implementations, FIG. 3A may represent a state of guide assembly 120 after guide rail 122 has been moved (e.g., pulled) into a helical configuration after the guide assembly has been deployed out of sheath 112, e.g., from a delivery state in which the guide rail lay substantially parallel with the compressed guide frame.

Once guide assembly 120 is positioned within the heart (e.g., once deployed out of the sheath), guide frame 124 can be expanded radially within the heart (FIG. 3B).

In some implementations, the fasteners (e.g., loops 140) can then be tightened in a manner that pulls guide rail 122 toward alignment along midsection 125, e.g., such that the guide rail extends circumferentially around at least part of the midsection. This is illustrated by the transition from FIG. 3B to FIG. 3C to FIG. 3D. The tightening of the loops (e.g., the tensioning of the longitudinal members that define the loops) holds the guide rail to guide frame 124 at midsection 125. As noted hereinabove, for implementations in which guide assembly 120 includes spacers 130, the spacers maintain spacing between guide rail 122 and guide frame 124, e.g., becoming sandwiched between the guide rail and the guide frame upon tightening of loops 140.

In some implementations, the tightening of the fasteners is achieved by, for each fastener (e.g., for each loop 140) sequentially, pulling one or both ends of the longitudinal member of that loop from outside the subject.

In some implementations, prior to guide assembly 120 reaching its deployed state (e.g., the state shown in FIG. 3D), different points along guide rail 122 can be disposed at different distances from midsection 125. In the example shown (e.g., in FIGS. 3A-C), whereas the distal end of guide rail 122 is disposed approximately at midsection 125, points along the guide rail that are progressively further from the distal end of the guide rail are disposed progressively further (e.g., progressively proximally and/or upstream) from the midsection.

In some implementations, loops 140 can have different exposed lengths (e.g., the length exposed outside of guide frame 124) to each other, e.g., in order to accommodate the resulting different distances between the guide rail and the exit sites 142 of the loops from the guide frame. Thus, during tightening of loops 140, the longitudinal member of each loop can be pulled by a different amount in order to take up the different exposed lengths so as to draw the guide rail into alignment with midsection 125.

In some implementations, the expansion of guide frame 124 and the positioning of guide rail 122 along midsection 125 are both performed within atrium 12 prior to moving the guide assembly into (or deeper into) the native valve, in order to position the guide rail along annulus 10. In some implementations, guide rail 122 can act as a flange for guide assembly 120, e.g., such that the downstream movement of the guide assembly causes the guide rail to abut the annulus, e.g., inhibiting further downstream movement of the guide assembly.

In some implementations, the expansion of guide frame 124 and/or the positioning of guide rail 122 along midsection 125 is performed subsequently to positioning the guide frame at the native valve, e.g., such that the guide frame may not require further repositioning prior to anchoring of helical member 165. In some implementations, the positioning of guide rail 122 along the midsection also positions the guide rail along the annulus, e.g., midsection 125 can be disposed along the annulus prior to the positioning of the guide rail.

In some implementations, it may be desired that helical member 165 be anchored along a shorter stretch of the annulus, e.g., rather than along a stretch whose length is predetermined by the configuration of system 100. For example, whereas the figures show system 100 being capable of anchoring helical member 165 in an arc that extends approximately halfway around the annulus, an operator may determine that it is desirable, for a particular subject, to anchor the helical member along less than half of the annulus.

In some implementations, the operator can position less of guide rail 122 along midsection 125 by tightening only a subset of the fasteners, and may then place only the positioned portion of the guide rail along the desired stretch or the annulus. In some implementations, this feature can be facilitated by imaging markers 123. For example, visualization of imaging markers 123 can facilitate determination of how much of guide rail 122 is positioned along midsection 125 and/or along the annulus.

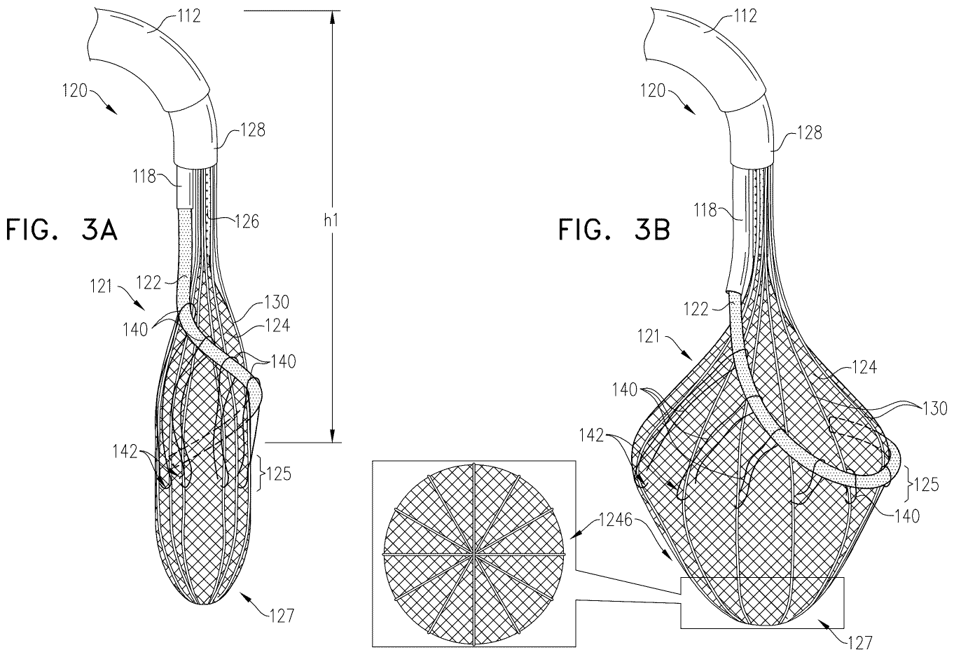

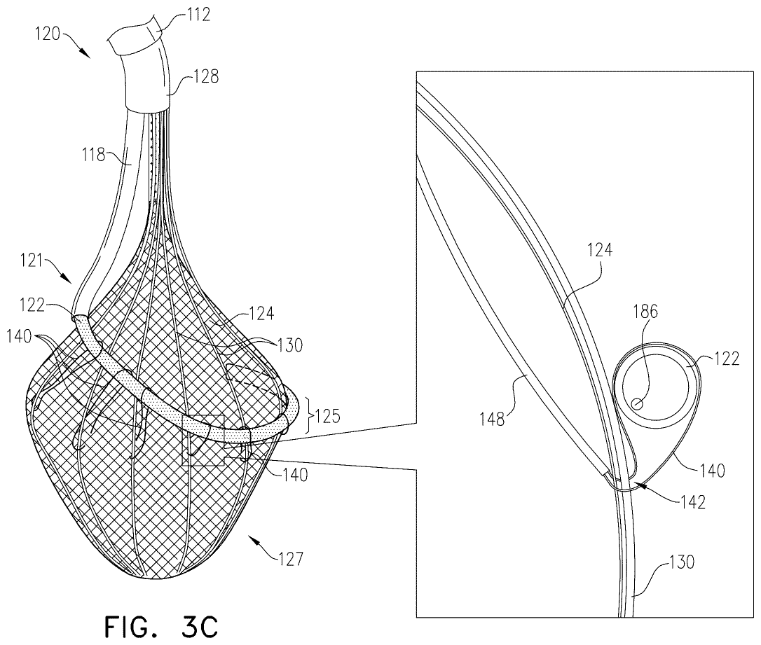

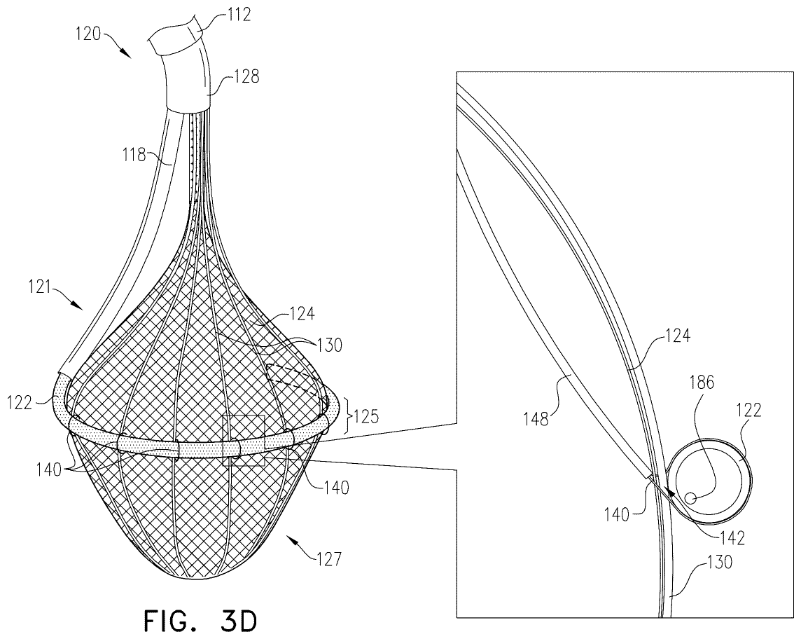

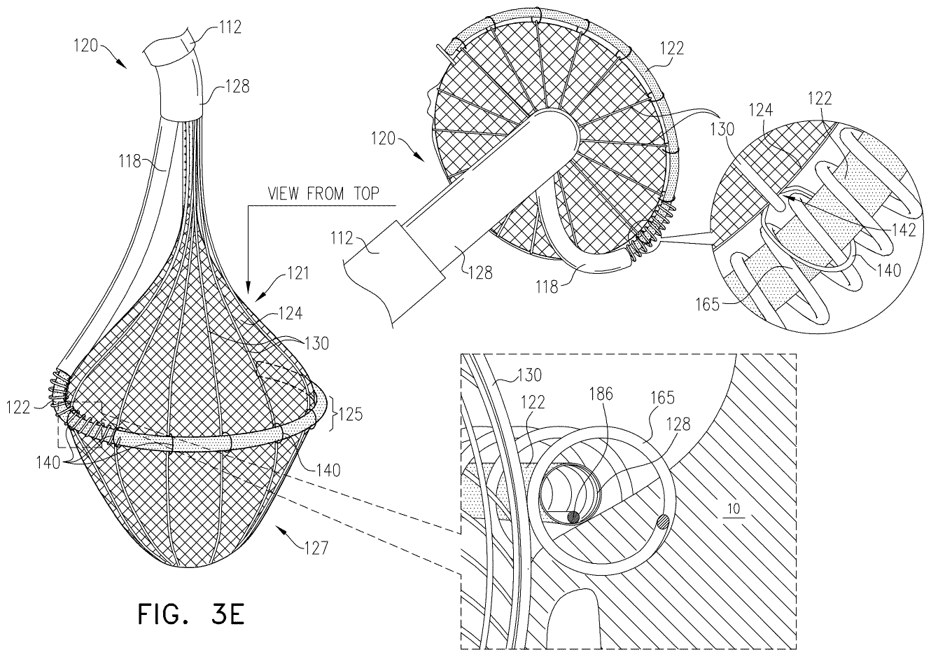

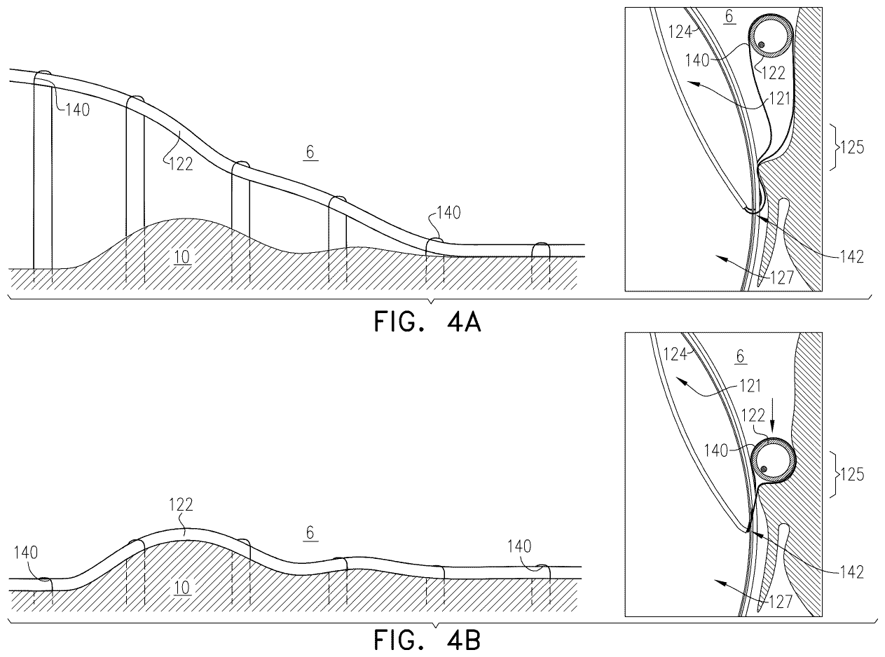

Reference is now made to FIGS. 4A and 4B, which are schematic illustrations of a technique of positioning guide rail 122 against annulus 10 of the valve such that the guide rail conforms to the contours of the valve, in accordance with some implementations.

The annulus may not have a uniform height—e.g., it may be saddle-shaped. A technique is thus disclosed to draw guide rail 122 uniformly against the atrial-facing surface of annulus 10—e.g., such that the guide rail lies conformally against the atrial-facing surface of the annulus.

In some implementations, prior to positioning the guide rail against the annulus, guide rail 122 is disposed around guide frame 124 within atrium 6, and exit sites 142 are disposed at downstream section 127 of the guide frame, e.g., within ventricle 8, such that annulus 10 is disposed between the exit sites and the guide rail (FIG. 4A). For example, in some implementations, prior to positioning the guide rail against the annulus, guide frame 124 is positioned within the heart such that each fastener 140 exits the guide frame at a respective exit site 142 within the ventricle. As shown in FIG. 4A, fastener 140 can extend upstream through the orifice of the valve and into the atrium where it loops around guide rail 122. In some implementations, the fastener 140 can loop back downstream to the same exit site—e.g., as shown.

In some implementations, to position the guide rail against the atrial-facing surface of the annulus, as shown by the transition between FIGS. 4A and 4B, fasteners 140 can be tightened. Tightening the fasteners 140 can draw respective segments of the guide rail downstream in a ventricular direction (e.g., by the fasteners pulling the guide rail towards their exit sites), until the guide rail 122 abuts (e.g., and is thereby stopped by) a respective segment of the annulus.

Thus, in some implementations, the guide rail as a whole conforms to the contours of the annulus, thereby advantageously desensitizing the system to the position (e.g., depth) within the valve of the guide frame and/or the exit sites of the fasteners. This may thus obviate any necessity of ensuring that midsection 125 of the guide frame (e.g., a section around which the exit sites are positioned) is positioned uniformly along the native valve.

Reference is now made to FIG. 5, which illustrates an implementation of a helical member 165a (e.g., a variant of helical member 165, optionally usable and/or for use with guide assembly 120 and/or delivery assembly 110). As shown in the figure, helical member 165a has a thickness that is greater toward its proximal end (e.g., head 169 of FIGS. 2F-I) than toward its distal tip 167a. For example, and as shown, the thickness of helical member 165a may be tapered to become progressively greater from distal tip 167a toward the proximal end.

In some implementations, the helical member may have a graduated thickness, e.g., the thickness of the helical member may increase in discrete graduations from the distal tip toward the proximal end.

In some implementations, this varying thickness advantageously provides greater steerability and/or flexibility at thinner, distal regions of the helical member (e.g., the regions that will follow the arced path along the annulus), while the thicker proximal regions are more suited to torque transfer. For instance, the torque that is required to rotate the entire helical member is applied at the proximal end (e.g., to head 169), and the proximal region of the helical member is sufficiently stiff to transfer this torque. Progressively distal regions of the helical member are required to transfer progressively less torque to the progressively smaller regions remaining distal thereto, and may therefore be made thinner, exchanging torque-transfer for flexibility.

Additionally and/or alternatively, in some implementations, helical member 165a may be comprised of different materials and/or structures that render the helical member more flexible and/or steerable towards the distal end than the proximal end e.g., thereby providing the abovementioned advantage of additional torque strength toward the proximal end of the helical member, while providing a more flexible and/or steerable section towards the distal end.

Reference is now made to FIGS. 6A-C, which are schematic illustrations of a guide assembly 120a, in accordance with some implementations. In some implementations, guide assembly 120a may be a variant, or substantially identical to, guide assembly 120 (e.g., guide assembly 120a may comprise a guide frame 124, a guide rail 122, and fasteners 140). In some implementations, guide assembly 120a may be a component of a delivery assembly 110a.

In some implementations, guide assembly 120a comprises a guide rail (e.g., guide rail 122) and a guide frame (e.g., guide frame 124). In some implementations, guide assembly includes a control shaft 126a that is coupled to the guide frame 124 at a distal end of the control shaft. Control shaft 126a can be a variant of control shaft 126. Delivery assembly 110a can be a variant, or substantively identical to, delivery assembly 110, unless noted otherwise.

In some implementations, delivery assembly 110a comprises a handle 150 at an extracorporeal portion thereof. Handle 150 may be used to control the delivery of guide assembly 120a to the heart, and/or to guide the implantation of helical member 165 along the annulus.

In some implementations, prior to positioning guide rail 122 in its guide arrangement around guide frame 124, it may be advantageous to maintain the guide rail (e.g., a distal end 132 thereof) fastened to the guide frame 124. For example, such a fastened (e.g., fixed) state may advantageously prevent slippage of fasteners 140 off the distal end of the guide rail (e.g., by inhibiting the guide rail from sliding out from the fasteners). Alternatively or additionally, such a fastened (e.g., fixed) state may advantageously maintain the guide rail in a particular delivery state. In some implementations, the guide rail may be fastened in a state that is favorable for transcatheter delivery, such as by minimizing the diameter of the guide assembly in its delivery state.

In some implementations, in order to provide such fastening of the guide rail to the guide frame, guide assembly 120a comprises a fixation wire 144 that fastens distal end 132 of guide rail 122 to the guide frame 124. For instance, the fixation wire can attach to a connector 133 of the guide rail in a manner that fastens the connector 133 to a connection location 146 on the guide frame.

In some implementations, connector 133 is an eyelet, and fixation wire 144 fastens connector 133 to connection location 146 on the guide frame by extending out of the guide frame and being threaded (e.g., looped) through the eyelet. In such a manner, during delivery and/or positioning (e.g., expansion) of the guide frame at the valve orifice, distal end 132 of guide rail 122 is maintained in a fixed state against guide frame, at connection location 146 (FIG. 6A).

For some implementations, and as shown, the eyelet of connector 133 is transverse to (e.g., lies across a cross-section of) the guide rail itself.

In some implementations, once the guide frame has been delivered to the heart, and/or once the guide frame has been expanded and/or positioned within the valve orifice being treated, distal end 132 can be released (e.g., allowed to move away) from connection location 146. In some implementations, the distal end 132 can be released by loosening and/or withdrawing fixation wire 144 out of connector 133 (FIG. 6B).

In some implementations, the distal end 132 can be released by actuating a controller 152 on handle 150. Releasing of distal end 132 from connection location 146 may allow for the positioning of guide rail 122 into the guide arrangement around guide frame 124 (e.g., via the tightening of fasteners 140). That is, in some implementations, tightening a distal-most fastener 140a of the fasteners may cause the distal end to pivot upstream towards the midsection. For example, releasing distal end 132 from connection location 146 can allow the distal end to move, e.g., pivot, into a position appropriate for the guide arrangement responsively to tightening of loops 140—e.g., moving upstream to the midsection of the guide frame.

FIG. 6C shows helical member 165 having been advanced along the guide rail 122 (e.g., via screwing of the helical member into and along annulus 10). Once the helical member has been implanted, guide rail 122 can be withdrawn from out of the helical member. For instance, the guide rail can be pulled proximally such that it slides out of a central channel defined by the helical member, e.g., as described with reference to FIG. 2E, mutatis mutandis. In some implementations, prior to withdrawing guide rail 122 from out of the helical member, the guide rail is first detached from guide frame 124, e.g., by unthreading (e.g., unlooping) fixation wire 144 from out of connector 133 (e.g., from out of the eyelet) of distal end 132.

In some implementations, in the delivery state, distal end 132 of guide rail 122 is fastened to upstream section 121 of the guide frame. In some such implementations, the releasing of distal end 132 from its connection location may allow the distal end to move downstream to the midsection of the guide frame responsively to tightening of loops 140.

In some implementations, in the delivery state, rather than distal end 132 of guide rail 122 being fastened to downstream section 127 of guide frame 124, it is fastened to midsection 125. In some such implementations, distal end 132 may not be released from its connection location while transitioning the guide rail into the guide arrangement.

In some implementations, rather than guide rail 122 extending alongside the control shaft and along an exterior of guide frame 124, the guide rail 122 of guide assembly 120a extends distally from control shaft 126a, into an interior of the guide frame (e.g., without being exposed from the control shaft or the guide frame), and extending distally through the interior of the guide frame and out of the guide frame at an exit site 1242 to curve around an exterior of the guide frame.

In some implementations, guide rail 122 extends through control shaft 126a, and extends out of the control shaft into the interior of the guide frame. In some such implementations, guide rail 122 may be coaxial within the control shaft.

In some implementations, guide rail 122 extends alongside an exterior of control shaft 126a, and, at a distal end thereof, into the interior of the guide frame.

The configuration of guide assembly 120a may advantageously allow for gentler curvature of the guide rail around the guide frame, which may, inter alia, facilitate improved torque application via driver 116 when the driver follows the contours of the guide rail.

Furthermore, having guide rail 122 and guide frame 124 extend out of a common control shaft 126a and/or having the guide rail extend directly into the guide frame may allow for a shorter height h2 of guide assembly 120a within the atrium, e.g., by allowing the distal end of control shaft 126a (e.g., and therefore guide frame 124 itself) to be positioned and manipulated higher up within the atrium—e.g., compared with some implementations of the arrangement described with reference to guide assembly 120. This effect may be increased if catheter 128 is consequently no longer required.

For example, as shown in FIG. 3A, a height h1 within the atrium of the arrangement shown with reference to guide assembly 120 (e.g., in which control shaft 126 extends alongside guide rail 122) may be greater than height h2 of guide assembly 120a within the atrium. Similarly, having guide rail 122 and guide frame 124 extend out of a common control shaft 126a and/or having the guide rail extend directly into the guide frame may allow for a more compact delivery state of the system (e.g., by the guide assembly having a smaller cross-section during delivery).

In some implementations, control shaft 126a is a hypotube.

In some implementations, guide assembly 120a comprises spacers 130 (e.g., in order to prevent helical member 165 from catching guide frame 124 during screwing of helical member 165 along the tissue). In some implementations, spacers 130 are additionally used to actuate (e.g., to expand) guide frame 124. In some implementations, spacers 130 are absent. In some such implementations, other spacing techniques may be used to shield and/or space the guide frame from the guide rail during screwing of the helical member (e.g., using any of the shielding and/or spacing techniques described hereinbelow).

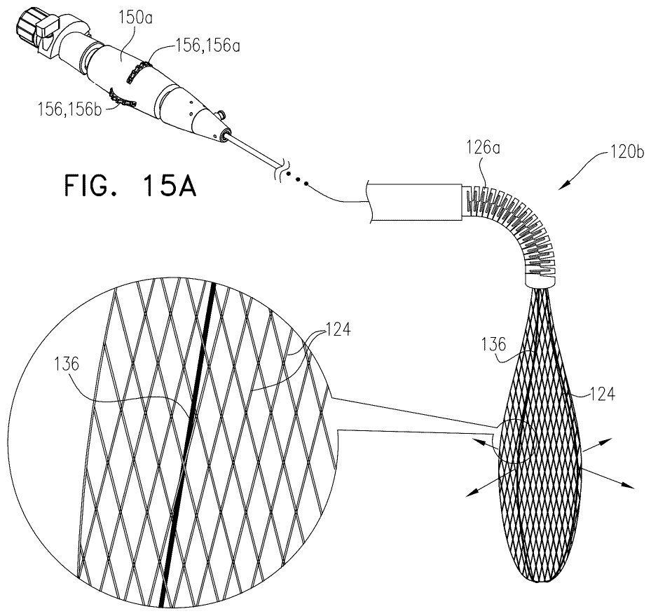

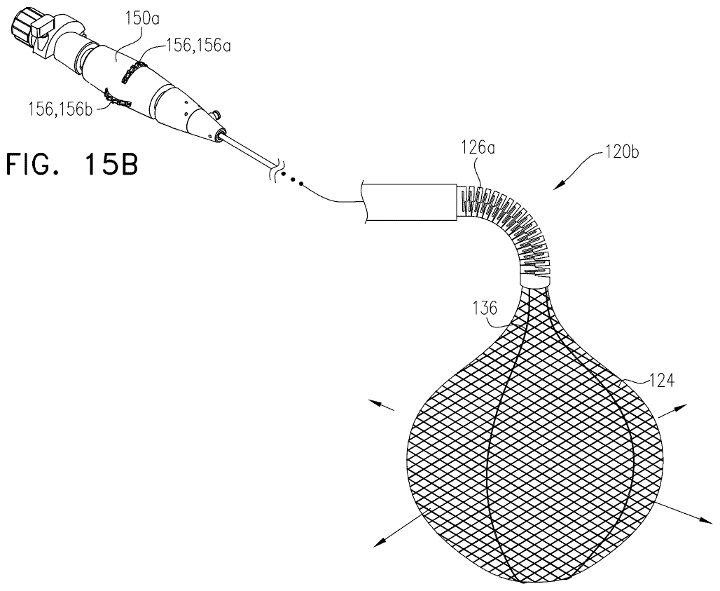

In some implementations, guide assembly 120a comprises actuator wires that are used to expand and/or compress the guide frame. For example, guide assembly may comprise actuator wires 136, e.g., as described with reference to FIGS. 15A-C.

In some implementations, guide assembly 120a further comprises a tube 118a that extends distally from control shaft 126a through the interior of guide frame 124 and out of the guide frame at exit site 1242. Guide rail 122 may extend through and out of tube 118a, such that only segments of the guide rail beyond tube 118a are exposed. Similarly, as helical member 165 is advanced over and along the guide rail, the helical member passes through the interior of guide frame 124 and out of exit site 1242 while within tube 118a, only becoming exposed after exiting the tube outside of the guide frame.

Thus, tube 118a may advantageously shield the guide frame and/or the surrounding tissue of the heart from sharpened distal tip 167 of helical member 165 during delivery of the helical member over and along the guide rail—e.g., preventing damage to tissue and/or ensnarement of the guide frame. In some implementations, tube 118a may extend from a distal end of control shaft 126a (e.g., may be attached to the distal end of the control shaft, such as where the guide frame is attached to the control shaft). Alternatively, tube 118a may extend through the control shaft and out of the distal end of the control shaft.

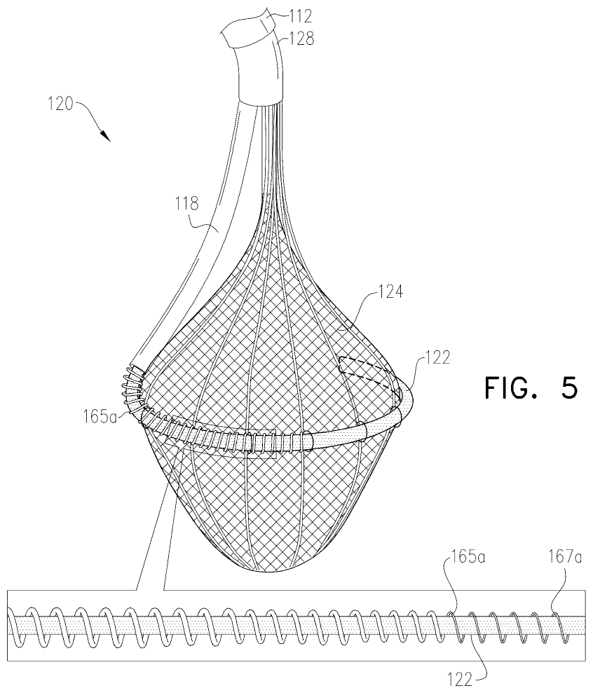

FIG. 7 shows an implementation in which a flexible sleeve 118b serves as a variant of tube 118a. In some implementations, sleeve 118b may be formed from a fabric or a film, and may comprise a polymer (e.g., a polyether block amide) or any other suitable material.

The flexibility of sleeve 118b may advantageously prevent inadvertent damage to the surrounding tissue and/or guide assembly that may occur via a traumatic distal end of a more rigid tube (e.g., by the distal end cutting tissue against which it contracts). Furthermore, in contrast to a non-fabric (e.g., metallic) tube that may become “bent out of shape” during its delivery towards the heart, sleeve 118b can regain its form upon positioning out of the delivery assembly and within the heart. Moreover, during delivery of the tube to the heart, a flexible sleeve may be more compact than a more rigid version e.g., by having a thinner wall and/or by being more easily compressible within a sheath (e.g., sheath 112).

In some implementations, sleeve 118b is attached to, and extends distally away from, a distal end of control shaft 126a. In some implementations, sleeve 118b extends through the control shaft, and out of the control shaft.

In some implementations, tube 118 and/or tube 118a may be modified to include a flexible (e.g., fabric) distal end. For instance, tubes 118 and/or 118a may be rigid (e.g., by being metallic and/or plastic), but may have a distal portion that is in the form of sleeve 118b (e.g., the portion of the tube that extends out of the control shaft).

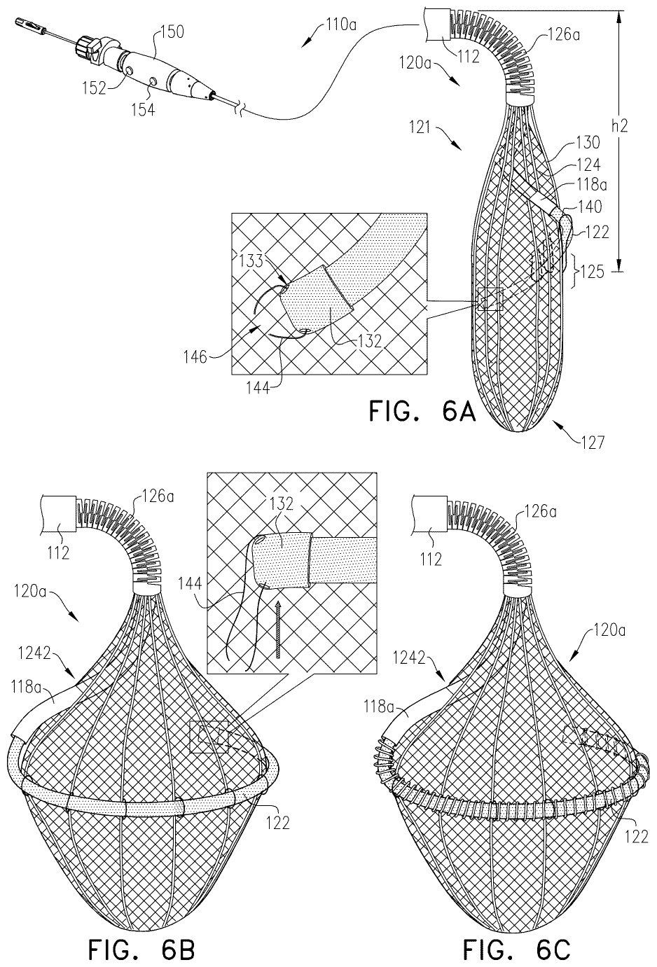

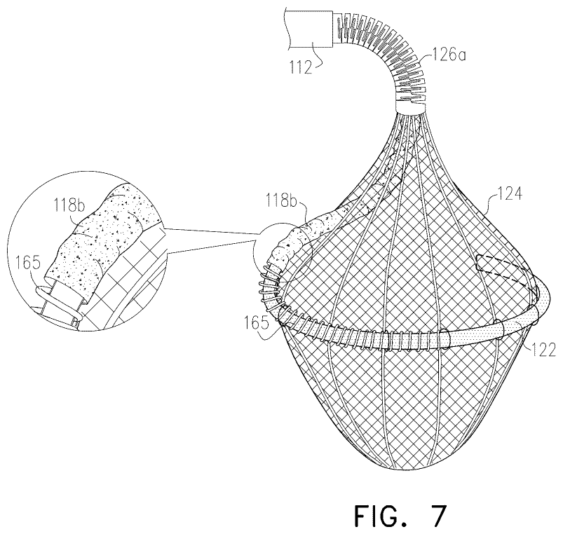

Reference is now made to FIGS. 8A-B, which illustrate a method of delivering helical member 165 towards the annulus, in accordance with some implementations. In some implementations, during advancement of the helical member over and along the guide rail 122 (e.g., at least the part of the guide rail that is exposed out of control shaft 126), the helical member is rotated in a direction that is opposite to the handedness of the helical member—i.e. in an “unscrewing” direction.

FIG. 8B schematically shows how the helical member would be anchored (e.g., screwed) into tissue of the annulus, being rotated, using driver 116, in a first direction. FIG. 8A schematically shows the prior advancement of the helical member towards the tissue, and the opposite direction of the arrows indicate the helical member being rotated in a second, opposite direction. This opposite rotation during advancement may advantageously protect the guide frame and/or tissue of the heart from sharpened tip 167 of helical member 165, and/or prevent the sharpened tip from undesirably catching onto surrounding tissue and/or guide frame 124.

For some implementations, the helical member 165 is rotated in this second direction only once the helical member is exposed out of the delivery assembly (e.g., out of control shaft 126) within the heart. Once helical member 165 (e.g., sharpened distal tip 167) arrives at (e.g., contacts) tissue of annulus 10, the helical member can be rotated in the “screwing” direction (e.g., in the first direction), to screw the helical member into the tissue (FIG. 8B).

In some implementations, once the user (e.g., a physician) has received feedback (e.g., haptic feedback via driver 116) that helical member 165 has reached the tissue and/or the first of fasteners 140, does the user begin to rotate the helical member in the tip-facing direction. For example, the first fastener may obstruct helical member 165 from being advanced in the absence of rotation in the first direction.

In some implementations, imaging may be performed to determine that there is contact between tip 167 and tissue of annulus 10, before screwing begins. In some implementations, the delivery assembly is configured (e.g., through the use of interlocks and/or safety features on handle 150) to enforce the above-described reverse rotation during advancement. For example, in a first mode of the delivery assembly the operator may operate a single controller (e.g., knob) that advances the helical member distally while rotating it in the unscrewing direction, while in a second mode of the delivery assembly the operator may operate a single controller (e.g., knob), which may be the same knob, to advance the helical member distally while rotating it in the screwing direction.

In some implementations, a switch 154 may be provided on handle 150 via which the operator can switch from the first mode to the second mode—e.g., once it has been determined that the helical member is appropriately positioned.

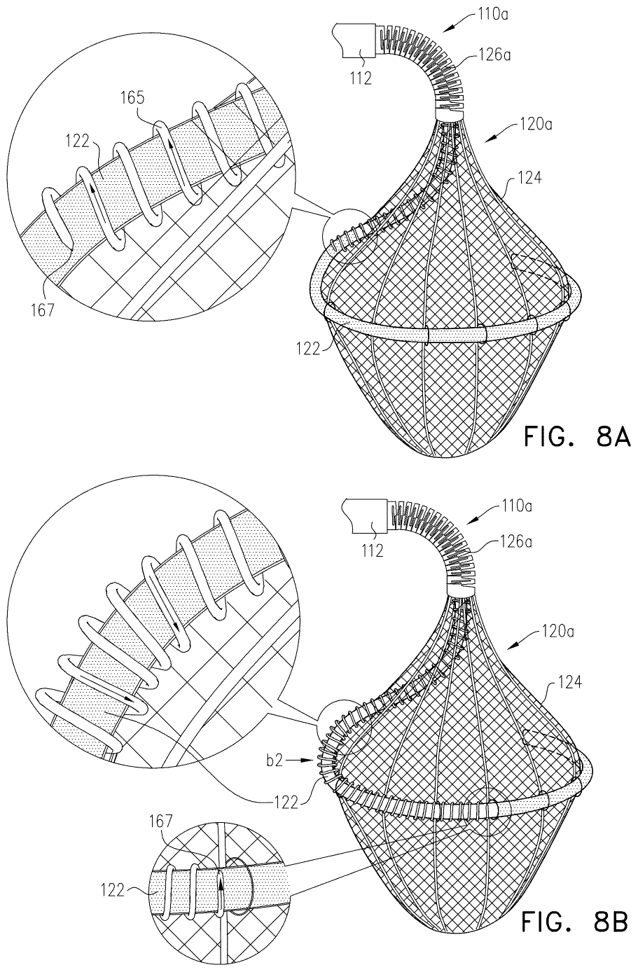

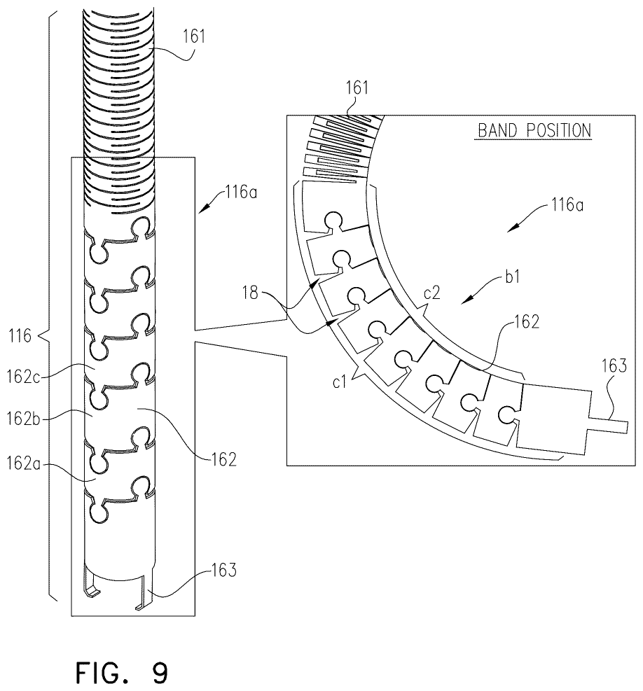

Reference is now made to FIG. 9, which is a schematic illustration of a driver 116a, in accordance with some implementations. Driver 116a may be a variant of driver 116, and may be used for implantation of (e.g., screwing of) a helical member, such as helical member 165, into and along tissue of a body orifice (e.g., into and along tissue of an anulus). Driver 116a can apply torque to the helical member while a drivehead 163 of the driver is engaged with a head (e.g., head 169) of the helical member. Driver 116a may be used with any of the guide assemblies mentioned herein.

In some implementations, driver 116a comprises and/or is formed from a tube 16 (e.g., a hypotube) that defines a driveshaft 161 along a significant part of the tube's length, drivehead 163 at its distal end (e.g., for reversibly engaging helical member 165), and a neck 162 (e.g., a neck portion) between the driveshaft and the drivehead. As described hereinbelow, neck 162 has different properties from driveshaft 161 with respect to bending and application of torque.

In some implementations, driveshaft 161 and neck 162 are formed from a single, unitary tube in a manner that confers the different properties on the driveshaft and the neck. For example, during manufacturing of driver 116a, different cut patterns may be made along the unitary tube, thereby forming (e.g., dividing the tube into) the driveshaft and the neck.

In some implementations, and as shown, driveshaft 161 defines multiple transverse slits (e.g., a first cut pattern) along its length, the driveshaft being bendable via deformation of the tube and the slits (i.e. straining of the material of the tube).

In some implementations, neck 162 defines a second pattern of cuts along its length that segment the neck into discrete interlocking vertebrae 162a, 162b, 162c, etc. (e.g., each having a “puzzle piece” form) that can articulate with respect to each other. For example, and as shown, neck 162 may form a bend b1 by the parts of the vertebrae along an outside circumference c1 of the arc moving apart to form gaps 18, and the parts of each of the vertebrae that lie along the inner circumference c2 moving together. Despite their articulation with respect to each other, vertebrae 162a-c may be rotationally locked with respect to each other, thereby allowing the transfer of torque.

In some implementations, drivehead 163 is also formed from tube 16, e.g., the drivehead is formed (e.g., cut and/or shaped) from the same unitary tube as the driveshaft and the neck. Alternatively, in some implementations, drivehead 163 is welded to the end of tube 16, e.g., the drivehead comprises and/or is formed from a different piece of stock material.

In some implementations, the cut pattern of driveshaft 161 may be particularly suited for the long transluminal path from the percutaneous site of entry into the subject to the heart of the subject. For example, the cut pattern may provide resilience, stability, and smoothness to driveshaft 161, which may be advantageous for transmission of torque along the long transluminal path—e.g., due to favorable (e.g., minimal) interactions between the driveshaft and other components of the delivery assembly, such as with guide rail 122 (coaxially inside the driveshaft) and with the control shaft (which may be coaxially outside the driveshaft).

The cut pattern of neck 162 may be particularly suited for the more distal region of driver 116a. For example, compared with the cut pattern of driveshaft 161, the cut pattern of neck 162 may provide greater torque strength (e.g., be less prone to failure) while applying torque under a high degree of contortion—i.e. when bent to a small radius of curvature, which may be advantageous for transmission of torque at the distal end of the guide assembly. For example, the guide rail may be bent at a tight bend (b2 shown in FIG. 8B) around and/or at guide frame 124, e.g., at the portion that the guide rail reaches the first fastener and/or the midsection of the guide frame.

Although driver 116a has been described as being usable and/or configured for use with a helical member that is screwed along the tissue (e.g., such that a screw axis of the anchor lies along the tissue), it is to be understood that driver 116a could be used to drive (e.g., screw) in any other types of tissue anchors into tissue, e.g., driver 116a could be used to screw a tissue anchor longitudinally into tissue.

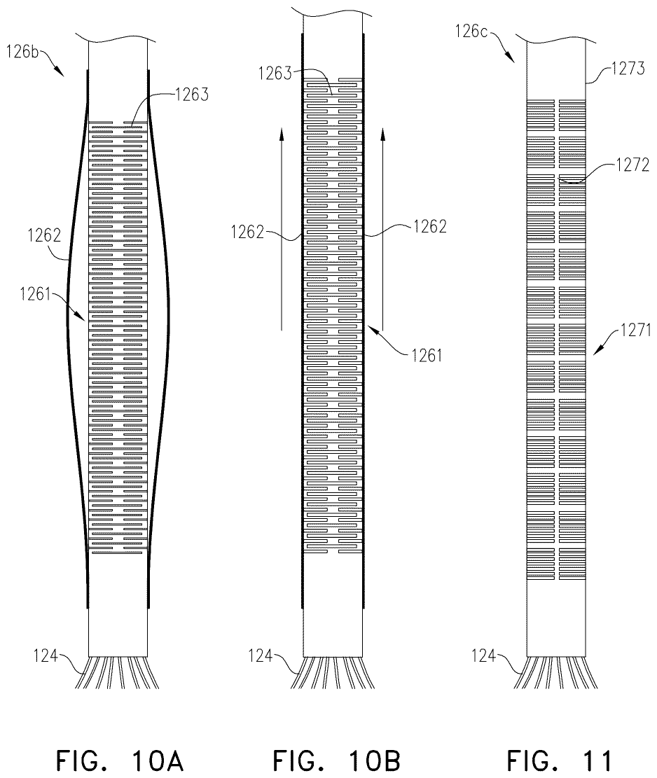

Reference is now made to FIGS. 10A-B, which illustrate a distal portion of a control shaft 126b, in accordance with some implementations. Control shaft 126b can be a variant of any of control shafts 126 and/or 126a, and can be used as a component of a guide assembly as described for these control shafts. It is to be understood that features of control shaft 126b may be provided on any of the control shafts described herein. For example, the distal portion shown in FIGS. 10A-B may correspond to the portion of control shaft 126a that is visible in FIGS. 6A-C.

In some implementations, it may be advantageous for the distal portion of a control shaft to have more flexibility than more proximal portions of the control shaft. This greater flexibility may allow the distal portion of the control shaft to assume a radius of curvature to position guide frame 124 at the annulus. The distal portion may thus be more flexible than more proximal portions of the control shaft.

In some implementations, the distal portion may be considered to define a flex zone 1261 in which cuts in the tube confer extra flexibility on the control shaft. For some implementations, the control shaft may be a tube 1263, with at least flex zone 1261 having a hypotube-type structure having multiple cuts along its length.

In some implementations, subsequently to implanting helical member 165 along the annulus, and/or once implant 160 has been fully implanted and/or contracted within the heart, guide frame 124 is withdrawn back into the delivery assembly (e.g., into sheath 112), and out of the heart and the subject. This may be achieved by pulling the control shaft proximally. Therefore, the control shaft may require tensile strength sufficient for such pulling. However, for the cut patterns in hypotube-type structures, a trade-off may exist between flexibility and tensile strength.

In order to enhance the tensile strength of flex zone 1261 (e.g., and without significantly reducing its flexibility), control shaft 126b may have a pair of strips 1262 that extend alongside the flex zone. In some implementations, strips 1262 can have greater tensile strength than flex zone 1261.

As shown in FIG. 10A, a first end of each strip 1262 is attached to the tube distally from the flex zone 1261, and a second end of each strip is attached to the tube proximally from the flex zone. In some implementations, at rest, (e.g., before the distal portion is pulled proximally), the strips 1262 lie slack alongside the flex zone 1261. In some implementations, the strips can be bonded (e.g., welded) to tube 1263 at each end of the strip.

Once implantation of helical member 165 and/or implant 160 is complete (and optionally guide rail 122 has been withdrawn proximally back into the delivery assembly), guide frame 124 is withdrawn from out of the heart and into sheath 112 by pulling the control shaft. As illustrated in FIG. 10B, pulling the control shaft axially stretches flex zone 1261, eliminating the slack in strips 1262 (e.g., tensioning the strips), which resist further stretching of the flex zone. As shown, strips 1262 may press (e.g., medially) against flex zone 1261 when tensioned. Strips 1262 may thereby advantageously provide enhanced tensile strength to flex zone 1261 (e.g., preventing failure and/or breakage of the distal portion during the pulling of the guide frame back into the sheath) while also advantageously allowing the control shaft to benefit from the flexibility provided by flex zone 1261 itself—e.g., without overly restricting flexing of the flex zone.

In the example shown, the distal portion of control shaft 126b has two strips 1262. However, it is to be understood that a single strip 1262, or more (e.g., 3, 4, or more) strips may be used.

Reference is now made to FIG. 11, which, similarly to FIGS. 10A-B, illustrate a distal portion of a control shaft 126c, the distal portion defining a flex zone 1271 having at least one reinforcing strip 1272 extending along its length, in according to some implementations. Control shaft 126c can be a variant of control shaft 126 and/or 126a, and can be used as a component of a guide assembly as described for these control shafts. It is to be understood that features of control shaft 126c may be provided on any of the control shafts described herein. For example, the distal portion shown may correspond to the portion of control shaft 126a that is visible in FIGS. 6A-C.

Similarly to flex zone 1261, flex zone 1271 may be more flexible than the rest of the control shaft. For some implementations, the control shaft may be a tube 1273, with at least flex zone 1271 having a hypotube-type structure having multiple cuts along its length.

In order to provide enhanced tensile strength to flex zone 1271, strip 1272 may be formed by leaving an uncut axial strip along the distal portion (i.e. lying parallel with the axis of tube 1273). For example, flex zone 1271 may define a cut pattern along the tube that includes multiple slits distributed along the distal portion, with each of the slits incompletely circumscribing the tube such that uncut axial strip 1272 remains, extending axially along the flex zone.

In some implementations, the cut pattern defines a second uncut axial strip along the flex zone, the second strip being disposed on an opposing side of the flex zone to the first strip (not shown). In some implementations, the cut pattern defines three, four, or more uncut axial strips along the flex zone, each strip being spaced apart from, and parallel to, its neighboring strips along the flex zone.

In some implementations, control shaft 126c may advantageously be manufactured with strips 1272 integrated—e.g., as part of the cut pattern that defines flex zone 1271, thereby obviating a need for an additional manufacturing step of attaching flex strips to the tube of the control shaft.

It is to be noted that although strips 1262 and 1272 are described in the context of the control shafts described herein, it is to be understood that they may be used to provide enhanced tensile strength to any catheter, sheath, tube (e.g., hypotube), delivery assembly, or guide assembly independent of the systems and methods described herein.

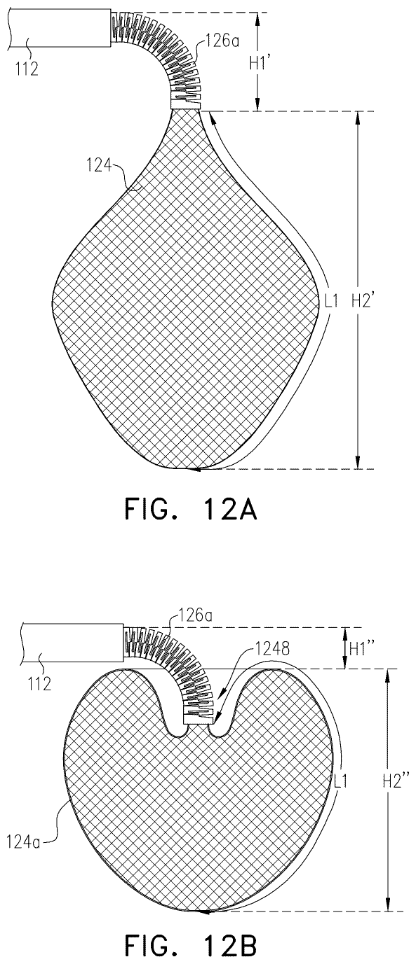

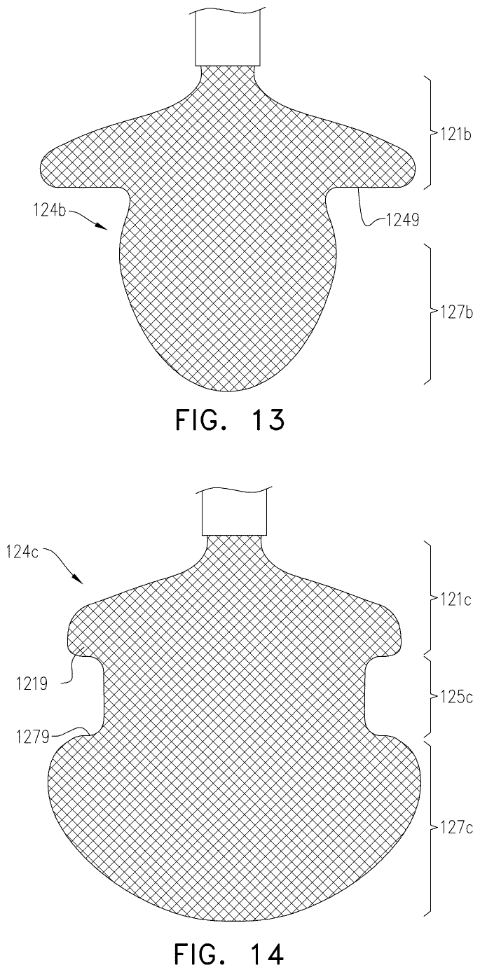

Reference is now made to FIGS. 12A-B, which illustrate various guide frames, in accordance with some implementations. For simplicity, FIG. 12A shows guide frame 124 of guide assembly 120a (e.g., without guide rail 122 or spacers 130)—e.g., in order to facilitate comparison with guide frame 124a shown in FIG. 12B. Guide frame 124a can be considered to be a variant of guide frame 124, and may be used in place of guide frame 124, mutatis mutandis. Moreover, features of guide frame 124a may be integrated into any of the other guide frames described herein.

FIGS. 12A-B show their respective guide frames attached to control shaft 126a at a proximal end of the guide frame. As shown in FIG. 12B, guide frame 124a has an invaginating part at its proximal end, which invaginates upon deployment and/or expansion of the guide frame, such that the guide frame (e.g., a proximal part thereof) forms an invagination 1248 where it is sunken into itself. Because control shaft 126a is coupled to guide frame 124a at invagination 1248, the guide assembly may require less space within the atrium. That is, once expanded, at least part of guide frame 124a that is disposed within the atrium (e.g., an upstream section) is invaginated, with control shaft 126b extending into the invagination.

In some implementations, guide frame 124a may be formed by heat-setting (e.g., shrinking) the frame into itself and/or to control shaft 126b, and/or using other manufacturing methods. This may allow for a larger portion of the guide frame to be positioned within the atrium—e.g., as compared with the arrangement illustrated in FIG. 12A.

For example, a control-shaft-effective height H1″ of a guide assembly that comprises guide frame 124a may be smaller than control-shaft-effective-height H1′ of a guide assembly that comprises guide frame 124. In this context, “control-shaft-effective-height” refers to the height, along an atrioventricular axis, of the control shaft exposed within the atrium upstream of the guide frame. Similarly, a deployed height H2″ of guide frame 124a may be smaller than a deployed height H2′ of guide frame 124. It is to be noted that, in some implementations, invagination 1248 may provide these differences without a material difference between the guide frames in their surface-length L1. In this context, “surface-length” refers to the longitudinal distance along the surface of the guide frame, measured from the control shaft to the downstream end of the guide frame.

In some implementations, FIGS. 12A-B may be viewed as representing a manufacturing technique of guide frame 124a. For example, the guide frame may first be formed (FIG. 12A), and subsequently invaginated into itself (FIG. 12B).

In some implementations, the guide frame may optionally be heat-set to retain invagination 1248.

In some implementations, the invagination may be performed prior to or subsequent to the attachment of the control shaft 126a to the guide frame.

In some implementations, the invagination may cause the proximal part of the guide frame to become contoured—e.g., by being bent inwardly. In some implementations, invagination 1248 exists both in the compressed/delivery state of the guide frame and in the expanded state of the guide frame (e.g., in which the guide assembly is in its guide arrangement). In some implementations, in the compressed/delivery state of guide frame 124a, the proximal part of the guide frame is externalized, and invaginates upon expansion of the guide frame.

Reference is now made to FIGS. 13 and 14, which illustrate guide frames, in accordance with some implementations.

FIG. 13 schematically illustrates an implementation in which, in an expanded state of a guide frame 124b, an upstream section 121b of the guide frame is wider than a downstream section 127b of the guide frame, in accordance with some implementations. This may advantageously allow for upstream section 121b to act as a flange for guide frame 124b when the guide frame is positioned at the valve. For instance, in this configuration, the upstream section can abut against an upstream surface of the annulus of the valve, thereby preventing downstream movement of the guide frame. As shown, once expanded, guide frame 124b assumes a mushroom-shaped form, with upstream section 121b defining a ridge (e.g., shoulder) 1249 extending radially outwards (e.g., over a midsection of the guide frame).

In some implementations, in order to position guide frame 124b within the heart, the guide frame may be expanded within the atrium, and then moved downstream until ridge 1249 abuts (e.g., is stopped by) the atrial-facing surface of the annulus.

Alternatively, in some implementations, guide frame 124b is expanded within the ventricle, downstream of the valve being treated, and the expanded guide frame is then moved upstream, through the valve (e.g., such that ridge 1249 squeezes past the tissue) to protrude radially outwards over the upstream surface of the annulus.

In some implementations, guide frame 124b may be expanded at the valve, e.g., such that, as the guide frame expands (and forms the mushroom shape), ridge 1249 becomes pressed against the atrial-facing surface of the annulus.

FIG. 14 illustrates a guide frame 124c that includes an upstream section 121c (e.g., for positioning within an atrium) and a downstream section 127c (e.g., for positioning within a ventricle, downstream of the atrium) that are both wider than a midsection 125c of the guide frame. For example, once expanded, upstream section 121c may define a ridge 1219 (e.g., shoulder) that extends radially outwards (e.g., over midsection 125c of the guide frame), and downstream section 127c may define a ridge 1279 that extends radially outwards (e.g., under the midsection). That is, once guide frame 124c is expanded, midsection 125c may have a smaller circumference than both the upstream section and the downstream section, e.g., the midsection thereby assuming the form of a concave waist.

In some implementations, guide frame 124c may be expanded while midsection 125c is positioned in the vicinity of annulus 10, such that the annulus becomes sandwiched (e.g., gripped) between the upstream section and the downstream section. For example, during expansion of guide frame 124c, upstream section 121c may expand radially outwards, becoming wedged in the atrium (e.g., pressed against an atrial-facing surface of the annulus), and downstream section 127c may expand radially outwards, becoming wedged in the ventricle (e.g., pressed against a ventricular-facing surface of the annulus).

This sandwiching of the valve between the upstream section and the downstream section may prevent slippage or undesirable movement of the guide frame with respect to the tissue during anchoring of helical member 165 along the annulus.

In some implementations, upstream section 121c and downstream section 127c are expanded independently of each other.

For example, in some implementations, prior to expanding downstream section 127c, upstream section 121c is expanded within the atrium, and the guide frame downstream towards the valve, until ridge 1219 abuts the annulus (within the atrium). In some such implementations, downstream section 127c is then expanded, e.g., thereby forming ridge 1279 within the ventricle.

Alternatively, in some implementations, prior to expanding upstream section 121c, downstream section 127c is expanded within the ventricle, and the guide frame is moved upstream towards the valve until ridge 1279 abuts the annulus (within the ventricle). In some such implementations, upstream section 121c is then expanded, e.g., thereby forming ridge 1219 within the atrium.

In some implementations, in its guide arrangement, guide rail 122 may be positioned along midsection 125c (e.g., just below ridge 1219). In some implementations, in its guide arrangement, guide rail 122 may be positioned along an underside of ridge 1219 of upstream section 121c.

Reference is now made to FIGS. 15A-C, which illustrate an implementation in which multiple actuator wires 136 can be used, within the heart, to both expand the guide frame, and to reorient the guide frame.

FIGS. 15A-C illustrate a guide assembly 120b comprising guide frame 124, control shaft 126a, and a handle 150a at an extracorporeal portion thereof. Handle 150a may be used to control the delivery of guide assembly 120b to the heart, and/or to guide the implantation of helical member 165 along the annulus (not shown).

In some implementations, guide assembly 120b comprises a guide rail (e.g., guide rail 122), which is not shown for clarity.

Guide assembly 120b can be a variant of any of the guide assemblies mentioned herein, e.g., except when noted otherwise.

FIGS. 15A-C illustrates an implementation in which multiple actuator wires 136 are woven longitudinally along at least part of the guide frame such that tensioning the actuator wires radially expands the guide frame (as illustrated in the transition from FIG. 15A to 15B), e.g. by pulling the downstream section of the frame toward the upstream section of the frame, thereby foreshortening the frame. Actuator wires 136 can similarly be used to radially compress the guide frame back to its delivery state, e.g., at the end of the procedure. For example, wires 136 may have sufficient column strength and/or rigidity that pushing them distally pushes the downstream section of the frame away from the upstream section of the frame, thereby elongating the frame.

In some implementations, the guide frame is configured to radially expand responsively to balanced tension applied to the actuator wires. In some implementations, a controller 156a on handle 150a can be configured to apply such balanced tension to the actuator wires in order to expand the guide frame. In some implementations, this is achieved by pulling or pushing the actuator wires to apply balanced tension to the wires, e.g., applying tension to the wires such that each wire has a similar, or substantially equal, amount of tension therein.

In some implementations, the actuator wires are attached to a distal end of the guide frame, either by looping around the distal end and back up the opposite side of the guide frame (e.g., as illustrated in FIG. 3B with reference to spacers 130, mutatis mutandis), or by an end of each of the wires being attached to the distal end of the guide frame.

In some implementations, having multiple actuator wires 136 woven along the guide frame advantageously provides for a more uniform, evenly-rounded expansion of the guide frame, e.g., vis a vis using a single actuator wire, or only a pair of actuator wires. For example, tension in the wires may become evenly distributed along the wires during the application of balanced tension to the wires.

In some implementations, in addition to being used to expand/compress the guide assembly, actuator wires 136 can be used to intracardially reorient and/or pivot guide frame 124 with respect to control shaft 126a (e.g., as illustrated by the transition between FIG. 15B and FIG. 15C). For example, once guide frame 124 is positioned within the heart, the user (e.g., a physician) can adjust the position and/or orientation of the guide frame with respect to the valve, e.g., to better position the midsection of the guide frame along the annulus. In some implementations, the guide frame is configured to pivot responsively to differential tension applied to actuator wires 136.

In some implementations, a controller 156b on handle 150a can be configured to apply such differential tension to the actuator wires in order to pivot the guide frame. In some implementations, controller 156b can be in the form of a joystick, e.g., allowing the user to reorient (e.g., pivot) the guide frame within the heart. In some implementations, controller 156b has multiple actuators (e.g., knobs), each actuator being actuatable (e.g., rotatable) to pivot the guide frame in a single plane by tensioning only a subset of the wires.

In some implementations, and as described hereinabove, handle 150a (e.g. its controllers) can be configured with (i) a first actuation mode that applies the balanced tension to the actuator wires, and (ii) a second actuation mode that applies the differential tension to the actuator wires. In some implementations, these actuation modes are provided by separate controllers. In some implementations, these actuation modes are provided by a single controller that is switchable between the first actuation mode and the second actuation mode. In some implementations, the guide frame is expandable independently of the pivoting, and vice versa.

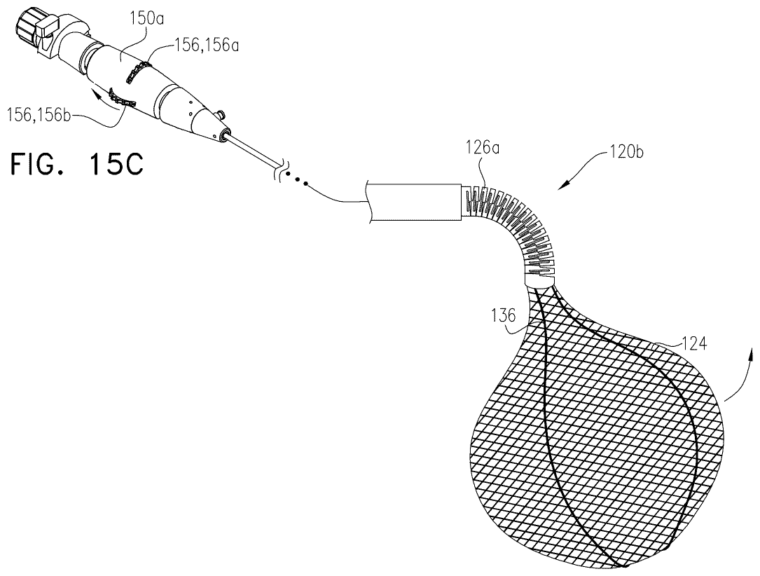

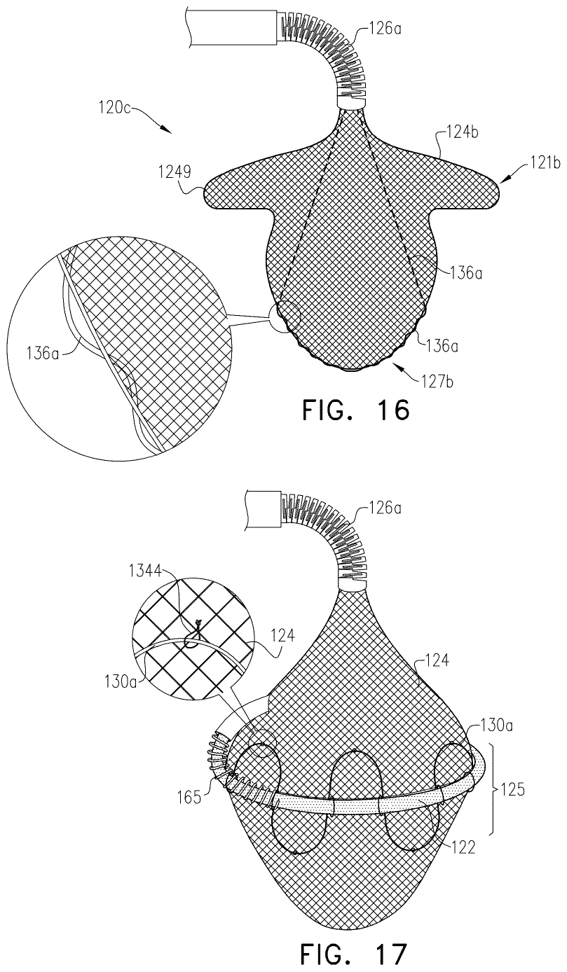

Reference is now made to FIG. 16, which illustrates an implementation in which multiple actuator wires 136a are used that are woven along a downstream section of the guide frame, e.g. but not along an upstream section of the guide frame. Actuator wires 136a can be considered to be variants of actuator wires 136, except when noted otherwise. FIG. 16 shows actuator wires 136a being used with a guide assembly 120c, which may be a variant of, or substantially identical, to any of the other guide assemblies described herein, except when noted otherwise. Guide assembly 120c comprises a guide frame (shown as, but not limited to, guide frame 124b), and a control shaft 126a which is attached to a proximal part of the guide frame. Actuator wires 136a extend, from control shaft 126a, distally through an interior of the upstream section 121b of the guide frame, and weavingly along the downstream section 127b of the guide frame.

The arrangement of actuator wires 136a illustrated herein can be used with any guide frame (e.g., with any of the guide frames described herein). However, the arrangement of actuator wires shown in FIG. 16 may be particularly advantageous when used with a guide frame that has an upstream section that is wider than the midsection and/or than the downstream section (e.g., wider than any part and/or a greatest width of the downstream section), such as described with reference to guide frame 124b (as shown) or guide frame 124c. In such implementations, having actuator wires 136a bypass the upstream section (e.g., by extending through the interior of the guide frame rather than weavingly along the guide frame) may advantageously allow the upstream section to expand toward its desired, pre-set shape (e.g., ridge 1249) without the actuator wires interfering with the expansion (e.g. by medially constraining the upstream section). In some implementations, the weaving of wires 136a along the downstream section provides a similar advantage to that described hereinabove with reference to actuator wires 136, e.g., applying balanced tension to the wires provides for an evenly-expanded guide frame, e.g., due to the forces becoming distributed evenly across the wires.

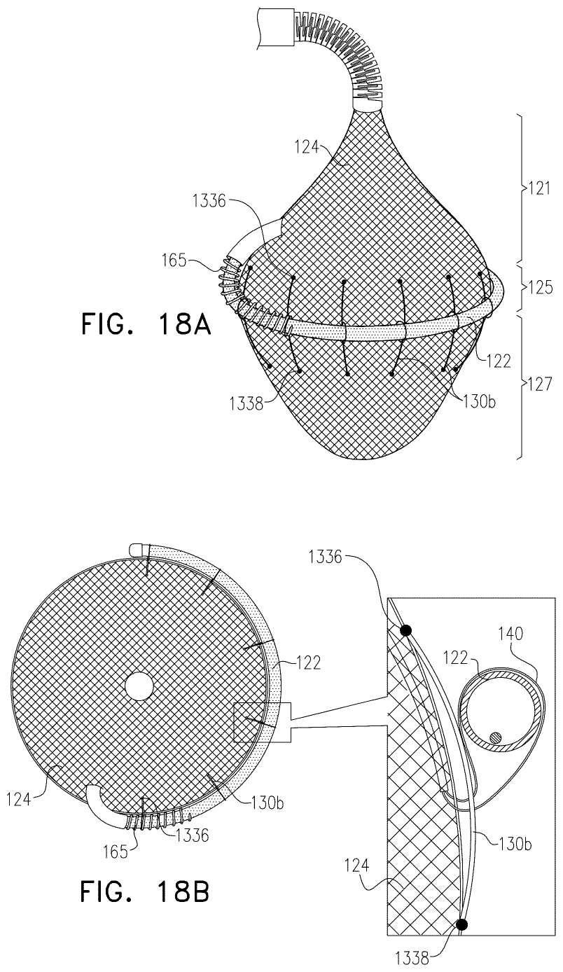

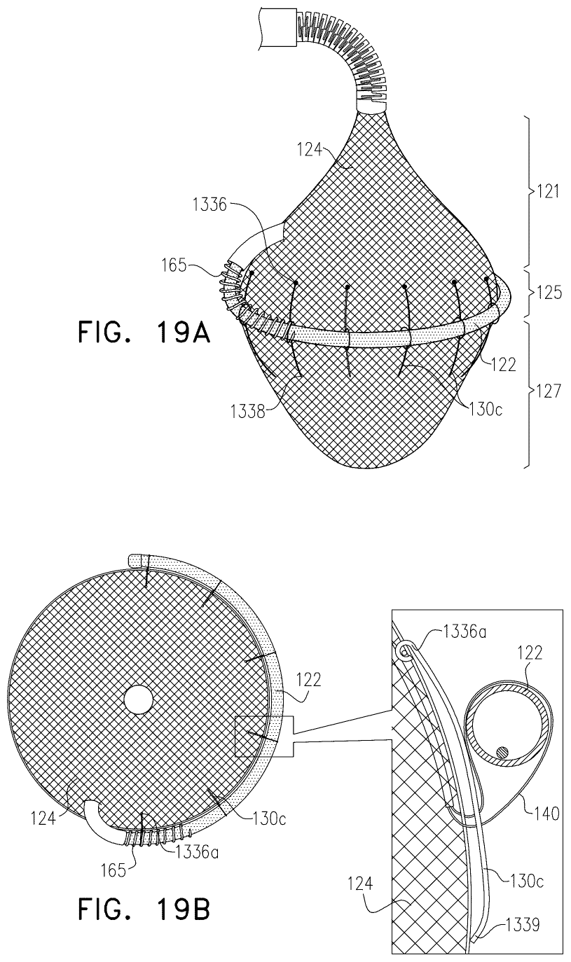

Reference is now made to FIGS. 17, 18A-B, and 19A-B which illustrate various systems and methods for spacing guide rail 122 from guide frame 124, e.g., in order to prevent helical member 165 from catching onto the guide frame during screwing of the helical member along the tissue.

FIG. 17 illustrates a spacer 130a that extends in a serpentine manner around midsection 125 of guide frame 124. Spacer 130a can be used in place of any of the other spacing techniques described herein, and/or can be used with any of the guide frames and/or guide assemblies described herein.

Spacer 130a can be formed from a single elongate member, such as a wire, a ribbon, a string, and/or a rope that extends in a serpentine (e.g., wave) form around midsection 125. Spacer 130a has a sufficient width to space the guide rail from the guide frame.

Spacer 130a can be secured to guide frame 124 by intermittently weaving along the guide frame, or, as shown, via connectors 1344 (e.g. loops). In some implementations, the spacer is secured to the guide frame at each “peak” and “trough” of the spacer (i.e. of its serpentine form). In some implementations, during expansion of guide frame 124, the wavelength of spacer 130a increases, and during compression of the guide frame, the wavelength decreases. In some such implementations, the segments of spacer 130a between its peaks and troughs are substantially loose with respect to guide frame 124—e.g. to facilitate expansion and compression of the guide frame. In some implementations, having spacer 130a extend only along part of the guide frame may advantageously provide a narrower delivery state, e.g., vis a vis an implementation in which spacers extend along the entire length of the delivery assembly.

FIGS. 18A-B illustrate an arrangement in which multiple spacers 130b are used that are positioned along only a part of the guide frame. Spacers 130b can be similar to spacers 130, except that they do not extend along the entire length of guide frame 124. Rather, each spacer 130b extends longitudinally alongside part of the upstream section 121, the entire midsection 125, and part of the downstream section 127, such that each spacer “floats” alongside the guide frame. In order to achieve this, each of the spacers has an upstream terminus 1336 that is attached to the guide frame at the upstream section, and a downstream terminus 1338 that is attached to the guide frame at the downstream section. In some implementations, and as illustrated in FIG. 18B, each spacer 130b curves radially away from guide frame 124 between termini 1336 and 1338, e.g., such that, at midsection 125, the spacer presses guide rail 122 and fastener 140 radially outward and away from the guide frame, thereby preventing helical member 165 from catching onto the guide frame. Having spacers 130b extend only along part of the guide frame may advantageously provide a narrower delivery state, e.g., vis a vis an implementation in which the spacers extend the entire length of the delivery assembly.

In some implementations, rather than each spacer 130b being in the form of a wire, each spacer is in the form of a ribbon. In some implementations, each ribbon contacts its neighboring ribbon, e.g., such that the ribbons collectively form a shield around the midsection. In some implementations, the ribbons do not completely cover the midsection, but nonetheless do shield a substantial portion of the midsection.

In some implementations, spacers 130b can be used in place of any of the other spacing techniques described herein, and/or can be used with any of the guide frames and/or guide assemblies described herein.

Similarly to FIGS. 18A-B, FIGS. 19A-B illustrate spacers 130c which are positioned along only a part of the guide frame. Spacers 130c can be substantively identical to spacers 130b, except that they are only attached to the guide frame at upstream section 121 of the guide frame. For example, each spacer 130c can be attached to upstream section 121 at an upstream terminus 1336a, but not to downstream section 127. In some implementations, upstream terminus 1336a can be in the form of a hook that hooks onto guide frame 124 at upstream section 121.

In some implementations, and as shown, each spacer 130c can extend, from upstream terminus 1336a, along midsection 125, to downstream section 127, where the spacer terminates in a free end 1339.

In some implementations, free end 1339 may be positioned (e.g., may rest) against the exterior of guide frame 124, as shown.

In some implementations, free end 1339 may protrude into guide frame 124 without being fixed to the guide frame. Such an arrangement may advantageously allow for the unhooking of spacers 130c from helical member 165, should the helical member catch onto any of the spacers during screwing of the helical member along guide rail 122 and the tissue. For example, once the implantation of helical member 165 along the annulus is complete, and guide rail 122 and fasteners 140 have been withdrawn from the heart, guide frame 124 can be medially compressed back towards its delivery state (e.g., the state of the guide frame illustrated in FIG. 6A) and withdrawn proximally back into a sheath (e.g., sheath 112). In some implementations, during such compression and withdrawal of the guide frame, spacers 130c (e.g., free ends 1339 thereof) can simply slide out from the helical member.

In some implementations, and as mentioned hereinabove with reference to spacers 130b, rather than each spacer 130c being in the form of a wire, each spacer is in the form of a ribbon. In some implementations, each ribbon contacts its neighboring ribbon, e.g., such that the ribbons collectively form a shield around the midsection. In some implementations, the ribbons do not completely cover the midsection, but nonetheless do shield a substantial portion of the midsection.

It is to be understood that spacers 130c can be used in place of any of the other spacing techniques described herein, and/or can be used with any of the guide frames and/or guide assemblies described herein.

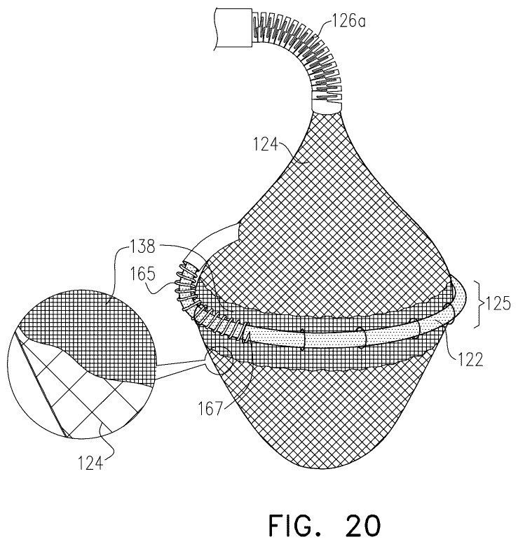

Reference is now made to FIGS. 20-23 which illustrate various implementations in which the guide assembly comprises a shield that is disposed around the midsection. In some implementations, the spacers (e.g. spacer wires) described elsewhere herein are configured to reduce a likelihood of helical member 165 catching or threading onto guide frame 124 by maintaining a radial distance between the guide frame and the helical member. In some implementations, the shields described hereinbelow are configured to have a similar advantageous effect, but by shielding (e.g. masking or obscuring) the guide frame. Thus, the spacers can be relatively thick and open and may not mask midsection 125, whereas the shields may be relatively thin and closed and may substantially mask the midsection.

In some implementations, the shields can be more flexible than guide frame 124, e.g. can themselves be unsuitable to serve as midsection 125. Thus, the shields and the guide frame can cooperate, with the guide frame providing structural support for the guide rail, and the shields providing shielding for the guide frame.

FIG. 20 illustrates one such an arrangement, in which a shield 138 is disposed around midsection 125. In some implementations, shield 138 is discrete from guide frame 124, and can be formed from a different material to that of the guide frame. In some implementations, the material can be and/or comprise an elastic material, a fabric, a film (e.g., a plastic or metallic film), and/or a fine-holed net (e.g., a metallic net). In some implementations, shield 138 is mounted over midsection 125 once the guide frame has been constructed. In some implementations, the shield is formed in place during the manufacture of the guide frame.

It is to be understood that shield 138 can be used in place of any of the other spacing techniques and/or shielding techniques described herein, and/or can be used with any of the guide frames and/or guide assemblies described herein.

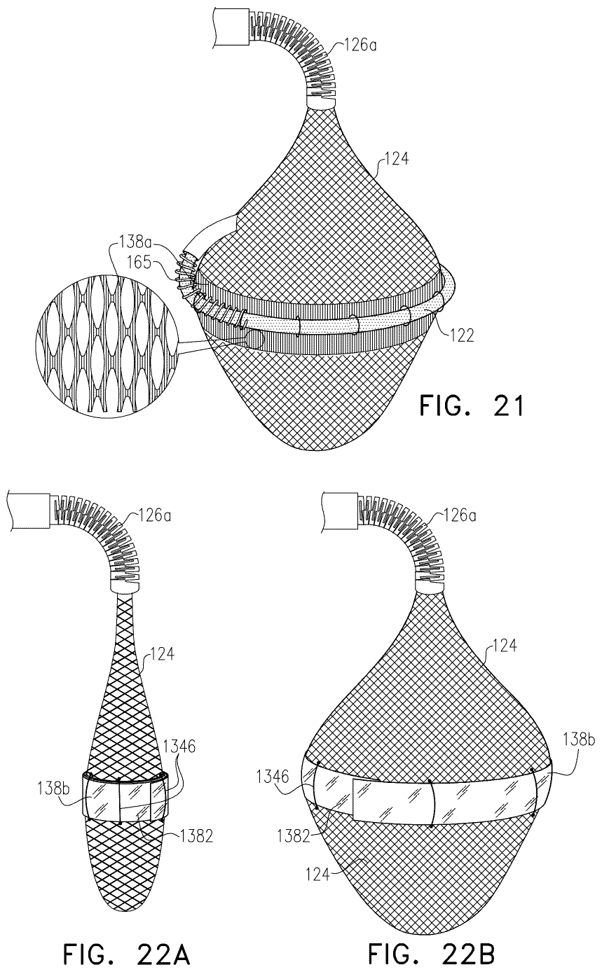

FIG. 21 illustrates a similar shield 138a to shield 138, except that is constructed from a stent-like or hypotube-type material. For example, shield 138a may be constructed from a metallic ribbon or tube that is cut into an array of interconnected struts and tessellated cells, thereby allowing the shield to expand along with guide frame 124. In some implementations, even when the shield is expanded, the cells of the shield may be smaller than the spaces between the struts of frame 124.

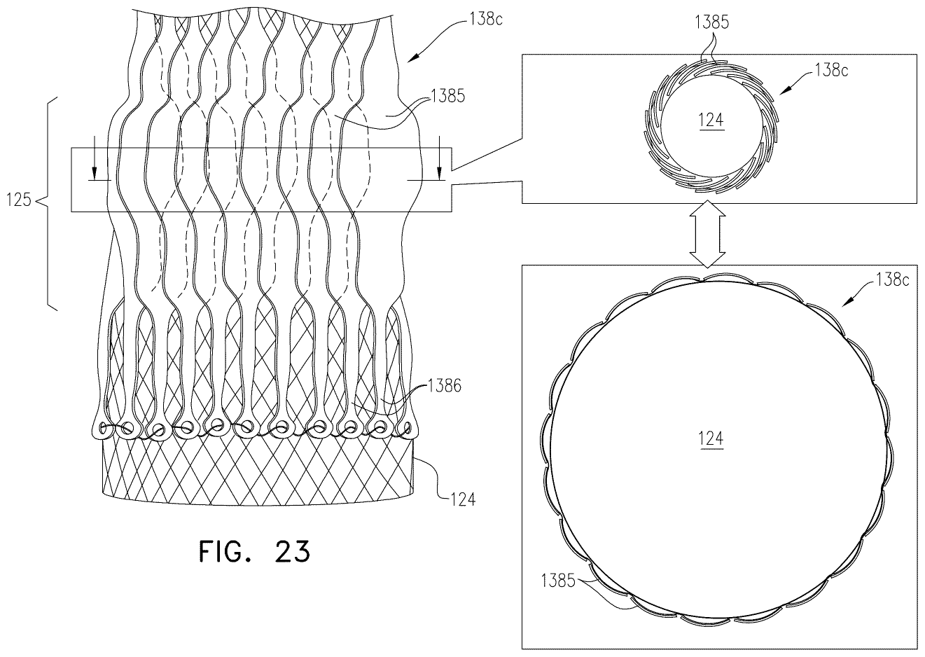

FIGS. 22A-B illustrate a shield 138b that is in the form of a ribbon 1382 that extends latitudinally around the midsection of guide frame 124. In some implementations, in the delivery state of the guide assembly, ribbon 1382 is wrapped multiple times around the guide frame (FIG. 22A), such that expanding the guide frame towards the expanded state causes the ribbon to slide over itself in a manner that reduces the number of times the ribbon is wrapped around the guide frame (FIG. 22B). In some implementations, ribbon 1382 is secured to guide frame 124 via multiple loops 1346 that extend out from the guide frame and around the ribbon, such that, during expansion and contraction of the guide frame, the ribbon can slide around the guide frame as described hereinabove, e.g. similarly to belt loops of a garment. Ribbon 1382 may be formed from a film of polymer or metal. In some implementations, ribbon 1382 can be biased toward its multiple-wrapped state such that the ribbon facilitates recompression of guide frame 124 for withdrawal from the subject.

In some implementations, shield 138b can be used in place of any of the other spacing techniques and/or shielding techniques described herein, and/or can be used with any of the guide frames and/or guide assemblies described herein.

Reference is now made to FIG. 23, which illustrates an implementation in which a shield 138c is defined by multiple ribbons 1385 that are distributed circumferentially around the midsection. In some implementations, in the delivery state, ribbons 1385 are imbricated (e.g., overlap with each other) around the midsection.

In some implementations, ribbons 1385 are configured to facilitate expansion of the guide frame towards the expanded state by the ribbons sliding over each other while collectively covering the midsection. That is, during expansion of guide frame 124, as midsection 125 radially expands outwards, each ribbon 1385 can slide away from its neighboring ribbons, e.g., as illustrated by the transition of the ribbons in the plane views of the guide frame in FIG. 23.

Ribbons 1385 can be formed (e.g. cut) from a film of a polymer or a metal.

In some implementations, in the expanded state of guide frame 124, ribbons 1385 remain imbricated (e.g., overlapping) around the midsection. In some implementations, and as shown in FIG. 23, in the expanded state of guide frame 124, ribbons 1385 are arranged edge-to-edge (e.g., touch each other, but do not overlap with each other) around the midsection.

In some implementations, and as shown, each ribbon 1385 is aligned longitudinally with respect to guide frame 124, i.e. its long axis is transverse to midsection 125. In some such implementations, and as shown, the middle of each ribbon is disposed at midsection 125, while the ends 1386 of each ribbon are disposed on either side of the midsection, i.e. one end at the upstream section of the guide frame, and the other end at the downstream section of the guide frame.

In some implementations, and as shown, each ribbon 1385 is wider toward its middle and narrower towards its ends 1386. In such implementations, the imbrication and/or sliding of the ribbons with respect to each other may be primarily (e.g., solely) toward the middle of the ribbons, e.g. the narrowness of the ends of the ribbons may be such that, in the expanded state of the guide frame, substantial gaps exist between them and they do not substantially shield the guide frame. However, this narrowness and lack of overlapping and sliding may advantageously allow ends 1386 to be secured to the guide frame. For example, and as shown, ribbons 1385 may be secured to guide frame 124 solely via attachment of ends 1386 to the guide frame. In the example shown, ends 1386 are stitched to the guide frame, but other attachment means may be used.

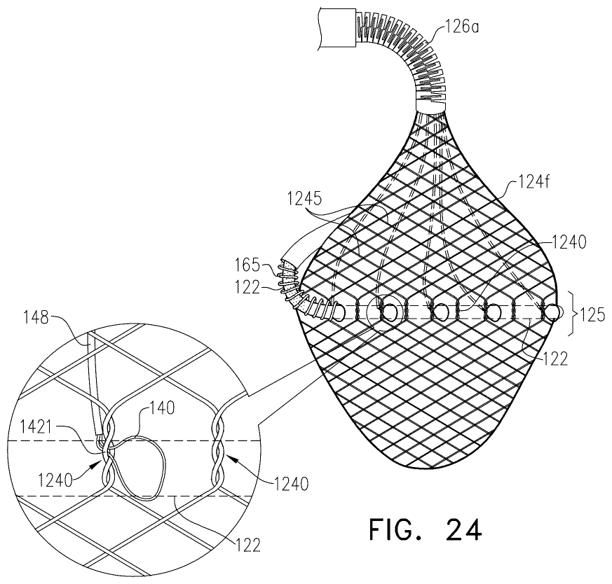

Reference is now made to FIG. 24, which illustrates another feature for reducing a likelihood of helical member 165 catching onto the guide frame—in this case a guide frame 124f. Guide frame 124f can be a variant, or substantially identical to, any of the guide frames described herein, e.g., except when noted otherwise. As described hereinabove with reference to guide frame 124, guide frame 124f can be a braided structure defined by a plurality of struts 1245. In some implementations, in order to prevent helical member 165 from catching onto the guide frame, at the midsection, each strut 1245 of the guide frame is twisted together with an adjacent strut to form a twisted-strut-pair 1240. Because each twisted-strut-pair 1240 is thicker than a single strut, this may advantageously decrease the likelihood of the helical member catching onto the guide frame. For example, twisted-strut-pair 1240 may be too thick to enter between guide rail 122 and tip 167 of helical member 165. Furthermore, each twisted-strut pair 1240 may be substantially parallel to the longitudinal axis of the guide frame, thereby advantageously further decreasing the chances of the helical member catching onto the midsection. In some implementations, each twisted-strut-pair 1240 is covered with a covering or sleeve, e.g., to further increase the thickness of the twisted-strut pairs.

In some implementations, at least some of the twisted-strut-pairs 1240 are shaped to define respective eyelets 1421 therethrough, providing exit sites 142 via which fasteners 140 exit the interior of the guide frame, to the exterior of the guide frame, where the fastener is engaged with (e.g., loops around) guide rail 122. For clarity, the part of guide rail 122 that is in-front of guide frame 124f is shown in phantom.

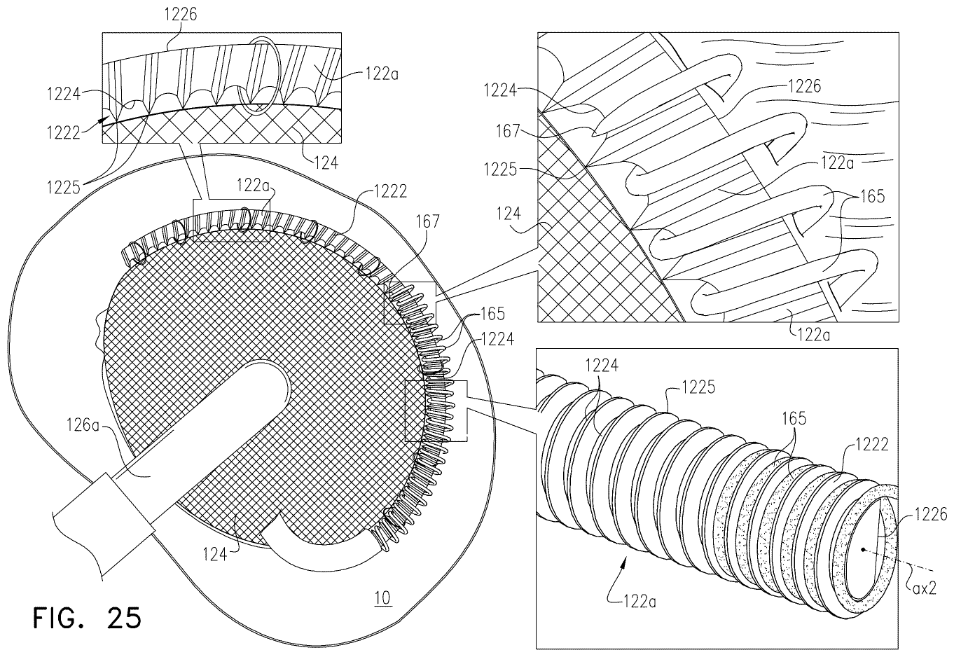

Reference is now made to FIG. 25, which illustrates a guide rail 122a that defines an external thread 1222. Helical member 165 is advanceable along guide rail 122a and the tissue by being rotated threadedly along the external thread. That is, external thread 1222 defines a helical groove 1224, and the driver is configured to screw the helical member helically along the thread while the helical member is recessed within the groove. This threaded engagement between guide rail 122a and helical member 165 may advantageously maintain spacing between guide frame 124 and sharpened distal tip 167 of the helical member. For example, while a crest 1225 of thread 1222 may contact guide frame 124 (e.g. may be pulled against the guide frame by the connectors of the guide assembly), the distal tip of the helical member is held away from the guide frame, remaining recessed within groove 1224 during screwing of the helical member along the tissue.

In some implementations, and as shown, guide rail 122a may have a tissue-facing surface 1226 along one side of the guide rail, and beyond which helical member 165 may reach. For example, guide rail 122a may define a central guide-rail axis ax2, and tissue-facing surface 1226 may, along the guide rail, be disposed closer than the external thread to the central guide-rail axis. In some implementations, once guide rail 122a is positioned in the guide arrangement, thread 1222 faces medially towards the guide frame, and tissue-facing surface 1226 faces radially away from the guide frame. For example, tissue-facing surface 1226 may be positioned against (e.g., facing) the tissue surface. Thus, as helical member 165 is screwed along guide rail 122a, as its tip 167 reaches away from surface 1226, it can penetrate the tissue.

In some implementations, the tissue-facing surface is unthreaded, and runs parallel with the external thread, e.g. parallel with axis ax2.

In some implementations, the tissue-facing surface is substantially flat, e.g., such that guide rail 122a has a D-shaped cross-section (e.g., as shown). In some implementations, the tissue-facing surface is concave. In some implementations, the tissue-facing surface is convex while having a larger radius of curvature than the external thread.

In some implementations, guide rail 122a can be used in place of any of the other spacing techniques and/or shielding techniques described herein, can be used in the place of guide rail 122, and/or can be used with any of the guide frames and/or guide assemblies described herein.

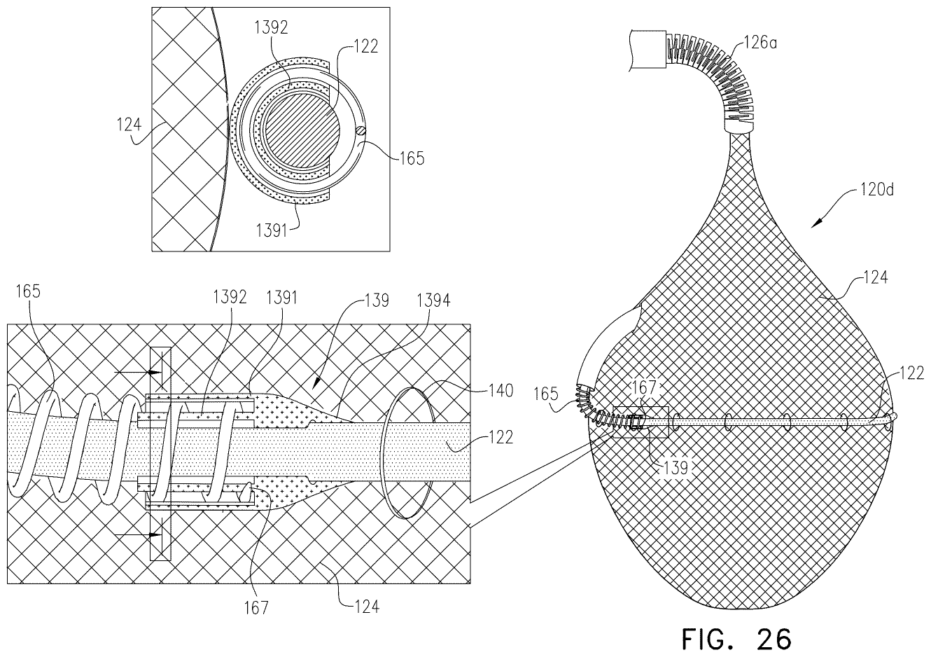

Reference is now made to FIG. 26, which illustrates a guide assembly 120d, which can be a variant of, or substantially identical to, any of the other guide assemblies described herein, except when noted otherwise. In some implementations, guide assembly 120d comprises a rider 139 that is slidably mounted on guide rail 122 such that as driver 116 advances helical member 165 along the guide rail, distal tip 167 of the helical member pushes the rider along the guide rail while the rider shields the guide frame from the distal tip of the helical member.

In some implementations, rider 139 defines a lobe 1391 that, as helical member 165 pushes the rider along the guide rail, remains disposed between distal tip 167 and guide frame 124 (i.e. on a frame side of guide rail 122), thereby shielding the guide frame from the sharpened tip. In some implementations, on an opposite side of the guide rail (e.g. on a tissue-facing side), lobe 1391 may be open or absent, such that helical member 165 can access the tissue. In some implementations, the lobe is rotationally locked with respect to the guide rail e.g., via keying between rider 139 and guide rail 122. This rotational locking may advantageously allow rider 139 to slide along guide rail 122 while maintaining lobe 1391 positioned between distal tip 167 and guide frame 124.

As shown, rider 139 can have a tapered and/or rounded leading part, e.g. a nose 1394, to facilitate sliding of the rider along the surface of guide frame 124 and/or past/through the connectors that may secure the guide rail in the guide arrangement. In some implementations, nose 1394 can be shaped to define an aperture through which guide rail 122 is threaded, thereby providing at least some of the slidable coupling of rider 139 to the guide rail.

In some implementations, lobe 1391 can trail nose 1394. In some implementations, lobe 1391 can curve at least partway around at least the distal few turns of helical member 165.

In addition to lobe 1391, rider 139 can comprise a collar 1392 that curves at least partway around guide rail 122. In some implementations, collar 1392 can provide at least some of the coupling of rider 139 to guide rail 122. For example, collar 1392 may curve more than halfway around the guide rail. Collar 1392 may be open or absent at the tissue-facing side of the guide rail, e.g. similarly to lobe 1391. In some implementations, as in the example shown, collar 1392 and lobe 1391 are substantially concentric with each other, and with the guide rail.

In some implementations, rider 139 can be used in place of any of the other spacing techniques described herein, and/or can be used with any of the guide frames, guide rails, and/or guide assemblies described herein.

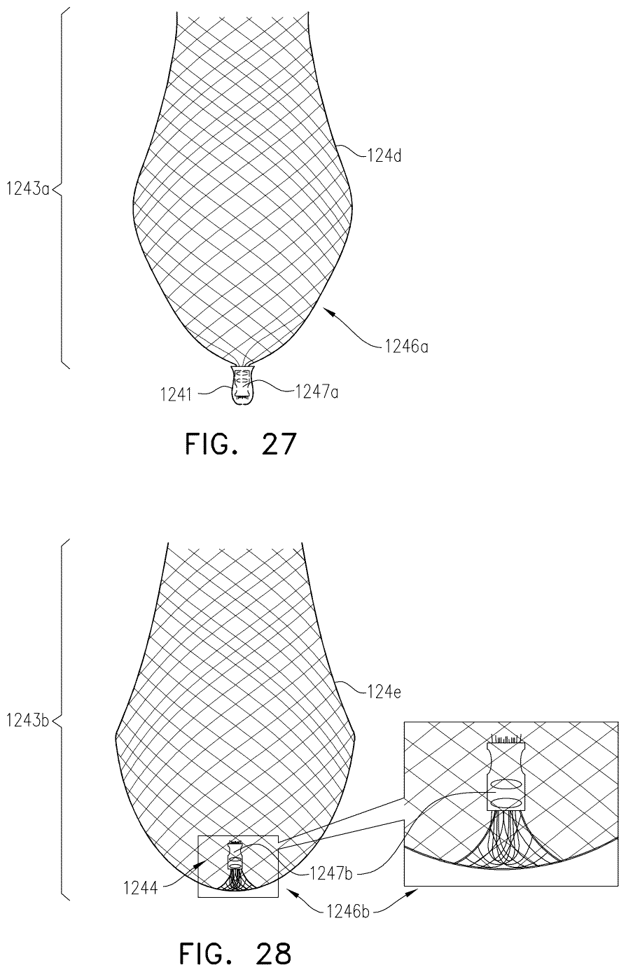

Reference is now made to FIG. 27 and to FIG. 28, which are schematic illustrations of various guide frames that each have an atraumatic distal end, in accordance with some implementations. As noted hereinabove, the guide frame may be formed from a braided filament, i.e. can be a braided guide frame. It may be advantageous for the guide frame to have an atraumatic distal end, in order to prevent inadvertent damage to the surrounding tissue (e.g., damage to the ventricular tissue in which the distal end of the guide frame may contact).

In some implementations, a braided guide frame may be formed by a manufacturing method in which a single filament (e.g., wire) is looped back and forth along the length of the guide-frame-to-be (e.g., looping around pegs of a jig), such that a distal end 1246 of the guide frame is defined by closed (i.e., atraumatic) loops (illustrated in FIG. 3B). That is, the guide frame would be formed from a single filament and therefore has only two filament ends, both of which are disposed away from the distal end of the guide frame (e.g., are disposed at the proximal end of the guide frame).

The guide frames illustrated in FIGS. 27 and 28 also have atraumatic distal ends, but may be less labor-intensive and/or less expensive to manufacture.

FIG. 27 illustrates a guide frame 124d that can be formed by cutting a portion 1243a of a braided tube (e.g., a tube formed from interwoven filaments) and, at one end of the cut portion, gathering together the cut ends and securing them together to form an atraumatic distal end 1246a. For example, the gathered cut ends may be heat-set, secured in a crimp 1247a (e.g., a ferrule). In some implementations, the gathered cut ends and/or the crimp can be covered in a coating or cap 1241 in order to form atraumatic distal end 1246a.

The opposite end of portion 1243a (e.g., opposite to distal end 1246a) may form a proximal end portion of guide frame 124d, by being attached (e.g., via heat-setting) directly to a distal end of a control shaft of the guide frame.

Similarly to FIG. 27, FIG. 28 illustrates a guide frame 124e that can be formed by cutting a portion 1243b of a braided tube (e.g., a tube formed from interwoven filaments—e.g., wires) and, at one end of the portion, gathering together the cut ends and invaginating (e.g., inverting) the gathered ends inwardly into the interior of the guide frame, to form an invagination 1244 therewithin. Invaginating the gathered ends deforms the distal part of the guide frame such that the distal part becomes atraumatically contoured, e.g., by each end of the wire deforming into the interior of the guide frame. In some implementations, the invagination can be secured with a crimp 1247b (e.g., a ferrule), in order to secure and maintain the invagination.

The opposite end of portion 1243b (e.g., opposite to distal end 1246b) may form a proximal end portion of guide frame 124e, by being attached (e.g., via heat-setting) directly to a distal end of a control shaft of the guide frame.

It is to be noted that each of guide frames 124d and 124e comprises multiple filaments (e.g., wires) and therefore many filament ends. Even though half of these filament ends are disposed at the distal end of the cut portion of the braid, they are subsequently made atraumatic by covering (e.g., guide frame 124d) or invagination (e.g., guide frame 124e). These manufacturing techniques may advantageously allow for machine-made lengths of the braid to be produced, which can be cut into portions that are individually formed into guide frames.

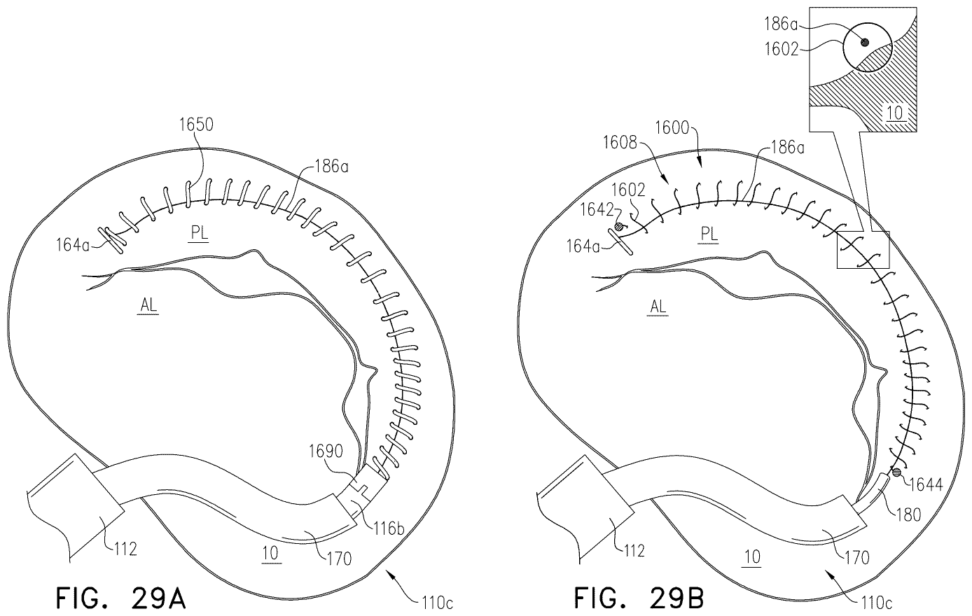

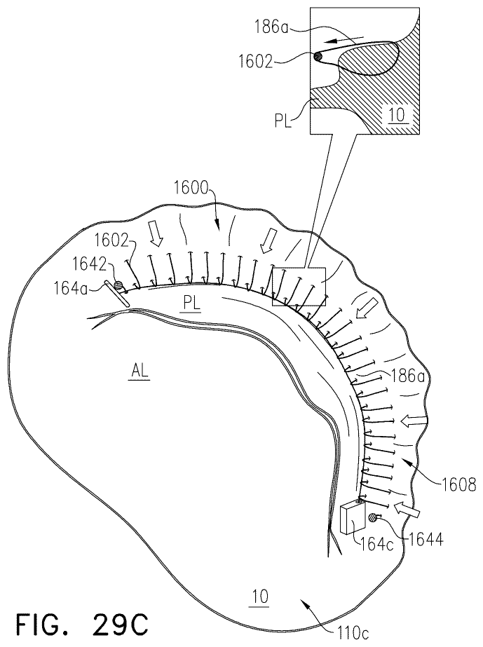

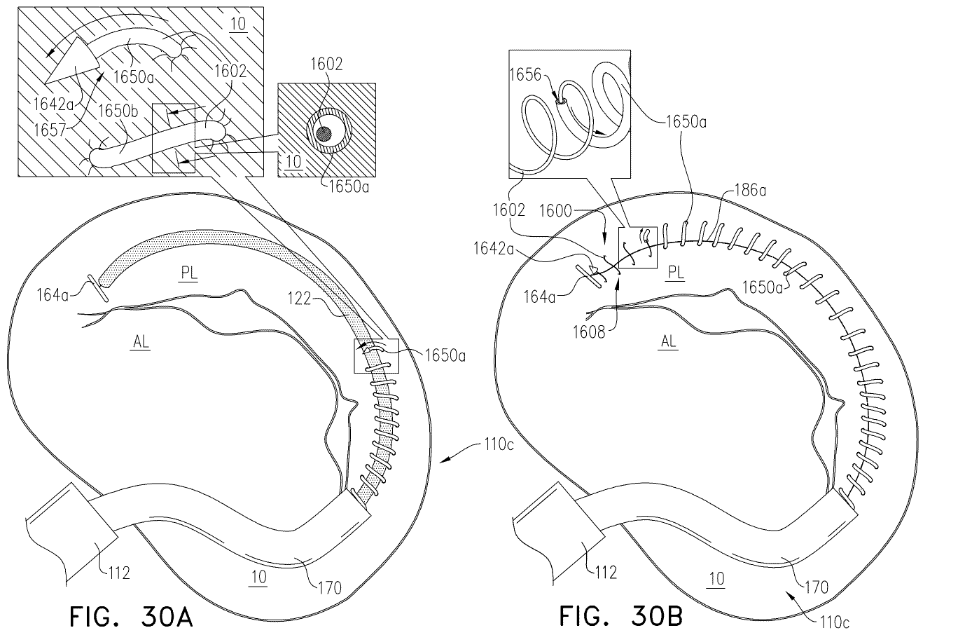

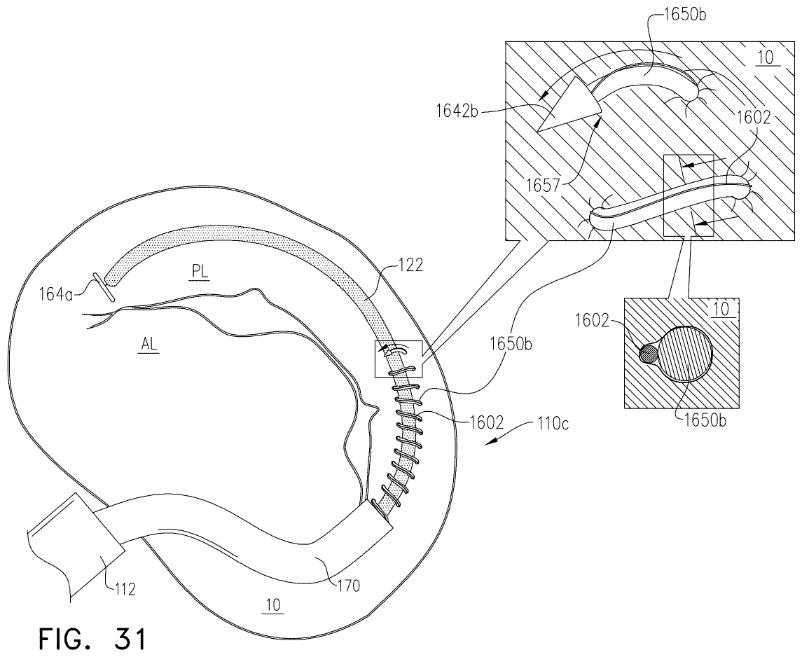

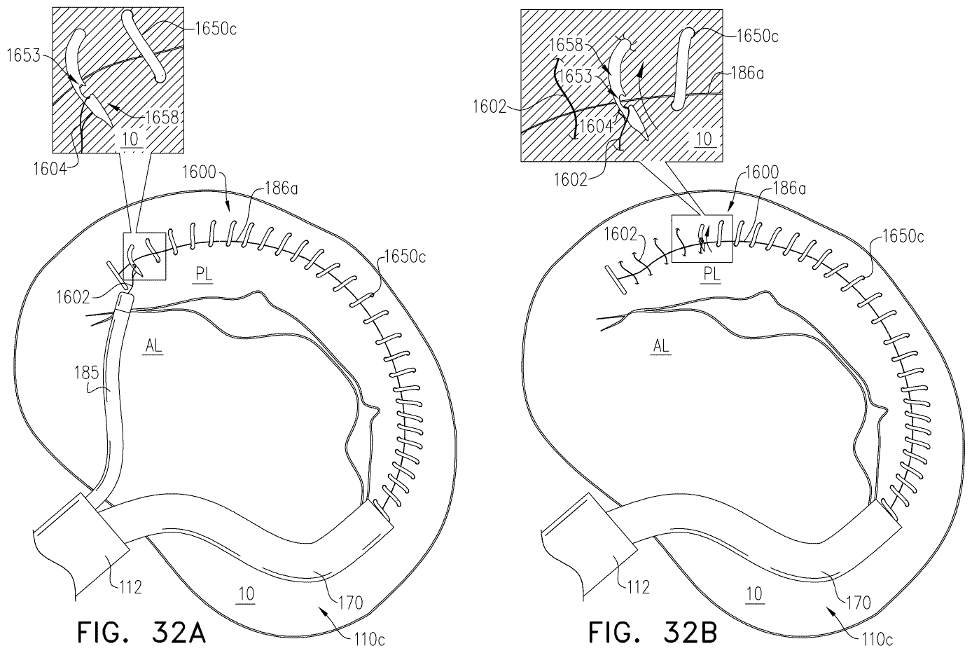

Reference is now made to FIGS. 29A-C, 30A-B, 31, and 32A-B, which illustrate systems and methods of using a flexible helical member 1650 to stitch a suture 1602 of an implant 1600 along a tissue, in accordance with some implementations. Although helical member 1650 may be similar in structure and/or design to helical member 165, e.g., both defining multiple turns and a helix defined by the turns, helical member 1650 is used to stitch a suture along the tissue, and is withdrawn from the subject following the procedure, in contrast to helical member 165 which remains implanted within the subject following the procedure. That is, helical member 1650 is part of a delivery assembly 110c, that is used to implant an implant 1600, rather than being part of an implant itself.

In some implementations, delivery assembly 110c can comprise a driver 116b that is usable and/or used to apply torque to helical member 1650, to screw the helical member along the tissue. In some implementations, the driver may be fixedly (e.g., permanently) attached to helical member 1650, such that the helical member is withdrawn along with the driver, e.g., as will be described hereinbelow. In some implementations, driver 116b may be attached or attachable (e.g., fixedly or reversibly) to helical member 1650 via a head 1690 of the helical member.

Driver 116b may be a variant of any of the drivers described herein, and/or may include features of any of the drivers described herein. In some implementations, helical member 1650 and driver 116b are formed from a unitary element (e.g., shaft)—e.g., the helical member may simply be a continuation of the driver, thereby obviating the need for attachment of the helical member to the driver.

In some implementations, delivery assembly 110c can comprise a tube (e.g., a catheter) 170, that extends distally from a delivery sheath (e.g., sheath 112) of the delivery assembly, to the tissue being treated. In some implementations, tube 170 may be a variant of, or substantively identical to, tubes 118 and/or 118a. Tube 170 may advantageously shield the surrounding tissue (and/or the guide frame, if present) from a sharpened distal tip of helical member 1650 during delivery of the helical member towards the tissue.

In some implementations, delivery assembly 110c may comprise a guide assembly for guiding the screwing of helical member 1650 along the annulus—e.g., as described hereinabove, mutatis mutandis. For example, such a guide assembly may include a guide rail (e.g., guide rail 122, not shown) over which helical member 1650 can be threaded, such that the helical member can be advanced over and along the guide rail during screwing of the helical member into the tissue.

In some implementations, the guide assembly with which delivery assembly 110c is used comprises a guide frame (e.g., guide frame 124, or any of the guide frames described herein), for positioning of the guide rail at the annulus (e.g., in a guide arrangement along the exterior of the guide frame), e.g., using any of the methods or systems described herein.

In some implementations, the guide assembly with which delivery assembly 110c is used does not comprise a guide frame—e.g., it may comprise any of the guide rails described herein that are positionable along the tissue without a guide frame (e.g., guide rails 222, 322, 422, 522, 622 and/or 722). The guide rail may be advanced along the annulus incrementally with the screwing in of helical member 1650.

Although, as noted above, a guide assembly may be used to guide implantation of implant 1600, for the sake of clarity FIGS. 29A-C do not show components of the guide assembly, such as a guide rail and/or a guide frame.

FIG. 29A illustrates helical member 1650 having been (temporarily) screwed into and along tissue of the annulus, such that part of each turn of the driver is embedded within the tissue, and another part of each turn lies above the surface of the tissue (e.g., as described for helical member 165 hereinabove). In some implementations, implant 1600 comprises a tensile member 186a that provides similar functionality to that of tensile member 186 shown with reference to implant 160.

For example, tensile member 186a may similarly be positioned, in a curved path, along the annulus (e.g., within a guide rail), such that helical member 1650 is screwed along the tensile member (e.g., around the tensile member) such that, after the helical member has been screwed into and along the tissue, the tensile member extends through a central channel around and along which the helical member extends, e.g., as shown in FIG. 29A. Helical member 1650 can be sufficiently flexible to be screwed along the curved path defined by the annulus.

In some implementations, when helical member 1650 is withdrawn (e.g., unscrewed) from the tissue (e.g., via rotation of driver 116b), suture 1602 remains and is implanted as a series of turns stitched along the annulus, with tensile member 186a remaining along an interior of (e.g., a central channel defined by) the series of turns defined by the suture (FIG. 29B). As shown in the figure, the reference numeral 1608 refers herein to the series of turns.

In some implementations, the series of turns 1608 can resemble a suture helix. In some implementations, the turns may be less regularly shaped and/or spaced than those of a true helix. Thus, for convenience, the term “suture helix” is also used herein to refer to the series of turns of the suture, and the reference numeral 1608 is also in conjunction with this term.

In some implementations, helical member 1650 stitches suture 1602 helically in an arc around at least part of the annulus, e.g., such that the suture becomes arranged in a curved path along the annulus. In some implementations, a suture-stopper 1642 (e.g., a lock) is attached to a distal end of suture 1602 to prevent suture helix 1608 from becoming unstitched from the tissue (e.g., by the suture being pulled proximally through the helical path). Similarly, in some implementations, a suture-stopper 1644 (e.g., a lock) may be locked to a proximal end of suture 1602 in order to prevent suture helix 1608 from becoming unstitched from the tissue (e.g., by the suture being pulled distally through the helical path).

In implementations in which a guide rail (e.g., guide rail 122) is used, the guide rail may be withdrawn from the heart prior to the withdrawal (e.g., unscrewing) of the helical member from the tissue (e.g., by sliding the guide rail from out from the helical member). In some such implementations, and as described hereinabove, this may be performed prior to the withdrawal of a guide frame that is used to position the guide rail in the guide arrangement along the tissue.

In some implementations, the guide rail (e.g., guide rail 122) is withdrawn subsequently to withdrawal (e.g., unscrewing) of the helical member from the heart (e.g., the guide rail is used to guide the withdrawal). In such implementations, the guide rail is withdrawn out from suture helix 1608.

Using a similar technique to the technique described with reference to implant 160, tensile member 186a is then tensioned, in order to adjust a dimension of the tissue by the tensile member pulling on suture 1602 e.g., thereby reducing a dimension of the annulus and improving coaptation of the leaflets of the valve (FIG. 29C).

In some implementations, implant 1600 may further comprise a stopper, such as first stopper 164a, that is fixed to a distal end of tensile member 186a, thereby preventing the distal end of the tensile member from sliding proximally through the suture helix 1608 that is stitched to the tissue, such that tensioning of tensile member 186a pulls the suture helix to contract the tissue. In some implementations, and as described hereinabove, first stopper 164a can be a toggle “T-shaped” stopper.

In some implementations, tensioning tensile member 186a adjusts (e.g., contracts) dimensions of the tissue by the tensile member pulling the suture helix medially/inwardly, and thus contracting the annulus (e.g., as illustrated by the arrows in FIG. 29C). That is, the tissue is contracted not by tightening suture 1602 (e.g., by pulling on an end of the suture), but rather by tensioning tensile member 186a (e.g., by pulling on an end of the tensile member), such that the tensile member pulls suture helix 1608 in a manner that draws the tissue medially/inwardly. The tensioning of tensile member 186a may reshape (e.g., distort) one or more turns (e.g., each turn) of suture helix 1608, e.g., rather than shrinking each turn of the helix (which might occur if suture 1602 were tensioned directly). This effect is schematically represented in FIGS. 29B-C by the reshaping the turns of the suture helix from being rounder (FIG. 29B) to being flatter and/or more oval (e.g., FIG. 29C).

In some implementations, delivery assembly 110c may comprise a tensioning tool, such as tensioning tool 180, that is advanced (e.g., through a sheath 112 of the delivery assembly) to facilitate application of the tension. For example, tensioning tool 180 may be used to apply a reference force during tensioning of tensile member 186a. In some implementations, a second stopper 164c can then be applied to the tensile member proximally from the proximal end of suture helix 1608, e.g., to maintain the tension on the tensile member. in some implementations, the second stopper may be a T-element, a knot and/or a bead.

In some implementations, tensioning tensile member 186a may pull the tensile member medially such that the tensile member becomes suspended over the valve orifice (e.g., and the leaflets of the valve). For example, suture 1602 may be stitched along an inner edge (e.g., lip) of the annulus (e.g., at the brink of the valve orifice) such that tensioning tensile member 186a pulls the tensile member and/or part of suture helix 1608 medially into the orifice (FIG. 29C, enlarged view).

In implementations in which suture 1602 is stitched along the annulus while tensile member is disposed within a lumen of the guide rail, the guide rail may be retracted proximally out from the suture helix prior to tensioning of the tensile member, leaving the tensile member exposed within the suture helix.

FIGS. 30A-B, 31, and 32A-B illustrate techniques for implementing the systems and methods described with reference to FIGS. 29A-C.

FIG. 30A-B illustrate an implementation in which a helical member 1650a, which may be a variant of helical member 1650, is in the form of a hollow helical needle defining a channel 1656 (e.g., lumen) therethrough (i.e. a helical channel). Suture 1602 is disposed within the channel 1656, such that screwing the driver along the tissue (e.g., guided by guide rail 122) stitches the suture into the tissue-albeit within the helical member (FIG. 30A). Once helical member 1650a has been screwed along the annulus (e.g., corresponding to the state shown in FIG. 29A), the helical member is unscrewed, withdrawing it over suture 1602, exposing the suture, and leaving the exposed suture stitched along the tissue in the form of a series of turns (e.g., suture helix) 1608 (FIG. 30B).

For the sake of clarity, FIG. 30B does not show guide rail 122. In some implementations, guide rail 122 is in fact withdrawn prior to the withdrawal/unscrewing of helical member 1650a. In some implementations, and as described hereinabove, guide rail 122 remains present during the withdrawal/unscrewing of helical member 1650a, and is withdrawn subsequently to the withdrawal/unscrewing.

In some implementations, a suture head 1642a may be fixedly attached to the distal end of the suture, in order to prevent the suture from being drawn back through, and unstitched from, the tissue by the unscrewing of helical member 1650a. For example, head 1642a can have a maximum diameter that is greater than that of helical member 1650a. In some implementations, suture head 1642a can prevent suture 1602 from becoming unstitched from the tissue (e.g., suture head 1642a may be a variant of stopper 1642, and/or can serve a similar purpose).

In some implementation, suture head 1642a can be in the form of a sharpened distal tip. For example, suture head 1642a may be in the form of a cone (e.g., having a pointed, sharpened tip), the base of the cone having a larger diameter than helical member 1650a. In some implementations, and as illustrated by FIG. 30A, during screwing in of helical member 1650a along the annulus, suture head 1642a is used to penetrate the tissue (e.g., serving as a sharpened distal tip of helical member 1650a). For example, suture head 1642a may be mounted on a distal end of helical member 1650a during advancement of the helical member along the tissue.

FIG. 31 illustrates a technique in which suture 1602 extends alongside a helical member 1650b (e.g., along an exterior of the helical member) such that screwing the helical member into and along the tissue (e.g., guided by guide rail 122) draws the suture through the tissue alongside the helical member.

For example, suture 1602 can be attached to a distal end of a helical member 1650b at an attachment site 1657, with the suture extending, from the attachment site, proximally alongside the helical member. Note the cross-sectional inset that shows suture 1602 alongside helical member 1650b, within the tunnel in tissue of annulus 10 that has been tunneled by the helical member. Once helical member 1650b has been screwed along the annulus (e.g., corresponding to the state shown in FIG. 29A), suture 1602 can be detached from the helical member, and the helical member can then be helically retracted (e.g., unscrewed) from the tissue, leaving the suture stitched along the annulus (e.g., corresponding to the state shown in FIG. 29B).

In some implementations, suture 1602 defines a suture head 1642b that is mounted and/or attached to helical member 1650b at an attachment site 1657—e.g., at a distal end of the helical member.

In some implementations, similarly to as described hereinabove with reference to suture head 1642a, suture head 1642b has a sharp tip and a base that is wider than helical member 1650b—e.g., the suture head may be conical or pyramidal.

In some implementations, suture head 1642b can be mounted on a distal end of helical member 1650b, with suture 1602 extending, from the suture head, proximally alongside the helical member. During screwing of helical member 1650b along the annulus, suture head 1642b can serve as a sharpened distal tip for the helical member, e.g., the suture head penetrates the tissue, and pulls suture 1602 along with it. Once the helical member has been screwed along the annulus (e.g., corresponding to the state illustrated in FIG. 29A), the helical member can be unscrewed from along the tissue, leaving head 1642b behind (e.g., the head is pulled off of the helical member by the tissue), and thereby leaving suture 1602 stitched along the tissue (e.g., corresponding to the state illustrated in FIG. 29B).

FIGS. 32A-B illustrate a technique in which a helical member 1650c is screwed along the annulus prior to implanting the suture 1602 in the annulus. For example, in some implementations, while the helical member remains screwed along the tissue, an end 1604 of the suture is advanced towards, and attached to, the distal end of the helical member (FIG. 32A). For example, suture 1602 may be advanced to the distal end of the helical member via a tool 185 and the tool can attach the suture to the distal end of the helical member at an attachment site 1658.

In some implementations, end 1604 of the suture is in the form of a loop, which is hooked onto a hook 1653 of the helical member in order to form the attachment. In some implementations, hook 1653 may be recessed or otherwise shielded, or may be gated like a carabiner, so as to prevent hooking and/or injuring the tissue.

In some implementations, while the suture remains attached to the distal end of the helical member, the helical member is unscrewed from the tissue such that the suture is drawn into and along the tissue by the withdrawal of the helical member (FIG. 32B). Suture 1602 thus is stitched along the tissue via the unscrewing (e.g., withdrawal) of helical member 1650c. The suture can then be unattached (e.g., unlooped) from the helical member, leaving the suture stitched along the tissue in suture helix 1608.

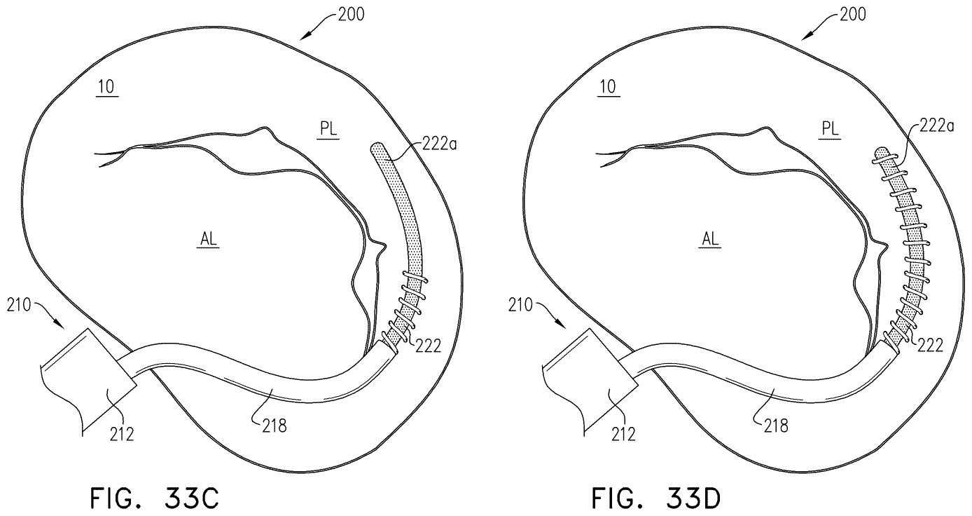

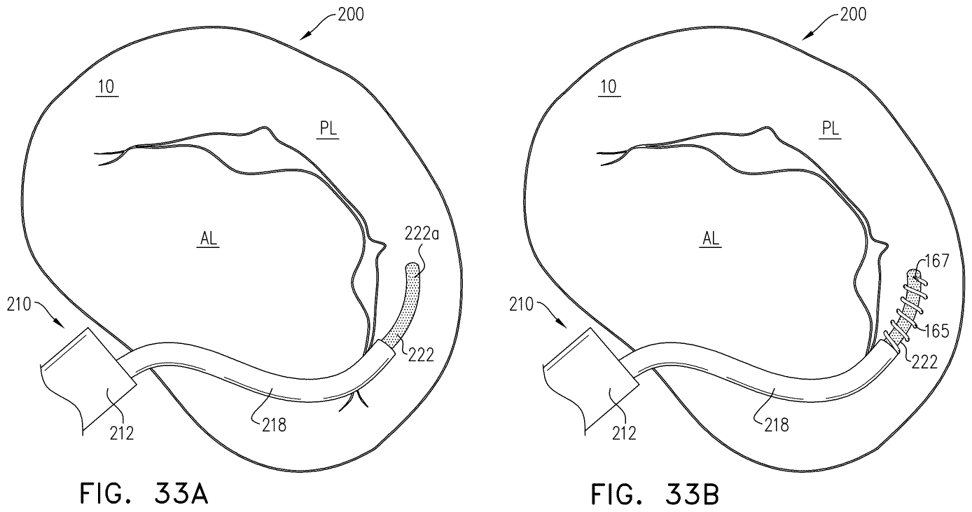

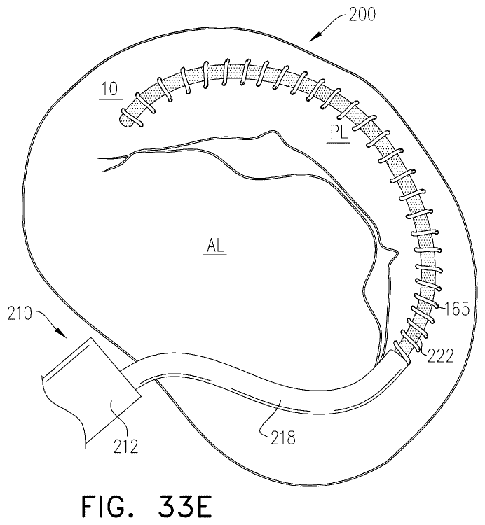

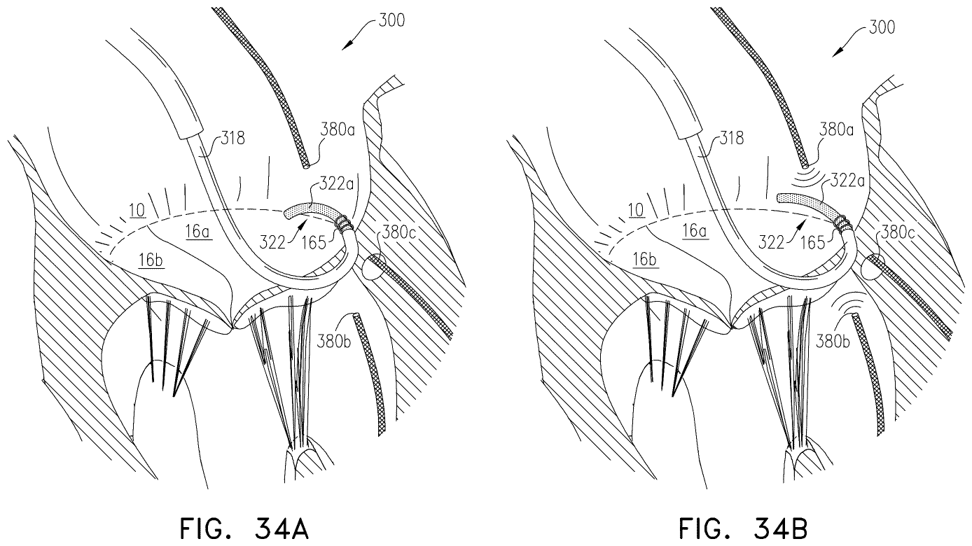

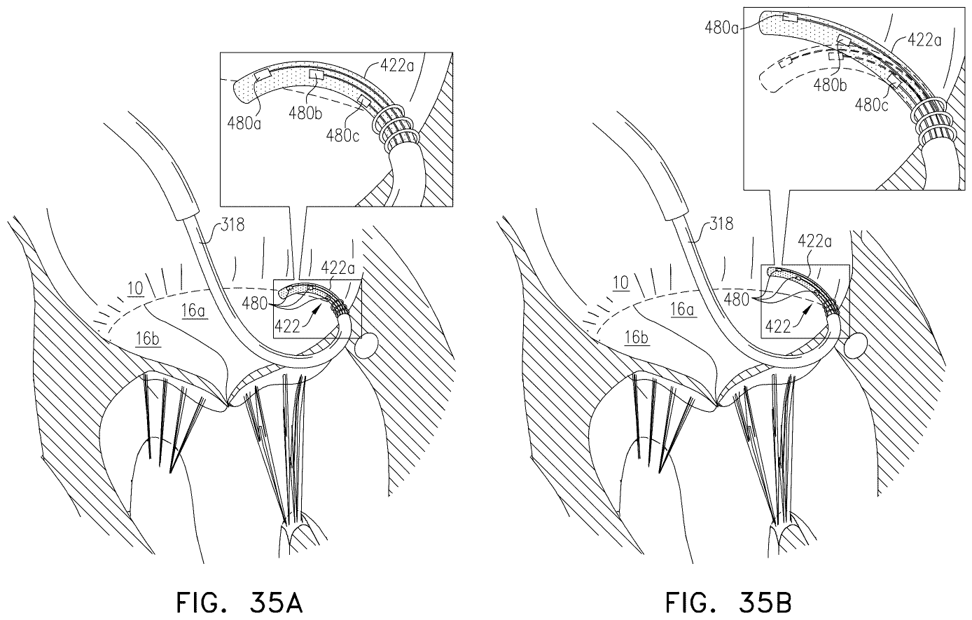

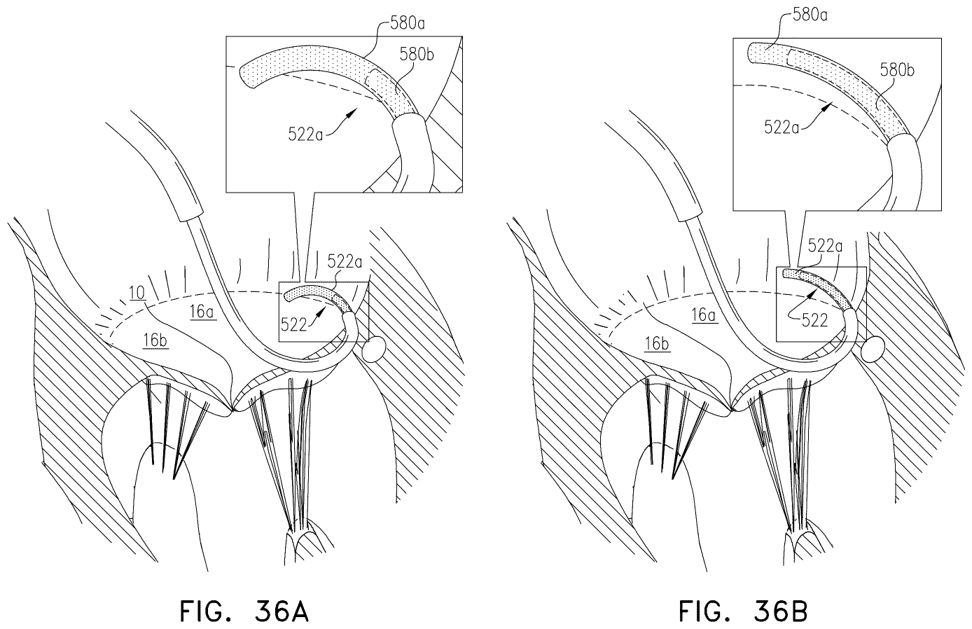

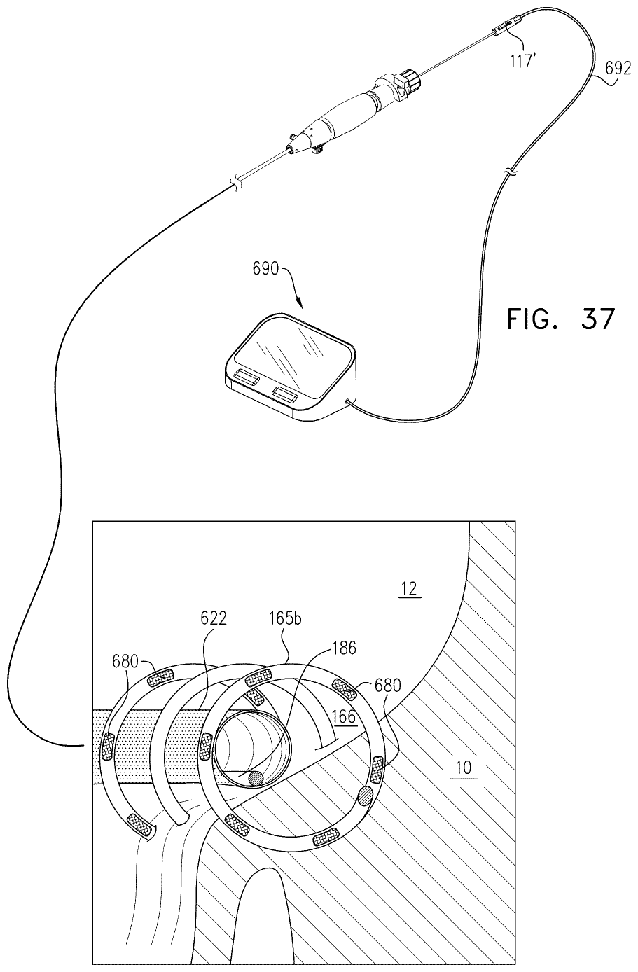

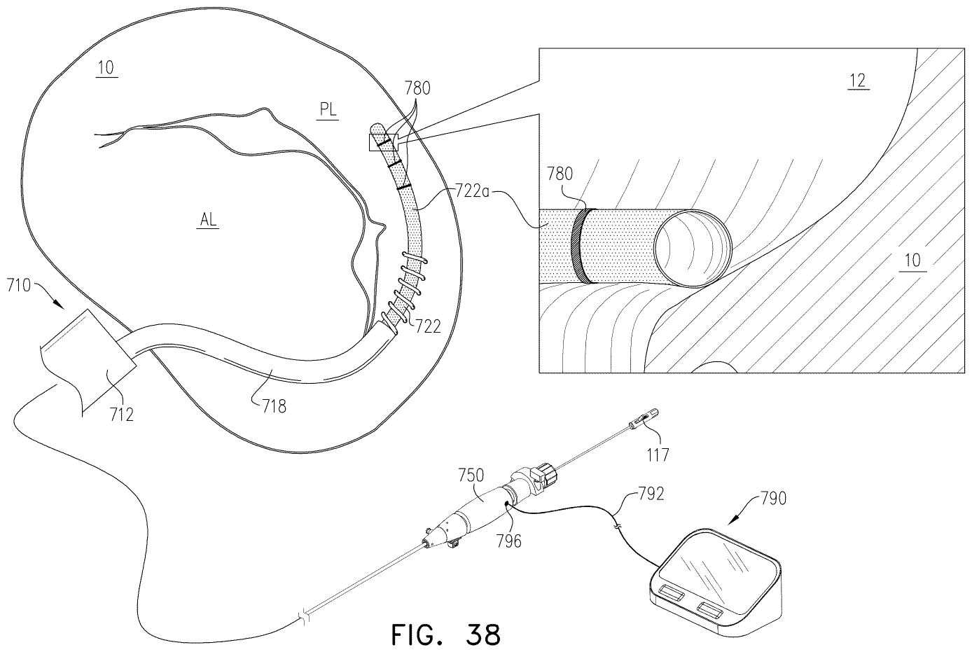

Reference is now made to FIGS. 33A-E, 34A-B, 35A-B, 36A-B, 37, and 38, which are schematic illustrations of systems and techniques for positioning a guide rail along a tissue, and for guiding a helical member along the tissue using the guide rail, in accordance with some implementations. In some implementations, these systems do not include a guide frame for the positioning of the guide rail. Rather, a guide rail (e.g., a variant of guide rail 122) is positioned around the annulus using any of the techniques described hereinbelow.

In some implementations, a guide frame (e.g., guide frame 124 as described hereinabove) can be used, and the below mentioned systems for positioning the guide rail can be used in conjunction with the guide frame.

FIGS. 33A-E show a system 200 that comprises a delivery assembly 210 comprising a guide rail 222 adapted to guide the implantation of a helical member of an implant, e.g., the helical member 165 of implant 160. Similarly to system 100, system 200 can be used at an atrioventricular valve of a heart, such as a mitral valve or a tricuspid valve e.g., in order to circumferentially reduce the size of the annulus.

As shown in FIGS. 33A-E, in contrast to the guide rail of system 100, guide rail 222 is advanced along annulus 10 incrementally with the anchoring of helical member 165. For example, for each such increment, a leading (e.g., distal) segment 222a of the guide rail can be advanced beyond helical member 165 and placed along a stretch of the annulus, and the helical member can subsequently be advanced helically over the leading segment such that it becomes anchored along that stretch of the annulus. This can be repeated iteratively until the helical member is anchored along the desired stretch of annulus (FIG. 33E). Such incremental advancement of the guide rail may advantageously provide enhanced control and/or positioning of the guide rail and/or the helical member, e.g., in place of guide frame 124 described hereinabove.

For example, FIGS. 33A-E can represent a series of steps that can be performed by the operator, to implant helical member 165 around an annulus 10 of a heart valve, in accordance with some implementations. FIG. 33A shows guide rail 222 having been advanced out of a tube 218 of delivery assembly 210, such that leading segment 222a extends along a portion of the stretch of the annulus to which helical member 165 is to be anchored.

In some implementations, helical member 165 is then advanced helically over and along leading segment 222a, such that the helical member becomes anchored (e.g., screwed) to the tissue along that stretch (FIG. 33B). Guide rail 222 can then be advanced again, such that leading segment 222a again extends beyond the distal tip 167 of helical member 165 (FIG. 33C), after which helical member 165 is again helically advanced and anchored (FIG. 33D). This process can be repeated iteratively until the helical member is anchored along as much of the annulus as is desired (FIG. 33E).

In some implementations, in order to position the leading segment of the guide rail into a desired alignment with respect to the annulus, (e.g., prior to the screwing of the helical member along the leading segment) the position of the leading segment can be determined and/or manipulated using any of the techniques described with reference to FIGS. 34A-B, 35A-B, and 36A-B. Each of these figures shows a guide rail that is adapted to guide the implantation of a helical member of an implant, e.g., the helical member 165 of implant 160.

FIGS. 34A-B are a schematic illustration of system 300 that comprises at least one electromagnet 380 that is used to guide a leading segment 322a of a guide rail 322 against the tissue, e.g., using magnetic repulsion and/or attraction between the electromagnet and leading segment 322a of the guide rail. For example, leading segment 322a can comprise a magnetic (e.g., ferromagnetic) material.

In the example shown, an electromagnet 380a has been positioned in the atrium upstream of the valve, an electromagnet 380b has been positioned in the ventricle downstream of the valve, and an electromagnet 380c has been positioned in a coronary blood vessel adjacent the valve (e.g., a coronary artery and/or the circumflex artery). Although the illustrated example shows three electromagnets being used, fewer (e.g., one or two) or more (e.g., four or five) electromagnets can be used, in these and/or in other suitable positions within the heart of the subject (e.g., within a heart of a living subject or within a heart of a simulation). Any/each of these electromagnets can be a component of a respective electromagnet tool configured to advance, position, and/or energize the electromagnet.

In some implementations, when multiple electromagnets 380 are used, they can be used in cooperation with each other in order to achieve a desired positioning of leading segment 322a. In some implementations, one or more of the electromagnets magnetically attract leading segment 322a. In some implementations, one or more of the electromagnets magnetically repel the leading segment.

To illustrate this, FIG. 34A shows leading segment 322a lying over a leaflet 16a of the valve. In FIG. 34B, this is corrected using electromagnets 380 to position the leading segment against the annulus and away from the leaflet. In some implementations, guide rail 322 can be biased toward a particular curvature (e.g., the curvature shown in FIG. 34A), and electromagnets 380 can at least partly overcome this biasing.

In some implementations, electromagnet(s) 380 can be moved incrementally along with guide rail 322, e.g., as helical member 165 is advanced. For example, for implementations in which an electromagnet 380c is positioned within a coronary blood vessel adjacent the valve, the electromagnet can be maintained adjacent the leading segment.

FIGS. 35A-B illustrate another example of positioning a leading segment of a guide rail 422 along an annulus of a valve. Guide rail 422 (e.g., a leading segment 422a thereof) comprises at least one heating element 480, that is adapted to adjust a curvature of the leading segment by heating the leading segment (e.g., via Joule heating). In some implementations, guide rail 422 (e.g., leading segment 422a thereof) can include or be made from a shape-memory alloy such as nitinol, with a transition temperature set to above body temperature, such that it curves or straightens responsively to the heating.

In some implementations, and as shown in FIGS. 35A-B, multiple heating elements 480a, 480b, and 480c are arranged along leading segment 422a of guide rail 422, such that the guide rail can be arranged into a variety of desired curvatures, responsively to heating a chosen subset of the heating elements.

In some implementations, heating elements 480 can be powered via one or more electrical conductors that extend proximally along guide rail 422, e.g., to an external power source.

To illustrate this, FIG. 35A shows leading segment 422a lying over a leaflet 16a of the valve. In FIG. 35B, this is corrected using heating element(s) 480 (e.g., all the heating elements, or only a subset of heating elements 480a, 480b and 480c) to position the leading segment against the annulus and away from the leaflet. In some implementations, guide rail 422 can be biased toward a particular curvature (e.g., the curvature shown in FIG. 35A), and heating element(s) 480 can at least partly overcome this biasing.

FIGS. 36A-B show another example of positioning the leading segment of the guide rail along the annulus using a guide rail 522. Guide rail 522 comprises an outer tube 580a and an inner shaft (e.g., an inner tube) 580b. Outer tube 580a is biased to assume a particular curvature, e.g., an at-rest curvature. Inner shaft 580b also has an at-rest curvature, but the at-rest curvature of the inner shaft is different from that of outer tube 580a. Axially sliding inner shaft 580b distally with respect to outer tube 580a alters the curvature of a leading segment 522a of guide rail 522, e.g., by increasing the influence of the inner shaft on the outer tube.

In some implementations, as in the example shown, the at-rest curvature of inner shaft 580b is less (e.g., has a greater radius of curvature) than that of outer tube 580a, and therefore axially sliding inner shaft 580b distally with respect to outer tube 580a reduces the curvature of leading segment 522a. However, it is to be understood that the opposite configuration is possible, as are other configurations.

To illustrate this, FIG. 36A shows leading segment 522a lying over a leaflet 16a of the valve. In FIG. 36B, this is corrected by sliding inner shaft 580b further distally through outer tube 580a to position the leading segment against the annulus and away from the leaflet.

In some implementations, the positioning of components of the guide assembly (e.g., the guide rail) along the annulus can be facilitated by imaging techniques, such as fluoroscopy and/or echocardiography. For example, the guide rail can include one or more imaging markers (e.g., radiopaque markers), such as markers 123 described hereinabove.

In some implementations, the positioning of components of the guide assembly (e.g., the guide rail) along the annulus can be guided by sensing electrical signals, e.g., using leading segment 222a. For example, this sensing can include detection of electrophysiological signals produced by the heart and/or bioimpedance of the tissues, e.g., as described with reference to FIG. 38 hereinbelow.

Similar techniques can be used to guide and/or monitor anchoring of the helical member itself, e.g., as described with reference to FIG. 37 hereinbelow. Each of FIGS. 37 and 38 show a guide rail that is adapted to guide the implantation of a helical member of an implant, e.g., a variant of the helical member 165 of implant 160.

In some implementations, electrodes on the guide assembly (e.g., on the guide rail) and/or on the helical member can be used for such detection. Such electrodes can be discrete components, but in some implementations components of the implant can themselves serve as electrodes (e.g., due to being inherently electrically conductive).

Reference is made to FIG. 37, which is a schematic illustration of a helical member 165b, which is a variant of helical member 165 that includes at least one electrode 680 disposed on the helical member, in accordance with some implementations.

In some implementations, electrode(s) 680 can facilitate electrical guidance for the advancement of the helical member, e.g., prior to and/or during driving of the helical member into the tissue. Electrodes 680 can be electrically connected to a data-processing system 690 that receives electrical signals detected by the electrodes.

In some implementations, based on this information or the electrical signals detected, data-processing system 690 can provide an indication of the location of helical member 165 within the heart and/or its depth within the tissue, which the operator (e.g., physician) can use to facilitate optimal anchoring and/or positioning of the helical member and/or of guide rail 622 (e.g., a leading segment thereof).

In some implementations, data-processing system 690 is adapted to provide an indication of what proportion of each turn of helical member 165 has become embedded within the tissue, using electrode(s) 680. In some implementations, and as shown in FIG. 37, multiple electrodes 680 (e.g., six, in the example shown) can be disposed around at least one turn of helical member 165, such that receiving an electrical signal from only a subset of the electrodes may be indicative that that turn of the helical member is partially embedded within the tissue. Similarly, receiving an electrical signal from all, or a larger-than-expected subset of electrodes 680, may be indicative that the helical member is embedded too deeply within the tissue.

Alternatively or additionally, the helical member can have an electrode at its tip (e.g., the electrode can serve as the tissue-piercing tip of the helical member).

In some implementations, the helical member itself can be used as an electrode via which a data-processing system, electrically connected to the helical member, can acquire electrical signals produced by the heart. For example, the helical member may not include discrete electrodes. For example, in some implementations, the entire helical member can be conductive, and can serve as a unitary electrode.

In some implementations, data-processing system 690 can be adapted to associate various electrical signals with corresponding locations of the electrode within the heart (e.g., different tissues of the heart, or different locations along an atrioventricular axis of the heart). For example, a signal acquired from electrode 680 of the helical member that is placed against tissue of the annulus 10 can be different from a signal acquired from the same helical member placed against tissue of atrium 12 or placed against tissue of the ventricle that is downstream of the valve, or placed against tissue of a leaflet of the valve.

In some implementations, a continuous signal is provided during the anchoring of helical member 165 along the tissue, such that data-processing system 690 can be able to detect changes in the signal as indicating that the helical member has been anchored sufficiently deep into the tissue at each turn, and/or whether the path of the helical member has deviated from its intended path along the annulus.

In some implementations, a sudden change in the signal and/or pattern of the signal can indicate a deviation of the helical member into tissue of the atrium or leaflets of the valve.

In some implementations, data-processing system 690 is electrically connected to the helical member (e.g., electrode(s) 680 thereof) via a driver (e.g., driver 116), used to drive the helical member into the tissue. For example, a wire 692 with a connector (e.g., a crocodile clip) at its end, can extend from data-processing system 690, and can be mechanically and electrically connected to (e.g., clipped onto) a part of an electrically conductive shaft of the driver that is disposed outside of the subject (e.g., to an anchor handle 117′, which can be considered to be a variant of anchor handle 117 described hereinabove). Thus, the part of the driver to which wire 692 is connectable serves as a terminal.

In some implementations, data-processing system 690 also receives an electrical signal (e.g., a second electrical signal) from an additional component of the implantation system (e.g., from the guide assembly of the implant, such as from guide rail 622 and/or an additional component of the implant itself), e.g., in addition to the electrical signal from helical member 165 (which can be referred to as the first electrical signal). From the first and second electrical signals, data-processing system 690 can derive a refined signal, which can be improved (e.g., to have a better signal-to-noise ratio) compared to the first signal alone.

Reference is made to FIG. 38, which is a schematic illustration of a guide rail 722 that has a leading segment 722a that comprises one or more electrodes 780, in accordance with some implementations. Additionally and/or alternatively to electrodes on the helical member (e.g., as described with reference to FIG. 37), in some implementations, in order to implant the helical member along the annulus as desired, it may be advantageous to determine, prior to the implantation, that components of the guide assembly are satisfactorily positioned at the heart, so that the helical member is more likely to become correctly positioned within the heart upon implantation.

In some implementations, as described hereinabove with reference to FIGS. 34A-24B hereinabove, it may be desirable to position a leading segment of a guide rail in a desired alignment along the annulus (e.g., in alignment with an atrial surface of the annulus), prior to driving the helical member through tissue of the annulus along that leading segment.

In some implementations, a guide rail 722 (e.g., a leading segment 722a thereof) can include at least one electrode 780 via which a data-processing system 790, electrically connected to the guide rail, can acquire electrical signals produced by the heart. In some implementations, and as shown, electrode(s) 780 can be in the form of a ring that encircles guide rail 722 (e.g., leading segment 722a thereof), such that placing the guide rail in contact with tissue of the heart also places the electrode in contact with the tissue.

In some implementations, multiple electrodes 780 are spaced along leading segment 722a. In some implementations, electrode(s) 780 can be the form of a wire that runs longitudinally along a part of guide rail 722 (e.g., leading segment 722a thereof).

In some implementations, the electrical connection between electrode(s) 780 and data-processing system 790 can be provided, for example, by the electrode(s) being electrically connected (e.g., via conductors) to a terminal 796 at the extracorporeal portion of the delivery assembly (e.g., at a handle 750 thereof), to which a wire 792, connected to the data-processing system, can be connected.

In some implementations, data-processing system 790 can be adapted to provide an indication of the angle or rotational orientation of guide rail 722 (e.g., leading segment 722a thereof) at the tissue, using electrode(s) 780.

In some implementations, multiple discrete electrodes 780 can be distributed circumferentially around the leading segment, such that receiving an electrical signal from a particular electrode of the multiple electrodes can be indicative of the position and/or orientation of the guide rail along the tissue. For example, an electrode 780 that is in contact with tissue can provide a signal indicative of such contact.

In some implementations, data-processing system 790 can thus receive electrical signals indicative of the position of the guide rail 722 (e.g., of leading segment 722a thereof), and, based on these signals, provide an indication of the location and/or orientation of the guide rail within the heart, which the operator (e.g., physician) can use to facilitate optimal positioning of the guide rail prior to anchoring helical member 165 along the guide rail (e.g., along the leading segment).

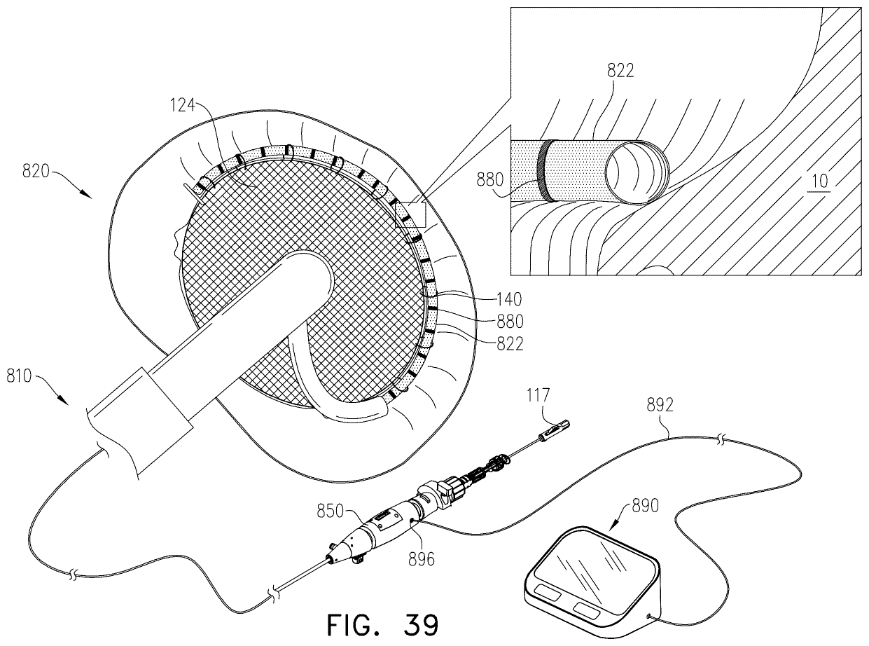

Reference is now made to FIG. 39, which is a schematic illustration of a guide assembly 820 that comprises a guide rail 822 having one or more electrodes 880 thereon, in accordance with some implementations. Guide assembly 820 can be considered to be a variant of guide assembly 120, e.g., guide assembly 820 comprises a guide frame 124 and can comprise loops 140 to secure guide rail 822 to the guide frame. In some implementations, guide rail 822 can be drawn into alignment with a midsection of the guide frame, by pulling on loops 140 e.g., as described with reference to FIGS. 3A-E hereinabove. In some implementations, in order to implant the helical member along the annulus as desired, it may be advantageous to determine, prior to the implantation, that the guide rail (or part thereof) lies against tissue, and optionally that the tissue is specifically tissue of the annulus.

In some implementations, guide rail 822 includes at least one electrode 880 via which a data-processing system 890, electrically connected to the guide rail, can acquire electrical signals produced by the heart. In some implementations, and as shown, electrode(s) 880 can be in the form of a ring that encircles guide rail 822, such that placing the guide rail in contact with tissue of the heart also places the electrode in contact with the tissue. In some implementations, multiple electrodes 880 are spaced along guide rail 822 (e.g., at least along a stretch of the guide rail along which a helical member is to be implanted). In a specific implementation, one or more of electrode(s) 880 can be the form of a wire that runs longitudinally along a part of guide rail 822.

In some implementations, guide assembly 820 is a component of a delivery assembly 810, that can be considered to be a variant of guide assembly 120, mutatis mutandis. The electrical connection between electrode(s) 880 and data-processing system 890 can be provided, for example, by the electrode(s) being electrically connected (e.g., via conductors) to a terminal 896 at an extracorporeal portion of delivery assembly 810 (e.g., at a handle 850 thereof), to which a wire 892, connected to the data-processing system, can be connected.

In some implementations, data-processing system 890 can be adapted to provide an indication of the angle or rotational orientation of guide rail 822 at the tissue, using electrode(s) 880. In some implementations, multiple discrete electrodes 880 can be distributed circumferentially around the guide rail, such that receiving an electrical signal from a particular electrode of the multiple electrodes can be indicative of the position and/or orientation of the guide rail along the tissue. For example, an electrode 880 that is in contact with tissue can provide a signal indicative of such contact.

In some implementations, data-processing system 890 can thus receive electrical signals indicative of the position of the guide rail 822, and, based on these signals, provide an indication of the location and/or orientation of the guide rail within the heart.

In some implementations, the operator (e.g., physician) can position the guide rail responsively to the electrical signals received from electrodes 880. For example, the operator can continue adjusting the alignment of guide rail 822 by pulling loops 140 from outside of the subject and/or by repositioning (e.g., tilting) guide assembly 820 at the heart, until it is indicated that all electrodes 880, or a particular subset thereof, are positioned as desired.

In some implementations, rather than guide rail 822 being drawn into the guide arrangement around guide frame 124 by tightening multiple loops from outside of the subject (e.g., as described with reference to FIGS. 3A-E), in some implementations, the guide assembly is configured such that it is the expansion of guide frame 124 that draws the guide rail into alignment along a midsection of the guide frame and/or positions the guide rail appropriately for being placed along the annulus, e.g., as described with reference to FIGS. 9A-I in International Patent Application (PCT) publication WO 2022/157592 to Herman et al., which is incorporated herein by reference.

As noted hereinabove (e.g., with reference to guide assembly 120), it may be desired that helical member 165 be implanted along only a specific stretch of the annulus. To facilitate this, the operator can manipulate guide rail 822 and/or guide assembly 820 such that some electrodes 880 indicate tissue contact whereas others do not. For example, and as shown, in some implementations, only a distal portion of guide rail 822 is positioned along the annulus, and a more proximal portion extends (e.g., at an oblique angle), from the annulus, upwardly through the atrium, and into a part of the delivery assembly (e.g., into a catheter thereof). In some implementations, the manipulation can be such that (i) the electrodes 880 disposed on the portion of guide rail 822 that is to be positioned along the desired stretch indicate tissue contact, and (ii) the electrodes 880 that are disposed on the portion of the guide rail that is to be positioned away from the annulus indicate no tissue contact.

In some implementations, guide rail 822 includes a plurality of imaging markers spaced along the guide rail, e.g., as described with reference to imaging markers 123. These imaging markers can comprise an electrically conductive material, such that the imaging markers serve, in addition to facilitating the visualization of the procedure, as electrodes 880 described herein.

In some implementations, the imaging markers and/or the electrodes can provide the user with information regarding the desired size of the implant, prior to delivery of the implant to the heart. For example, once guide rail 822 is positioned within the heart, a helical member of appropriate length can be selected based on the length of the guide rail that is positioned along the annulus, i.e., the length of the path along the annulus with which the guide rail is in contact. In some implementations, the extent of this length can be obtained by determining the proportion of the guide rail that is positioned along the annulus, e.g., by visualizing the imaging markers spaced along the guide rail, and/or detecting electrical signals via the electrodes that are spaced along the guide rail. It is to be noted that this technique could be used with guide assembly 120 using imaging markers 123 (e.g., exclusively of electrodes 880).

Reference is again made to FIGS. 37-39. In some implementations, the electrical signal detected by electrodes 680, 780 and/or 880 is an endogenous signal, e.g., an electrophysiological signal (e.g., an ECG signal) produced by the heart, and the electrode serves as a sensing electrode to detect such an endogenous signal. Additionally or alternatively, an exogenous electrical signal can be provided, e.g., via a reference electrode placed outside of the heart of the subject and/or via one or more of electrodes 680, 780, and/or 880 themselves. For example, data-processing system 690, 790, and/or 890 can drive the exogenous signal between two of the electrodes. In some implementations, the exogenous signal can be detected by electrodes 680, 780, and/or 880. In some implementations, the detected exogenous signal is used (e.g., by data-processing system 690, 790, and/or 890) to determine bioimpedance between at least two of the electrodes. In some implementations, the bioimpedance can then be used to determine a proximity and/or degree of contact between the electrode(s) and tissue, and/or the position of the electrode(s) within the heart, e.g., the nature and/or identity of the adjacent tissue (e.g., atrial wall, annulus, ventricular wall, leaflet). In the example shown in FIG. 37, such detection of an exogenous signal can be used to determine which and/or how many of electrodes 680 are disposed within the tissue.

In some implementations, for each or any of data-processing systems 690, 790 and 890, the data-processing system runs a program in which the electrical signals received from the corresponding electrode (e.g., electrodes 680, 780, and/or 880 respectively) serves as an input, and which responsively determines the location of the helical member and/or the guide rail within the heart.

In some implementations, by running the program, the data-processing system determines a location of the helical member and/or the guide rail along the atrioventricular axis of the heart. In some implementations, the data-processing system determines whether the helical member and/or the guide rail is disposed in a predefined discrete position (which can be one of multiple predefined discrete positions), such as within the atrium, at the annulus, or within the ventricle. Additionally or alternatively, the data-processing system can determine whether the anchor is in contact with a discrete type of tissue, such as atrial wall tissue, annulus tissue, leaflet tissue, or ventricular wall tissue. In some implementations, this can be achieved using, mutatis mutandis, techniques described in Provisional U.S. Patent Application 63/298,199 to Harush et al., filed Jan. 10, 2022, PCT Application PCT/IB2023/050073 to Harush et al., filed Jan. 5, 2023, and/or Provisional U.S. Patent Application 63/439,836 to Haberman Browns, et al., filed Jan. 18, 2023, which are each incorporated herein by reference for all purposes.

In some implementations, machine learning is employed in the building of the program. For example, building of the program can be facilitated by analyzing data (e.g., a labeled data set) that can include, for example, electrical signals received from the helical member, the guide rail, and/or data obtained from previously performed procedures. In some implementations, in order to train (e.g., further train) the program, the location outputted intra-procedurally by the program is compared to the actual location of the helical member and/or the guide rail (e.g., determined by other means).

In some implementations, each or any of data-processing systems 690, 790 and 890 can comprise a display for providing the operator with an output indicative of the location of the helical member and/or the guide rail that has been determined by the program run by the data-processing system. For example, the output can be indicative of a location along the atrioventricular axis of the heart.

In some implementations, the display can provide the output as an indication of a discrete position of the helical member and/or the guide rail. For example, the display can be configured to indicate that the helical member and/or the guide rail is contacting the atrial wall, tissue of the ventricle, leaflet tissue, or tissue of the annulus.

In some implementations, the data-processing system is configured to provide the output (e.g., on the display) as a binary output (e.g., a yes/no or go/no-go output), responsively to data-processing system determining that the helical member can be driven into the tissue at a potential anchoring site (e.g., by determining that the helical member and/or the guide rail is positioned satisfactorily at the tissue of the annulus).

In some implementations, and as shown, the data-processing system is, or is a component of, a discrete (e.g., purpose-made) device. In some implementations, the data-processing system is a general-purpose data-processing system (e.g., a processor of a general-purpose computer) programmed to run the program.

Techniques for positioning a replacement heart valve at an annulus of the heart, guided by electrodes disposed on the replacement valve, are also disclosed. Similarly to the technique described hereinabove, in which a plurality of electrodes are positioned around a guide rail such that the positioning of the guide rail along the annulus can be guided by the electrodes, an implant, such as a replacement heart valve, can include a plurality of electrodes that are distributed along a circumference (e.g., an outer circumference) of the implant or replacement valve such that the electrodes can be used to verify tissue-contact with the annulus. For example, before and/or during implantation of the implant or replacement valve at the heart, a controller (e.g., a surgeon, an interventionalist, and/or a data-processing system) can receive information regarding the desired implantation site of the implant within the heart. It may be important for the implant or replacement valve to be secured precisely, according to the surgical plan, in order for the implant to serve the desired purpose. For example, it may be desirable to secure the implant or replacement valve at a particular height within the native valve.

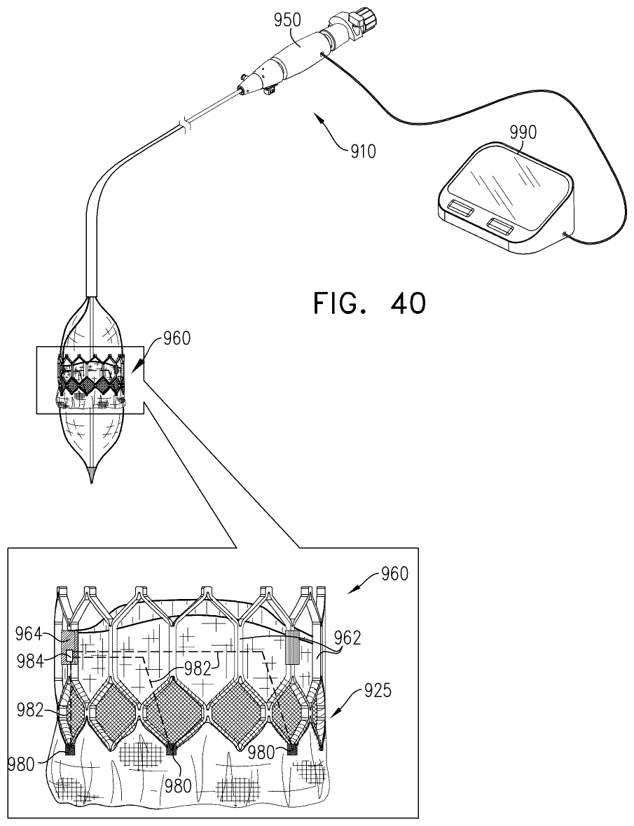

Reference is made to FIG. 40, which shows an implant configured as replacement heart valve 960 having a plurality of electrodes 980 disposed thereon adapted to guide the positioning of the valve within the heart, in accordance with some implementations. In the example shown, valve 960 has a frame that defines (or is defined by) a plurality of struts 962. The frame provides structural support and/or anchoring to the native tissue. The frame can define a waist 925, that becomes disposed circumferentially within an annulus of the heart once the valve is positioned at the desired height within the valve. In the example shown, electrodes 980 are spaced along an outer circumference of waist 925. As shown, each electrode 980 can be connected, via at least one connector 982 (e.g., a respective connector, or a common connector), and via a delivery tool 910 for the valve, to a data-processing system 990 that is adapted to receive electrical signals detected by the electrodes. These electrical signals can be exogenous (e.g., applied by one or more of electrodes 980, or by a skin-patch electrode) or endogenous (e.g., ECG signals). In some implementations, electrodes 980 are electrically connected to one or more of an implant, anchor, power source, display, etc.

In some implementations, and as shown, valve 960 comprises a common terminal 984, to which delivery tool 910 is electrically connected during delivery and implantation of the valve, thereby providing DPS 990 with its electrical connection to electrodes 980 (e.g., utilizing multiplexing). In the example shown, terminal 984 is located at and/or supported by an artificial commissure 964 of valve 960.

In some implementations, the data-processing system can be adapted to associate various electrical signals with corresponding locations of electrodes 980 within the heart (e.g., different tissues of the heart, or different heights along the valve), such that the operator can verify that the implant or replacement valve is satisfactorily positioned, e.g., prior to anchoring the implant or replacement valve to the annulus. Once the operator has determined that the implant/valve is satisfactorily positioned, further expansion and/or securement of implant/valve 960 to the annulus can be performed, e.g., so that implant/valve 960 becomes implanted within the heart.

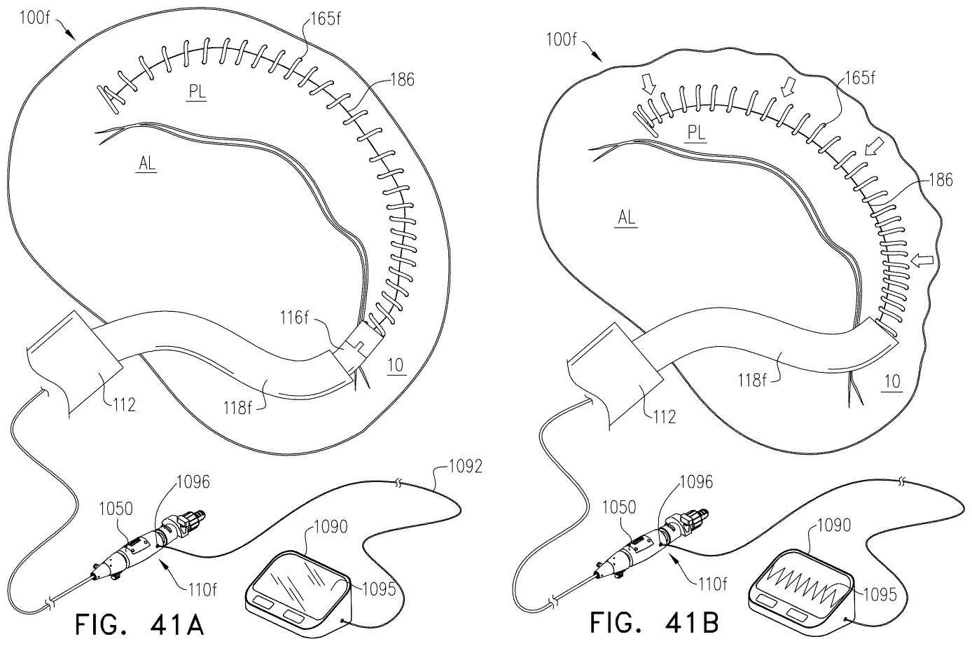

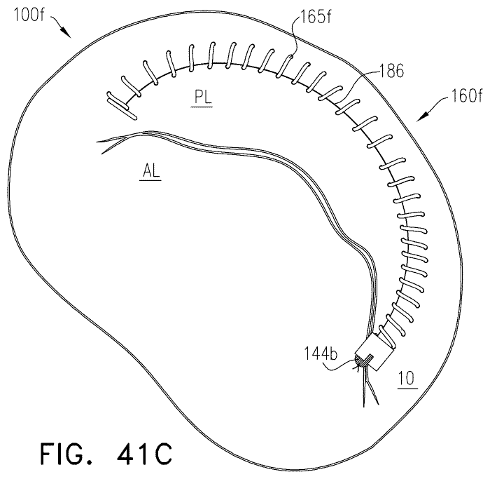

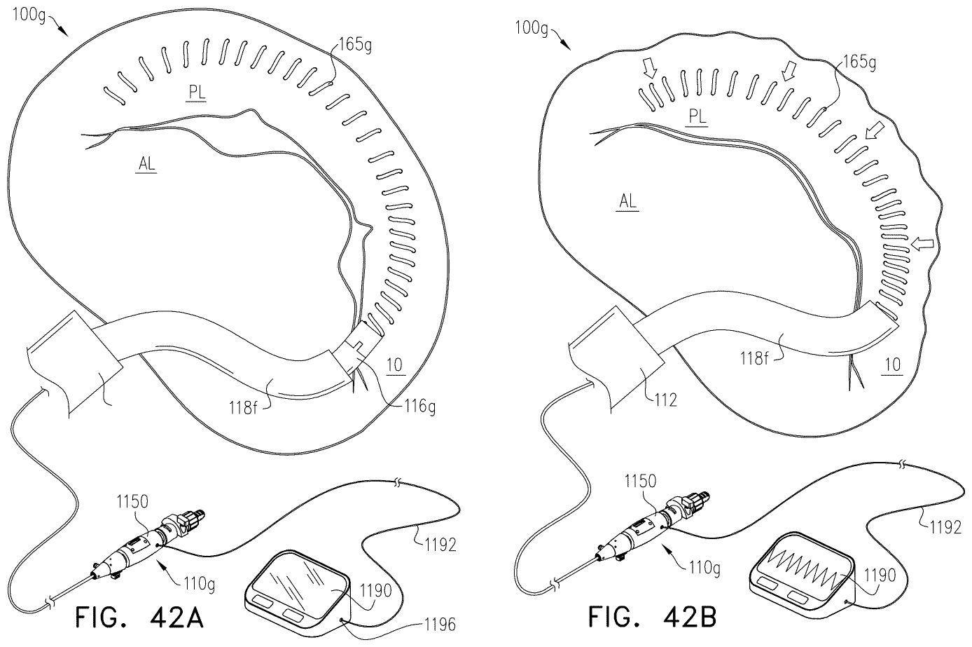

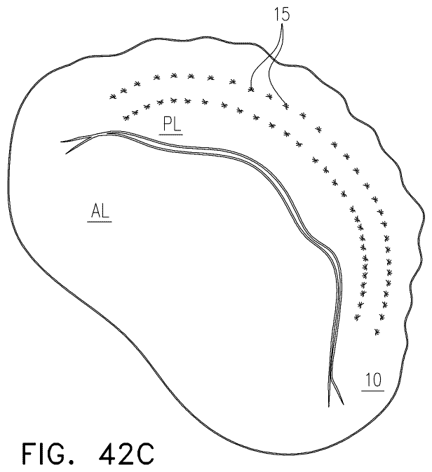

Reference is now made to FIGS. 41A-C which illustrate a system 100f that uses electrical energy to contract a helical member implanted within the heart, in accordance with some implementations. System 100f can comprise an implant 160f which can be a variant of, or substantially identical to, implant 160, e.g., except when noted otherwise. In some implementations, implant 160f comprises a helical member 165f. Although helical member 165f can be similar in structure and/or design to helical member 165, e.g., both defining multiple turns and a helix defined by the turns, in some implementations, helical member 165f is adapted to contract responsively to application of energy, e.g. electrical energy, electromagnetic energy, or magnetic energy. In some implementations, the manner of contraction is that turns of the helical member move toward each other. For example, in some implementations, helical member 165f is conductive, and/or heat set to shrink responsively to the energy. In some implementations, helical member 165f can be constructed from a shape-memory material e.g., nitinol, such that application of the energy reverts the helical member to its pre-set shape.

In some implementations, system 100f comprises a delivery assembly 110f that can comprise a driver 116f that can be used to apply torque to helical member 165f, to screw the helical member along the tissue. In some implementations, driver 116f is additionally used to conduct the electrical energy to helical member 165f, e.g., as will be explained in more detail hereinbelow.

In some implementations, delivery assembly 110f may comprise a guide assembly for guiding the screwing of helical member 165f along the annulus—e.g., as described hereinabove, mutatis mutandis. For example, such a guide assembly may include a guide rail (e.g., guide rail 122, not shown) over which helical member 165f can be advanced, such that the helical member can be advanced over and along the guide rail during screwing of the helical member into the tissue.

In some implementations, the guide assembly with which delivery assembly 110f is used comprises a guide frame (e.g., guide frame 124, or any of the guide frames described herein), for positioning of the guide rail at the annulus (e.g., in a guide arrangement along the exterior of the guide frame), e.g., using any of the methods or systems described herein.

In some implementations, the guide assembly with which delivery assembly 110f is used does not comprise a guide frame, e.g., it can comprise any of the guide rails described herein that are positionable along the tissue without a guide frame (e.g., guide rails 222, 322, 422, 522, 622 and/or 722). In some implementations, the guide rail can be advanced along the annulus incrementally with the screwing in of helical member 165f.

Although, as noted above, a guide assembly may be used to guide implantation of implant 160f, for the sake of clarity, FIGS. 41A-C do not show components of the guide assembly, such as a guide rail and/or a guide frame.

In some implementations, delivery assembly 110f can comprise a tube (e.g., a catheter) 118f, that extends distally from a delivery sheath (e.g., sheath 112) of the delivery assembly, to the tissue being treated. In some implementations, tube 118f can be a variant of, or substantively identical to, tubes 118, 118a and/or 118b. Tube 118f may advantageously shield the surrounding tissue (and/or the guide frame, if present) from a sharpened distal tip of helical member 165f during delivery of the helical member towards the tissue.

In some implementations, delivery assembly 110f further comprises an extracorporeal power generator 1090, electrically connectable to helical member 165f (e.g., via a handle 1050 of the delivery assembly), the power generator being adapted to apply electrical energy to the helical member while the helical member is implanted, within the heart, along the annulus. In some implementations, the electrical connection between the power generator and helical member 165f is facilitated by anchor driver 116f, e.g., the anchor driver is electrically conductive and conducts the electrical energy, from handle 1050 to the helical member. In some implementations, power generator 1090 is connected to helical member 165f via a conductor (e.g., a wire) that extends alongside, or within, the anchor driver.

In some implementations, delivery assembly 110f can be electrically connected to power generator 1090 via a terminal 1096 on handle 1050, e.g. by connecting a wire 1092 from the power generator to the terminal.

In some implementations, power generator 1090 can comprise a display (e.g., display 1095) that outputs information regarding the energy being applied to the helical member.

In some implementations, and similarly to as described hereinbelow with reference to FIG. 37, helical member 165f can additionally serve as a sensing electrode, or can comprise one or more sensing electrodes thereon. In some implementations, prior to applying the electrical energy to the helical member, the helical member is used as a sensing electrode to provide information to a data-processing system that outputs an indication of the location of helical member 165f within the heart and/or its depth within the tissue, which the operator (e.g., physician) can use to facilitate optimal anchoring and/or positioning of the helical member. In some implementations, power generator 1090 may comprise such a data-processing system, and may comprise a display 1095 that outputs the information.

Once it is decided that helical member 165f is satisfactorily positioned, the electrical energy can be applied to the helical member, which is configured to responsively contract, such that the turns of the helical member are drawn closer to each other (FIG. 41B), thereby reducing a dimension of the annulus and improving coaptation of the leaflets of the valve.

FIGS. 41A-C can represent a series of steps that can be performed by a user (e.g., physician), to implant implant 160f around an annulus 10 of a heart valve, in accordance with some implementations.

FIG. 41A illustrates helical member 165f having been screwed into and along tissue of the annulus, such that part of each turn of the helical member is embedded within the tissue, and another part of each turn lies above the surface of the tissue (e.g., as described for helical member 165 hereinabove). In some implementations, implant 160f comprises tensile member 186 that provides similar functionality to that of tensile member 186 shown with reference to implant 160. For example, tensile member 186 can similarly be positioned, in a curved path, along the annulus (e.g., within a guide rail), such that helical member 165f is screwed along the tensile member (e.g., around the tensile member). In some implementations, after the helical member has been screwed into and along the tissue, the tensile member extends through a central channel around and along which the helical member extends, e.g., as shown in FIG. 41A. Helical member 165f can be sufficiently flexible to be screwed along the curved path defined by the annulus.

In some implementations, the suitability of the position and/or orientation of helical member 165f is then determined, e.g., using helical member 165f as a sensing electrode, using a discrete electrode positioned on the helical member, and/or via imaging of the helical member.

Once helical member 165f is satisfactorily positioned, the energy can be applied to the helical member, which is configured to responsively contract, such that the turns of the helical member are drawn closer to each other (FIG. 41B), thereby reducing a dimension of the annulus and improving coaptation of the leaflets of the valve.

In some implementations, the contraction of helical member 165f remains only while the energy continues to be applied. In some implementations, while the energy continues to be applied to the helical member, tensile member 186 can be contracted, and a lock 144b locked to the tensile member, thus mechanically locking (e.g., maintaining) the implant in its contracted state e.g., as described hereinabove with reference to implant 160 mutatis mutandis. In some implementations, tensile member 186 is not present, and a different mechanical locking means is used.

In some implementations, upon cessation of the electrical energy to helical member 165c, the helical member returns to its original size and shape, however, the contracted state of the annulus is maintained via lock 144b, and/or a lock applied to the helical member.

In some implementations, helical member 165f is heat set such that it remains in its contracted state even after cessation of the application of the energy. In some such implementations, no lock is applied to the helical member once it is contracted. In such implementations, tensile member 186 may therefore be absent.

In some implementations, helical member 165f conducts at least some of the energy to the surrounding tissue (e.g. as electrical/RF energy, or as heat), which may change a property of the tissue, e.g. by heating the tissue. For example, the conducted energy may cause the tissue to contract (e.g. augmenting the mechanical contraction provided by the contraction of the helical member) or may merely “set” the tissue in the contracted state achieved via the contraction of the helical member.

FIG. 41C illustrates the valve in its improved state, with implant 160f implanted along the annulus, and delivery assembly 110f having been withdrawn from the heart.

Reference is now made to FIGS. 42A-C, which illustrate an implementation in which, similarly to FIGS. 41A-C, electrical energy is applied to the helical member in order to contract tissue 10 of the annulus. However, unlike FIGS. 41A-C, in FIGS. 42A-C, the helical member is not left implanted along the tissue, and is subsequently withdrawn from the tissue. FIGS. 42A-C illustrate an implementation in which sufficient electrical energy is applied to the helical member to permanently contract (e.g., shrink) the tissue of the annulus, e.g., by irreversibly changing the tissue properties of the annulus, such that, even after removal of the helical member, the annulus remains in its contracted, improved state.

FIGS. 42A-C illustrate a system 100g that can be a variant of, or substantially identical to, system 100f, e.g., except when noted otherwise. System 100g comprises a helical member 165g that, in some implementations, is adapted to contract responsively to application of applied energy (e.g., such that turns of the helical member move toward each other). For example, in some implementations, helical member 165g is heat set to shrink responsively to the applied energy. In some implementations, helical member 165g can be constructed from a shape-memory material e.g., nitinol, such that application of electrical/heat energy reverts the helical member to its pre-set shape.

Although helical member 165g can be similar in structure and/or design to helical member 165f, e.g., both defining multiple turns and a helix defined by the turns, in some implementations, system 100g is configured to apply the energy via helical member 165g, and to then withdraw (e.g. unscrew) the helical member from the tissue following the procedure, in contrast to helical member 165f which remains implanted within the subject following the procedure. That is, helical member 165g is part of a delivery assembly 110g, rather than being part of an implant itself. In some implementations, system 100g does not comprise an implant at all, rather delivery assembly 110g is used at the valve, and the delivery assembly is then withdrawn, leaving the valve in an improved state without any additional components implanted therein.

In some implementations, delivery assembly 110g can comprise a guide assembly for guiding the screwing of helical member 165g along the annulus, e.g., as described hereinabove, mutatis mutandis. For example, such a guide assembly may include a guide rail (e.g., guide rail 122, not shown) over which helical member 165g can be advanced, such that the helical member can be advanced over and along the guide rail during screwing of the helical member into the tissue.

In some implementations, the guide assembly with which delivery assembly 110g is used comprises a guide frame (e.g., guide frame 124, or any of the guide frames described herein), for positioning of the guide rail at the annulus (e.g., in a guide arrangement along the exterior of the guide frame), e.g., using any of the methods or systems described herein.

In some implementations, the guide assembly with which delivery assembly 110g is used does not comprise a guide frame, e.g., it can comprise any of the guide rails described herein that are positionable along the tissue without a guide frame (e.g., guide rails 222, 322, 422, 522, 622 and/or 722). The guide rail can be advanced along the annulus incrementally with the screwing in of helical member 165g.

In some implementations, delivery assembly 110g comprises tube 118f, that extends distally from a delivery sheath (e.g., sheath 112) of the delivery assembly, to the tissue being treated. As noted hereinabove, tube 118f may advantageously shield the surrounding tissue (and/or the guide frame, if present) from a sharpened distal tip of helical member 165g during delivery of the helical member towards the tissue.

Although, as noted above, a guide assembly can be used to guide the screwing of helical member 165g along the tissue of the annulus, for the sake of clarity FIGS. 42A-C do not show components of the guide assembly, such as a guide rail and/or a guide frame.

In some implementations, delivery assembly 110g can comprise a driver 116g that can be used to apply torque to helical member 165g, to screw the helical member along the tissue. In some implementations, the driver can be fixedly (e.g., permanently) attached to helical member 165g, such that the helical member is withdrawn along with the driver, e.g., as will be described hereinbelow. In some implementations, driver 116g can be attached or attachable (e.g., fixedly or reversibly) to helical member 165g via a head of the helical member.

In some implementations, driver 116g can be a variant of any of the drivers described herein, and/or can include features of any of the drivers described herein.

In some implementations, helical member 165g and driver 116g are formed from a unitary element (e.g., shaft), e.g., the helical member may simply be a continuation of the driver, thereby obviating the need for attachment of the helical member to the driver.

In some implementations, delivery assembly 110g further comprises an extracorporeal power generator 1190, electrically connectable to helical member 165g (e.g., via a handle 1150 of the delivery assembly), the power generator being adapted to apply electrical energy to the helical member while the helical member is implanted, within the heart, along the annulus. Delivery assembly 110g may be electrically connected to power generator 1190 via a terminal 1196 on handle 1150. e.g. by connecting a wire 1192 from the power generator to the terminal.

In some implementations, and as described hereinabove with reference to driver 116f, the electrical connection between the power generator and helical member 165g is facilitated by anchor driver 116g, e.g., the anchor driver is electrically conductive and conducts the energy from handle 1150 to the helical member. In some implementations, power generator 1190 is connected to helical member 165g via a conductor (e.g., a wire) that extends alongside, or within, the anchor driver.

FIG. 42A illustrates helical member 165g having been (temporarily) screwed into and along tissue of the annulus, such that part of each turn of the helical member is embedded within the tissue, and another part of each turn lies above the surface of the tissue (e.g., as described for helical member 165 and/or 165f hereinabove).

In some implementations, and similarly to as described hereinabove with reference to FIGS. 41A-C, the suitability of the position and/or orientation of helical member 165g is then determined, e.g., using the helical member as a sensing electrode, using a discrete electrode positioned on the helical member, and/or via imaging of the helical member.

Energy can then be applied to helical member 165g, to chronically contract the annulus (FIG. 42B). In some implementations, helical member 165g does not contract, but simply conducts the energy to the tissue. In some implementations, helical member 165g does contract, and energy is applied to the contracted helical member until the tissue is chronically altered. Helical member 165g can then be withdrawn (e.g., unscrewed) from the tissue (e.g., via rotation of driver 116g), leaving the annulus in its contracted, improved state (FIG. 42C). Reference numeral 15 indicates the sites at which helical member 165g penetrated and applied energy to the tissue.

In implementations in which a guide rail (e.g., guide rail 122) is used, the guide rail may be withdrawn from the heart prior to the withdrawal (e.g., unscrewing) of the helical member from the tissue (e.g., by sliding the guide rail from out from the helical member). In some such implementations, and as described hereinabove, this can be performed prior to the withdrawal of a guide frame that is used to position the guide rail in the guide arrangement along the tissue.

In some implementations, the guide rail (e.g., guide rail 122) is withdrawn subsequently to withdrawal (e.g., unscrewing) of the helical member from the heart (e.g., the guide rail is used to guide the withdrawal).

In the present disclosure, the term data-processing system can refer to, be part of, or include an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor (shared, dedicated, group) that executes code; memory (shared, dedicated, or group) that stores code executed by a processor; other suitable hardware components, such as optical, magnetic, or solid state drives, that provide the described functionality; or a combination of some or all of the above, such as in a system-on-chip. The term code, as used above, can include software, firmware, and/or microcode, and can refer to programs, routines, algorithms, functions, classes, and/or objects. The term shared processor encompasses a single processor that executes some or all code from multiple modules. The term group processor encompasses a processor that, in combination with additional circuitry (e.g., processors), executes some or all code from one or more modules. The term shared memory encompasses a single memory that stores some or all code from multiple modules. The term group memory encompasses a memory that, in combination with additional memories, stores some or all code from one or more modules. The term memory can be subset of the term computer-readable medium. The term computer-readable medium does not encompass transitory electrical and electromagnetic signals propagating through a medium and can therefore be considered tangible and non-transitory. Non-limiting examples of a non-transitory tangible computer readable medium include nonvolatile memory, volatile memory, magnetic storage, and optical storage.

Although the figures of this disclosure illustrate an implant being implanted along a mitral valve of a heart, it should be understood that the techniques and systems described hereinabove can also be used for valves other than the mitral valve, for example, for the tricuspid valve of the heart. Furthermore, the techniques can be used for parts of the heart other than a valve, e.g., for ventriculoplasty.

### EXAMPLE APPLICATIONS (SOME NON-LIMITING EXAMPLES OF THE CONCEPTS HEREIN ARE RECITED BELOW)

Example 1. A system for use with real or simulated tissue of a real or simulated heart, the system comprising: (A) an implant; and/or (B) a delivery assembly comprising: (i) a guide assembly, having a distal part that is transluminally advanceable to the heart while in a delivery state, the guide assembly comprising: (a) a guide frame, intracardially expandable toward an expanded state, (b) a guide rail, and/or (c) multiple fasteners that are intracardially tightenable, from a proximal extracorporeal portion of the delivery assembly, in a manner that draws the guide rail into a guide arrangement around at least part of the guide frame, and/or (ii) a driver, configured to advance the implant along the guide rail in the guide arrangement.
Example 2. The system according to example 1, wherein each of the fasteners is a suture.
Example 3. The system according to any one of examples 1-2, wherein the guide rail defines a plurality of imaging markers.
Example 4. The system according to any one of examples 1-3, wherein the delivery assembly is configured to facilitate the guide assembly withdrawing the guide rail and the guide frame from the heart while the implant remains in the heart.
Example 5. The system according to any one of examples 1-4, wherein, in the delivery state, the guide rail is disposed alongside the guide frame.
Example 6. The system according to any one of examples 1-5, wherein the guide rail is a hypotube.
Example 7. The system according to any one of examples 1-6, wherein the implant has an axial length of 5-12 cm.
Example 8. The system according to any one of examples 1-7, wherein the guide assembly is configured to expand the guide frame prior to the guide rail becoming drawn into the guide arrangement.
Example 9. The system according to any one of examples 1-8, wherein the guide assembly is configured to expand the guide frame subsequently to the guide rail becoming drawn into the guide arrangement.
Example 10. The system according to any one of examples 1-9, wherein the guide frame is self-expanding.
Example 11. The system according to any one of examples 1-10, wherein the implant is sterile.
Example 12. The system according to any one of examples 1-11, wherein at least the distal part of the guide assembly is sterile.
Example 13. The system according to any one of examples 1-12, wherein the driver is sterile.
Example 14. The system according to any one of examples 1-13, wherein the multiple fasteners are tightenable independently of a state of expansion of the guide frame.
Example 15. The system according to any one of examples 1-14, wherein the guide rail comprises a series of electrodes, spaced along the guide rail, and electrically connected to an extracorporeal portion of the delivery assembly.
Example 16. The system according to example 15, wherein at least one of the electrodes is radiopaque.
Example 17. The system according to example 15, wherein each of the electrodes is a ring electrode that is positioned around the guide rail.
Example 18. The system according to example 15, further comprising a data-processing system, electrically connectable to the one or more electrodes by being connected to a terminal at the extracorporeal portion of the delivery assembly, and configured: (i) to receive an electrical signal from the one or more electrodes, and/or (ii) to, responsively to the electrical signal, provide an output indicative of a position of the guide rail within the heart.
Example 19. The system according to example 18, wherein: (i) the position includes a proximity of the guide rail to a tissue surface, and/or (ii) the data-processing system is configured to, responsively to the electrical signal, provide the output indicative of the proximity of the guide rail to the tissue surface.
Example 20. The system according to example 18, wherein: (i) the position includes contact of the guide rail with a tissue surface, and/or (ii) the data-processing system is configured to, responsively to the electrical signal, provide the output indicative of the contact of the guide rail to the tissue surface.
Example 21. The system according to example 18, wherein: (i) the position is a position along an atrioventricular axis of the heart, and/or (ii) the data-processing system is configured to, responsively to the electrical signal, provide the output indicative of the position of the guide rail along the atrioventricular axis.
Example 22. The system according to example 18, wherein: (i) the output is indicative of a tissue-type with which the guide rail is in contact, and/or (ii) the data-processing system is configured to, responsively to the electrical signal, provide the output indicative of the tissue-type.
Example 23. The system according to example 18, wherein: (i) the electrical signal is an ECG signal, and/or (ii) the data-processing system is configured to receive the ECG signal.
Example 24. The system according to example 18, wherein: (i) the electrical signal is an exogenous signal, and/or (ii) the data-processing system is configured to receive the exogenous signal.
Example 25. The system according to example 24, wherein: (i) based on the exogenous signal, the data-processing system is configured to determine a bioimpedance of the tissue, and/or (ii) the data-processing system is configured to provide the output responsively to the bioimpedance.
Example 26. The system according to example 24, wherein the data-processing system is configured to drive the exogenous signal between at least two of the electrodes of the series.
Example 27. The system according to any one of examples 1-26, wherein the guide assembly comprises multiple spacers, arranged around the guide frame, and configured to maintain a spacing between the guide rail and the guide frame.
Example 28. The system according to example 27, wherein: (i) in the guide arrangement, the guide rail lies around a midsection of the guide frame, the midsection disposed longitudinally between an upstream section of the frame and a downstream section of the frame, and/or (ii) the multiple spacers are collectively defined by an elongate member that extends in a serpentine manner around the midsection of the guide frame.
Example 29. The system according to example 27, wherein: (i) in the guide arrangement, the guide rail lies around a midsection of the guide frame, the midsection disposed longitudinally between an upstream section of the frame and a downstream section of the frame, and/or (ii) each spacer extends longitudinally alongside part of the upstream section, the entire midsection, and part of the downstream section.
Example 30. The system according to example 29, wherein each of the spacers has: (i) an upstream terminus that is attached to the guide frame at the upstream section, and/or (ii) a downstream terminus that is disposed at the downstream section.
Example 31. The system according to example 30, wherein each of the spacers is in the form of a ribbon.
Example 32. The system according to example 30, wherein the downstream terminus is attached to the guide frame at the downstream section.
Example 33. The system according to example 30, wherein the downstream terminus is unattached to the guide frame at the downstream section.
Example 34. The system according to example 27, wherein: (i) in the guide arrangement, the guide rail lies around a midsection of the guide frame, the midsection disposed axially between an upstream section of the frame and a downstream section of the frame, and/or (ii) the spacers collectively form a shield around the midsection, the shield covering the midsection.
Example 35. The system according to example 27, wherein the spacers are actuatable to expand the guide frame.
Example 36. The system according to example 35, wherein each of the spacers is woven along the guide frame.
Example 37. The system according to example 27, wherein the spacers are actuatable to compress the guide frame.
Example 38. The system according to example 27, wherein each of the spacers is a wire, extending longitudinally alongside the guide frame.
Example 39. The system according to example 27, wherein the guide assembly comprises multiple wires, extending distally alongside the guide frame, around a distal end of the guide frame, and returning proximally alongside the guide frame, such that each of the multiple wires defines a pair of the spacers on opposing sides of the guide frame.
Example 40. The system according to any one of examples 1-39, wherein each of the multiple fasteners defines a loop around the guide rail.
Example 41. The system according to example 40, wherein each of the loops is looped around a respective strut of the guide frame.
Example 42. The system according to example 40, wherein each of the multiple fasteners is configured to release the guide rail by being unlooped from around the guide rail.
Example 43. The system according to example 40, wherein each fastener of the multiple fasteners is defined by a longitudinal member that extends, from the extracorporeal portion to the distal part, where the longitudinal member forms the loop.
Example 44. The system according to example 40, wherein: (i) the guide assembly is positionable in a position within the heart such that: (a) the guide rail is disposed along an exterior of the guide frame, at an upstream section of the guide frame, and/or (b) one or more fasteners of the multiple fasteners exits the guide frame at a downstream section of the guide frame, and extends along the exterior of the guide frame to the guide rail, and/or (ii) the one or more fasteners are intracardially tightenable in a manner that draws the guide rail into the guide arrangement by the one or more fasteners pulling the guide rail toward the downstream section.
Example 45. The system according to example 44, wherein: (i) in the position of the guide assembly within the heart: (a) the upstream section and the guide rail are upstream of the tissue, and/or (b) the downstream section is downstream of the tissue, and/or (ii) while the guide assembly remains in the position, the one or more fasteners are intracardially tightenable in a manner that draws the guide rail into the guide arrangement by the one or more fasteners pulling the guide rail against an upstream surface of the tissue.
Example 46. The system according to example 43, wherein, in the delivery state, each of the multiple fasteners has an exposed length by which the fastener extends out of the guide frame to the guide rail, the multiple fasteners having different exposed lengths to each other.
Example 47. The system according to example 46, wherein: (i) the multiple fasteners are arranged in a series around the guide frame, and/or (ii) in the delivery state, along the series, each successive fastener has a greater exposed length than the preceding fastener.
Example 48. The system according to any one of examples 1-47, wherein the tissue is tissue of an annulus of a valve of the heart, and the guide assembly is configured to position the guide rail, in the guide arrangement, against the annulus.
Example 49. The system according to example 48, wherein the guide assembly is configured to position the guide rail against the annulus subsequently to the guide rail being positioned in the guide arrangement.
Example 50. The system according to example 48, wherein the guide assembly is configured to position the guide frame to traverse the valve with an upstream section of the guide frame upstream of the valve and a downstream section of the guide frame downstream of the valve.
Example 51. The system according to example 50, wherein: (i) one or more fasteners of the multiple fasteners extends out of the guide frame at the downstream section of the guide frame, (ii) the guide assembly is positionable in the heart such that the guide rail is upstream of the annulus, and/or (iii) the one or more fasteners are intracardially tightenable in a manner that draws the guide rail against the annulus by the fasteners pulling the guide rail against an upstream surface of the annulus.
Example 52. The system according to example 50, wherein the guide assembly is configured to expand the guide frame while the guide frame remains traversing the valve.
Example 53. The system according to example 50, wherein the guide assembly is configured to expand the guide frame prior to positioning the guide frame to traverse the valve.
Example 54. The system according to any one of examples 1-53, wherein the guide assembly is configured to apply an expanding force to the guide frame in order to intracardially expand the guide frame toward the expanded state.
Example 55. The system according to example 54, wherein the guide assembly comprises a mechanical actuator that is actuatable to apply the expanding force.
Example 56. The system according to example 54, wherein the guide assembly comprises a balloon that is inflatable to apply the expanding force.
Example 57. The system according to any one of examples 1-56, wherein the implant comprises a flexible helical member that defines a plurality of turns.
Example 58. The system according to example 57, wherein the helical member has a constant pitch.
Example 59. The system according to example 57, wherein the helical member has a sharpened distal tip.
Example 60. The system according to example 59, wherein: (i) the implant has a head at a proximal end of the helical member, (ii) the driver is configured to engage the implant by engaging the head, and/or (iii) the helical member has a thickness that is greater toward the proximal end than toward the distal tip.
Example 61. The system according to example 60, wherein the thickness of the helical member is tapered to become progressively greater from the distal tip toward the proximal end.
Example 62. The system according to example 60, wherein the helical member has a stiffness that is greater toward the proximal end than toward the distal tip.
Example 63. The system according to example 59, wherein: (i) the driver is configured to anchor the implant along the tissue, over and along the guide rail, by rotating the helical member in a first direction such that the distal tip penetrates the tissue, and/or (ii) the helical member is deliverable towards the tissue over and along the guide rail while rotating the helical member in a second direction, the second direction being opposite to the first direction.
Example 64. The system according to example 57, wherein: (i) the guide assembly is configured to position, along a surface of the tissue, the guide rail in the guide arrangement, and/or (ii) the driver is configured to, while the guide rail in the guide arrangement is positioned along the surface of the tissue, anchor the implant along the tissue by screwing the helical member along the guide rail and the tissue such that part of each turn of the helical member becomes embedded within the tissue, and another part of each turn lies above a surface of the tissue.
Example 65. The system according to example 64, wherein the guide rail defines a groove, and the driver is configured to anchor the implant along the tissue by screwing the helical member over and along the guide rail while the helical member is threadedly engaged with, and recessed within, the groove.
Example 66. The system according to example 64, wherein: (i) the extracorporeal portion of the delivery assembly is electrically connected to the helical member and is adapted to apply electrical energy to the helical member, and/or (ii) the helical member is configured to contract responsively to the application of electrical energy, in a manner that draws the turns of the helical member closer to each other.
Example 67. The system according to example 66, wherein the extracorporeal portion comprises a power source, configured to provide the electrical energy.
Example 68. The system according to example 66, wherein the extracorporeal portion: (i) comprises a terminal, configured to be electrically and mechanically connected to a power source, and/or (ii) is configured to derive the electrical energy from the power source.
Example 69. The system according to example 66, wherein the driver is electrically conductive and electrically connects the extracorporeal portion to the helical member.
Example 70. The system according to example 64, wherein the helical member is sufficiently flexible to follow a curvature of the guide rail in the guide arrangement.
Example 71. The system according to example 64, wherein the guide rail is configured to limit a depth of penetration of the helical member into the tissue.
Example 72. The system according to example 64, wherein the implant further comprises a tensile member.
Example 73. The system according to example 72, wherein, in the guide arrangement, the tensile member extends through the guide rail.
Example 74. The system according to example 73, wherein the multiple turns circumscribe a central channel of the helical member, and wherein the delivery assembly is configured to, while the helical member remains anchored along the tissue, retract the guide rail out of the helical member, leaving the tensile member extended through the central channel, the tensile member configured to, upon being tensioned, axially contract the helical member.
Example 75. The system according to example 74, further comprising a stopper coupled to a distal end of the tensile member, such that tension applied to the tensile member longitudinally contracts the helical member by the stopper inhibiting sliding of the tensile member through the central channel.
Example 76. The system according to example 74, further comprising a tensioning tool that is configured to contract the tissue along which the helical member is anchored by axially contracting the helical member by applying tension to the tensile member.
Example 77. The system according to example 76, further comprising a stopper, wherein the tensioning tool is configured to fix the tension in the tensile member by locking the stopper to the tensile member.
Example 78. The system according to example 57, wherein the driver is configured to advance the implant along the guide rail by sliding the helical member over and along the guide rail.
Example 79. The system according to example 57, wherein the multiple turns circumscribe a central channel, and the guide rail extends through the central channel.
Example 80. The system according to example 79, wherein the central channel has a diameter, and the guide rail has a thickness that is at least 50 percent of the diameter of the central channel.
Example 81. The system according to example 57, wherein the driver is configured to advance the implant along the guide rail while screwing the helical member into and along the tissue.
Example 82. The system according to example 57, wherein the driver is engaged with a proximal end of the helical member and is configured to screw the implant into the tissue by applying torque to the proximal end of the implant.
Example 83. The system according to example 82, wherein the implant comprises a head coupled to a proximal end of the helical member, the driver being reversibly engaged with the head.
Example 84. The system according to any one of examples 1-83, wherein: (i) the guide frame has a longitudinal axis, and is intracardially expandable toward the expanded state by being expanded radially away from the longitudinal axis, and/or (ii) in the guide arrangement, the guide rail extends latitudinally around at least part of the guide frame.
Example 85. The system according to example 84, wherein, in the delivery state, the guide rail is disposed parallel with the longitudinal axis.
Example 86. The system according to any one of examples 1-85, further comprising a sheath, the distal part of the guide assembly being transluminally advanceable to the heart while in the delivery state within the sheath.
Example 87. The system according to example 86, wherein, in the guide arrangement, the guide rail extends, from the sheath, through an interior of the guide frame, and out of the guide frame at an exit site to lie around an exterior of the guide frame.
Example 88. The system according to example 86, wherein an invaginating part of the guide frame is configured to invaginate to form an invagination upon the guide frame being intracardially expanded toward its expanded state.
Example 89. The system according to example 88, wherein the guide assembly comprises a control shaft that is coupled to the guide frame at the invaginating part of the guide frame.
Example 90. The system according to example 86, wherein: (i) in the delivery state, the guide frame is constrained within the sheath, and/or (ii) the guide frame is configured to automatically self-expand within the heart upon becoming deployed out of the sheath.
Example 91. The system according to any one of examples 1-90, wherein the guide frame comprises a braided filament.
Example 92. The system according to example 91, wherein the filament is a wire.
Example 93. The system according to any one of examples 1-92, wherein each of the fasteners is defined by a longitudinal member that extends, from the extracorporeal portion to the distal part, where the fastener is engaged with the guide rail.
Example 94. The system according to example 93, wherein: (i) the guide frame defines an interior and has an exterior, and/or (ii) at the distal part, each of the longitudinal members extends from the interior, through the guide frame to the exterior, where the fastener is engaged with the guide rail.
Example 95. The system according to example 94, wherein: (i) the guide frame has a longitudinal axis, and is intracardially expandable toward the expanded state by being expanded radially away from the longitudinal axis, and/or (ii) at the distal part, each of the longitudinal members extends from the interior, laterally through the guide frame to the exterior, where the fastener is engaged with the guide rail.
Example 96. The system according to example 95, wherein: (i) in the guide arrangement, the guide rail lies around a midsection of the guide frame, the midsection disposed axially between an upstream section of the frame and a downstream section of the frame, (ii) the guide frame is a braided structure defined by a plurality of struts, (iii) at the midsection, each strut is twisted together with an adjacent strut to form a twisted-strut-pair, each twisted-strut-pair defining an eyelet therethrough, and/or (iv) each longitudinal member extends, from the interior, through a respective one of the eyelets, to the exterior, where the fastener is engaged with the guide rail.
Example 97. The system according to example 96, wherein each twisted-strut-pair is parallel with the longitudinal axis.
Example 98. The system according to example 94, wherein: (i) the guide assembly includes multiple rods, each of the multiple rods being tubular, and/or (ii) at the distal part, each of the longitudinal members extends out of a respective rod, through the guide frame to the exterior, where the fastener is engaged with the guide rail.
Example 99. The system according to example 98, wherein each of the rods has a distal opening that is disposed at an inner surface of the guide frame.
Example 100. The system according to example 98, wherein the multiple rods are flexible.
Example 101. The system according to example 98, wherein the multiple rods are longitudinally incompressible.
Example 102. The system according to example 98, wherein the multiple rods extend distally within the interior.
Example 103. The system according to any one of examples 1-102, wherein: (i) the guide assembly is configured to position the guide rail, in the guide arrangement, along a surface of the tissue, and/or (ii) the driver is configured to anchor the implant along the tissue by advancing the implant helically along the guide rail in the guide arrangement.
Example 104. The system according to example 103, wherein the driver has: (i) at a distal end of the driver, a drivehead that is reversibly engaged with the implant, (ii) a driveshaft, the driver being configured to advance the implant helically along the guide rail by torque being applied to the driveshaft, and/or (iii) a neck that connects the driveshaft to the drivehead in a manner that transfers torque from the driveshaft to the drivehead.
Example 105. The system according to example 104, wherein the driveshaft is 50-150 cm long.
Example 106. The system according to example 105, wherein the neck is 0.5-5 cm long.
Example 107. The system according to example 104, wherein both the driveshaft and the neck are formed from a unitary tube that has a first cut pattern along the neck, and a second cut pattern cut along the driveshaft, the second cut pattern being different from the first cut pattern.
Example 108. The system according to example 107, wherein the unitary tube further forms the drivehead.
Example 109. The system according to example 107, wherein the first cut pattern segments the neck into discrete vertebrae, and wherein the neck is bendable via movement of the vertebrae with respect to each other.
Example 110. The system according to example 109, wherein the vertebrae are articulatably coupled to each other via the first cut pattern.
Example 111. The system according to example 109, wherein the second cut pattern includes multiple transverse slits along the driveshaft, and wherein the driveshaft is bendable via deformation of the tube and the slits.
Example 112. The system according to example 109, wherein the first cut pattern includes multiple tortuous cuts distributed along the neck, each of the tortuous cuts completely circumscribing the tube.
Example 113. The system according to example 112, wherein the second cut pattern includes multiple slits distributed along the driveshaft, each of the slits incompletely circumscribing the tube.
Example 114. The system according to any one of examples 1-113, wherein the delivery assembly further comprises a fixation wire that is connected to a connector of the guide rail in a manner that fastens the connector to a connection location on the guide frame.
Example 115. The system according to example 114, wherein the connector is an eyelet defined by a distal end portion of the guide rail, and wherein the fixation wire fastens the connector to the connection location on the guide frame by extending out of the guide frame and looping through the eyelet.
Example 116. The system according to example 114, wherein the fixation wire is intracardially withdrawable from the connector of the guide rail to decouple the guide rail from the guide frame.
Example 117. The system according to example 114, wherein the connector is disposed at a distal end portion of the guide rail.
Example 118. The system according to example 117, wherein: (i) each of the fasteners is defined by a longitudinal member that extends, from the extracorporeal portion to the distal part of the guide assembly, where the fastener loops around the guide rail, and/or (ii) the fastening, by the fixation wire, of the connector to the connection location inhibits the guide rail from sliding out from the fasteners.
Example 119. The system according to example 117, wherein: (i) in the guide arrangement, the guide rail lies around a midsection of the guide frame, the midsection disposed axially between an upstream section of the frame and a downstream section of the guide frame, (ii) in the delivery state, the connection location is disposed at the downstream section, and/or (iii) the delivery assembly is adapted to transition the guide rail towards the guide arrangement by moving the distal end portion towards the midsection.
Example 120. The system according to example 119, wherein: (i) the multiple fasteners are arranged in a series around the guide frame, and/or (ii) the delivery assembly is adapted to move the distal end portion towards the midsection by tightening a distalmost fastener of the series.
Example 121. The system according to example 119, wherein the fixation wire is intracardially loosenable to facilitate the movement of the distal end portion away from the connection location and toward the midsection.
Example 122. The system according to any one of examples 1-121, wherein, in the delivery state, the guide rail extends distally through an interior of the guide frame, and out of the guide frame at an exit site to lie along an exterior of the guide frame.
Example 123. The system according to example 122, wherein: (i) the guide frame has a longitudinal axis, and is intracardially expandable toward the expanded state by being expanded radially away from the longitudinal axis, and/or (ii) in the guide arrangement, the guide rail extends distally through the interior of the guide frame, and out of the guide frame at the exit site to curve around the exterior of the guide frame and the longitudinal axis.
Example 124. The system according to any one of examples 1-123, wherein, in the guide arrangement, the guide rail extends distally through an interior of the guide frame, and out of the guide frame at an exit site to lie around an exterior of the guide frame.
Example 125. The system according to example 124, wherein: (i) the delivery assembly further comprises a tube, the guide rail extending through the tube, and/or (ii) in the guide arrangement: (a) the tube extends distally through the interior of the guide frame, and out of the guide frame at the exit site, (b) within the tube, the guide rail extends distally through the interior of the guide frame and out of the guide frame at the exit site, and/or (c) at the exterior of the guide frame, the guide rail exits the tube to lie, exposed from the tube, around the exterior of the guide frame.
Example 126. The system according to example 125, wherein the tube has a distal section that exits the guide frame at the exit site, and at least the distal section of the tube is a flexible sleeve.
Example 127. The system according to any one of examples 1-126, wherein: (i) the delivery assembly further comprises a tube, the guide rail extending through the tube, and/or (ii) in the guide arrangement, the guide rail extends distally through and out of the tube to lie, exposed from the tube, around an exterior of the guide frame.
Example 128. The system according to example 127, wherein at least a distal section of the tube is a flexible sleeve.
Example 129. The system according to example 128, wherein the sleeve comprises a polymer.
Example 130. The system according to example 128, wherein the sleeve is a fabric sleeve.
Example 131. The system according to any one of examples 1-130, wherein: (i) the guide assembly is configured to position the guide rail, in the guide arrangement, along a surface of the tissue, (ii) the implant comprises a suture, and/or (iii) the driver is configured to stitch the suture along the tissue by advancing the suture helically along the guide rail in the guide arrangement.
Example 132. The system according to example 131, wherein: (i) the implant further comprises a tensile member, (ii) the driver is configured to stitch the suture along the tissue such that the suture defines a series of turns along the tissue, with the tensile member extending along an interior of the series of turns, and/or (iii) the implant is configured such that tensioning of the tensile member adjusts a dimension of the tissue by the tensile member pulling on the suture.
Example 133. The system according to example 132, wherein: (i) the driver is configured to stitch the suture such that the series of turns are disposed in a curved path along the tissue, the curved path having a radius of curvature, and/or (ii) the implant is configured such that tensioning of the tensile member adjusts the dimension of the tissue by reducing the radius of curvature of the curved path.
Example 134. The system according to example 132, wherein: (i) the driver is configured to stitch the suture such that the series of turns are disposed in a curved path along the tissue, the curved path having a length, and/or (ii) the implant is configured such that tensioning of the tensile member adjusts the dimension of the tissue by reducing the length of the curved path.
Example 135. The system according to example 132, wherein the tensile member is disposed within a lumen of the guide rail, and wherein, the delivery assembly is configured to retract the guide rail proximally out from the series of turns, leaving the tensile member exposed within the series of turns.
Example 136. The system according to example 132, wherein: (i) the driver comprises a helical member, and is adapted to stitch the suture along the tissue by driving the helical member helically along the guide rail in the guide arrangement, such that the helical member becomes temporarily stitched along the tissue, and/or (ii) the delivery assembly is configured to leave the suture stitched along the tissue with the tensile member extending along the interior of the series of turns by: (a) unstitching the helical member from the tissue by retracting the helical member helically, and/or (b) retracting the guide rail linearly.
Example 137. The system according to example 131, wherein the driver comprises a helical member that is adapted to stitch the suture along the tissue by driving the helical member helically along the guide rail in the guide arrangement.
Example 138. The system according to example 137, wherein the suture is attached to an exterior of the helical member.
Example 139. The system according to example 137, wherein the suture is detachable from the helical member once the suture is stitched along the tissue.
Example 140. The system according to example 137, wherein the suture is attached to a distal end portion of the helical member, and wherein the driver is adapted to stitch the suture along the tissue by withdrawing the helical member from the tissue such that the suture is drawn into and along the tissue by the withdrawal of the helical member.
Example 141. The system according to example 137, wherein the driver is adapted to stitch the suture into the tissue alongside the helical member.
Example 142. The system according to example 137, wherein the helical member defines multiple turns, and wherein the driver is configured to advance the implant along the tissue by screwing the helical member and the suture into the tissue such that part of each turn of the helical member becomes embedded within the tissue, and another part of each turn lies above the surface of the tissue.
Example 143. The system according to example 137, wherein the helical member is a hollow helical needle defining a channel therethrough, and wherein the driver is configured to stitch the suture along the tissue by screwing the helical member along the tissue while the suture is disposed within the channel.
Example 144. The system according to example 143, wherein the driver is configured to be unscrewed from the tissue, leaving the suture stitched along the tissue.
Example 145. The system according to any one of examples 1-144, wherein: (i) in the guide arrangement, the guide rail lies around a midsection of the guide frame, the midsection disposed axially between an upstream section of the frame and a downstream section of the frame, and/or (ii) in the expanded state of the guide frame, the upstream section is wider than the downstream section.
Example 146. The system according to example 145, wherein: (i) the guide assembly further comprises a control shaft that extends from the extracorporeal portion and is coupled to the upstream section of the guide frame, (ii) the guide assembly comprises multiple actuator wires, operably coupled to the extracorporeal portion, and/or (iii) each of the actuator wires extends, from the control shaft, distally through an interior of the upstream section of the guide frame, and weavingly along the downstream section of the guide frame, such that tensioning the actuator wires by operation of the extracorporeal portion radially expands the guide frame.
Example 147. The system according to example 145, wherein the upstream section is wider than the midsection.
Example 148. The system according to example 145, wherein, in the expanded state, the guide frame is mushroom-shaped.
Example 149. The system according to example 145, wherein, in the expanded state, the upstream section protrudes radially outwards over the midsection.
Example 150. The system according to example 149, wherein: (i) the driver is configured to implant the implant along the tissue, over and along the guide rail, and/or (ii) the guide frame is positionable within the heart, in the expanded state, such that the upstream section protrudes radially outwards over an upstream surface of the tissue.
Example 151. The system according to example 150, wherein the guide assembly is configured to move the guide frame, in the expanded state, in a downstream direction until the upstream section protrudes radially outwards over the upstream surface of the tissue.
Example 152. The system according to example 150, wherein the guide assembly is configured to move the guide frame, in the expanded state, in an upstream direction such that the upstream section squeezes past the tissue to protrude radially outwards over the upstream surface of the tissue.
Example 153. The system according to any one of examples 1-152, wherein: (i) the guide frame defines an upstream section and a downstream section, and a concave waist disposed axially between the upstream section and the downstream section, and/or (ii) in the expanded state of the guide frame, the waist has a smaller circumference than both the upstream section and the downstream section.
Example 154. The system according to example 153, wherein: (i) the guide assembly further comprises a control shaft that extends from the extracorporeal portion and is coupled to the upstream section of the guide frame, (ii) the guide assembly comprises multiple actuator wires, operably coupled to the extracorporeal portion, and extending via the control shaft to the guide frame, and/or (iii) each of the actuator wires extends, from the control shaft, distally through an interior of the upstream section of the guide frame, and weavingly along the downstream section of the guide frame, such that tensioning the actuator wires by operation of the extracorporeal portion radially expands the guide frame.
Example 155. The system according to example 153, wherein, in the guide arrangement, the guide rail lies around the waist of the guide frame.
Example 156. The system according to example 153, wherein the guide assembly is adapted to position the guide frame at the tissue such that the tissue becomes sandwiched between the upstream section and the downstream section.
Example 157. The system according to example 153, wherein the guide assembly is adapted to position the guide frame at the tissue such that the tissue becomes gripped between the upstream section and the downstream section.
Example 158. The system according to any one of examples 1-157, wherein a distal end of the guide frame is invaginated to form an invagination.
Example 159. The system according to example 158, wherein the invagination is secured by a crimp.
Example 160. The system according to any one of examples 1-159, wherein the guide frame defines a plurality of struts, and wherein the struts are gathered together at a distal end of the guide frame to form an atraumatic distal end of the guide frame.
Example 161. The system according to example 160, wherein the distal end is coated with a coating.
Example 162. The system according to example 160, wherein the distal end is secured by a crimp.
Example 163. The system according to example 160, wherein the struts are invaginated to form the atraumatic distal end.
Example 164. The system according to any one of examples 1-163, wherein the guide assembly comprises a control shaft, a distal end of the control shaft being attached to the guide frame in a manner that facilitates transluminal control of the guide frame via the control shaft.
Example 165. The system according to example 164, wherein: (i) the control shaft comprises a tube, (ii) at a distal region of the control shaft, the control shaft defines a flex zone in which cuts in the tube confer flexibility to the tube, (iii) the control shaft further comprises a strip that has greater tensile strength than the flex zone, and/or (iv) a first end of the strip is attached to the tube distally from the flex zone, and a second end of the strip is attached to the tube proximally from the flex zone, such that the strip lies slack alongside the flex zone.
Example 166. The system according to example 165, wherein each of the first end of the strip and the second end of the strip are attached to the tube by welding.
Example 167. The system according to example 165, wherein: (i) the strip is a first strip, (ii) the control shaft further comprises a second strip having greater tensile strength than the flex zone, and/or (iii) a first end of the second strip is attached to the tube distally from the flex zone, opposite the first end of the first strip, and a second end of the second strip is attached to the tube proximally from the flex zone, opposite the second end of the first strip, such that the strip lies slack alongside the flex zone, on an opposing side of the flex zone to the first strip.
Example 168. The system according to example 165, wherein the tube is a hypotube.
Example 169. The system according to example 165, wherein the delivery assembly is configured to facilitate withdrawal of the guide frame via pulling of the control shaft in a manner that tensions the strip.
Example 170. The system according to example 164, wherein: (i) the control shaft comprises a tube, and/or (ii) at a distal region of the control shaft, the control shaft defines a flex zone in which a cut pattern in the tube confers flexibility to the tube, the cut pattern including multiple slits distributed along the flex zone, each of the slits incompletely circumscribing the tube such that an uncut axial strip remains along the flex zone.
Example 171. The system according to example 170, wherein: (i) the strip is a first strip, and/or (ii) the cut pattern defines a second uncut axial strip along the flex zone, the second strip being disposed on an opposing side of the flex zone to the first strip.
Example 172. The system according to example 170, wherein the tube is a hypotube.
Example 173. The system according to any one of examples 1-172, wherein the guide assembly comprises multiple actuator wires, operably coupled to the extracorporeal portion, woven longitudinally along at least part of the guide frame, and attached to a downstream part of the guide frame, such that tensioning the actuator wires from the extracorporeal portion radially expands the guide frame.
Example 174. The system according to example 173, wherein: (i) in the guide arrangement, the guide rail lies around a midsection of the guide frame, the midsection disposed longitudinally between an upstream section of the frame and a downstream section of the frame, (ii) the guide assembly further comprises a control shaft that extends from the extracorporeal portion and is coupled to the upstream section of the guide frame, and/or (iii) each of the actuator wires extends, from the control shaft, distally through an interior of the upstream section of the guide frame, and weavingly along the downstream section of the guide frame.
Example 175. The system according to example 174, wherein in the expanded state of the guide frame, at least part of the upstream section is wider than the downstream section.
Example 176. The system according to example 174, wherein in the expanded state of the guide frame, at least part of the upstream section is wider than the midsection.
Example 177. The system according to example 173, wherein: (i) the guide assembly further comprises a control shaft that extends from the extracorporeal portion and is coupled to the upstream section of the guide frame, and/or (ii) the guide assembly is configured such that the guide frame is pivotable with respect to the control shaft via differential tensioning of the actuator wires.
Example 178. The system according to example 177, wherein the extracorporeal portion: (i) comprises at least one controller to which the actuator wires are operatively coupled, and/or (ii) is configured to differentially actuate the actuator wires via actuation of the at least one controller.
Example 179. The system according to example 178, wherein: (i) the guide frame is configured to radially expand responsively to balanced tension in the actuator wires, and/or (ii) the extracorporeal portion is configured to apply the balanced tension to the actuator wires.
Example 180. The system according to example 179, wherein the at least one controller is configured with: (i) a first actuation mode that applies the balanced tension to the actuator wires, and/or (ii) a second actuation mode that applies the differential tension to the actuator wires.
Example 181. The system according to any one of examples 1-180, wherein: (i) in the guide arrangement, the guide rail lies around a midsection of the guide frame, the midsection disposed axially between an upstream section of the frame and a downstream section of the frame, and/or (ii) the guide assembly comprises a shield that is disposed around the midsection such that, in the guide arrangement, the shield is disposed between the guide rail and the guide frame.
Example 182. The system according to example 181, wherein the shield comprises an elastic material.
Example 183. The system according to example 181, wherein the shield is defined by a fabric.
Example 184. The system according to example 181, wherein the shield is defined by a film.
Example 185. The system according to example 181, wherein the shield is defined by a net.
Example 186. The system according to example 181, wherein the shield has a hypotube-type structure.
Example 187. The system according to example 181, wherein the shield is a ribbon that, in the delivery state of the guide assembly, is wrapped around the guide frame, and wherein expanding the guide frame towards the expanded state causes the ribbon to slide over itself in a manner that reduces the wrapping around the guide frame.
Example 188. The system according to example 181, wherein the shield is defined by multiple ribbons distributed circumferentially around the midsection.
Example 189. The system according to example 188, wherein, in the guide arrangement, each ribbon contacts its neighboring ribbons, such that the ribbons collectively cover the midsection.
Example 190. The system according to example 188, wherein: (i) in the delivery state, the ribbons are imbricated around the midsection, and/or (ii) the ribbons are configured to facilitate expansion of the guide frame towards the expanded state by the ribbons sliding over each other while collectively covering the midsection.
Example 191. The system according to example 190, wherein, in the expanded state, the ribbons remain imbricated around the midsection.
Example 192. The system according to example 190, wherein, in the expanded state, the ribbons are arranged edge-to-edge around the midsection.
Example 193. The system according to example 188, wherein each ribbon is polymeric.
Example 194. The system according to example 188, wherein each ribbon is metallic.
Example 195. The system according to any one of examples 1-194, wherein: (i) in the guide arrangement, the guide rail lies around a midsection of the guide frame, the midsection disposed axially between an upstream section of the frame and a downstream section of the frame, (ii) the guide frame is a braided structure defined by a plurality of struts, and/or (iii) at the midsection, each strut is twisted together with an adjacent strut to form a twisted-strut-pair.
Example 196. The system according to example 195, wherein each twisted-strut-pair is covered with a covering.
Example 197. The system according to example 195, wherein the guide frame defines a longitudinal axis between the upstream section and the downstream section, and wherein each twisted-strut-pair is substantially parallel to the longitudinal axis.
Example 198. The system according to any one of examples 1-197, wherein: (i) the guide rail defines an external thread, and/or (ii) the driver is configured to advance the implant along the guide rail by advancing the implant threadedly along the external thread.
Example 199. The system according to example 198, wherein: (i) the external thread defines a groove, (ii) the implant comprises a helical member defining a plurality of turns, and/or (iii) the driver is configured to screw the helical member helically along the thread while the helical member is recessed within the groove.
Example 200. The system according to example 198, wherein: (i) the guide rail defines a central guide-rail axis, and has a tissue-facing surface that, along the guide rail, is disposed closer than the external thread to the central guide-rail axis, and/or (ii) in the guide arrangement, the external thread faces medially toward the guide frame, and the tissue-facing surface faces radially away from the guide frame.
Example 201. The system according to example 200, wherein the tissue-facing surface is unthreaded, and runs parallel with the external thread.
Example 202. The system according to example 200, wherein the tissue-facing surface is substantially flat.
Example 203. The system according to example 200, wherein the tissue-facing surface is concave.
Example 204. The system according to any one of examples 1-203, wherein the guide assembly comprises a rider that is slidably mounted to the guide rail such that, as the driver advances the implant along the guide rail, a leading end of the implant pushes the rider along the guide rail, while the rider shields the guide frame from the leading end of the implant.
Example 205. The system according to example 204, wherein: (i) the implant comprises a helical member defining a sharpened tip at the leading end, and/or (ii) the rider defines a lobe that, as the implant pushes the rider along the guide rail, the lobe remains disposed between the leading end and the guide frame, thereby shielding the guide frame from the sharpened tip.
Example 206. The system according to example 205, wherein the lobe is rotationally locked with respect to the guide rail.
Example 207. The system according to example 206, wherein the lobe is rotationally locked with respect to the guide rail via keying between the rider and the guide rail.
Example 208. A method usable with a real or simulated heart of a real or simulated subject, the method comprising: (A) transluminally advancing a guide frame to the heart while the guide frame is secured to a guide rail via multiple fasteners that extend out of the guide frame to the guide rail; (B) expanding the guide frame within the heart; (C) drawing the guide rail into a guide arrangement around at least a part of the guide frame by tightening at least one of the multiple fasteners; and/or (d) while the guide rail remains in the guide arrangement, positioning an implant along a real or simulated tissue of the heart, guided by the guide rail.
Example 209. The method according to example 208, further comprising sterilizing the implant.
Example 210. The method according to any one of examples 208-209, further comprising sterilizing the guide frame.
Example 211. The method according to any one of examples 208-210, further comprising sterilizing the guide rail.
Example 212. The method according to any one of examples 208-211, wherein drawing the guide rail into the guide arrangement comprises drawing the guide rail into the guide arrangement by tightening: (i) by a first amount, a first of the multiple fasteners, and/or (ii) by a second amount, a second of the multiple fasteners, the second amount being greater than the first amount.
Example 213. The method according to any one of examples 208-212, wherein the method further comprises, subsequently to positioning the implant along the tissue, withdrawing the guide rail and the guide frame from the heart while the implant remains disposed along the tissue.
Example 214. The method according to any one of examples 208-213, wherein advancing the guide frame to the heart comprises advancing the guide frame to the heart while the guide rail is disposed alongside the guide frame.
Example 215. The method according to any one of examples 208-214, wherein: (i) transluminally advancing the guide frame to the heart comprises transluminally advancing the guide frame to the heart while the guide frame is constrained in a delivery state within a sheath, and/or (ii) expanding the guide frame within the heart comprises deploying the guide frame out of the sheath such that the guide frame automatically self-expands within the heart.
Example 216. The method according to any one of examples 208-215, wherein the guide rail defines multiple imaging markers, and wherein drawing the guide rail into the guide arrangement comprises drawing the guide rail into the guide arrangement guided by at least one image that includes the imaging markers.
Example 217. The method according to example 216, wherein the method further comprises, prior to positioning the implant along the tissue, determining a desired size of the implant guided by the at least one image that includes the imaging markers.
Example 218. The method according to example 216, wherein the method further comprises, prior to positioning the implant along the tissue, selecting the implant from a selection of implants, responsively to at least one image that includes the imaging markers.
Example 219. The method according to example 216, wherein the method further comprises, prior to positioning the implant along the tissue, adjusting a size of the implant responsively to at least one image that includes the imaging markers.
Example 220. The method according to any one of examples 208-219, wherein the method further comprises determining a position of the guide rail within the heart responsively to an electrical signal detected via the guide rail.
Example 221. The method according to example 220, wherein the method further comprises, prior to positioning the implant along the tissue, selecting the implant from a selection of implants, responsively to the electrical signal.
Example 222. The method according to example 220, wherein the method further comprises, prior to positioning the implant along the tissue, adjusting a size of the implant responsively to the electrical signal.
Example 223. The method according to example 220, wherein: (i) the guide rail defines multiple imaging markers, (ii) the electrical signal is detected via the imaging markers serving as electrodes, and/or (iii) drawing the guide rail into the guide arrangement comprises drawing the guide rail into the guide arrangement guided by at least one image that includes the imaging markers.
Example 224. The method according to example 220, wherein the electrical signal is an endogenous electrical signal, and wherein determining the position of the guide rail responsively to the electrical signal comprises determining the position of the guide rail responsively to the endogenous electrical signal.
Example 225. The method according to example 224, wherein the endogenous electrical signal is an ECG signal, and wherein determining the position of the guide rail responsively to the endogenous electrical signal comprises determining the position of the guide rail responsively to the ECG signal.
Example 226. The method according to example 220, wherein the electrical signal is an exogenous electrical signal, and wherein determining the position of the guide rail responsively to the electrical signal comprises determining the position of the guide rail responsively to the exogenous electrical signal.
Example 227. The method according to example 226, further comprising applying the exogenous electrical signal to the subject.
Example 228. The method according to example 226, wherein determining the position of the guide rail responsively to the exogenous electrical signal comprises determining the position of the guide rail responsively to sensing a bioimpedance of the tissue.
Example 229. The method according to example 220, wherein the method further comprises, subsequently to drawing the guide rail into the guide arrangement around the part of the guide frame and prior to positioning the implant along the tissue, responsively to the electrical signal, positioning the guide rail within the heart.
Example 230. The method according to example 229, wherein positioning the guide rail within the heart comprises determining a position of the guide rail along an atrioventricular axis of the heart.
Example 231. The method according to example 229, wherein determining the position of the guide rail within the heart comprises verifying contact between at least a portion of the guide rail and the tissue.
Example 232. The method according to example 231, wherein: (i) the guide rail has a series of electrodes spaced along the guide rail, the electrical signal is one of multiple electrical signals received via the series of electrodes, and/or (ii) positioning the guide rail within the heart comprises positioning the guide rail within the heart responsively to the multiple electrical signals.
Example 233. The method according to example 232, wherein: (a) positioning the implant along the tissue comprises positioning the implant along a stretch of the tissue, and/or (b) adjusting the position of the guide rail comprises adjusting the position of the guide rail such that (i) electrical signals received from a first portion of the guide rail that is to be positioned along the stretch indicate a presence of tissue contact, and/or (ii) electrical signals received from a second portion of the guide rail that is to be positioned away from the tissue indicate an absence of tissue contact.
Example 234. The method according to any one of examples 208-233, wherein: (i) the guide frame has a longitudinal axis, (ii) expanding the guide frame comprises expanding the guide frame radially away from the longitudinal axis, and/or (iii) in the guide arrangement, the guide rail extends latitudinally around at least part of the guide frame.
Example 235. The method according to example 234, wherein advancing the guide frame to the heart comprises advancing the guide frame to the heart while the guide rail is disposed parallel with the longitudinal axis.
Example 236. The method according to any one of examples 208-235, wherein expanding the guide frame within the heart comprises applying an expanding force to the guide frame within the heart.
Example 237. The method according to example 236, wherein applying the expanding force comprises actuating a mechanical actuator within the heart.
Example 238. The method according to example 236, wherein applying the expanding force comprises inflating a balloon within the heart.
Example 239. The method according to example 208, wherein the implant comprises a helical member defining a plurality of turns, and wherein positioning the implant along the tissue comprises screwing the helical member into and along the tissue in a manner in which the helical member becomes progressively threaded around the guide rail.
Example 240. The method according to example 239, wherein the method further comprises applying energy to the helical member while the helical member remains screwed into and along the tissue such that, responsively to the application of the energy, the helical member contracts in a manner that draws the turns of the helical member toward to each other.
Example 241. The method according to example 240, wherein applying the energy to the helical member comprises applying electrical energy to the helical member from an extracorporeal power source that is electrically connected to the helical member.
Example 242. The method according to example 239, wherein screwing the helical member into the tissue comprises screwing the helical member into the tissue by applying torque to a proximal end of the helical member.
Example 243. The method according to example 242, wherein: (i) the helical member defines a sharpened distal tip, (ii) the helical member has a thickness that is greater toward the proximal end than toward the distal tip, and/or (iii) applying torque to the proximal end of the helical member comprises applying torque to the proximal end toward which the thickness of the helical member is greater.
Example 244. The method according to example 243, wherein the thickness of the helical member is tapered to become progressively greater from the distal tip toward the proximal end, and wherein screwing the helical member into the tissue comprises screwing the tapered helical member into the tissue.
Example 245. The method according to example 242, wherein: (i) the helical member defines a sharpened distal tip, (ii) the helical member has a stiffness that is greater toward the proximal end than toward the distal tip, and/or (iii) applying torque to the proximal end of the helical member comprises applying torque to the proximal end toward which the stiffness of the helical member is greater.
Example 246. The method according to example 242, wherein: (i) applying torque to the proximal end of the helical member comprises applying torque to the proximal end of the helical member in a first direction such that a distal tip of the helical member penetrates the tissue, and/or (ii) the method further comprises, prior to screwing the helical member along the tissue, delivering the helical member towards the tissue over and along the guide rail while rotating the helical member in a second direction, the second direction being opposite to the first direction.
Example 247. The method according to example 239, wherein screwing the helical member into the tissue comprises screwing the helical member into the tissue such that a screw axis of the helical member is disposed along a surface of the tissue.
Example 248. The method according to example 239, wherein the method further comprises, subsequently to positioning the implant along the tissue, retracting the guide rail from out of the implant, leaving the implant implanted in the heart.
Example 249. The method according to example 248, wherein retracting the guide rail from out of the implant comprises sliding the guide rail proximally through the implant.
Example 250. The method according to example 239, wherein: (i) the implant includes a helical member defining multiple turns that circumscribe a central channel, and/or (ii) screwing the implant into and along the tissue comprises screwing the helical member into the tissue, such that part of each turn of the helical member becomes embedded within the tissue, and another part of each turn lies above a surface of the tissue.
Example 251. The method according to example 250, wherein the helical member defines a sharpened distal tip and wherein screwing the helical member into the tissue comprises repeatedly driving the sharpened distal tip into and out of the tissue.
Example 252. The method according to example 250, wherein the implant further includes a tensile member, and wherein the method further comprises, subsequently to implanting the helical member along the tissue, axially contracting the helical member by tensioning the tensile member.
Example 253. The method according to example 250, wherein the helical member comprises a head, and wherein screwing the helical member into the tissue comprises screwing the helical member into the tissue using a driver that is engaged with the head.
Example 254. The method according to any one of examples 208-253, wherein: (i) the tissue is tissue of an annulus of a valve of the heart, and/or (ii) positioning the implant along the tissue, guided by the guide rail comprises advancing the implant along the tissue of the annulus, guided by the guide rail.
Example 255. The method according to example 254, wherein positioning the implant along the tissue comprises screwing the implant into and along an atrial surface of the tissue of the annulus.
Example 256. The method according to example 254, wherein expanding the guide frame comprises expanding the guide frame while the guide frame traverses the valve with an upstream section of the guide frame upstream of the valve and a downstream section of the guide frame downstream of the valve.
Example 257. The method according to example 256, wherein: (i) the guide frame has a midsection between the upstream section and the downstream section, and/or (ii) expanding the guide frame comprises expanding the guide frame such that the midsection presses radially against the valve.
Example 258. The method according to example 254, wherein: (i) the heart has an atrium upstream of the valve, and a ventricle downstream of the valve, and/or (ii) drawing the guide rail into the guide arrangement comprises drawing the guide rail into the guide arrangement while an upstream section of the guide frame is disposed within the atrium.
Example 259. The method according to example 258, wherein drawing the guide rail into the guide arrangement comprises drawing the guide rail into the guide arrangement while a downstream section of the guide frame is disposed in the ventricle.
Example 260. The method according to example 259, wherein: (i) each fastener of the multiple fasteners extends out of the guide frame at the downstream section of the guide frame, and/or (ii) drawing the guide rail into the guide arrangement by tightening the at least one fastener comprises drawing the guide rail into the guide arrangement while the guide rail is within the atrium, such that the fasteners pull the guide rail against an upstream surface of the annulus.
Example 261. The method according to example 258, wherein drawing the guide rail into the guide arrangement comprises drawing the guide rail into the guide arrangement while a downstream section of the guide frame is disposed in the atrium.
Example 262. The method according to example 261, further comprising, subsequently to drawing the guide rail into the guide arrangement, advancing the guide frame ventricularly through the valve until the guide rail abuts the annulus.
Example 263. The method according to example 258, wherein expanding the guide frame within the heart comprises expanding the guide frame within the atrium, and wherein the method further comprises, prior to drawing the guide frame into the guide arrangement, advancing a downstream section of the guide frame into the ventricle.
Example 264. The method according to example 254, wherein the method further comprises, subsequently to positioning the implant along the annulus, contracting the annulus by contracting the implant.
Example 265. The method according to example 264, wherein contracting the implant comprises applying tension to a tensile member extending through a central channel of the implant.
Example 266. The method according to example 265, wherein applying tension to the tensile member comprises pulling the tensile member using a tensioning tool.
Example 267. The method according to example 265, further comprising locking the tension in the tensile member by locking a stopper to the tensile member.
Example 268. The method according to example 267, further comprising trimming excess tensile member that is proximal to the stopper.
Example 269. The method according to example 265, wherein: (i) drawing the guide rail into the guide arrangement comprises drawing the guide rail into the guide arrangement while the tensile member is extended through a lumen of the guide rail, (ii) advancing the implant along the tissue comprises advancing the implant over and along the guide rail such that the guide rail progressively becomes disposed within the central channel of the implant, and/or (iii) the method further comprises, subsequently to advancing the implant along the tissue, and prior to contracting the implant, withdrawing the guide rail and out of the central channel, leaving the tensile member extending through the central channel.
Example 270. The method according to any one of examples 208-269, wherein positioning the implant along the tissue comprises advancing the implant along the guide rail.
Example 271. The method according to example 270, wherein the implant includes a helical member defining a plurality of turns, and wherein advancing the implant along the guide rail comprises advancing the implant along the guide rail by screwing the helical member into the tissue, such that part of each turn of the helical member becomes embedded within the tissue, and another part of each turn lies above a surface of the tissue.
Example 272. The method according to any one of examples 208-271, wherein: (i) each fastener of the multiple fasteners defines a loop around the guide rail, and/or (ii) tightening the multiple fasteners comprises, for each fastener of the multiple fasteners, tightening the respective loop of the fastener.
Example 273. The method according to example 272, wherein drawing the guide rail into the guide arrangement comprises drawing the guide rail into the guide arrangement while: (a) the guide rail is disposed along an exterior of the guide frame, at an upstream section of the guide frame, and/or (b) each fastener of the multiple fasteners exits the guide frame at a downstream section of the guide frame, and extends along the exterior of the guide frame to the guide rail, such that tightening the at least one fastener pulls the guide rail toward the downstream section.
Example 274. The method according to example 272, wherein: (i) each fastener of the multiple fasteners is defined by a longitudinal member that extends, from outside of the subject to the heart, where the longitudinal member forms the loop, and/or (ii) for each fastener of the multiple fasteners, tightening the respective loop of the fastener comprises pulling the longitudinal member from outside of the subject.
Example 275. The method according to example 274, wherein advancing the guide frame comprises advancing the guide frame while each of the multiple fasteners has a respective exposed length by which the fastener extends out of the guide frame to the guide rail, the multiple fasteners having different exposed lengths to each other.
Example 276. The method according to example 275, wherein: (i) the multiple fasteners are arranged in a series around the guide frame, and/or (ii) advancing the guide frame comprises advancing the guide frame while, along the series, each successive fastener has a greater exposed length than the preceding fastener.
Example 277. The method according to example 274, wherein drawing the guide rail into the guide arrangement comprises, for each fastener of the multiple fasteners, pulling the longitudinal member from outside the subject until the loop of the fastener draws the guide rail against the guide frame.
Example 278. The method according to example 274, wherein the method further includes, subsequently to positioning the implant along the tissue, for each of the fasteners, opening the loop.
Example 279. The method according to example 278, wherein opening the loop comprises unlooping the loop from around the guide rail.
Example 280. The method according to example 278, wherein opening the loop comprises unlooping the loop from around the implant.
Example 281. The method according to any one of examples 208-280, wherein drawing the guide rail into the guide arrangement comprises drawing the guide rail into the guide arrangement such that multiple spacers, disposed alongside the guide frame, become sandwiched between the guide rail and the guide frame.
Example 282. The method according to example 281, wherein each of the spacers is defined by a wire, and wherein drawing the guide rail into the guide arrangement comprises drawing the guide rail into the guide arrangement such that the wire becomes sandwiched between the guide rail and the guide frame.
Example 283. The method according to example 281, further comprising, while the guide rail remains in the guide arrangement, retracting the spacers from between the guide rail and the guide frame.
Example 284. The method according to example 281, wherein expanding the guide frame comprises expanding the guide frame by actuating the spacers.
Example 285. The method according to example 284, wherein the method further comprises, subsequently to positioning the implant along the tissue, compressing the guide frame by actuating the spacers.
Example 286. The method according to any one of examples 208-285, wherein positioning the implant along the tissue comprises advancing the implant helically along the guide rail in the guide arrangement such that the implant becomes implanted along a surface of the tissue.
Example 287. The method according to example 286, wherein advancing the implant helically along the guide rail comprises advancing the implant helically along the guide rail by applying torque to the implant using a driver, a drivehead of the driver being reversibly engaged with the implant, the driver having (i) a driveshaft, and/or (ii) a neck that connects the driveshaft to the drivehead.
Example 288. The method according to example 287, wherein both the driveshaft and the neck are formed from a unitary tube having a first cut pattern along the neck, and a second cut pattern cut along the driveshaft, the second cut pattern being different from the first cut pattern, and wherein transluminally advancing the implant to the tissue while the implant is reversibly engaged with the driver comprises transluminally advancing the implant to the tissue along a tortuous path while the implant is reversibly engaged with the driver such that the neck bends responsively to the tortuous path, facilitated by the first cut pattern.
Example 289. The method according to example 288, wherein the unitary tube further forms the drivehead, and wherein applying torque to the unitary tube comprises applying torque to the driveshaft of the unitary tube while the drivehead of the unitary tube is engaged with the implant.
Example 290. The method according to example 288, wherein the first cut pattern segments the neck into discrete vertebrae, and wherein transluminally advancing the implant to the tissue while the implant is reversibly engaged with the driver such that the neck bends responsively to the tortuous path comprises transluminally advancing the implant to the tissue while the implant is reversibly engaged with the driver such that vertebrae move with respect to each other, responsively to the tortuous path.
Example 291. The method according to any one of examples 208-290, wherein transluminally advancing the guide frame to the heart comprises transluminally advancing the guide frame to the heart while a fixation wire that is connected to a connector of the guide rail fastens the connector to a connection location on the guide frame.
Example 292. The method according to example 291, wherein: (i) the connector is an eyelet defined by a distal end portion of the guide rail, (ii) the fixation wire fastens the connector to the connection location on the guide frame by extending out of the guide frame and looping through the eyelet, and/or (iii) the method further comprises, prior to drawing the guide rail into the guide arrangement, intracardially loosening the fixation wire to facilitate movement of the guide rail away from the connection location and into the guide arrangement.
Example 293. The method according to example 291, wherein the method further comprises intracardially withdrawing the fixation wire from the connector of the guide rail to decouple the guide rail from the guide frame.
Example 294. The method according to example 291, wherein: (i) the connector is disposed at a distal end portion of the guide rail, (ii) in the guide arrangement, the guide rail lies around a midsection of the guide frame, the midsection disposed axially between an upstream section of the frame and a downstream section of the frame, (iii) during advancement of the guide frame to the heart, the connection location is disposed at the downstream section, and/or (iv) drawing the guide rail into the guide arrangement comprises moving the distal end portion towards the midsection.
Example 295. The method according to example 294, wherein: (i) each of the fasteners is defined by a longitudinal member that extends, from an extracorporeal end of the longitudinal member to the guide frame, where the fastener loops around the guide rail, (ii) the fastening, by the fixation wire, of the connector to the connection location inhibits the guide rail from sliding out from the fasteners, and/or (iii) transluminally advancing the guide frame to the heart comprises transluminally advancing the guide frame to the heart while the guide rail is inhibited, by the fastening, from sliding out from the fasteners.
Example 296. The method according to example 294, wherein: (i) the multiple fasteners are arranged in a series around the guide frame, and/or (ii) moving the distal end portion towards the midsection comprises tightening a distalmost fastener of the series.
Example 297. The method according to example 294, further comprising intracardially loosening the fixation wire to facilitate the movement of the distal end portion away from the connection location and toward the midsection.
Example 298. The method according to any one of examples 208-297, wherein transluminally advancing the guide frame to the heart comprises transluminally advancing the guide frame to the heart while the guide rail extends distally through an interior of the guide frame, and out of the guide frame at an exit site to lie along an exterior of the guide frame.
Example 299. The method according to any one of examples 208-298, wherein: (i) the guide frame has a longitudinal axis, (ii) expanding the guide frame comprises expanding the guide frame radially away from the longitudinal axis, and/or (iii) positioning the implant along the tissue comprises positioning the implant along the tissue while the guide rail extends distally through an interior of the guide frame, and laterally out of the guide frame at an exit site to lie along an exterior of the guide frame.
Example 300. The method according to example 299, wherein positioning the implant along the tissue comprises advancing the implant over and along the guide rail such that the implant passes through the interior of the guide frame, out of the guide frame at the exit site, and along the exterior of the guide frame and the tissue.
Example 301. The method according to example 300, wherein: (i) drawing the guide rail into the guide arrangement comprises drawing the guide rail into the guide arrangement such that: (a) within the interior of the guide frame, the guide rail is ensheathed within a tube that extends to the exterior of the guide frame, and/or (b) at the exterior of the guide frame, the guide rail is exposed from the tube; and/or (ii) advancing the implant over and along the guide rail comprises advancing the implant over and along the guide rail such that: (a) within the interior of the guide frame, the implant advances over and along the guide rail within the tube, and/or (b) at the exterior of the guide frame, the implant exits the tube to advance over and along the guide rail, exposed from the tube.
Example 302. The method according to any one of examples 208-301, wherein: (i) the implant comprises a suture, and/or (ii) positioning the implant along the tissue comprises stitching the suture along the tissue by advancing the suture helically along the guide rail in the guide arrangement.
Example 303. The method according to example 302, wherein: (i) the implant further comprises a tensile member, (ii) stitching the suture along the tissue comprises stitching the suture along the tissue and the tensile member such that the suture defines a series of turns along the tissue, with the tensile member extending along an interior of the series of turns, and/or (iii) the method further comprises tensioning the tensile member to adjust a dimension of the tissue by the tensile member pulling on the suture.
Example 304. The method according to example 303, wherein: (i) stitching the suture along the tissue comprises stitching the suture along the tissue such that the series of turns are disposed in a curved path along the tissue, the curved path having a radius of curvature, and/or (ii) tensioning the tensile member to adjust the dimension of the tissue comprises tensioning the tensile member to adjust the dimension of the tissue by reducing the radius of curvature of the curved path.
Example 305. The method according to example 303, wherein: (i) stitching the suture along the tissue comprises stitching the suture along the tissue such that the series of turns are disposed in a curved path along the tissue, the curved path having a length, and/or (ii) tensioning the tensile member to adjust the dimension of the tissue comprises tensioning the tensile member to adjust the dimension of the tissue by reducing the length of the curved path.
Example 306. The method according to example 303, wherein stitching the suture along the tissue comprises stitching the suture along the tissue while the tensile member is disposed within a lumen of the guide rail, and wherein, the method further comprises retracting the guide rail proximally out from the series of turns, leaving the tensile member exposed within the series of turns.
Example 307. The method according to example 303, wherein: (i) stitching the suture along the tissue comprises stitching the suture along the tissue using a helical member to stitch the suture helically along the guide rail in the guide arrangement, such that the helical member becomes temporarily stitched along the tissue, and/or (ii) the method further comprises unstitching the helical member from the tissue by retracting the helical member helically, leaving the suture stitched along the tissue with the tensile member extending along the interior of the series of turns.
Example 308. The method according to example 302, wherein: (i) stitching the suture along the tissue comprises stitching the suture along the tissue using a helical member to stitch the suture helically along the guide rail in the guide arrangement, such that the helical member becomes temporarily stitched along the tissue, and/or (ii) the method further comprises unstitching the helical member from the tissue by retracting the helical member helically, leaving the suture stitched along the tissue.
Example 309. The method according to example 308, wherein stitching the suture along the tissue comprises stitching the suture along the tissue while the suture is attached to an exterior of the helical member.
Example 310. The method according to example 308, wherein the method further comprises detaching the suture from the helical member once the suture is stitched along the tissue.
Example 311. The method according to example 308, wherein the suture is attached to a distal end portion of the helical member, and wherein stitching the suture along the tissue comprises stitching the suture along the tissue by withdrawing the helical member from the tissue such that the suture is drawn into and along the tissue by the withdrawal of the helical member.
Example 312. The method according to example 308, wherein stitching the suture along the tissue comprises using the helical member to stitch the suture along the tissue while the suture is alongside the helical member.
Example 313. The method according to example 308, wherein the helical member defines multiple turns, and wherein stitching the suture along the tissue comprises stitching the suture along the tissue by screwing the helical member and the suture into the tissue such that part of each turn of the helical member becomes embedded within the tissue, and another part of each turn lies above a surface of the tissue.
Example 314. The method according to example 308, wherein the helical member is a hollow helical needle defining a channel therethrough, and wherein stitching the suture along the tissue comprises screwing the helical member along the tissue while the suture is disposed within the channel.
Example 315. The method according to any one of examples 208-314, wherein: (i) in the guide arrangement, the guide rail lies around a midsection of the guide frame, the midsection disposed axially between an upstream section of the frame and a downstream section of the frame, and/or (ii) expanding the guide frame within the heart comprises expanding the guide frame within the heart such that the upstream section is wider than the downstream section.
Example 316. The method according to example 315, wherein expanding the guide frame within the heart comprises expanding the guide frame within the heart such that the upstream section is wider than the midsection.
Example 317. The method according to example 315, wherein expanding the guide frame within the heart comprises expanding the guide frame within the heart such that the upstream section protrudes radially outwards over the midsection.
Example 318. The method according to example 317, wherein positioning the implant along the tissue comprises positioning the implant along the tissue while the upstream section protrudes radially outwards over an upstream surface of the tissue.
Example 319. The method according to example 318, wherein the method further comprises, subsequently to expanding the guide frame, moving the guide frame in a downstream direction until the upstream section protrudes radially outwards over the upstream surface of the tissue.
Example 320. The method according to example 318, wherein the method further comprises, subsequently to expanding the guide frame, moving the guide frame in an upstream direction such that the upstream section squeezes past the tissue to protrude radially outwards over the upstream surface of the tissue.
Example 321. The method according to any one of examples 208-320, wherein: (i) the guide frame defines an upstream section and a downstream section, and a concave waist disposed axially between the upstream section and the downstream section, and/or (ii) expanding the guide frame comprises expanding the guide frame by expanding the upstream section and the downstream section to a greater extent than the waist, such that the waist has a smaller circumference than both the upstream section and the downstream section.
Example 322. The method according to example 321, wherein positioning the implant along the tissue comprises positioning the implant along the tissue while the guide rail lies around the waist of the guide frame.
Example 323. The method according to example 321, wherein expanding the guide frame within the heart comprises expanding the guide frame at the tissue such that the tissue becomes gripped between the upstream section and the downstream section.
Example 324. The method according to example 321, wherein the method further comprises, subsequently to expanding the guide frame within the heart, positioning the guide frame within the heart such that the tissue becomes sandwiched between the upstream section and the downstream section.
Example 325. The method according to any one of examples 208-324, wherein expanding the guide frame within the heart comprises expanding the guide frame within the heart such that an invaginating part of the guide frame invaginates to form an invagination.
Example 326. The method according to example 325, wherein a distal part of a control shaft is coupled to the guide frame at the invaginating part of the guide frame, and wherein transluminally advancing the guide frame to the heart comprises transluminally advancing the guide frame to the heart while the guide frame is coupled to the distal part of the control shaft.
Example 327. The method according to any one of examples 208-326, wherein expanding the guide frame within the heart comprises expanding the guide frame by extracorporeally tensioning multiple actuator wires that are woven longitudinally along at least part of the guide frame.
Example 328. The method according to example 327, wherein: (i) in the guide arrangement, the guide rail lies around a midsection of the guide frame, the midsection disposed axially between an upstream section of the frame and a downstream section of the frame, (ii) transluminally advancing the guide frame to the heart comprises transluminally advancing the guide frame to the heart using a control shaft that is coupled to the upstream section of the guide frame, (iii) each of the actuator wires extends, distally through an interior of the upstream section of the guide frame, and weavingly along the downstream section of the guide frame, and/or (iv) expanding the guide frame within the heart comprises expanding the downstream section of the guide frame by tensioning the actuator wires.
Example 329. The method according to example 328, wherein expanding the guide frame within the heart comprises expanding the guide frame such that at least part of the upstream section is wider than the downstream section.
Example 330. The method according to example 328, expanding the guide frame within the heart comprises expanding the guide frame such that at least part of the upstream section is wider than the midsection.
Example 331. The method according to example 327, wherein: (i) transluminally advancing the guide frame to the heart comprises transluminally advancing the guide frame to the heart using a control shaft that is coupled to a proximal part of the guide frame, and/or (ii) the method further comprises pivoting the guide frame with respect to the control shaft by differentially tensioning the actuator wires.
Example 332. The method according to example 331, wherein expanding the guide frame within the heart comprises expanding the guide frame by applying balanced tension to the actuator wires.
Example 333. The method according to any one of examples 208-332, wherein: (i) in the guide arrangement, the guide rail lies around a midsection of the guide frame, the midsection disposed axially between an upstream section of the frame and a downstream section of the frame, and/or (ii) expanding the guide frame within the heart comprises expanding the guide frame such that a shield that is disposed around the midsection expands along with the guide frame.
Example 334. The method according to example 333, wherein expanding the guide frame such that the shield expands along with the guide frame comprises expanding the guide frame such that the shield elastically expands along with the guide frame.
Example 335. The method according to example 333, wherein: (i) the shield is a ribbon, (ii) transluminally advancing the guide frame to the heart comprises transluminally advancing the guide frame to the heart while the ribbon is wrapped around the guide frame, and/or (iii) expanding the guide frame comprises expanding the guide frame such that the ribbon slides over itself in a manner that reduces the wrapping around the guide frame.
Example 336. The method according to example 333, wherein: (i) the shield is defined by multiple ribbons distributed circumferentially around the midsection, (ii) transluminally advancing the guide frame to the heart comprises transluminally advancing the guide frame to the heart while the ribbons are imbricated around the midsection, and/or (iii) expanding the guide frame within the heart comprises expanding the guide frame within the heart such that the ribbons slide over each other while collectively covering the midsection.
Example 337. The method according to any one of examples 208-336, wherein: (i) the guide rail defines an external thread, and/or (ii) positioning the implant along the tissue, guided by the guide rail comprises advancing the implant threadedly along the external thread.
Example 338. The method according to example 337, wherein: (i) the external thread defines a groove, (ii) the implant comprises a helical member defining a plurality of turns, and/or (iii) advancing the implant threadedly along the external thread comprises advancing the implant threadedly along the external thread while the helical member is recessed within the groove.
Example 339. The method according to any one of examples 208-338, wherein positioning the implant along the tissue, guided by the guide rail comprises advancing the implant along the guide rail while a leading end of the implant pushes a rider that is slidably mounted to the guide rail along the guide rail, while the rider shields the guide frame from the leading end of the implant.
Example 340. The method according to example 339, wherein: (i) the implant comprises a helical member defining a sharpened tip at the leading end, and/or (ii) advancing the implant along the guide rail comprises advancing the implant along the guide rail while a lobe of the rider remains disposed between the leading end and the guide frame, thereby shielding the guide frame from the sharpened tip.
Example 341. A system usable with real or simulated tissue of a real or simulated heart of a real or simulated subject, the system comprising: (i) an implant; and/or (ii) a delivery assembly comprising: (a) a guide assembly, comprising a guide rail that has a leading segment, the implant being mounted on the guide rail, and/or (b) a driver, engaged with the implant, (iii) the delivery assembly configured to iteratively secure the implant along the tissue by iteratively: (a) advancing the leading segment distally out of a distal end of the implant into a position along a stretch of the tissue, and/or (b) securing the leading segment to the stretch of the tissue by the driver screwing the implant into the tissue along the stretch.
Example 342. The system according to example 341, wherein the implant is sterile.
Example 343. The system according to any one of examples 341-342, wherein the guide assembly is sterile.
Example 344. The system according to any one of examples 341-343, wherein the guide rail is sterile.
Example 345. The system according to any one of examples 341-344, wherein the driver is sterile.
Example 346. The system according to any one of examples 341-345, wherein the leading segment comprises one or more imaging markers to facilitate determination of a position of the leading segment within the heart.
Example 347. The system according to any one of examples 341-346, further comprising a catheter within which the delivery assembly and the implant are transluminally advanceable to the heart.
Example 348. The system according to any one of examples 341-347, wherein the driver is configured to screw the implant into the tissue along the stretch by screwing the implant over and along the leading segment while the leading segment remains in the position along the stretch.
Example 349. The system according to any one of examples 341-348, wherein the guide assembly is configured to slide the guide rail proximally out of the implant.
Example 350. The system according to any one of examples 341-349, wherein the delivery assembly is configured to manipulate the leading segment into an alignment with respect to the stretch of the tissue.
Example 351. The system according to example 350, wherein: (i) the leading segment includes a magnetic material, and/or (ii) the system further comprises an electromagnet, advanceable to the heart, and configured to manipulate the leading segment into the alignment.
Example 352. The system according to example 351, wherein the system comprises an electromagnet tool that comprises the electromagnet, and that is configured to energize the electromagnet in a manner that manipulates the leading segment into the alignment by magnetically attracting the leading segment toward the electromagnet.
Example 353. The system according to example 351, wherein the system comprises an electromagnet tool that comprises the electromagnet, and that is configured to energize the electromagnet in a manner that manipulates the leading segment into the alignment by magnetically repelling the leading segment away from the electromagnet.
Example 354. The system according to example 351, wherein the system comprises an electromagnet tool that comprises the electromagnet, and that is configured to advance the electromagnet into an atrium of the heart, and to manipulate the leading segment from the atrium.
Example 355. The system according to example 351, wherein the system comprises an electromagnet tool that comprises the electromagnet, and that is configured to advance the electromagnet into a ventricle of the heart, and to manipulate the leading segment from the ventricle.
Example 356. The system according to example 351, wherein the system comprises an electromagnet tool that comprises the electromagnet, and that is configured to advance the electromagnet into a coronary blood vessel of the heart, and to manipulate the leading segment from the coronary blood vessel.
Example 357. The system according to example 350, wherein the leading segment comprises a shape-memory alloy, a curvature of the leading segment being adjustable by heating the leading segment.
Example 358. The system according to example 357, wherein the guide rail comprises multiple heating elements distributed along the leading segment, and drivable to electrically heat the leading segment.
Example 359. The system according to example 358, wherein each of the heating elements is drivable independently of the other heating elements so as to adjust the curvature of only a corresponding part of the leading segment.
Example 360. The system according to example 350, wherein the leading segment comprises: (i) an outer tube, and/or (ii) an inner shaft: (a) disposed inside the outer tube, (b) having a different at-rest curvature to the outer tube, and/or (c) axially slidable with respect to the outer tube.
Example 361. The system according to example 360, wherein the inner shaft has a greater at-rest curvature than the outer tube.
Example 362. The system according to example 360, wherein the outer tube has a greater at-rest curvature than the inner shaft.
Example 363. The system according to any one of examples 341-362, wherein the leading segment comprises one or more electrodes electrically connected to an extracorporeal portion of the delivery assembly.
Example 364. The system according to example 363, further comprising a data-processing system, electrically connectable to the one or more electrodes by being connected to a terminal at the extracorporeal portion of the delivery assembly, and configured: (i) to receive an electrical signal from the one or more electrodes, and/or (ii) to, responsively to the electrical signal, provide an output indicative of a position of the leading segment within the heart.
Example 365. The system according to example 364, wherein: (i) the position includes a proximity of the leading segment to a tissue surface, and/or (ii) the data-processing system is configured to, responsively to the electrical signal, provide the output indicative of the proximity of the leading segment to the tissue surface.
Example 366. The system according to example 364, wherein: (i) the position includes contact of the leading segment to a tissue surface, and/or (ii) the data-processing system is configured to, responsively to the electrical signal, provide the output indicative of the contact of the leading segment to the tissue surface.
Example 367. The system according to example 364, wherein: (i) the position is a position along an atrioventricular axis of the heart, and/or (ii) the data-processing system is configured to, responsively to the electrical signal, provide the output indicative of the position along the atrioventricular axis.
Example 368. The system according to example 364, wherein: (i) the output is indicative of a tissue-type with which the leading segment is in contact, and/or (ii) the data-processing system is configured to, responsively to the electrical signal, provide the output indicative of the tissue-type.
Example 369. The system according to example 364, wherein: (i) the electrical signal is an ECG signal, and/or (ii) the data-processing system is configured to receive the ECG signal.
Example 370. The system according to example 364, wherein: (i) the electrical signal is an exogenous signal, and/or (ii) the data-processing system is configured to receive the exogenous signal.
Example 371. The system according to example 370, wherein: (i) based on the exogenous signal, the data-processing system is configured to determine a bioimpedance, and/or (ii) the data-processing system is configured to provide the output responsively to the bioimpedance.
Example 372. The system according to example 370, wherein: (i) the one or more electrodes are multiple electrodes, and/or (ii) the data-processing system is configured to drive the exogenous signal between at least two of the electrodes.
Example 373. The system according to any one of examples 341-372, wherein the implant comprises one or more electrodes electrically connected to an extracorporeal portion of the delivery assembly.
Example 374. The system according to example 373, wherein the one or more electrodes are electrically connected to an extracorporeal portion of the driver.
Example 375. The system according to example 373, further comprising a data-processing system, electrically connectable to the one or more electrodes by being connected to a terminal at the extracorporeal portion of the delivery assembly, and configured: (i) to receive an electrical signal from the one or more electrodes, and/or (ii) to, responsively to the electrical signal, provide an output indicative of a position of the implant within the heart.
Example 376. The system according to example 375, wherein: (i) the position includes a proximity of the implant to a tissue surface, and/or (ii) the data-processing system is configured to, responsively to the electrical signal, provide the output indicative of the proximity of the implant to the tissue surface.
Example 377. The system according to example 375, wherein: (i) the position includes an orientation of the implant with respect to the tissue, and/or (ii) the data-processing system is configured to, responsively to the electrical signal, provide the output indicative of the orientation of the implant with respect to the tissue.
Example 378. The system according to example 375, wherein: (i) the position includes a depth of the implant within the tissue, and/or (ii) the data-processing system is configured to, responsively to the electrical signal, provide the output indicative of the depth of the implant within the tissue.
Example 379. The system according to example 375, wherein: (i) the position is a position along an atrioventricular axis of the heart, and/or (ii) the data-processing system is configured to, responsively to the electrical signal, provide the output indicative of the position along the atrioventricular axis.
Example 380. The system according to example 375, wherein: (i) the output is indicative of a tissue-type with which the implant is in contact, and/or (ii) the data-processing system is configured to, responsively to the electrical signal, provide the output indicative of the tissue-type.
Example 381. The system according to example 375, wherein: (i) the electrical signal is an ECG signal, and/or (ii) the data-processing system is configured to receive the ECG signal.
Example 382. The system according to example 375, wherein: (i) the electrical signal is an exogenous signal, and/or (ii) the data-processing system is configured to receive the exogenous signal.
Example 383. The system according to example 382, wherein: (i) based on the exogenous signal, the data-processing system is configured to determine a bioimpedance, and/or (ii) the data-processing system is configured to provide the output responsively to the bioimpedance.
Example 384. The system according to example 382, wherein: (i) the one or more electrodes are multiple electrodes, and/or (ii) the data-processing system is configured to drive the exogenous signal between at least two of the electrodes.
Example 385. The system according to any one of examples 341-384, wherein the implant comprises a helical member defining a plurality of turns and circumscribing a central channel.
Example 386. The system according to example 385, wherein the implant comprises a head that is coupled to the helical member, the driver being engaged with the head of the implant, and configured to screw the implant into the tissue by applying torque to the head.
Example 387. The system according to example 385, wherein the helical member defines a sharpened tip.
Example 388. The system according to example 385, wherein the implant further comprises a tensile member disposed within the central channel, and configured to axially contract the helical member upon tensioning of the tensile member.
Example 389. The system according to example 388, wherein the tensile member extends along a lumen defined by the guide rail.
Example 390. A method of implanting an implant along a real or simulated tissue of a real or simulated heart of a real or simulated subject, the method comprising: (i) positioning a leading segment of a guide rail along a first stretch of the tissue; (ii) securing the leading segment to the first stretch by screwing the implant into the tissue along the first stretch, (iii) subsequently advancing the leading segment of the guide rail distally out of a distal end of the implant and along a second stretch of the tissue, and/or (iv) securing the leading segment to the second stretch by screwing the implant into the tissue along the second stretch.
Example 391. The method according to example 390, further comprising sterilizing the implant.
Example 392. The method according to any one of examples 390-391, further comprising sterilizing the guide rail.
Example 393. The method according to any one of examples 390-392, wherein, for each of the first stretch and the second stretch, screwing the implant into the tissue along the stretch comprises screwing the implant over and along the leading segment while the leading segment is disposed along the stretch.
Example 394. The method according to any one of examples 390-393, wherein screwing the implant into the tissue along the first stretch and the second stretch comprises screwing the implant into the tissue in a manner in which the implant becomes progressively threaded around the guide rail.
Example 395. The method according to example 394, wherein the method further comprises, subsequently to screwing the implant into the tissue along the second stretch, retracting the guide rail from out of the implant by sliding the guide rail proximally through the implant, such that the implant remains implanted in the heart.
Example 396. The method according to example 394, wherein: (i) the implant includes a helical member defining a plurality of turns and circumscribing a central channel, and/or (ii) screwing the implant into the tissue along the first stretch and the second stretch comprises screwing the helical member into the tissue, such that: (a) part of each turn of the helical member becomes embedded within the tissue, and another part of each turn lies above a surface of the tissue, and/or (b) the leading segment becomes disposed within the central channel.
Example 397. The method according to example 396, wherein the helical member defines a sharpened distal tip and wherein screwing the helical member into the tissue comprises screwing the helical member into the tissue facilitated by the sharpened distal tip.
Example 398. The method according to example 396, wherein the implant further includes a tensile member that is disposed within the central channel, and wherein the method further comprises, subsequently to screwing the implant into the tissue along the second stretch, tensioning the tensile member to reduce a circumference of the tissue.
Example 399. The method according to example 396, wherein the implant comprises a head that is reversibly engageable by a driver, and wherein screwing the helical member into the tissue comprises screwing the helical member into the tissue, while the driver is engaged with the head, using the driver.
Example 400. The method according to any one of examples 390-399, wherein the method further comprises transluminally advancing the guide rail to the heart while housed in a catheter.
Example 401. The method according to example 400, wherein positioning the leading segment along the first stretch comprises positioning the leading segment along the first stretch by exposing the leading segment out of the catheter.
Example 402. The method according to any one of examples 390-401, wherein the method further comprises, for each of the first stretch and the second stretch, while the leading segment remains positioned along the stretch, and prior to securing the leading segment to the stretch, determining a position of the leading segment within the heart.
Example 403. The method according to example 402, wherein determining a position of the leading segment within the heart comprises determining the position of the leading segment within the heart by imaging the leading segment within the heart.
Example 404. The method according to example 403, wherein imaging the leading segment within the heart comprises imaging the leading segment within the heart using fluoroscopy.
Example 405. The method according to example 402, wherein determining a position of the leading segment within the heart comprises determining the position of the leading segment within the heart by sensing an electrical signal using the leading segment.
Example 406. The method according to example 405, wherein the electrical signal is an endogenous electrical signal, and wherein sensing the electrical signal comprises sensing the endogenous electrical signal.
Example 407. The method according to example 406, wherein the endogenous electrical signal is an ECG signal, and wherein sensing the endogenous electrical signal comprises sensing the ECG signal.
Example 408. The method according to example 405, wherein the electrical signal is an exogenous electrical signal, and wherein sensing the electrical signal comprises sensing the exogenous electrical signal.
Example 409. The method according to example 408, further comprising applying the exogenous electrical signal to the subject.
Example 410. The method according to example 408, wherein sensing the exogenous electrical signal comprises sensing bioimpedance.
Example 411. The method according to any one of examples 390-410, wherein the method further comprises, subsequently to positioning the leading segment along the first stretch and prior to screwing the implant into the tissue along the first stretch: (i) receiving electrophysiological signals produced by the heart that are indicative of a position of the leading segment within the heart, and/or (ii) responsively to the received signals, determining the position of the leading segment within the heart.
Example 412. The method according to example 411, wherein determining the position of the leading segment within the heart comprises determining the position of the leading segment along an atrioventricular axis of the heart.
Example 413. The method according to example 411, wherein responsively to the received signals, determining the position of the leading segment within the heart comprises responsively to the received signals, determining whether there is contact between the leading segment and the tissue.
Example 414. The method according to example 411, wherein the leading segment defines an electrode, and wherein receiving electrophysiological signals produced by the heart comprises receiving electrophysiological signals produced by the heart via the electrode.
Example 415. The method according to example 414, wherein the electrode is a ring electrode that is positioned around the leading segment, and wherein receiving electrophysiological signals produced by the heart comprises receiving electrophysiological signals produced by the heart via the ring electrode.
Example 416. The method according to any one of examples 390-415, wherein the method further comprises, subsequently to positioning the leading segment along the first stretch and prior to screwing the implant into the tissue along the first stretch: (i) receiving electrophysiological signals produced by the heart that are indicative of the position of the implant within the heart, and/or (ii) responsively to the received signals, determining a position of the implant within the heart.
Example 417. The method according to example 416, wherein determining the position of the implant within the heart comprises determining the position of the implant along an atrioventricular axis of the heart.
Example 418. The method according to example 416, wherein: (i) the implant includes a helical member defining a plurality of turns and circumscribing a central channel, (ii) screwing the implant into the tissue along the first stretch and the second stretch comprises screwing the helical member into the tissue, such that part of each turn of the helical member becomes embedded within the tissue, and another part of each turn lies above a surface of the tissue, (iii) receiving electrophysiological signals produced by the heart comprises receiving electrophysiological signals produced by the heart via an electrode that is mounted on a turn of the plurality of turns of the helical member, and/or (iv) responsively to the received signals, determining the position of the implant within the heart comprises responsively to the received signals, determining whether the electrode is disposed within the tissue.
Example 419. The method according to example 416, wherein the implant defines an electrically conductive portion, and wherein receiving electrophysiological signals produced by the heart comprises receiving electrophysiological signals produced by the heart via the electrically conductive portion.
Example 420. The method according to any one of examples 390-419, wherein positioning the leading segment along the first stretch comprises manipulating the leading segment into a desired alignment with respect to the first stretch.
Example 421. The method according to example 420, wherein the leading segment includes a magnetic material, and wherein manipulating the leading segment comprises manipulating the leading segment using an electromagnet.
Example 422. The method according to example 421, wherein manipulating the leading segment using the electromagnet comprises magnetically repelling the leading segment from the electromagnet.
Example 423. The method according to example 421, wherein manipulating the leading segment using the electromagnet comprises magnetically attracting the leading segment toward the electromagnet.
Example 424. The method according to example 421, wherein the electromagnet is a first electromagnet of a series of electromagnets, and wherein manipulating the leading segment comprises manipulating the leading segment using the series of electromagnets disposed at various positions within the heart.
Example 425. The method according to example 421, wherein the method further comprises positioning the electromagnet in an atrium of the heart, and wherein manipulating the leading segment using the electromagnet comprises manipulating the leading segment using the electromagnet while the electromagnet is disposed in the atrium.
Example 426. The method according to example 421, wherein the method further comprises positioning the electromagnet in a ventricle of the heart, and wherein manipulating the leading segment using the electromagnet comprises manipulating the leading segment using the electromagnet while the electromagnet is disposed in the ventricle.
Example 427. The method according to example 421, wherein the method further comprises positioning the electromagnet in a coronary blood vessel of the heart adjacent the first stretch, and wherein manipulating the leading segment using the electromagnet comprises manipulating the leading segment using the electromagnet while the electromagnet is disposed in the coronary blood vessel.
Example 428. The method according to example 427, wherein the method further comprises advancing the electromagnet along the coronary blood vessel such that the electromagnet becomes adjacent the second stretch, and positioning the leading segment along the second stretch manipulating the leading segment into a desired alignment with respect to the second stretch using the electromagnet.
Example 429. The method according to example 420, wherein manipulating the leading segment into a desired alignment with respect to the tissue comprises adjusting a curvature of the leading segment by electrically heating the leading segment.
Example 430. The method according to example 429, wherein the guide rail includes multiple heating elements distributed along the leading segment, and wherein electrically heating the leading segment comprises electrically heating the leading segment by driving one or more of the multiple heating elements.
Example 431. The method according to example 430, wherein adjusting the curvature of the leading segment comprises adjusting the curvature of only a part of the leading segment by heating only the part of the leading segment by driving only a subset of the multiple heating elements.
Example 432. The method according to example 420, wherein: (i) the leading segment includes: (a) an outer tube, and/or (b) an inner shaft, disposed inside the outer tube, and having a different at-rest curvature to the outer tube, and/or (ii) manipulating the leading segment into the desired alignment with respect to the tissue comprises adjusting a curvature of the leading segment by axially sliding the inner shaft with respect to the outer tube.
Example 433. The method according to example 432, wherein the outer tube has a greater at-rest curvature than the inner shaft, and wherein adjusting the curvature of the leading segment comprises reducing a curvature of the leading segment by axially sliding the inner shaft distally with respect to the outer tube.
Example 434. The method according to example 432, wherein the inner shaft has a greater at-rest curvature than the outer tube, and wherein adjusting the curvature of the leading segment comprises increasing a curvature of the leading segment by axially sliding the inner shaft distally with respect to the outer tube.
Example 435. A method for use with a simulated heart, the method comprising: (i) transluminally advancing a guide frame to the simulated heart while the guide frame is secured to a guide rail via multiple fasteners that extend out of the guide frame to the guide rail; (ii) expanding the guide frame within the simulated heart; (iii) drawing the guide rail into a guide arrangement around at least part of the guide frame by tightening at least one of the multiple fasteners; and/or (iv) while the guide rail remains in the guide arrangement, positioning an implant along a simulated tissue of the simulated heart, guided by the guide rail.
Example 436. A method for implanting an implant along a simulated tissue of a simulated heart, the method comprising: (i) positioning a leading segment of a guide rail along a first stretch of the simulated tissue; (ii) securing the leading segment to the first stretch by screwing the implant into the simulated tissue along the first stretch, (iii) subsequently advancing the leading segment of the guide rail distally out of a distal end of the implant and along a second stretch of the simulated tissue, and/or (iv) securing the leading segment to the second stretch by screwing the implant into the simulated tissue along the second stretch.
Example 437. A system usable with real or simulated tissue of a real or simulated heart, the system comprising: (A) a guide assembly, transluminally advanceable to the heart, and comprising: (i) a guide frame, deployable at a site within the heart, (ii) one or more fasteners, secured to the guide frame, and/or (iii) a guide rail, threaded through the fasteners and positionable around the guide frame such that the guide rail lies along the guide frame in a guide arrangement, the guide rail having a series of electrodes arranged along the guide rail, and/or (b) a data-processing system, electrically connected to each electrode of the series, and configured: (i) to receive an electrical signal from each electrode of the series, and/or (ii) to, responsively to the electrical signal, provide an output indicative of a position of the guide rail within the heart.
Example 438. The system according to example 437, wherein at least one of the electrodes is radiopaque.
Example 439. The system according to any one of examples 437-438, wherein: (i) each electrode of the series is electrically connected to an extracorporeal portion of the guide assembly, (ii) the extracorporeal portion comprises an electrical terminal, and/or (iii) the data-processing system is electrically connected to the electrodes by being electrically connected to the terminal.
Example 440. The system according to any one of examples 437-439, wherein each of the electrodes of the series is a ring electrode that is positioned around the guide rail.
Example 441. The system according to any one of examples 437-440, wherein: (i) the position includes a proximity of the guide rail to a tissue surface, and/or (ii) the data-processing system is configured to, responsively to the electrical signal, provide the output indicative of the proximity of the guide rail to the tissue surface.
Example 442. The system according to any one of examples 437-441, wherein: (i) the position includes contact of the guide rail with a tissue surface, and/or (ii) the data-processing system is configured to, responsively to the electrical signal, provide the output indicative of the contact of the guide rail to the tissue surface.
Example 443. The system according to any one of examples 437-442, wherein: (i) the position includes verification of contact of the guide rail with tissue of an annulus of the heart, and/or (ii) the data-processing system is configured to, responsively to the electrical signal, provide the output indicative of verification of contact of the guide rail with tissue of the annulus of the heart.
Example 444. The system according to any one of examples 437-443, wherein: (i) the position is a position along an atrioventricular axis of the heart, and/or (ii) the data-processing system is configured to, responsively to the electrical signal, provide the output indicative of the position of the guide rail along the atrioventricular axis.
Example 445. The system according to any one of examples 437-444, wherein: (i) the output is indicative of a tissue-type with which the guide rail is in contact, and/or (ii) the data-processing system is configured to, responsively to the electrical signal, provide the output indicative of the tissue-type.
Example 446. The system according to any one of examples 437-445, wherein: (i) the electrical signal is an ECG signal, and/or (ii) the data-processing system is configured to receive the ECG signal.
Example 447. The system according to any one of examples 437-446, wherein the guide frame is intracardially expandable toward an expanded state in a manner which draws the guide rail into the guide arrangement along the guide frame.
Example 448. The system according to any one of examples 437-447, wherein: (i) the electrical signal is an exogenous signal, and/or (ii) the data-processing system is configured to receive the exogenous signal.
Example 449. The system according to example 448, wherein: (i) based on the exogenous signal, the data-processing system is configured to determine a bioimpedance of the tissue, and/or (ii) the data-processing system is configured to provide the output responsively to the bioimpedance.
Example 450. The system according to example 448, wherein the data-processing system is configured to drive the exogenous signal between at least two of the electrodes of the series.
Example 451. The system according to any one of examples 437-450, wherein: (i) the fasteners are arranged in a series around the guide frame, and/or (ii) the guide assembly has a delivery state in which: (a) the guide assembly is transluminally advanceable to the heart, and/or (b) along the series, each successive fastener has a greater exposed length than the preceding fastener.
Example 452. The system according to example 451, wherein the fasteners are intracardially tightenable, responsively to the output, from a proximal extracorporeal portion of the guide assembly, in a manner that draws the guide rail into the guide arrangement along the guide frame.
Example 453. The system according to any one of examples 437-452, further comprising an implant, and a driver, configured to advance the implant along the guide rail while the guide rail is in the guide arrangement.
Example 454. The system according to example 453, wherein the implant is a helical member.
Example 455. A method usable with a real or simulated heart of a real or simulated subject, the method comprising: (i) transluminally advancing a guide frame and a guide rail to the heart; (ii) expanding the guide frame within the heart; (iii) determining a position of the guide rail within the heart responsively to an electrical signal detected via the guide rail; and/or (iv) responsively to the determining, drawing the guide rail into a guide arrangement around at least part of the guide frame.
Example 456. The method according to example 455, wherein the method further comprises, while the guide rail remains in the guide arrangement, positioning an implant along a real or simulated tissue of the heart, guided by the guide rail.
Example 457. The method according to example 456, wherein the method further comprises, prior to positioning the implant along the tissue, adjusting a size of the implant responsively to the electrical signal.
Example 458. The method according to any one of examples 455-457, wherein: (i) transluminally advancing the guide frame and the guide rail to the heart comprises transluminally advancing the guide frame and the guide rail to the heart while the guide frame is secured to the guide rail via multiple fasteners, each fastener of the multiple fasteners extending out of the guide frame and securing the guide rail within a loop of the fastener, and/or (ii) drawing the guide rail into the guide arrangement comprises, for each fastener of the multiple fasteners, responsively to the determining, pulling the fastener from outside the subject until the loop of the fastener draws the guide rail against the guide frame.
Example 459. The method according to example 458, wherein: (i) the guide rail has a series of electrodes spaced therealong, (ii) the electrical signal is a first electrical signal of multiple electrical signals, each of the multiple electrical signals being detected by a corresponding electrode of the series, and/or (iii) determining the position of the guide rail comprises determining the position of the guide rail responsively to the multiple electrical signals.
Example 460. The method according to example 459, wherein the first electrical signal is detected by a first electrode of the series, and wherein drawing the guide rail into the guide arrangement comprises drawing the guide rail into the guide arrangement by pulling: (i) by a first amount, a first of the multiple fasteners, responsively to the first electrical signal, and/or (ii) by a second amount, a second of the multiple fasteners, responsively to a second electrical signal detected by a second electrode of the series.
Example 461. The method according to example 460, wherein: (i) pulling the first of the multiple fasteners comprises pulling the first of the multiple fasteners until the first electrical signal indicates tissue contact between the first electrode and the tissue, and/or (ii) pulling the second of the multiple fasteners comprises pulling the second of the multiple fasteners until the second electrical signal indicates tissue contact between the second electrode and the tissue.
Example 462. The method according to any one of examples 455-461, wherein drawing the guide rail into the guide arrangement comprises determining that (i) electrical signals received from a first portion of the guide rail indicate a presence of tissue contact, and/or (ii) electrical signals received from a second portion of the guide rail indicate an absence of tissue contact.
Example 463. The method according to example 462, wherein, in the guide arrangement of the guide rail, the first portion is distal to the second portion.
Example 464. The method according to example 463, wherein, in the guide arrangement of the guide rail, the second portion is disposed, within the heart, upstream of the first portion.
Example 465. A method usable with a real or simulated heart, the method comprising: (i) transluminally advancing a replacement heart valve to the heart, the replacement heart valve having a series of electrodes disposed along an outer circumference thereof; (ii) expanding the replacement heart valve within the heart; (iii) receiving an output indicative of an electrical signal detected via the series of electrodes; and/or (iv) positioning the replacement heart valve within the heart, responsively to the indication.
Example 466. A system usable with real or simulated tissue of a real or simulated heart, the system comprising: (A) a delivery assembly, (B) a replacement heart valve, having a series of electrodes spaced along an outer circumference thereof, and transluminally advanceable to the heart via the delivery assembly, and/or (c) a data-processing system, electrically connected to each electrode of the series via the delivery assembly, and configured: (i) to receive an electrical signal from the series of electrodes, and/or (ii) responsively to the electrical signal, to provide an output indicative of a position of the replacement heart valve within the heart.
Example 467. The system according to example 466, wherein the electrical signal is a first electrical signal of multiple electrical signals, each of the multiple electrical signals being detected by a corresponding electrode of the series, and the data-processing system is configured to provide the output responsively to the multiple electrical signals.
Example 468. The system according to any one of examples 466-467, wherein: (i) the position includes a proximity of the outer circumference to a tissue surface, and/or (ii) the data-processing system is configured to, responsively to the electrical signal, provide the output indicative of the proximity of the outer circumference to the tissue surface.
Example 469. The system according to any one of examples 466-468, wherein: (i) the position includes contact of the outer circumference with a tissue surface, and/or (ii) the data-processing system is configured to, responsively to the electrical signal, provide the output indicative of the contact of the outer circumference to the tissue surface.
Example 470. The system according to any one of examples 466-469, wherein: (i) the position includes verification of contact of the outer circumference with tissue of an annulus of the heart, and/or (ii) the data-processing system is configured to, responsively to the electrical signal, provide the output indicative of verification of contact of the outer circumference with tissue of the annulus of the heart.
Example 471. The system according to any one of examples 466-470, wherein: (i) the position is indicative of a height of the outer circumference within a valve of the heart, and/or (ii) the data-processing system is configured to, responsively to the electrical signal, provide the output indicative of a height of the outer circumference within a valve of the heart.
Example 472. The system according to any one of examples 466-471, wherein: (i) the output is indicative of a tissue-type with which the outer circumference is in contact, and/or (ii) the data-processing system is configured to, responsively to the electrical signal, provide the output indicative of the tissue-type.
Example 473. The system according to any one of examples 466-472, wherein: (i) the electrical signal is an ECG signal, and/or (ii) the data-processing system is configured to receive the ECG signal.
Example 474. The system according to any one of examples 466-473, wherein: (i) the electrical signal is an exogenous signal, and/or (ii) the data-processing system is configured to receive the exogenous signal.
Example 475. The system according to example 474, wherein: (i) based on the exogenous signal, the data-processing system is configured to determine a bioimpedance of the tissue, and/or (ii) the data-processing system is configured to provide the output responsively to the bioimpedance.
Example 476. The system according to example 474, wherein the data-processing system is configured to drive the exogenous signal between at least two of the electrodes of the series.
Example 477. The system according to any one of examples 466-476, wherein the replacement heart valve has a frame that defines a plurality of struts.
Example 478. The system according to example 477, wherein: (i) the frame defines a waist that is adapted to become disposed circumferentially within at an annulus of the heart, and/or (ii) the outer circumference is an outer circumference of the waist, the series of electrodes being spaced therealong.
Example 479. A system for use with real or simulated tissue of a real or simulated subject, the system comprising: (i) a tissue anchor, comprising a helical tissue-engaging element and an anchor head; and/or (ii) a driver, configured to transluminally screw the tissue-engaging element into the tissue by applying torque to the anchor head, the driver formed from a unitary tube that defines: (a) at a distal end of the driver, a drivehead configured to reversibly engage the anchor head, (b) a driveshaft, configured to receive the torque from a proximal end of the driver, the driveshaft defining a first pattern of cuts that includes multiple transverse slits along the driveshaft, the driveshaft being bendable via deformation of the tube and the slits, and/or (c) a neck that connects the driveshaft to the drivehead in a manner that transfers the torque from the driveshaft to the drivehead, the neck: (i) defining a second pattern of cuts that segments the neck into discrete vertebrae, and/or (ii) being bendable via movement of the vertebrae with respect to each other.
Example 480. A system usable with real or simulated tissue of a real or simulated heart, the system comprising: (A) an implant; and/or (b) a delivery assembly comprising: (i) a guide assembly comprising, at a distal part of the guide assembly, a guide frame and a guide rail, the guide assembly: (a) having a delivery state in which the distal part is transluminally advanceable to the heart, and/or (b) being intracardially transitionable into a guide state in which the guide rail is in a guide arrangement in which the guide rail extends: (I) through an interior of the guide frame, exiting the guide frame at an exit site, and/or (ii) from the exit site, around an exterior of the guide frame, and/or (ii) a driver, configured to advance the implant along the guide rail in the guide arrangement.
Example 481. The system according to example 480, wherein the delivery assembly is configured to facilitate the guide assembly withdrawing the guide rail and the guide frame from the heart while the implant remains in the heart.
Example 482. The system according to any one of examples 480-481, wherein the guide assembly comprises multiple fasteners that are intracardially tightenable, from a proximal extracorporeal portion of the delivery assembly, in a manner that draws the guide rail into the guide arrangement around at least part of the exterior of the guide frame.
Example 483. The system according to any one of examples 480-482, wherein: (i) the tissue is tissue of an annulus of a valve of the heart, and/or (ii) the driver is configured to advance the implant along the guide rail in the guide arrangement while the guide rail is disposed along the annulus.
Example 484. The system according to example 483, wherein the driver is configured to advance the implant along the guide rail by screwing the implant into and along an atrial surface of the tissue of the annulus.
Example 485. The system according to any one of examples 480-484, wherein the system comprises a flexible helical member that defines a plurality of turns.
Example 486. The system according to example 485, wherein: (i) the guide assembly is configured to position, along a surface of the tissue, the guide rail in the guide arrangement, and/or (ii) the driver is configured to, while the guide rail in the guide arrangement is positioned along the surface of the tissue, anchor the implant along the tissue by screwing the helical member along the guide rail and the tissue such that part of each turn of the helical member becomes embedded within the tissue, and another part of each turn lies above a surface of the tissue.
Example 487. The system according to example 486, wherein the implant comprises the helical member.
Example 488. The system according to example 486, wherein: (i) the implant comprises a suture, and/or (ii) the driver is configured to, while the guide rail in the guide arrangement is positioned along the surface of the tissue, stitch the suture along the tissue by screwing the helical member along the guide rail and the tissue, such that the suture defines multiple turns.
Example 489. The system according to example 488, wherein: (i) the implant comprises a tensile member that extends through each of the turns of the suture, and/or (ii) the system further comprises a tensioning tool that is adapted to adjust a dimension of the tissue by tensioning the tensile member such that the tensile member pulls on each of the turns of the suture.
Example 490. The system according to any one of examples 480-489, wherein: (i) the guide frame has a longitudinal axis, and is intracardially expandable toward an expanded state by being expanded radially away from the longitudinal axis, and/or (ii) in the guide arrangement, the guide rail extends distally through the interior of the guide frame, and out of the guide frame at the exit site to curve around the exterior of the guide frame and the longitudinal axis.
Example 491. The system according to any one of examples 480-490, wherein: (i) the delivery assembly further comprises a tube, the guide rail extending through the tube, and/or (ii) in the guide arrangement: (a) the tube extends distally through the interior of the guide frame, and out of the guide frame at the exit site, (b) within the tube, the guide rail extends distally through the interior of the guide frame and out of the guide frame at the exit site, and/or (c) at the exterior of the guide frame, the guide rail exits the tube to lie, exposed from the tube, around the exterior of the guide frame.
Example 492. The system according to example 491, wherein the tube has a distal section that exits the guide frame at the exit site, and at least the distal section of the tube is a flexible sleeve.
Example 493. The system according to any one of examples 480-492, wherein: (i) the guide assembly comprises a control shaft, a distal end of the control shaft being attached to the guide frame in a manner that facilitates transluminal control of the guide frame via the control shaft, and/or (ii) the guide rail extends distally from the control shaft, into the interior of the guide frame.
Example 494. The system according to example 493, wherein: (i) the guide rail extends, from an extracorporeal portion thereof, distally through the control shaft, to the interior of the guide frame, and/or (ii) the implant is transluminally advanceable towards the tissue: (a) over and along the guide rail, distally through the control shaft, and/or (b) out of the control shaft into the interior of the guide frame over and along the guide rail, exiting the guide frame at the exit site, and along the guide rail to the tissue.
Example 495. An apparatus for use with a tissue, the apparatus comprising: (i) an implant that comprises a helical member that defines: (a) a sharpened distal tip, and/or (b) a proximal end, the helical member having a thickness that is greater toward the proximal end than toward the distal tip, and/or (ii) a driver, configured to screw the implant along the tissue, distal tip first, by applying torque to the proximal end of the helical member such that part of each turn of the helical member becomes embedded within the tissue, and another part of each turn lies above a surface of the tissue.
Example 496. A method for use with a real or simulated tissue, the method comprising: (i) transluminally positioning a guide rail along a surface of the tissue; (ii) using a flexible helical member, stitching a suture along the tissue by advancing the helical member helically along the guide rail, such that the suture defines a series of turns along the tissue, with part of each turn embedded within the tissue, and another part of each turn above the surface of the tissue; and/or (iii) subsequently, adjusting a dimension of the tissue using a tensile member that is disposed along the surface of the tissue and that extends through each of the turns of the suture, by tensioning the tensile member such that the tensile member pulls on each of the turns of the suture.
Example 497. The method according to example 496, wherein stitching the suture along the tissue comprises stitching the suture along the tissue while the tensile member is disposed along a lumen of the guide rail, and wherein the method further comprises retracting the guide rail proximally out from the series of turns, leaving the tensile member exposed within the series of turns.
Example 498. The method according to any one of examples 496-497, wherein stitching the suture along the tissue comprises using the helical member to stitch the suture along the tissue while the suture is alongside the helical member.
Example 499. The method according to any one of examples 496-498, wherein the helical member is a hollow helical needle defining a channel therethrough, and wherein stitching the suture along the tissue comprises screwing the helical member along the tissue while the suture is disposed within the channel.
Example 500. The method according to any one of examples 496-499, wherein adjusting the dimension of the tissue using the tensile member comprises tensioning the tensile member such that the tensile member reshapes each turn of the series of turns.
Example 501. The method according to any one of examples 496-500, wherein tensioning the tensile member such that the tensile member reshapes each turn of the series of turns comprises tensioning the tensile member such that each turn of the series of turns transitions from a rounder shape to a more oval shape.
Example 502. The method according to any one of examples 496-501, wherein: (i) the tissue is tissue of an annulus of a valve of a heart, the annulus circumscribing an orifice of the valve, and/or (ii) stitching the suture along the tissue comprises stitching the suture into and along tissue of the annulus, along the guide rail.
Example 503. The method according to example 502, wherein adjusting the dimension of the tissue using the tensile member comprises tensioning the tensile member to reduce a dimension of the annulus.
Example 504. The method according to example 502, wherein adjusting the dimension of the tissue using the tensile member comprises tensioning the tensile member such that the tensile member becomes suspended over the valve orifice.
Example 505. The method according to any one of examples 496-504, wherein stitching the suture along the tissue comprises stitching the suture along the tissue while the suture is attached to an exterior of the helical member.
Example 506. The method according to example 505, wherein the method further comprises detaching the suture from the helical member once the suture is stitched along the tissue.
Example 507. The method according to example 505, wherein the suture is attached to a distal end portion of the helical member, and wherein stitching the suture along the tissue comprises stitching the suture along the tissue by withdrawing the helical member from the tissue such that the suture is drawn into and along the tissue by the withdrawal of the helical member.
Example 508. The method according to any one of examples 496-507, wherein: (i) stitching the suture along the tissue comprises stitching the suture along the tissue such that the helical member becomes temporarily stitched along the tissue, and/or (ii) the method further comprises unstitching the helical member from the tissue by retracting the helical member helically, leaving the suture stitched along the tissue with the tensile member extending along an interior of the series of turns.
Example 509. The method according to any one of examples 496-508, wherein stitching the suture along the tissue and the tensile member comprises stitching the suture along the tissue and the tensile member such that the tensile member extends along an interior of the series of turns.
Example 510. The method according to example 509, wherein: (i) stitching the suture along the tissue comprises stitching the suture along the tissue such that the suture defines a helix that is disposed in a curved path along the tissue, the curved path having a radius of curvature, and/or (ii) adjusting the dimension of the tissue using the tensile member comprises tensioning the tensile member to adjust the dimension of the tissue by reducing the radius of curvature of the curved path.
Example 511. The method according to example 509, wherein: (i) stitching the suture along the tissue comprises stitching the suture along the tissue such that the suture defines a helix that is disposed in a curved path along the tissue, the curved path having a length, and/or (ii) adjusting the dimension of the tissue using the tensile member comprises tensioning the tensile member to adjust the dimension of the tissue by reducing the length of the curved path.
Example 512. A system for use with real or simulated tissue of a real or simulated heart, the system comprising: (A) a suture; (B) a tensile member; and/or (c) a delivery assembly comprising: (i) a guide assembly, comprising a guide rail, the guide assembly configured to transluminally advance the guide rail to the heart, and to position the guide rail into a guide arrangement along a surface of the tissue, (ii) a flexible helical member, and/or (iii) a driver configured to, while the guide rail is in the guide arrangement, stitch the suture along the tissue by advancing the flexible helical member helically along the guide rail, such that the suture defines a series of turns along the tissue, with part of each turn embedded within the tissue, another part of each turn above the surface of the tissue, and the tensile member disposed along the surface of the tissue, extending through the series of turns.
Example 513. The system according to example 512, wherein: (i) the tissue is tissue of a real or simulated annulus of a real or simulated valve of the heart, the annulus circumscribing a real or simulated orifice of the valve, and/or (ii) the driver is configured to stitch the suture into and along tissue of the annulus, circumferentially around the valve orifice.
Example 514. The system according to example 513, wherein the driver is configured to transluminally advance the helical member towards the heart over and along the guide rail.
Example 515. The system according to any one of examples 512-514, wherein: (i) the guide assembly further comprises a guide frame, intracardially expandable toward an expanded state, and/or (ii) the guide assembly is configured to position the guide rail into the guide arrangement circumferentially around at least part of the guide frame.
Example 516. The system according to example 515, wherein the guide assembly further comprises multiple fasteners that are intracardially tightenable, from a proximal extracorporeal portion of the delivery assembly, in a manner that draws the guide rail into the guide arrangement around at least part of the guide frame.
Example 517. The system according to example 515, wherein: (i) the guide frame has a longitudinal axis, and is intracardially expandable toward an expanded state by being expanded radially away from the longitudinal axis, and/or (ii) in the guide arrangement, the guide rail extends distally through an interior of the guide frame, and out of the guide frame at an exit site to curve around an exterior of the guide frame and the longitudinal axis.
Example 518. The system according to any one of examples 512-517, wherein: (i) the system further comprises a tensioning tool, and/or (ii) the tensioning tool is configured to adjust a dimension of the tissue by tensioning the tensile member such that the tensile member pulls on each of the turns of the suture.
Example 519. The system according to example 518, wherein: (i) the driver is configured to stitch the suture such that the suture becomes disposed in a helix that is disposed in a curved path along the tissue, the curved path having a radius of curvature, and/or (ii) the tensioning tool is configured to adjust a dimension of the tissue by tensioning the tensile member such that the radius of curvature of the curved path is reduced.
Example 520. The system according to example 518, wherein: (i) the driver is configured to stitch the suture such that the suture becomes disposed in a helix that is disposed in a curved path along the tissue, the curved path having a length, and/or (ii) the tensioning tool is configured to adjust a dimension of the tissue by tensioning the tensile member such that the length of the curved path is reduced.
Example 521. The system according to example 518, wherein the tensile member is disposed along a lumen of the guide rail, and wherein, the delivery assembly is configured to retract the guide rail proximally out from the series of turns, leaving the tensile member exposed along the series of turns.
Example 522. The system according to example 521, wherein: (i) the driver is configured to stitch the suture along the tissue such that the helical member becomes temporarily stitched along the tissue, and/or (ii) the delivery assembly is configured to leave the suture stitched along the tissue with the tensile member extending along the series of turns by: (a) unstitching the helical member from the tissue by retracting the helical member helically, and/or (b) retracting the guide rail linearly.
Example 523. The system according to any one of examples 512-522, wherein the driver is configured to stitch the suture along the tissue while the suture is attached to the helical member.
Example 524. The system according to example 523, wherein the suture is detachable from the helical member once the suture is stitched along the tissue.
Example 525. The system according to example 523, wherein the suture is attached to a distal end portion of the helical member, and wherein the driver is adapted to stitch the suture along the tissue by withdrawing the driver from the tissue such that the suture is drawn into and along the tissue by the withdrawal of the driver.
Example 526. The system according to any one of examples 512-525, wherein the driver is adapted to stitch the suture into the tissue alongside the helical member.
Example 527. The system according to any one of examples 512-526, wherein the helical member is a hollow helical needle defining a channel therethrough, and wherein the driver is configured to stitch the suture along the tissue by screwing the helical member along the tissue while the suture is disposed within the channel.
Example 528. The system according to example 527, wherein the helical member is configured to be unscrewed from the tissue, leaving the suture stitched along the tissue.
Example 529. A system for use with a real or simulated tissue of a real or simulated heart, the system comprising: (A) a suture; (B) a tensile member; and/or (c) a delivery assembly comprising: (i) a guide assembly, comprising a guide rail, the guide assembly configured to transluminally advance the guide rail to the heart, and to position the guide rail into a guide arrangement along a surface of the tissue, (ii) a flexible helical member, and/or (iii) a driver, coupled to the flexible helical member, wherein the delivery assembly is configured to (a) arrange the suture in a series of turns stitched along the tissue by, while the guide rail is in the guide arrangement, the driver advancing the flexible helical member helically along the guide rail, and/or (b) dispose the tensile member along the surface of the tissue, extending through the series of turns.
Example 530. A system for use with a real or simulated tissue of a real or simulated heart, the system comprising: (A) an implant comprising a suture; and/or (b) a delivery assembly comprising: (i) a guide assembly, having a distal part that is transluminally advanceable to the heart while in a delivery state, the guide assembly comprising: (a) a guide frame, intracardially expandable toward an expanded state, and/or (b) a guide rail, the guide assembly configured to position the guide rail into a guide arrangement circumferentially around at least part of the guide frame, and/or (ii) a flexible helical member defining multiple turns, configured to stitch the suture along the tissue by advancing the suture helically over and along the guide rail, while the guide rail is in the guide arrangement, such that the suture defines a series of turns along the tissue, with part of each turn of the suture embedded within the tissue, and another part of each turn of the suture lying above a surface of the tissue.
Example 531. The system according to example 530, wherein: (i) the implant further comprises a tensile member, (ii) the flexible helical member is configured to stitch the suture along the tissue such that the suture defines a series of turns along the tissue, with the tensile member extending along an interior of the series of turns, and/or (iii) the implant is configured such that tensioning of the tensile member adjusts a dimension of the tissue by the tensile member pulling on the suture.
Example 532. The system according to example 531, wherein the flexible helical member is configured to stitch the suture along the tissue by advancing the suture helically over and along the guide rail while the guide rail is in the guide arrangement and the tensile member is disposed along a lumen of the guide rail.
Example 533. The system according to any one of examples 530-532, wherein the guide assembly comprises multiple fasteners that are intracardially tightenable via an extracorporeal portion of the delivery assembly, in a manner that draws the guide rail into the guide arrangement around at least part of the guide frame.
Example 534. A system, comprising: (A) a tube, transluminally advanceable to a real or simulated body orifice, the tube defining a flex zone in which cuts in the tube confer flexibility to the tube, (B) a first strip that has greater tensile strength than the flex zone, a first end of the first strip being attached to the tube distally from the flex zone, and a second end of the first strip being attached to the tube proximally from the flex zone, such that, in a relaxed state of the tube, the first strip lies slack alongside the flex zone, and/or (c) a second strip that has greater tensile strength than the flex zone, a first end of the second strip being attached to the tube distally from the flex zone, and a second end of the second strip being attached to the tube proximally from the flex zone, such that, in a relaxed state of the tube, the second strip lies slack alongside the flex zone.
Example 535. The system according to example 534, wherein each of the first end of the first strip and the second end of the first strip are attached to the tube by welding.
Example 536. The system according to any one of examples 534-535, wherein the tube is a hypotube.
Example 537. The system according to any one of examples 534-536, wherein pulling the tube proximally tensions each of the first strip and the second strip, such that each strip presses against the flex zone.
Example 538. A method comprising manufacturing a guide frame for use with a cardiovascular system by: (A) obtaining a frame defined by a braid arrangement that is formed from multiple wires braided together, the frame having a proximal part and a distal part, wherein each of the wires has a first end at the proximal part of the frame and helically extends distally along the frame, (B) at the distal part of the frame, invaginating the braid arrangement such that a second end of each of the wires becomes disposed in an interior of the frame and the distal part of the frame becomes atraumatically contoured, and/or (c) binding the second ends of the wires together in the interior of the frame.
Example 539. The method according to example 538, further comprising, subsequently to manufacturing the guide frame, coupling a guide rail along an exterior of the guide frame using multiple fasteners that extend out of the guide frame to the guide rail.
Example 540. A method comprising manufacturing a guide frame for use with a cardiovascular system by: (i) obtaining a frame defined by a braid arrangement, the frame having a proximal part and a distal part, (ii) at the proximal part of the frame, invaginating the braid arrangement to form an invagination such that the proximal part of the frame becomes contoured and disposed within an interior of the frame, and/or (iii) heat setting the proximal part to set the invagination such that expanding the guide frame via a flexible control shaft having a distal end that is attached to the invagination disposes the control shaft within the invagination.
Example 541. The method according to example 540, further comprising, subsequently to manufacturing the guide frame, coupling a guide rail along an exterior of the guide frame using multiple fasteners that extend out of the guide frame to the guide rail.
Example 542. An apparatus for use in a real or simulated cardiovascular system of a real or simulated subject, the apparatus comprising: (i) a guide frame having an invaginating part, and being expandable into an expanded state in which the invaginating part forms an invagination, and/or (ii) a flexible control shaft, having a distal end that is coupled to the guide frame at the invaginating part, and configured to: (a) transluminally advance the frame through the cardiovascular system, and/or (b) expand the frame into its expanded state within the cardiovascular system such that, in the expanded state, the distal end of the control shaft is disposed within the invagination.
Example 543. An apparatus for use in a real or simulated cardiovascular system of a real or simulated subject, the apparatus comprising: (A) a frame defined by a braid arrangement that is formed from multiple wires braided together, the frame having a proximal part and a distal part, wherein: (i) each of the wires has a first end at the proximal part of the frame and helically extends distally along the frame, (ii) at the distal part of the frame, the braid arrangement is invaginated such that a second end of each of the wires is disposed in an interior of the frame and the distal part of the frame is atraumatically contoured, and/or (iii) the second ends of the wires are bound together in the interior of the frame; and/or (b) a flexible control shaft, coupled to the proximal part of the frame and configured to: (i) transluminally advance the frame distally through the cardiovascular system, and/or (ii) expand the frame within the cardiovascular system.
Example 544. An apparatus for use with a real or simulated tissue of a real or simulated heart, the apparatus comprising: (A) a helical member defining multiple turns; (B) a driver, configured to screw the helical member along the tissue such that part of each turn of the helical member becomes embedded within the tissue, and another part of each turn lies above a surface of the tissue; and/or (c) an extracorporeal portion, electrically connected to the helical member via the driver, and adapted to alter the tissue by applying electrical energy to the tissue via the helical member while the helical member remains screwed along the tissue, the driver being configured to unscrew the helical member from the tissue, leaving the tissue altered.
Example 545. The apparatus according to example 544, wherein the helical member comprises nitinol.
Example 546. The apparatus according to any one of examples 544-545, wherein: (i) the tissue is tissue of an annulus of a valve of the heart, and/or (ii) the driver is configured to screw the helical member along the annulus.
Example 547. The apparatus according to any one of examples 544-546, wherein the helical member is heat set to contract to a predetermined shape upon application of the electrical energy to the helical member.
Example 548. The apparatus according to any one of examples 544-547, wherein the helical member is constructed from a shape-memory material.
Example 549. The apparatus according to any one of examples 544-548, wherein the extracorporeal portion comprises a power source, configured to provide the electrical energy.
Example 550. The apparatus according to any one of examples 544-549, wherein the extracorporeal portion: (i) comprises a terminal, configured to be electrically and mechanically connected to a power source, and/or (ii) is configured to derive the electrical energy from the power source.
Example 551. The apparatus according to any one of examples 544-550, wherein the apparatus further comprises a guide assembly comprising a guide rail, and wherein the driver is configured to screw the helical member along the tissue by advancing the helical member helically over and along the guide rail while the guide rail is disposed along the tissue.
Example 552. The apparatus according to example 551, wherein the guide assembly further comprises a guide frame, and multiple fasteners that are intracardially tightenable via the extracorporeal portion in a manner that draws the guide rail along the tissue, around at least part of the guide frame.
Example 553. An apparatus for use with a real or simulated tissue of a real or simulated heart, the apparatus comprising: (i) a helical member defining multiple turns; (ii) a guide assembly, having a distal part that is transluminally advanceable to the heart while in a delivery state, the guide assembly comprising: (a) a guide frame, intracardially expandable toward an expanded state, and/or (b) a guide rail, intracardially arrangeable into a guide arrangement around at least part of the guide frame, the guide assembly being positionable within the heart such that the guide rail, in the guide arrangement, is disposed along the tissue, and/or (c) a driver, configured to screw the helical member along the guide rail and the tissue while the guide rail is in the guide arrangement; and/or (iii) an extracorporeal portion, electrically connected to the helical member via the driver, and adapted to alter the tissue by applying electrical energy to the helical member while the helical member remains screwed along the tissue.
Example 554. The apparatus according to example 553, wherein the helical member comprises nitinol.
Example 555. The apparatus according to any one of examples 553-554, wherein the helical member is heat set to contract to a predetermined shape upon application of the electrical energy to the helical member.
Example 556. The apparatus according to any one of examples 553-555, wherein the extracorporeal portion is adapted to alter the tissue by applying the electrical energy to the tissue via the helical member.
Example 557. The apparatus according to any one of examples 553-556, wherein the helical member is constructed from a shape-memory material.
Example 558. The apparatus according to any one of examples 553-557, wherein the extracorporeal portion comprises a power source, configured to provide the electrical energy.
Example 559. The apparatus according to any one of examples 553-558, wherein the extracorporeal portion: (i) comprises a terminal, configured to be electrically and mechanically connected to a power source, and/or (ii) is configured to derive the electrical energy from the power source.
Example 560. The apparatus according to any one of examples 553-559, wherein: (i) the apparatus comprises an implant comprising the helical member, and/or (ii) the implant further comprises a lock, lockable to the implant to maintain the tissue altered.
Example 561. The apparatus according to example 560, wherein: (i) the helical member is configured to contract toward a contracted state responsively to the electrical energy, (ii) the implant further comprises a tensile member, (iii) the driver is configured to screw the helical member along the tissue such that the tensile member extends along an interior of the series of turns, (iv) the implant is configured such that, while the helical member remains contracted, the tensile member is tensionable to a tensioned state in a manner that maintains the helical member in the contracted state, and/or (v) the lock is lockable to the tensile member to maintain the tensile member in the tensioned state.
Example 562. The apparatus according to example 560, wherein: (i) the helical member is configured to contract toward a contracted state responsively to the electrical energy, and/or (ii) the lock is lockable to the helical member, to maintain the helical member in the contracted state.
Example 563. A method for use with a real or simulated tissue, the method comprising: (i) screwing a flexible helical member along the tissue, such that the helical member defines a series of turns along the tissue, with part of each turn embedded within the tissue, and another part of each turn above a surface of the tissue; (ii) subsequently, altering the tissue by applying electrical energy to the helical member while the helical member remains screwed along the tissue, and/or (iii) subsequently, unscrewing the helical member from the tissue, leaving the tissue altered.
Example 564. The method according to example 563, wherein applying electrical energy to the helical member comprises applying electrical energy to the helical member from an extracorporeal power source that is electrically connected to the helical member.
Example 565. The method according to any one of examples 563-564, wherein altering the tissue by applying the electrical energy comprises irreversibly altering the tissue by applying the electrical energy.
Example 566. A method for use with a real or simulated tissue of a real or simulated heart, the method comprising: (i) transluminally advancing a guide frame to the heart while the guide frame is secured to a guide rail; (ii) expanding the guide frame within the heart; (iii) drawing the guide rail into a guide arrangement around at least a part of the guide frame; (iv) while the guide rail remains in the guide arrangement, screwing a flexible helical member along the tissue, such that the helical member defines a series of turns along the tissue, with part of each turn embedded within the tissue, and another part of each turn above a surface of the tissue; and/or (v) subsequently, altering the tissue by applying electrical energy to the helical member while the helical member remains screwed along the tissue.
Example 567. The method according to example 566, wherein: (i) altering the tissue by applying the electrical energy comprises applying the electrical energy such that the helical member contracts toward a contracted state in which the turns of the helical member are closer to each other, thereby altering the tissue, and/or (ii) the method further comprises, while the electrical energy is applied to the helical member, locking the helical member in the contracted state, such that the tissue remains altered.
Example 568. The method according to example 567, wherein locking the helical member in the contracted state comprises: (i) tensioning a tensile member that is disposed along the surface of the tissue and that extends through each of the turns of the helical member, and/or (ii) locking the tension in the tensile member by applying a lock to the tensile member, to maintain the helical member in the contracted state.
Example 569. The method according to example 567, wherein locking the helical member in the contracted state comprises mechanically locking the helical member in the contracted state.
Example 570. A system for use in a real or simulated cardiovascular system of a subject, the system comprising: (A) an extracorporeal handle; (B) a frame being transluminally advanceable into the cardiovascular system, the frame having a proximal part and a distal part; (C) a control shaft that extends from the extracorporeal handle, and that is coupled to the proximal part of the frame, and/or (D) a plurality of actuator wires: (i) extending from the control shaft, (ii) weaving distally along at least part of the frame, (iii) attached to a distal part of the frame, and/or (iv) actuatable from the handle to: (a) radially expand the frame towards an expanded state, and/or (b) pivot the frame with respect to the control shaft independently of the expansion of the frame.
Example 571. The system according to example 570, wherein: (i) the frame is a guide frame, (ii) the system further comprises a guide rail, drawable into a guide arrangement along at least part of the guide frame, (iii) in the guide arrangement, the guide rail lies around a midsection of the guide frame, the midsection disposed longitudinally between an upstream section of the frame and a downstream section of the frame, and/or (iv) each of the actuator wires extends, from the control shaft, distally through an interior of the upstream section of the guide frame, and weavingly along the downstream section of the guide frame.
Example 572. The system according to example 571, wherein in the expanded state of the guide frame, at least part of the upstream section is wider than the downstream section.
Example 573. The system according to example 571, wherein in the expanded state of the guide frame, at least part of the upstream section is wider than the midsection.
Example 574. The system according to any one of examples 570-573, wherein the frame is pivotable with respect to the control shaft via differential tensioning of the actuator wires.
Example 575. The system according to example 574, wherein the extracorporeal handle: (i) comprises at least one controller to which the actuator wires are operatively coupled, and/or (ii) is configured to differentially tension the actuator wires via actuation of the at least one controller.
Example 576. The system according to example 575, wherein: (i) the frame is configured to radially expand responsively to balanced tension in the actuator wires, and/or (ii) the extracorporeal handle is configured to apply the balanced tension to the actuator wires.
Example 577. The system according to example 576, wherein the at least one controller is configured with: (i) a first actuation mode that applies the balanced tension to the actuator wires, and/or (ii) a second actuation mode that applies the differential tension to the actuator wires.
Example 578. A method for use in a real or simulated cardiovascular system of a subject, the method comprising: (i) using a handle that is attached to a frame via a control shaft, transluminally advancing the frame to the cardiovascular system, while a plurality of actuator wires extend, from the control shaft to a distal part of the frame where each actuator wire is attached; (ii) from the handle, actuating the plurality of actuator wires to: (a) radially expand the frame within the cardiovascular system, and/or (b) pivot the frame with respect to the control shaft, independently of the expansion of the frame.
Example 579. The method according to example 578, wherein actuating the plurality of actuator wires to pivot the frame with respect to the control shaft comprises actuating the plurality of actuator wires via differential tensioning of the actuator wires to pivot the frame with respect to the control shaft.
Example 580. The method according to any one of examples 578-579, wherein actuating the plurality of actuator wires to radially expand the frame comprises actuating the plurality of actuator wires via application of balanced tension to the actuator wires to radially expand the frame.
Example 581. The method according to any one of examples 578-580, wherein actuating the plurality of actuator wires to radially expand the frame comprises actuating the plurality of actuator wires to radially expand the frame prior to actuating the actuator wires to pivot the frame.
Example 582. The method according to any one of examples 578-581, wherein actuating the plurality of actuator wires to radially expand the frame comprises actuating the plurality of actuator wires to radially expand the frame subsequently to actuating the actuator wires to pivot the frame.
Example 583. The method according to any one of examples 578-582, wherein: (i) the frame is a guide frame that is a component of a guide assembly, (ii) the guide assembly further comprises a guide rail, drawable into a guide arrangement along at least part of the guide frame, (iii) in the guide arrangement, the guide rail lies around a midsection of the guide frame, the midsection disposed longitudinally between an upstream section of the frame and a downstream section of the frame, (iv) each of the actuator wires extends, from the control shaft, distally through an interior of the upstream section of the guide frame, and weavingly along the downstream section of the guide frame, and/or (v) actuating the actuator wires comprises actuating the actuator wires that weave along the downstream section of the guide frame.
Example 584. The method according to example 583, wherein expanding the guide frame comprises expanding the guide frame such that at least part of the upstream section is wider than the downstream section.
Example 585. The method according to example 583, wherein expanding the guide frame comprises expanding the guide frame such that at least part of the upstream section is wider than the midsection.
Example 586. A system for use with real or simulated tissue of a real or simulated heart, the system comprising: (A) an implant; and/or (b) a delivery assembly comprising: (i) a driver, configured to advance the implant along the tissue, and/or (ii) a guide assembly, having a distal part that is transluminally advanceable to the heart while in a delivery state, the guide assembly comprising: (a) a guide frame, intracardially expandable toward an expanded state, (b) a guide rail, intracardially arrangeable into a guide arrangement around at least part of the guide frame, the driver being adapted to advance the implant along the tissue by advancing the implant along the guide rail in the guide arrangement, and/or (c) a rider, slidably mounted on the guide rail such that, as the driver advances the implant along the tissue, a leading end of the implant pushes the rider along the guide rail, while the rider shields the guide frame from the leading end of the implant.
Example 587. The system according to example 586, wherein: (i) the implant comprises a helical member defining a sharpened tip at the leading end, and/or (ii) the rider defines a lobe that, as the implant pushes the rider along the guide rail, the lobe remains disposed between the leading end and the guide frame, thereby shielding the guide frame from the sharpened tip.
Example 588. The system according to example 587, wherein the lobe is rotationally locked with respect to the guide rail.
Example 589. The system according to example 588, wherein the lobe is rotationally locked with respect to the guide rail via keying between the rider and the guide rail.
Example 590. A system for use with real or simulated tissue of a real or simulated heart, the system comprising: (A) a helical member defining a plurality of turns; and/or (b) a delivery assembly comprising: (i) a guide assembly, having a distal part that is transluminally advanceable to the heart while in a delivery state, the guide assembly comprising: (a) a guide frame, intracardially expandable toward an expanded state, (b) a guide rail, intracardially arrangeable into a guide arrangement around at least part of a midsection of the guide frame, and/or (c) an expandable shield that is disposed around at least the part of the midsection such that: (I) expanding the guide frame towards the expanded state expands the midsection and the shield, and/or (ii) in the guide arrangement, the shield is disposed between the guide rail and the guide frame at the midsection, and/or (ii) a driver, configured to helically advance the helical member along the guide rail in the guide arrangement.
Example 591. The system according to example 590, wherein: (i) in the expanded state of the guide frame, the guide frame is at least twice as large as it is in the delivery state of the guide assembly, and/or (ii) in the expanded state of the guide frame, the shield covers at least a majority of a circumference of the guide frame.
Example 592. The system according to any one of examples 590-591, wherein the shield comprises an elastic material.
Example 593. The system according to any one of examples 590-592, wherein the shield is defined by a fabric.
Example 594. The system according to any one of examples 590-593, wherein the shield is defined by a film.
Example 595. The system according to any one of examples 590-594, wherein the shield is defined by a net.
Example 596. The system according to any one of examples 590-595, wherein the shield has a hypotube-type structure.
Example 597. The system according to any one of examples 590-596, wherein the shield is constructed from an array of interconnected struts and tessellated cells.
Example 598. The system according to any one of examples 590-597, wherein the shield is a ribbon that, in the delivery state of the guide assembly, is wrapped around the guide frame, and wherein expanding the guide frame towards the expanded state causes the ribbon to slide over itself in a manner that reduces the wrapping around the guide frame.
Example 599. The system according to any one of examples 590-598, wherein the shield is defined by multiple ribbons distributed circumferentially around the midsection.
Example 600. The system according to example 599, wherein, in the guide arrangement, each ribbon contacts its neighboring ribbons, such that the ribbons collectively cover the midsection.
Example 601. The system according to example 599, wherein each ribbon is polymeric.
Example 602. The system according to example 599, wherein each ribbon is metallic.
Example 603. The system according to example 599, wherein: (i) in the delivery state, the ribbons are imbricated around the midsection, and/or (ii) expanding the guide frame towards the expanded state expands the shield by the ribbons sliding over each other while collectively covering the midsection.
Example 604. The system according to example 603, wherein, in the expanded state, the ribbons remain imbricated around the midsection.
Example 605. The system according to example 603, wherein, in the expanded state, the ribbons are arranged edge-to-edge around the midsection.
Example 606. A method usable with a real or simulated heart of a real or simulated subject, the method comprising: (i) transluminally advancing a guide frame to the heart while: (a) the guide frame is secured to a guide rail via multiple fasteners that extend out of the guide frame to the guide rail, and/or (b) an expandable shield is disposed around a midsection of the guide frame; (ii) expanding the guide frame within the heart in a manner that expands the midsection and the shield; and/or (iii) drawing the guide rail into a guide arrangement around at least a part of the midsection by tightening at least one of the multiple fasteners such that the shield becomes disposed between the guide rail and the guide frame.
Example 607. A system for use with real or simulated tissue of a real or simulated heart, the system comprising: (A) a helical member defining a plurality of turns; and/or (b) a delivery assembly comprising: (i) a guide assembly, having a distal part that is transluminally advanceable to the heart while in a delivery state, the guide assembly comprising: (a) a guide frame, intracardially expandable toward an expanded state, and/or (b) a guide rail, intracardially arrangeable into a guide arrangement around at least part of the guide frame, and defining an external thread, and/or (ii) a driver, configured to helically advance the helical member threadedly along the external thread while the guide rail is in the guide arrangement.
Example 608. The system according to example 607, wherein: (i) the external thread defines a groove, and/or (ii) the driver is configured to screw the helical member helically along the thread while the helical member is recessed within the groove.
Example 609. The system according to any one of examples 607-608, wherein: (i) the guide rail defines a central guide-rail axis, and has a tissue-facing surface that, along the guide rail, is disposed closer than the external thread to the central guide-rail axis, and/or (ii) in the guide arrangement, the external thread faces medially toward the guide frame, and the tissue-facing surface faces radially away from the guide frame.
Example 610. The system according to example 609, wherein the tissue-facing surface is unthreaded, and runs parallel with the external thread.
Example 611. The system according to example 609, wherein the tissue-facing surface is substantially flat.
Example 612. The system according to example 609, wherein the tissue-facing surface is concave.
Example 613. A method for use with a real or simulated tissue of a real or simulated heart, the method comprising: (i) transluminally advancing a guide frame to the heart while the guide frame is secured to a guide rail that defines an external thread, (ii) positioning the guide rail into a guide arrangement around at least part of the guide frame, such that at least part of the guide rail becomes positioned along the tissue, and/or (iii) while the guide rail is in the guide arrangement, intracardially screwing a flexible helical member into the tissue by advancing the helical member threadedly along the external thread, such that the helical member defines a series of turns along the tissue, with part of each turn embedded within the tissue, and another part of each turn above a surface of the tissue.
Example 614. The method according to example 613, wherein: (i) the external thread defines a groove, and/or (ii) advancing the helical member threadedly along the external thread comprises advancing the helical member threadedly along the external thread while the helical member is recessed within the groove.
Example 615. The method according to example 614, wherein: (i) the guide rail defines a central guide-rail axis, and has a tissue-facing surface that, along the guide rail, is disposed closer than the external thread to the central guide-rail axis, and/or (ii) positioning the guide rail into the guide arrangement around at least part of the guide frame comprises positioning the guide rail into the guide arrangement around at least part of the guide frame such that the external thread faces medially toward the guide frame, and the tissue-facing surface faces radially away from the guide frame.
Example 616. The method according to example 615, wherein positioning the guide rail into the guide arrangement around at least part of the guide frame comprises positioning the guide rail into the guide arrangement around at least part of the guide frame such that a crest of the external thread contacts the guide frame.
Example 617. The method according to example 616, wherein advancing the helical member threadedly along the external thread comprises iteratively rotating the helical member threadedly such that, during each rotation of the helical member, a distal tip of the helical member: (i) leaves the groove toward the tissue as the distal tip reaches the tissue-facing surface, (ii) penetrates the tissue at the tissue-facing surface, (iii) exits the tissue toward the external thread, and/or (iv) re-enters the groove as the distal tip returns to the external thread.

Any of the various systems, assemblies, devices, apparatuses, etc. in this disclosure can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, apparatus, etc. (e.g., with heat, radiation, ethylene oxide, hydrogen peroxide, etc.). The scope of the present disclosure includes, in some implementations, sterilizing one or more of any of the various systems, devices, apparatuses, etc. in this disclosure.

The techniques, methods, operations, steps, etc. described or suggested herein or in the references incorporated herein, and any methods of using the systems, assemblies, apparatuses, devices, etc. herein, can be performed on a living subject (e.g., human, other animal, etc.) or on a simulation (e.g., a cadaver, cadaver heart, simulator, imaginary person, etc.). When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc. The term “simulation” covers use on a cadaver, computer simulator, imaginary person (e.g., if they are just demonstrating in the air on an imaginary heart), etc.

Various implementations of systems, devices, methods, etc. are disclosed herein, and any combination of their features, components, and options can be made unless specifically excluded. For example, various descriptions of an implant can be used with any appropriate delivery assembly, and/or delivered and implanted by any appropriate method, even if a specific combination is not explicitly described. Moreover, components of any of the delivery assemblies described herein may be used with any of the other delivery assemblies described herein, mutatis mutandis. Likewise, the different constructions and features of devices and systems can be mixed and matched, such as by combining any guide rail type/feature, guide frame type/feature, anatomical site, etc., even if not explicitly disclosed. In short, individual components of the disclosed systems can be combined unless mutually exclusive or physically impossible.

Although the operations of some of the disclosed methods are described in a particular, sequential order for convenient presentation, it should be understood that this manner of description encompasses rearrangement, unless a particular ordering is required by specific language set forth below. For example, operations described sequentially can in some cases be rearranged or performed concurrently. Moreover, for the sake of simplicity, the attached figures may not show the various ways in which the disclosed systems, apparatuses, devices, methods, etc. can be used in conjunction with other systems, apparatuses, devices, methods, etc.

The present invention is not limited to the examples that have been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1-51. (canceled)

52. A system usable with tissue of a heart, the system comprising:
a flexible helical member that defines a plurality of turns;
a frame, intracardially expandable into an expanded state;
a guide rail, intracardially transitionable into a guide arrangement around the frame such that the guide rail extends:
distally through an interior of the frame, exiting the frame at an exit site, and
from the exit site, circumferentially around an exterior of the frame; and
a driver, configured to advance the helical member over and along the guide rail while the guide rail is in the guide arrangement.

53. The system according to claim 52, wherein the guide rail is withdrawable from the heart while the helical member remains in the heart.

54. The system according to claim 52, wherein the frame is withdrawable from the heart while the helical member remains in the heart.

55. The system according to claim 52, wherein:
the frame has a longitudinal axis, and is intracardially expandable toward the expanded state by being expanded radially away from the longitudinal axis,
in a delivery state of the guide rail, the guide rail extends substantially parallel with the longitudinal axis, and
in the guide arrangement, the guide rail extends latitudinally around at least part of the frame and the longitudinal axis.

56. The system according to claim 52, wherein the guide rail is a component of a guide assembly, and wherein the guide assembly comprises multiple fasteners that are intracardially tightenable, from a proximal extracorporeal portion of the system, in a manner that draws the guide rail into the guide arrangement around at least part of the exterior of the frame.

57. The system according to claim 52, further comprising a guide assembly, wherein the guide assembly comprises multiple fasteners that are adapted to intracardially route the guide rail into the guide arrangement around at least part of the exterior of the frame.

58. The system according to claim 52, wherein:
the helical member has a head at a proximal end of the helical member, and a sharpened distal tip at a distal end of the helical member,
the driver is configured to engage the helical member by engaging the head, and
the helical member has a thickness that is greater toward the proximal end than toward the distal tip.

59. The system according to claim 52, wherein:
the frame defines an upstream section, a downstream section, and a midsection disposed axially between the upstream section and the downstream section, and
in the expanded state of the frame, the midsection has a smaller circumference than both the upstream section and the downstream section.

60. The system according to claim 52, wherein:
the system is configured to position the guide rail along a surface of the tissue, and
the driver is configured to, while the guide rail in the guide arrangement is positioned along the surface of the tissue, anchor the helical member along the tissue by screwing the helical member along the guide rail and the tissue such that part of each turn of the helical member becomes embedded within the tissue, and another part of each turn lies above the surface of the tissue.

61. The system according to claim 60, wherein:
the system further comprises a suture, and
the driver is configured to, while the guide rail in the guide arrangement is positioned along the surface of the tissue, stitch the suture along the tissue by screwing the helical member along the guide rail and the tissue, such that the suture defines multiple turns.

62. The system according to claim 60, wherein:
the tissue is tissue of an annulus of a valve of the heart, and
the driver is configured to advance, while the guide rail is in the guide arrangement, the helical member along the guide rail and the tissue such that the helical member becomes anchored along the annulus.

63. The system according to claim 62, wherein:
the surface of the tissue is an atrial surface of the tissue of the annulus, and
the driver is configured to advance the helical member over and along the guide rail in a manner that screws the helical member into and along the atrial surface of the tissue of the annulus.

64. The system according to claim 52, wherein:
the system further comprises a control shaft, a distal end of the control shaft being attached to the frame in a manner that facilitates transluminal control of the frame via the control shaft, and
the guide rail extends distally from the control shaft, into the interior of the frame.

65. The system according to claim 64, wherein the guide rail extends distally through the control shaft and into the interior of the frame.

66. The system according to claim 65, wherein the helical member is transluminally advanceable over and along the guide rail towards the tissue:
within the control shaft, over and along the guide rail distally towards the heart, and
out of the control shaft and into the interior of the frame, exiting the frame at the exit site, and along the guide rail and the tissue.

67. The system according to claim 65, wherein, when the guide rail is in the guide arrangement around the frame, the guide rail extends, from an extracorporeal portion of the guide rail, distally through the control shaft, and, within the heart, out of the control shaft and into the interior of the frame, and out of the frame to curve circumferentially around the exterior of the frame.

68. The system according to claim 52, further comprising a tube, the guide rail extending within the tube.

69. The system according to claim 68, wherein:
the tube extends along the exterior of the frame, and
the tube is a flexible sleeve.

70. The system according to claim 52, further comprising:
a tensile member, the driver being adapted to advance the helical member over and along the tensile member and the tissue, and
a tensioning tool that is adapted to adjust a dimension of the tissue by tensioning the tensile member such that the tensile member pulls on each of the turns of the helical member.

71. The system according to claim 70, wherein, in the guide arrangement, the tensile member extends within the guide rail, such that the driver is configured to advance the helical member over and along the tensile member by advancing the helical member over and along the guide rail.
